(19) United States
(12) Patent Application Publication
(10) Pub. No.: US 2016/0218295 A1
(43) Pub. Date: Jul. 28, 2016

(54) AMINE DERIVATIVES, MATERIAL FOR ORGANIC ELECTROLUMINESCENT DEVICE COMPRISING THE SAME, AND ORGANIC ELECTROLUMINESCENT DEVICE USING THE SAME

(71) Applicant: SAMSUNG DISPLAY CO., LTD., Yongin-si (KR)

(72) Inventor: Koushin Matsuoka, Yokohama (JP)

(21) Appl. No.: 14/949,726

(22) Filed: Nov. 23, 2015

(30) Foreign Application Priority Data
Jan. 23, 2015 (JP) 2015-011304

(51) Int. Cl. H01L 51/00; C07D 405/14; C07D 235/20; C07D 235/18

(52) U.S. Cl. CPC H01L 51/0061; C07D 235/18; C07D 405/14; C07D 235/20; H01L 51/5056

(57) ABSTRACT

A novel amine derivative, a material for an organic electroluminescent (EL) device capable of lowering the driving voltage and enhancing the emission efficiency of the organic EL device, and an organic EL device using the same. According to an embodiment of the present disclosure, an amine derivative is represented by following Formula 1:

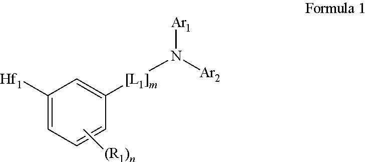

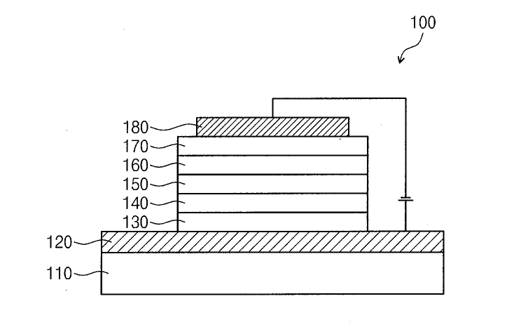

### CROSS-REFERENCE TO RELATED APPLICATION

This application claims priority to and the benefit of Japanese Patent Application No. 2015-011304, filed on Jan. 23, 2015, the entire content of which is incorporated herein by reference.

### BACKGROUND

In recent years, organic electroluminescent (EL) displays have been actively developed, and organic EL devices that are self-luminescent devices utilized in the organic EL displays also have been actively developed.

An example structure of an existing organic EL device includes an anode, a hole transport layer disposed on the anode, an emission layer disposed on the hole transport layer, an electron transport layer disposed on the emission layer, and a cathode disposed on the electron transport layer.

In the organic EL device, holes and electrons injected from the anode and the cathode recombine within the emission layer to generate excitons, and then the generated excitons are transited into the basal state to emit light. As a hole transport material, which may be utilized in the hole transport layer, an amine derivative having a benzimidazole structure in which a carbon located at the 2 position of the benzimidazole structure binds to a nitrogen atom of an amine (e.g., amine group) through an m-phenylene group has been disclosed.

### SUMMARY

However, there is a limitation in an organic electroluminescent (EL) device utilizing the existing amine derivative as a hole transport material, that is, high driving voltage and low emission efficiency. Therefore, a material is required which is capable of lowering the driving voltage and enhancing the emission efficiency of an organic EL device.

Aspects according to one or more embodiments of the present disclosure are directed toward an amine derivative, a material for an organic electroluminescent device including the same, and an organic electroluminescent device utilizing the same.

Herein, the present disclosure is derived by contemplating the limitation of the existing amine derivative, and the present disclosure provides a novel amine derivative, a novel and improved material for an organic EL device capable of lowering the driving voltage and also enhancing the emission efficiency of the organic EL device, and an organic EL device utilizing the same.

According to an embodiment of the present disclosure, an amine derivative is represented by Formula 1.

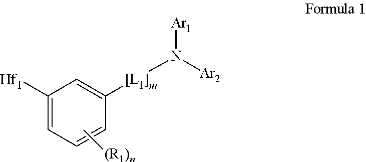

In Formula 1, Ar₁ and Ar₂ are each independently a substituted or unsubstituted aryl group having 6 to 30 carbon atoms for forming a ring, or a substituted or unsubstituted heteroaryl group having 2 to 30 carbon atoms for forming a ring; L₁ is a divalent linking group; R₁ is a substituted or unsubstituted alkyl group having 1 to 10 carbon atoms in a straight chain, branched chain, or ring form; a substituted or unsubstituted aryl group having 6 to 20 carbon atoms for forming a ring; a substituted or unsubstituted heteroaryl group having 1 to 20 carbon atoms for forming a ring; or an alkyl group, an aryl group, or a heteroaryl group formed through condensation of adjacent substituents; n and m are each independently an integer from 0 to 4; and Hf₁ is represented by Formula 2-1 or 2-2.

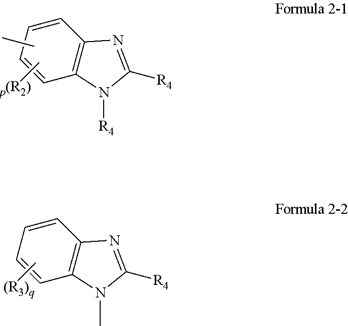

In Formula 2-2 or 2-2, R₂, R₃, and R₄ are each independently a substituted or unsubstituted alkyl group having 1 to 10 carbon atoms in a straight chain, branched chain, or ring form; a substituted or unsubstituted aryl group having 6 to 20 carbon atoms for forming a ring; a substituted or unsubstituted heteroaryl group having 1 to 20 carbon atoms for forming a ring; or an aryl group or a heteroaryl group formed through condensation of adjacent substituents; p is an integer from 0 to 3; and q is an integer from 0 to 4.

According to an aspect of the current embodiment, by utilizing the amine derivative, an organic EL device may have a lowered driving voltage and an enhanced emission efficiency.

In an embodiment, L₁ may be a substituted or unsubstituted arylene group having 6 to 30 carbon atoms for forming a ring, or a substituted or unsubstituted heteroarylene group having 2 to 30 carbon atoms for forming a ring.

According to an aspect of the current embodiment, by utilizing the amine derivative, an organic EL device may have a lowered driving voltage and an enhanced emission efficiency.

In an embodiment, Ar₁ and Ar₂ may be each independently an aryl group or a heteroaryl group which includes a substituent, and may not include a nitrogen atom.

In an embodiment, Ar₁ and Ar₂ are each independently a non-nitrogen atom-containing aryl group or heteroaryl group which is unsubstituted or substituted with a substituent which does not include a nitrogen atom.

According to an aspect of the current embodiment, by utilizing the amine derivative, an organic EL device may have a lowered driving voltage and an enhanced emission efficiency.

In an embodiment, Ar₁ may be a non-nitrogen atom-containing aryl group or heteroaryl group which is unsubstituted or substituted with a substituent which does not include a nitrogen atom, and Ar₂ may be represented by Formula 3.

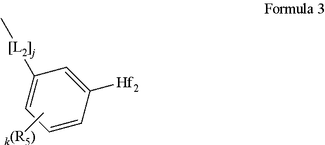

In Formula 3, L₂ is a divalent linking group; R₅ is a substituted or unsubstituted alkyl group having 1 to 10 carbon atoms in a straight chain, branched chain, or ring form; a substituted or unsubstituted aryl group having 6 to 20 carbon atoms for forming a ring; a substituted or unsubstituted heteroaryl group having 1 to 20 carbon atoms for forming a ring; or an alkyl group, an aryl group, or a heteroaryl group formed through condensation of adjacent substituents; j and k are each independently an integer from 0 to 4; and Hf₂ is represented by Formula 4-1 or 4-2.

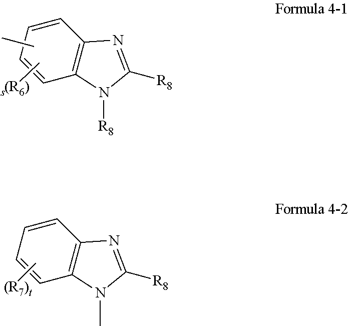

In Formula 4-1 or 4-2, R₆, R₇, and R₈ are each independently a substituted or unsubstituted alkyl group having 1 to 10 carbon atoms in a straight chain, branched chain, or ring form; a substituted or unsubstituted aryl group having 6 to 20 carbon atoms for forming a ring; a substituted or unsubstituted heteroaryl group having 1 to 20 carbon atoms for forming a ring; or an aryl group or a heteroaryl group formed through condensation of adjacent substituents; s is an integer from 0 to 3; and t is an integer from 0 to 4.

According to an aspect of the current embodiment, by utilizing the amine derivative, an organic EL device may have a lowered driving voltage and an enhanced emission efficiency.

According to an embodiment of the present disclosure, a material for an organic EL device includes an amine derivative represented by Formula 1.

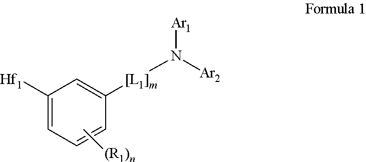

In Formula 1, Ar₁ and Ar₂ are each independently a substituted or unsubstituted aryl group having 6 to 30 carbon atoms for forming a ring or a substituted or unsubstituted heteroaryl group having 2 to 30 carbon atoms for forming a ring; L₁ is a divalent linking group; R₁ is a substituted or unsubstituted alkyl group having 1 to 10 carbon atoms in a straight chain, branched chain, or ring form; a substituted or unsubstituted aryl group having 6 to 20 carbon atoms for forming a ring; a substituted or unsubstituted heteroaryl group having 1 to 20 carbon atoms for forming a ring; or an alkyl group, an aryl group, or a heteroaryl group formed through condensation of adjacent substituents; n and m are each independently an integer from 0 to 4; and Hf₁ is represented by Formula 2-1 or 2-2.

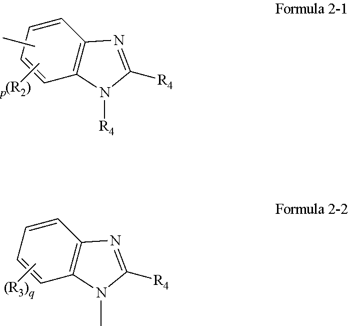

In Formula 2-1 and 2-2, R₂, R₃, and R₄ are each independently a substituted or unsubstituted alkyl group having 1 to 10 carbon atoms in a straight chain, branched chain, or ring form; a substituted or unsubstituted aryl group having 6 to 20 carbon atoms for forming a ring; a substituted or unsubstituted heteroaryl group having 1 to 20 carbon atoms for forming a ring; or an aryl group or a heteroaryl group formed through condensation of adjacent substituents; p is an integer from 0 to 3; and q is an integer from 0 to 4.

According to an aspect of the current embodiment, an organic EL device may have a lowered driving voltage and an enhanced emission efficiency.

In an embodiment, L₁ may be a substituted or unsubstituted arylene group having 6 to 30 carbon atoms for forming a ring, or a substituted or unsubstituted heteroarylene group having 2 to 30 carbon atoms for forming a ring.

According to an aspect of the current embodiment, an organic EL device may have a lowered driving voltage and an enhanced emission efficiency.

In an embodiment, Ar₁ and Ar₂ may be each independently a non-nitrogen atom-containing aryl group or heteroaryl group which is unsubstituted or substituted with a substituent which does not include a nitrogen atom.

According to an aspect of the current embodiment, an organic EL device may have a lowered driving voltage and an enhanced emission efficiency.

In an embodiment, Ar₁ may be a non-nitrogen atom-containing aryl group or heteroaryl group which is unsubstituted or substituted with a substituent which does not include a nitrogen, and Ar₂ may be represented by Formula 3.

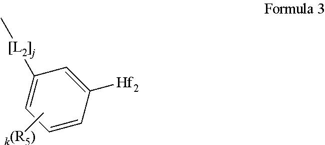

In Formula 3, L₂ is a divalent linking group; R₅ is a substituted or unsubstituted alkyl group having 1 to 10 carbon atoms in a straight chain, branched chain, or ring form; a substituted or unsubstituted aryl group having 6 to 20 carbon atoms for forming a ring; a substituted or unsubstituted heteroaryl group having 1 to 20 carbon atoms for forming a ring; or an alkyl group, an aryl group, or a heteroaryl group formed through condensation of adjacent substituents; j and k are each independently an integer from 0 to 4; and Hf₂ is represented by Formula 4-1 or 4-2.

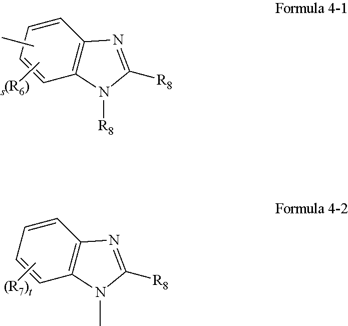

In Formula 4-1 or 4-2, R₆, R₇, and R₈ are each independently a substituted or unsubstituted alkyl group having 1 to 10 carbon atoms in a straight chain, branched chain, or ring form; a substituted or unsubstituted aryl group having 6 to 20 carbon atoms for forming a ring; a substituted or unsubstituted heteroaryl group having 1 to 20 carbon atoms for forming a ring; or an aryl group or a heteroaryl group formed through condensation of adjacent substituents; s is an integer from 0 to 3; and t is an integer from 0 to 4.

According to an aspect of the current embodiment, an organic EL device may have a lowered driving voltage and an enhanced emission efficiency.

According to an embodiment of the present disclosure, an organic EL device includes an anode; an emission layer; and at least one layer between the anode and the emission layer, the at least one layer comprising the material for an organic EL device disclosed above.

According to an aspect of the current embodiment, an organic EL device may have a lowered driving voltage and an enhanced emission efficiency.

### BRIEF DESCRIPTION OF THE DRAWING

The accompanying drawing is included to provide a further understanding of embodiments of the present disclosure, and is incorporated in and constitutes a part of this specification. The drawing illustrates example embodiments of the present disclosure and, together with the description, serves to explain principles of embodiments of the present disclosure. The drawing is a cross-sectional diagram of an organic electroluminescent device according to an embodiment of the present disclosure.

### DETAILED DESCRIPTION

Exemplary embodiments of the present disclosure will be described below in more detail with reference to the accompanying drawing. The present disclosure may, however, be embodied in different forms and should not be construed as limited to the embodiments set forth herein. Rather, these embodiments are provided so that this disclosure will be thorough and complete, and will fully convey the scope of the present disclosure to those skilled in the art.

Hereinafter, exemplary embodiments of the present disclosure will be described in more detail with reference to the accompanying drawing. In addition, like reference numerals refer to like elements throughout.

<1. Configuration of Amine Derivative and Material for Organic Electroluminescent Device Including the Same>

As a result of thoroughly examining a material for an organic electroluminescent (EL) device capable of enhancing the emission efficiency and lowering the driving voltage of an organic EL device, the present inventors have conceived of an amine derivative and a material for an organic EL device according to one or more embodiments of the present disclosure. The material for an organic EL device including the amine derivative may enhance the emission efficiency and lower the driving voltage of the organic EL device, for example, when the material is utilized as a hole transport material. Herein, a configuration of the amine derivative according to an embodiment of the present disclosure and the material for an organic EL device including the same will be described first. The material for an organic EL device according to an embodiment of the present disclosure includes an amine derivative represented by following Formula 1.

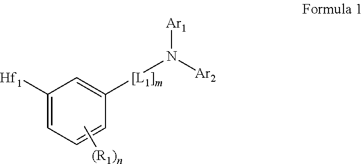

In the amine derivative represented by Formula 1, a nitrogen atom of the amine (e.g., amine group) binds to Hf₁ through an m-phenylene group. In addition, Hf₁, which will be described in more detail in the following section, has a benzimidazole structure in which a carbon located at 4 to 7 positions or the nitrogen located at the 1 position of the imidazole structure binds to the nitrogen atom of the amine (e.g., amine group) through an m-phenylene group.

In Formula 1, Ar₁ and Ar₂ are each independently a substituted or unsubstituted aryl group having 6 to 30 carbon atoms for forming a ring, or a substituted or unsubstituted heteroaryl group having 2 to 30 carbon atoms for forming a ring. Further, Ar₁ and Ar₂ may be the same as or different from each other, and also may bind to each other to form a ring.

In one embodiment, Ar₁ and Ar₂ are each independently an aryl group or a heteroaryl group which includes a substituent and does not include a nitrogen atom (e.g., Ar₁ and Ar₂ are each free of nitrogen atoms). For example, Ar₁ and Ar₂ may be each independently a non-nitrogen atom-containing aryl group or heteroaryl group which is unsubstituted or substituted with a substituent which does not include a nitrogen atom (e.g., Ar₁ and Ar₂ are each free of nitrogen atoms).

In Formula 1, Ar₁ and Ar₂ may be each independently a substituted or unsubstituted phenyl group, biphenyl group, terphenyl group, naphthyl group, anthryl group, phenanthrenyl group, fluorenyl group, indenyl group, pyrenyl group, fluoranthenyl group, triphenylenyl group, perylenyl group, naphthylphenyl group, biphenylenyl group, etc. However, the embodiment of the present disclosure is not limited thereto.

In addition, in Formula 1, Ar₁ and Ar₂ may be each independently a substituted or unsubstituted pyridyl group, quinolyl group, isoquinolyl group, indolyl group, benzoxazolyl group, benzothiazolyl group, quinoxalyl group, benzoimidazolyl group, indazolyl group, benzofuranyl group, isobenzofuranyl group, dibenzofuranyl group, phenoxazinyl group, phanothiazinyl group, acridinyl group, phenazinyl group, benzothiophenyl group, dibenzothiophenyl group, phenazasilinyl group, etc., but Ar₁ and Ar₂ are not limited thereto.

In Formula 1, a substituent of the aryl group or heteroaryl group to form Ar₁ and Ar₂ may be an aryl group or a heteroaryl group having 1 to 20 carbon atoms for forming a ring among the aryl groups and heteroaryl groups listed as an aryl group and heteroaryl group to form Ar₁ and Ar₂. In addition, the substituent may be an alkyl group (e.g., a methyl group, an ethyl group, a tert-butyl group, etc.), an alkyloxy group, an aryloxy group, an alkylthio group, an arylthio group, a dialkylamino group, a diaryamino group, a silyl group (e.g., a trialkylsilyl group, an alkyldiarylsilyl group, a dialkylarylsily group, a triarylsilyl group, etc.), etc. Moreover, the substituent may also be substituted with the same substituent, and may bind to each other to form a ring.

In Formula 1, L₁ is a divalent linking group. Examples of the divalent linking group may include an alkylene group, an alkenylene group, an alkynylene group, an arylene group, a heteroarylene group, —O—, —S—, —SO—, —SO₂—, —CO—, —NR₉—, —SiR₉R₉— etc., but L₁ is not limited thereto. Here, R₉ is each independently a substituted or unsubstituted alkyl group having 1 to 16 carbon atoms in a straight chain, branched chain, or ring form; a substituted or unsubstituted aryl group having 6 to 36 carbon atoms for forming a ring; a substituted or unsubstituted heteroaryl group having 2 to 32 carbon atoms for forming a ring; or an aryl group or a heteroaryl group formed through condensation of adjacent substituents. Because the detailed description thereof is the same as that of R₁ (to be described later), the description is not repeated herein.

Examples of the alkylene group may include an alkylene group having 1 to 16 carbon atoms in a straight chain, branched chain, or ring form, such as a methylene group, an ethylene group, a dimethylmethylene group, a 1,4-cyclohexylene group, etc.

Examples of the alkenylene group may include an alkenylene group having 2 to 16 carbon atoms in a straight chain, branched chain, or ring form, such as a vinylene group, a butadienylene group, a 1,2-cyclohexenylene group, etc.

Examples of the alkynylene group may include an alkynylene group having 2 to 4 carbon atoms in a straight chain, branched chain, or ring form, such as an acetylenylene group, a diacetylenylene group, etc.

The arylene group may be a substituted or unsubstituted arylene group having 6 to 30 carbon atoms for forming a ring, or a substituted or unsubstituted heteroarylene group having 2 to 30 carbon atoms for forming a ring. Moreover, the substituted or unsubstituted arylene group and heteroarylene group may be a divalent group prepared by removing one hydrogen atom from the aryl groups and heteroaryl groups listed as an aryl group and heteroaryl group to form Ar₁ and Ar₂. For example, the substituted or unsubstituted arylene group and heteroarylene group may be a phenylene group, a biphenylene group, a naphthylene group, a pyridylene group, a quinolylene group, etc. In addition, a substituent of the arylene group and heteroarylene group may be the same substituent of the aryl group and heteroaryl group to form Ar₁ and Ar₂.

In one embodiment, L₁ is an alkylene group, an alkenylene group, an alkynylene group, an arylene group, a heteroarylene group, —O—, —S—, —CO—, or —NR₉—. For example, L₁ is an alkenylene group, an alkynylene group, an arylene group, a heteroarylene group, —O—, —S—, or —NR₉—. In another embodiment, L₁ is a substituted or unsubstituted arylene group having 6 to 30 carbon atoms for forming a ring, or a substituted or unsubstituted heteroarylene group having 2 to 30 carbon atoms for forming a ring.

In Formula 1, R₁ is a substituted or unsubstituted alkyl group having 1 to 10 carbon atoms in a straight chain, branched chain, or ring form; a substituted or unsubstituted aryl group having 6 to 20 carbon atoms for forming a ring; a substituted or unsubstituted heteroaryl group having 1 to 20 carbon atoms for forming a ring; or an aryl group or a heteroaryl group formed through condensation of adjacent substituents.

The alkyl group having 1 to 10 carbon atoms may be a straight chain alkyl group (e.g., a methyl group, an ethyl group, a propyl group, a butyl group, an octyl group, a decyl group, etc.), a branched chain alkyl group (e.g., t-butyl group, etc.), or a ring form alkyl group (e.g., a cyclopropyl group, a cyclobutyl group, a cyclopentyl group, a cyclohexyl group, a cyclooctyl group, etc.). The branched chain alkyl group may be, for example, an alkyl group having 4 to 10 carbon atoms in a branched chain form; and the ring form alkyl group may be, for example, an alkyl group having 3 to 10 carbon atoms in a ring form.

The aryl group having 6 to 20 carbon atoms for forming a ring and the heteroaryl group having 1 to 20 carbon atoms for forming a ring may be respectively an aryl group having 6 to 20 carbon atoms for forming a ring and a heteroaryl group having 1 to 20 carbon atoms for forming a ring among the functional groups listed as an aryl group and heteroaryl group to form Ar₁ and Ar₂. The heteroaryl group may be, for example, a heteroaryl group having 2 to 20 carbon atoms for forming a ring.

n and m are each independently an integer from 0 to 4. When n is 2 or more, plurality of R₁s may be the same as or different from each other; and likewise, when m is 2 or more, plurality of L₁s may be the same as or different from each other. In one embodiment, m is 1 or 2, for example, m is 1. In one embodiment, n is an integer from 0 to 2, for example, n is 0.

In Formula 1, Hf₁ is represented by following Formula 2-1 or 2-2.

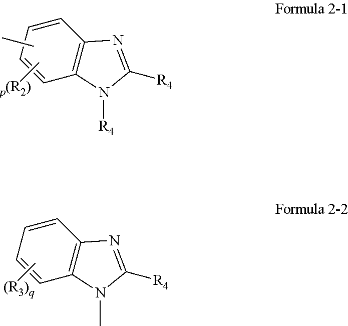

Hf₁, as described above, has a benzimidazole structure in which a carbon located at 4 to 7 positions (Formula 2-1) or a nitrogen located at the 1 position (Formula 2-2) of the benzimidazole structure binds to a nitrogen atom of an amine (e.g., amine group) through an m-phenylene group.

In Formula 2-1 or 2-2, R₂, R₃, and R₄ are each independently a substituted or unsubstituted alkyl group having 1 to 10 carbon atoms in a straight chain, branched chain, or ring form; a substituted or unsubstituted aryl group having 6 to 20 carbon atoms for forming a ring; a substituted or unsubstituted heteroaryl group having 1 to 20 carbon atoms for forming a ring; or an alkyl group; an aryl group or a heteroaryl group formed through condensation of adjacent substituents.

In one embodiment, the alkyl group having 1 to 10 carbon atoms, the substituted or unsubstituted aryl group having 6 to 20 carbon atoms for forming a ring, and the substituted or unsubstituted heteroaryl group having 1 to 20 carbon atoms for forming a ring (for R₂, R₃, and R₄) may be the same as a corresponding functional group for R₁ of Formula 1.

p is an integer from 0 to 3, and q is an integer from 0 to 4. When p is 2 or more, plurality of R₂s may be the same as or different from each other; and likewise, when q is 2 or more, plurality of R₃s may be the same as or different from each other. In addition, when p and q are 2 or more, adjacent R₂s or R₃s may bind to each other to form a ring. In one embodiment, p and q are each independently 0 or 1, for example, p and q are each 0.

Further, in Formula 1, Ar₁ may be an aryl group or a heteroaryl group which includes a substituent and does not include a nitrogen atom (e.g., Ar₁ is free of nitrogen atoms); and Ar₂ may be represented by following Formula 3. In one embodiment, Ar₁ is a non-nitrogen atom-containing aryl group or heteroaryl group which is unsubstituted or substituted with a substituent which does not include a nitrogen atom; and Ar₂ may be represented by following Formula 3.

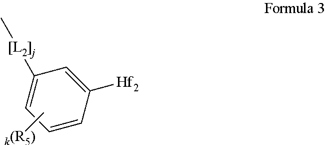

In Formula 1, Ar₁ may be a non-nitrogen atom-containing aryl group or heteroaryl group among the above disclosed aryl groups and heteroaryl groups to form Ar₁ and Ar₂. In addition, a substituent of Ar₁ may be a non-nitrogen atom-containing substituent among the above disclosed substituents of the aryl groups and heteroaryl groups to form Ar₁ and Ar₂.

In Formula 3, similar to L₁, examples of L₂, which is a divalent linking group, may include an alkylene group, an alkenylene group, an alkynylene group, an arylene group, a heteroarylene group, —O—, —S—, —SO—, —SO₂—, —CO—, —NR₉—, —SiR₉R₉—, etc., but L₂ is not limited thereto. Because detailed description of L₂ is the same as that of L₁, the description is not repeated again. In one embodiment, L₂ is an alkylene group, an alkenylene group, an alkynylene group, an arylene group, a heteroarylene group, —O—, —S—, —CO—, or —NR₉—; for example, L₂ is an alkenylene group, an alkynylene group, an arylene group, a heteroarylene group, —O—, —S—, or —NR₉—. In one embodiment, L₂ is a substituted or unsubstituted arylene group having 6 to 30 carbon atoms for forming a ring, or a substituted or unsubstituted heteroarylene group having 2 to 30 carbon atoms for forming a ring.

In Formula 3, R₅ is a substituted or unsubstituted alkyl group having 1 to 10 carbon atoms in a straight chain, branched chain, or ring from; a substituted or unsubstituted aryl group having 6 to 20 carbon atoms for forming a ring; a substituted or unsubstituted heteroaryl group having 1 to 20 carbon atoms for forming a ring; or an aryl group or a heteroaryl group formed through condensation of adjacent substituents.

The alkyl group having 1 to 10 carbon atoms, the aryl group having 6 to 20 carbon atoms for forming a ring, and the heteroaryl group having 1 to 20 carbon atoms for forming a ring (for R₅) may be the same as a corresponding group described above for R₁ in Formula 1.

j and k are each independently an integer from 0 to 4. When k is 2 or more, plurality of R₅s may be the same as or different from each other; and likewise, when j is 2 or more, plurality of L₂s may be the same as or different from each other. In one embodiment, j is 1 or 2, for example, j is 1. In one embodiment, k is an integer from 0 to 2, for example, k is 0.

In Formula 3, Hf₂ is represented by following Formula 4-1 or 4-2.

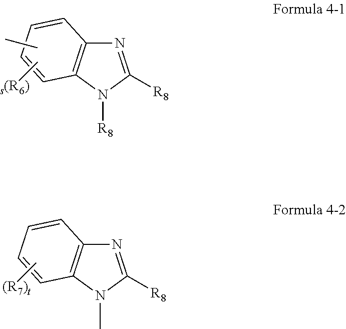

Similar to Hf₁ of Formula 2-1 and 2-2, Hf₂ also has a benzimidazole structure in which a carbon located at 4 to 7 positions (Formula 4-1) or the nitrogen located at the 1 position (Formula 4-2) of the benzimidazole structure binds to a nitrogen atom of an amine (e.g., amine group) through an m-phenylene group.

In Formula 4-1 and 4-2, R₆, R₇, and R₈ are each independently a substituted or unsubstituted alkyl group having 1 to 10 carbon atoms in a straight chain, branched chain, or ring form; a substituted or unsubstituted aryl group having 6 to 20 carbon atoms for forming a ring; a substituted or unsubstituted heteroaryl group having 1 to 20 carbon atoms for forming a ring; or an alkyl group, an aryl group or a heteroaryl group formed through condensation of adjacent substituents.

For example, the alkyl group having 1 to 10 carbon atoms, the substituted or unsubstituted aryl group having 6 to 20 carbon atoms for forming a ring, and the substituted or unsubstituted heteroaryl group having 1 to 20 carbon atoms for forming a ring (for R₆, R₇, and R₈) may be the same as a corresponding functional group for R₁ of Formula 1.

s is an integer from 0 to 3, and t is an integer from 0 to 4. When s is 2 or more, plurality of R₆s may be the same as or different from each other; and likewise, when t is 2 or more, plurality of R₇s may be the same as or different from each other. In addition, when s and t are each independently 2 or more, adjacent R₆s or R₇s may bind to each other to form a ring. In one embodiment, s and t are each independently 0 or 1, for example, s is 0 and t is 0.

In one embodiment, Ar₂ is represented by Formula 3, L₁ and L₂ are the same linking group, m=j, n=k, and Hf₁ and Hf₂ are the same. In one embodiment, R₁ and R₅ are also the same. In other word, in the amine derivative represented by Formula 1, substituents other than Ar₁ that bind to a nitrogen atom of an amine (e.g., amine group) are the same (e.g., same as each other).

The amine derivative represented by Formula 1 according to an embodiment of the present disclosure may more appropriately enhance the emission efficiency of an organic EL device when an emission layer includes a blue-emitting material or a green-emitting material.

Further, in an organic EL device, a material for an organic EL device including the amine derivative represented by Formula 1 according to an embodiment of the present disclosure is appropriately included in at least one of the layers disposed between an anode and an emission layer of the organic EL device. For example, the material for an organic EL device including the amine derivative represented by Formula 1 may be appropriately included in a hole transport layer and/or a hole injection layer of the organic EL device. When the amine derivative according to an embodiment of the present disclosure is utilized in the hole transport layer, the amine derivative may be utilized in a layer which is (e.g., laminated) adjacent to the emission layer (e.g., when the hole transport layer has a single layer structure); or a layer located closely to the emission layer in a multilayer structure of the hole transport layer (e.g., the amine derivative is utilized in the layer closest to the emission layer when the hole transport layer has a multilayer structure). However, a layer, which includes the amine derivative represented by Formula 1 is not limited to the above mentioned example. For example, the amine derivative represented by Formula 1 may be included in any one of the organic layers (inserted) between an anode and a cathode of the organic EL device, and may be, for example, included in the emission layer.

As described in the following examples, the organic EL device utilizing the material for an organic EL device having the above-mentioned configuration may significantly enhance the emission efficiency and lower the driving voltage of the organic EL device. Examples of the amine derivative included in the material for an organic EL device are listed below. However, the amine derivative according to an embodiment of the present disclosure is not limited to the following compounds. The amine derivative represented by Formula 1 may be at least one of the following compounds 1 to 130, but it is not limited thereto.

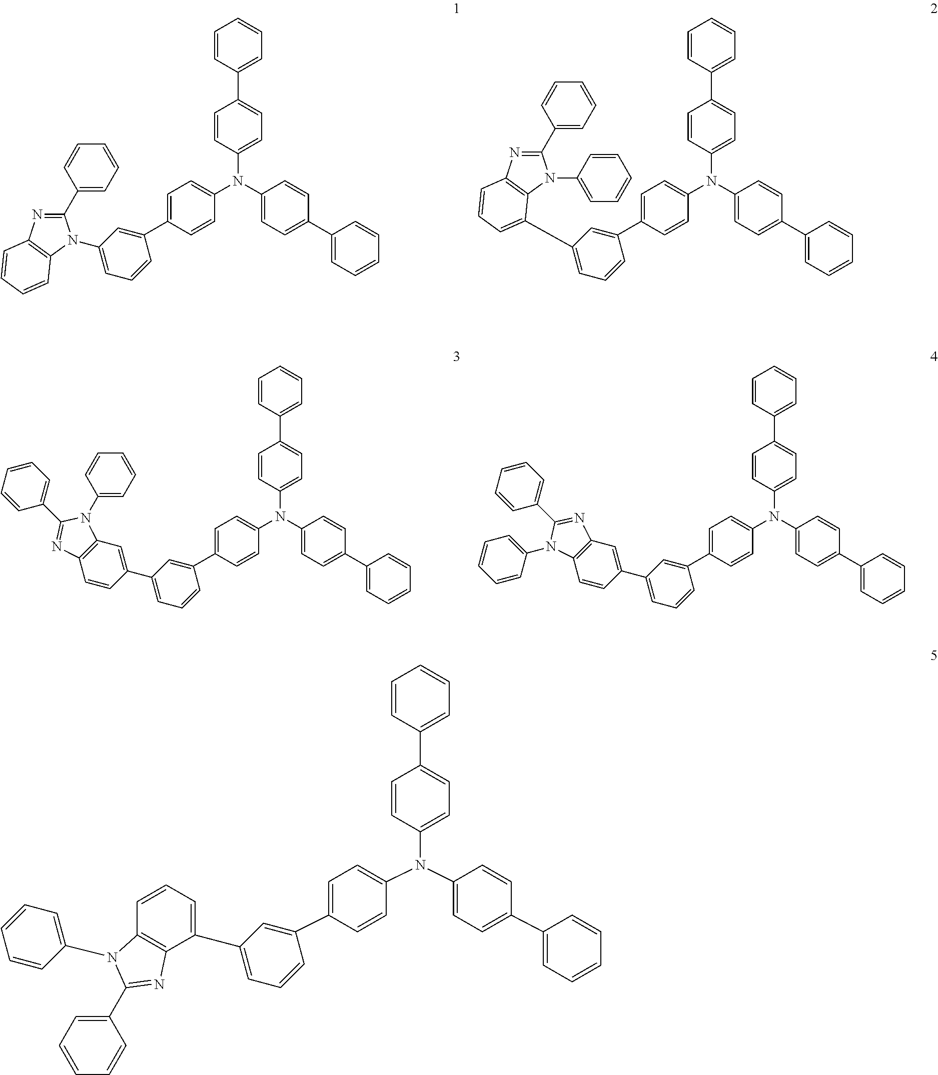 [SMILES: c1ccc(-c2ccc(N(c3ccc(-c4ccccc4)cc3)c3ccc(-c4cccc(-n5c(-c6ccccc6)nc6ccccc65)c4)cc3)cc2)cc1 ; c1ccc(-c2ccc(N(c3ccc(-c4ccccc4)cc3)c3ccc(-c4cccc(-c5cccc6nc(-c7ccccc7)n(-c7ccccc7)c56)c4)cc3)cc2)cc1 ; c1ccc(-c2ccc(N(c3ccc(-c4ccccc4)cc3)c3ccc(-c4cccc(-c5ccc6nc(-c7ccccc7)n(-c7ccccc7)c6c5)c4)cc3)cc2)cc1 ; c1ccc(-c2ccc(N(c3ccc(-c4ccccc4)cc3)c3ccc(-c4cccc(-c5ccc6c(c5)nc(-c5ccccc5)n6-c5ccccc5)c4)cc3)cc2)cc1 ; c1ccc(-c2ccc(N(c3ccc(-c4ccccc4)cc3)c3ccc(-c4cccc(-c5cccc6c5nc(-c5ccccc5)n6-c5ccccc5)c4)cc3)cc2)cc1]

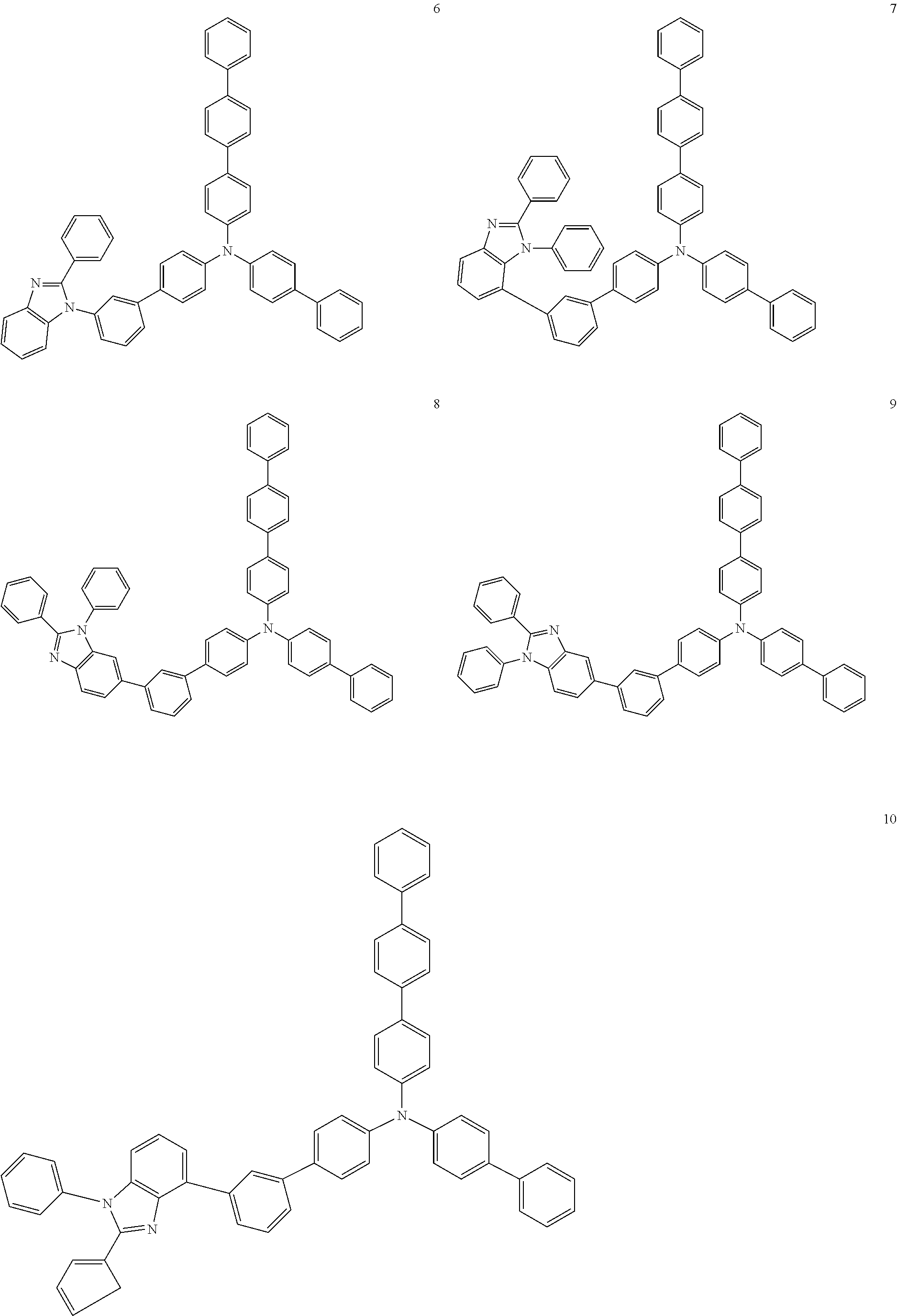

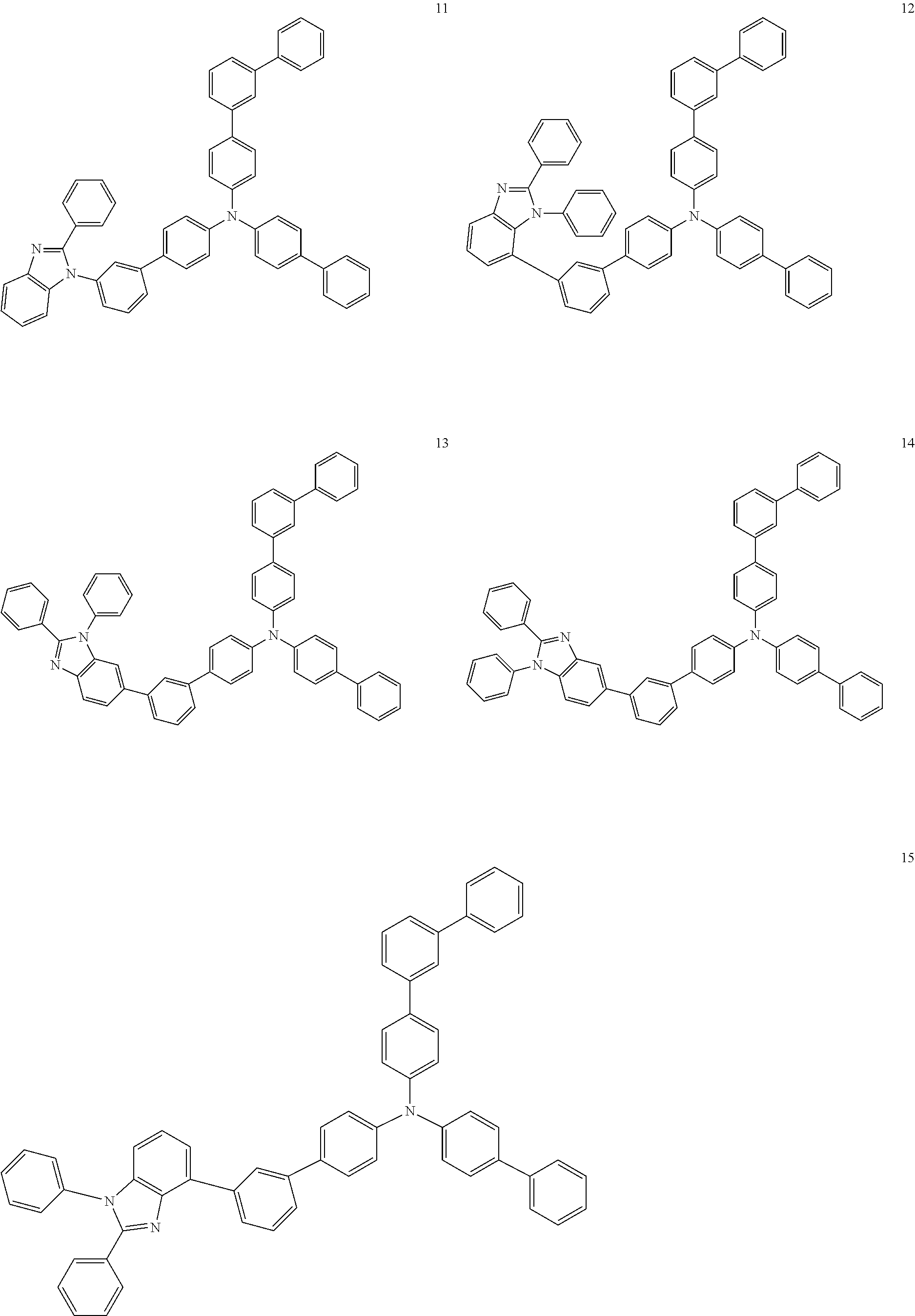

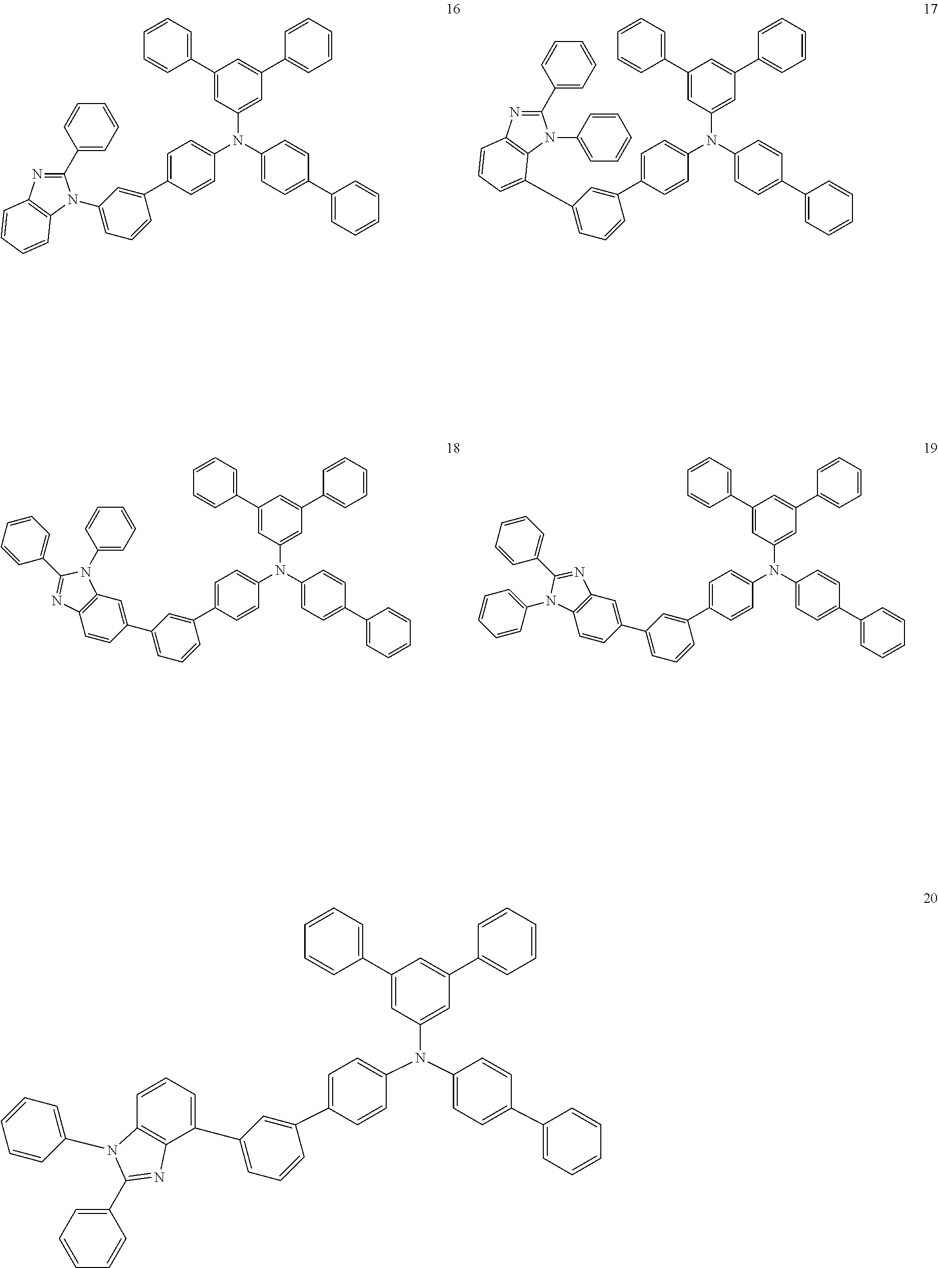

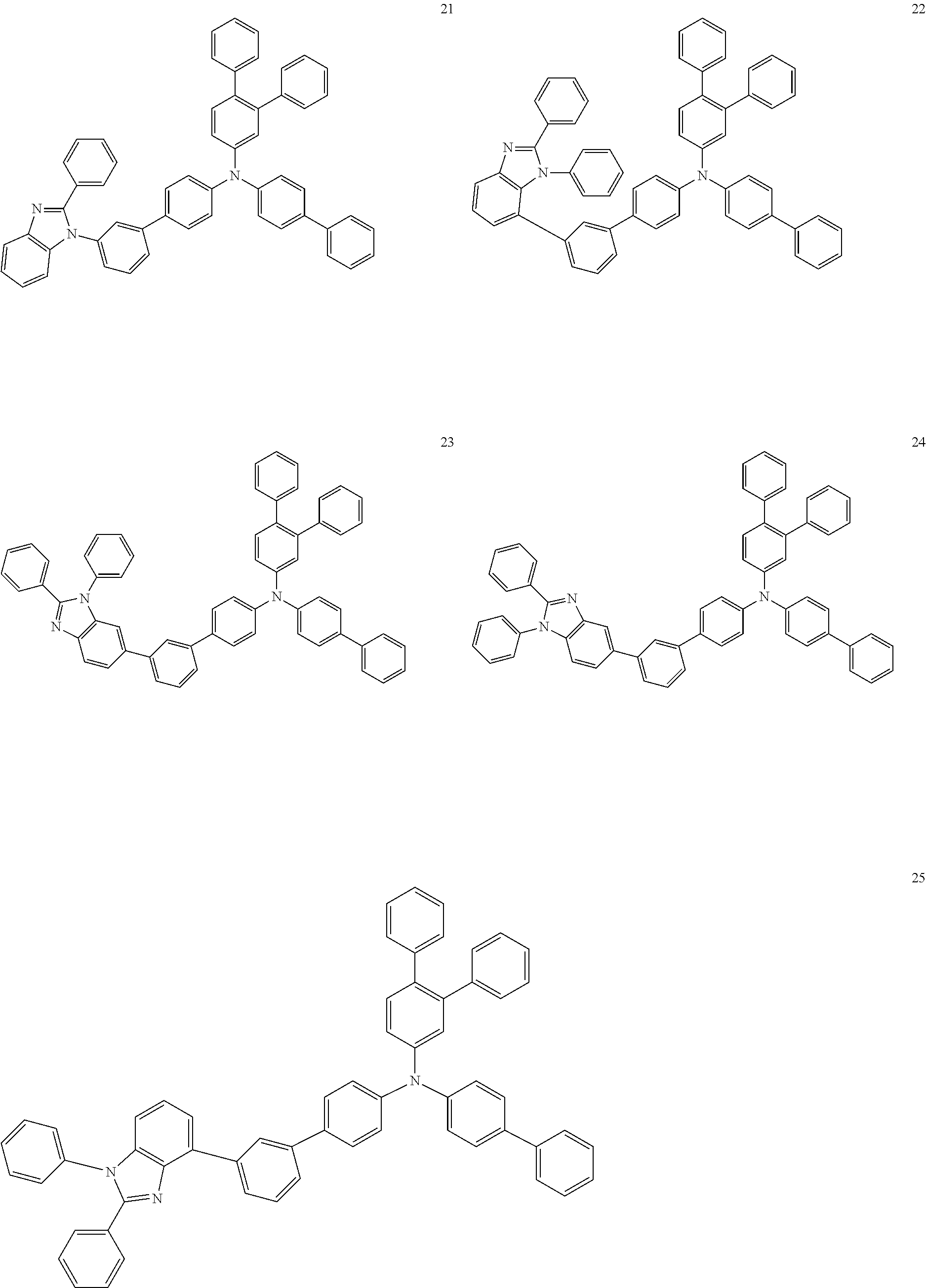

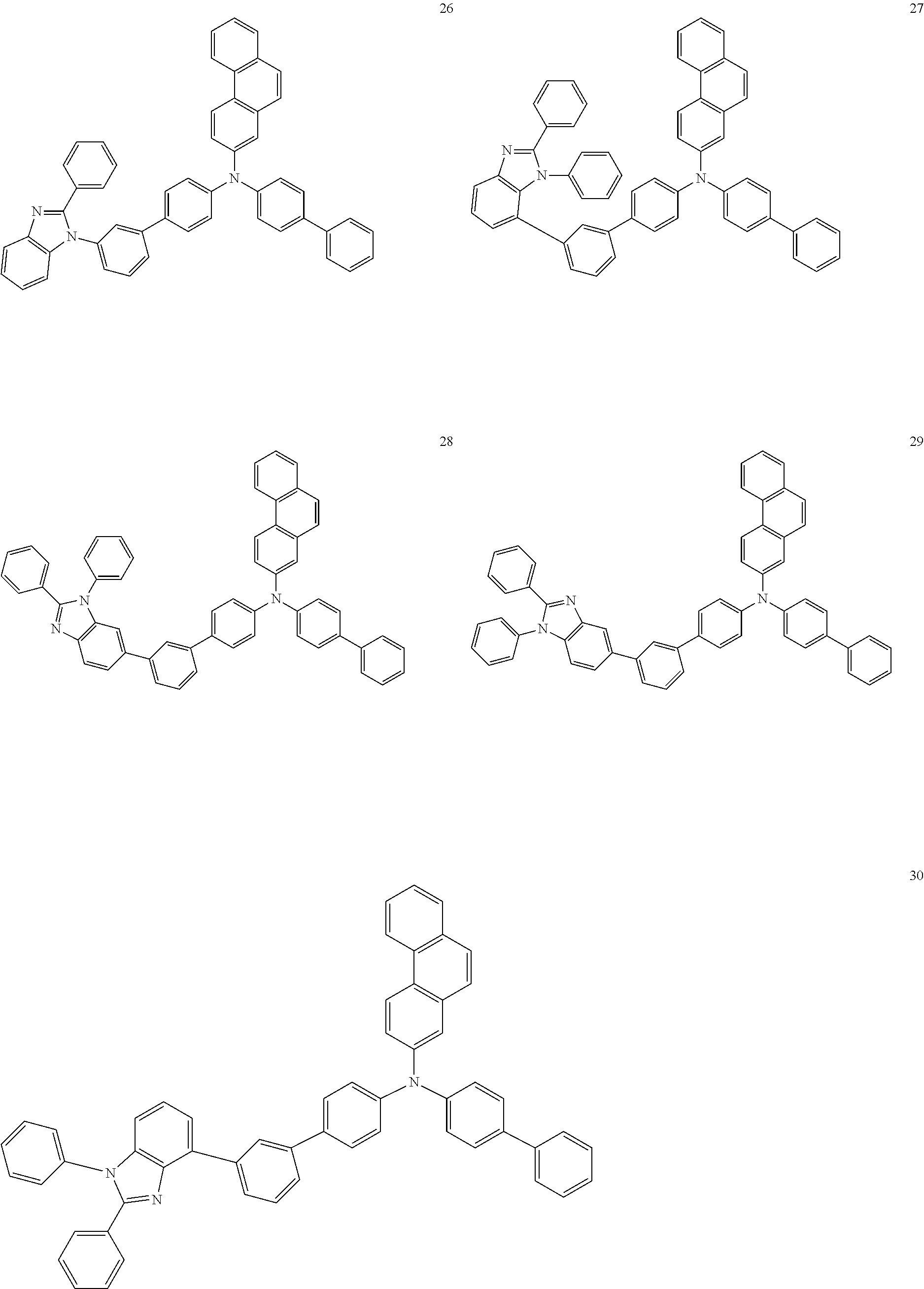

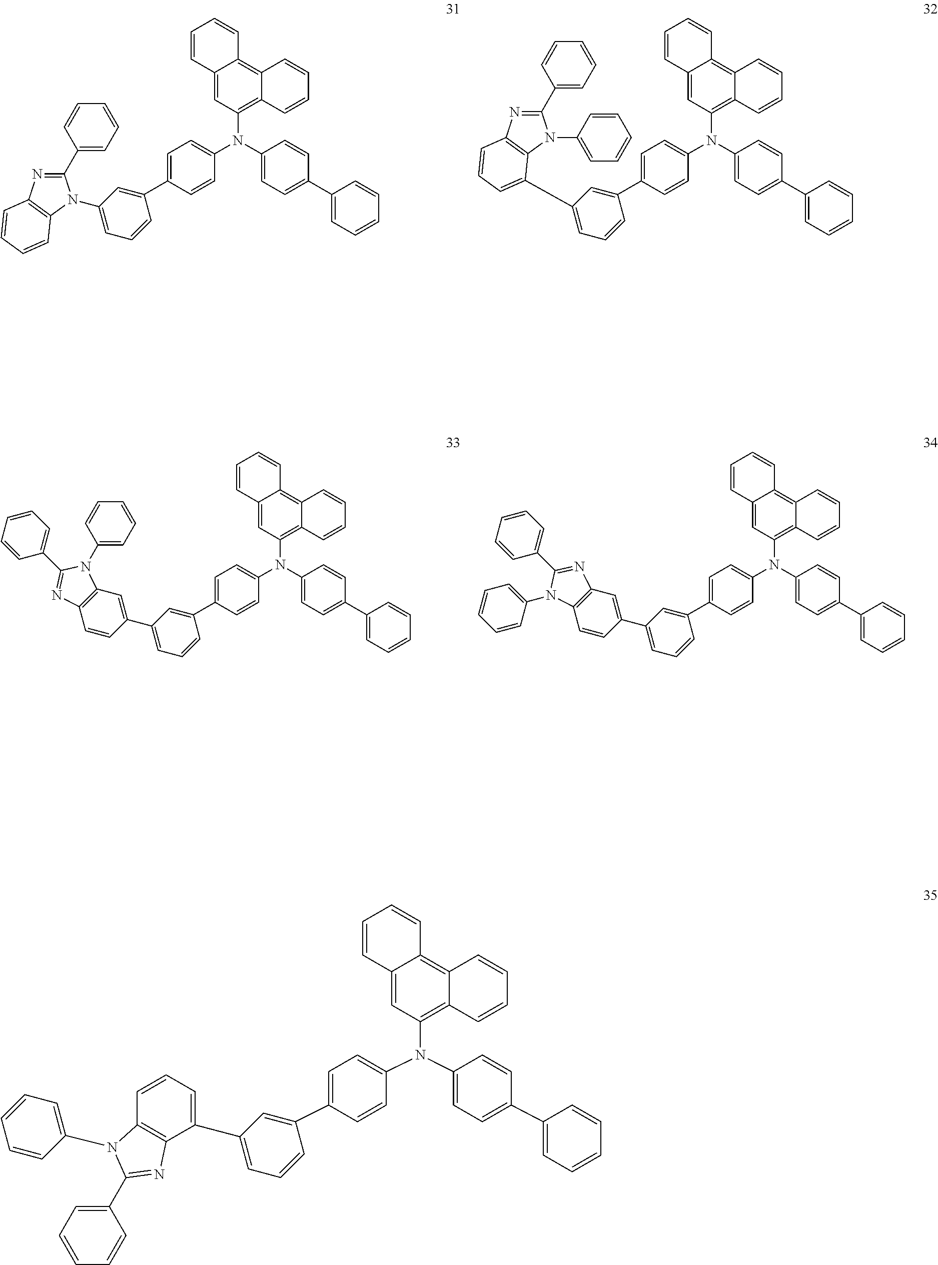

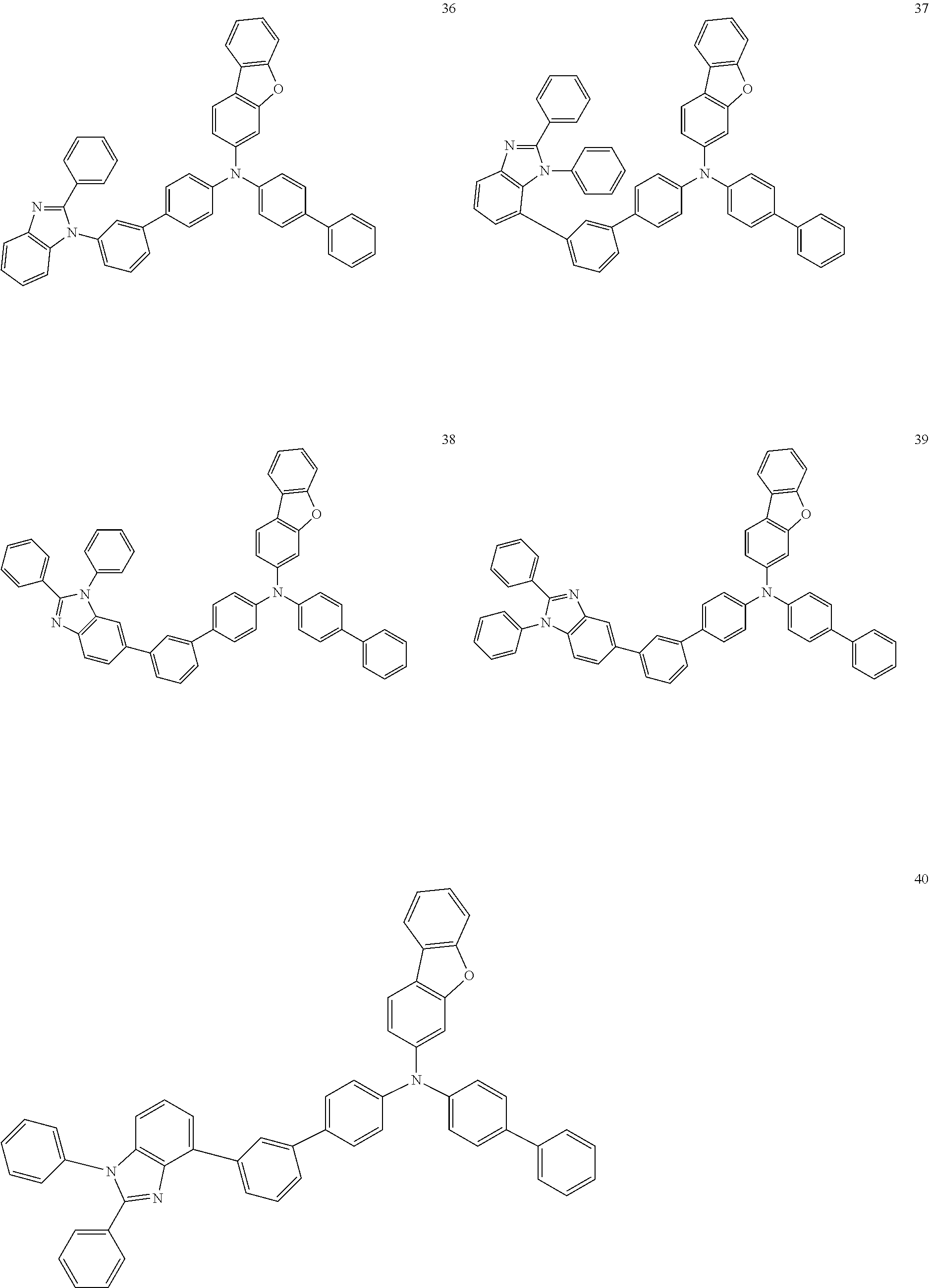

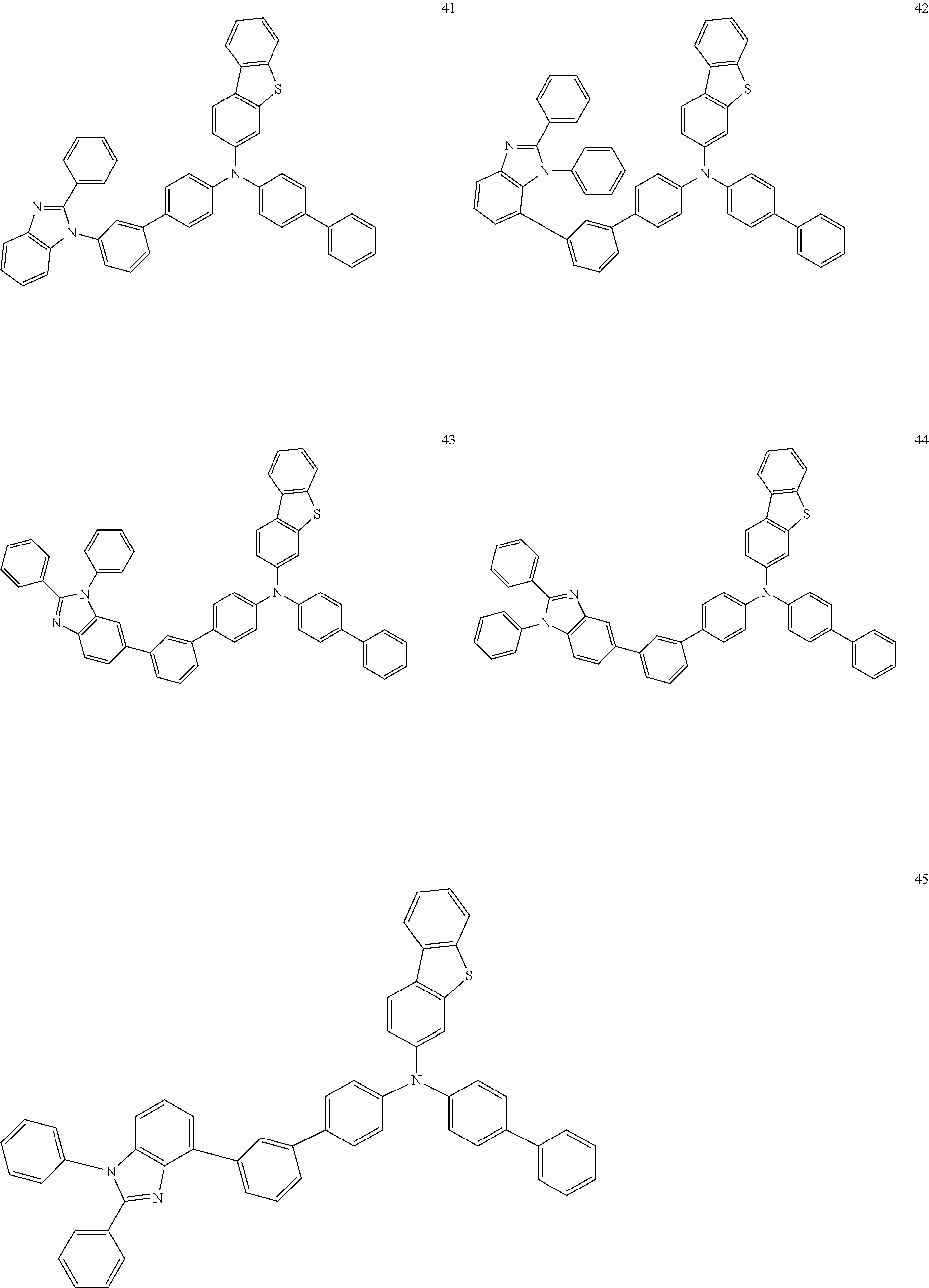

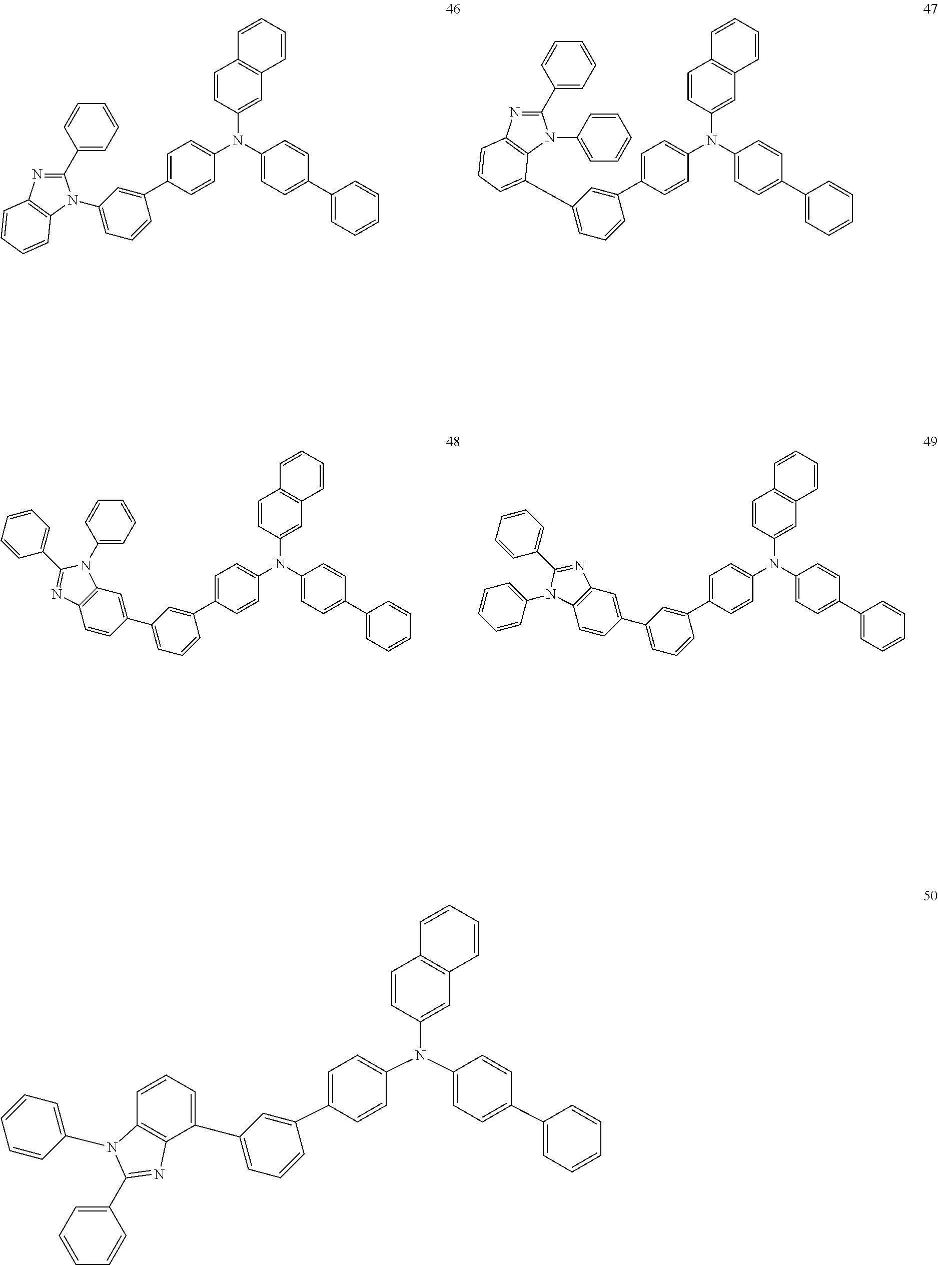 [SMILES: c1ccc(-c2ccc(N(c3ccc(-c4cccc(-n5c(-c6ccccc6)nc6ccccc65)c4)cc3)c3ccc4ccccc4c3)cc2)cc1 ; c1ccc(-c2ccc(N(c3ccc(-c4cccc(-c5cccc6nc(-c7ccccc7)n(-c7ccccc7)c56)c4)cc3)c3ccc4ccccc4c3)cc2)cc1 ; c1ccc(-c2ccc(N(c3ccc(-c4cccc(-c5ccc6nc(-c7ccccc7)n(-c7ccccc7)c6c5)c4)cc3)c3ccc4ccccc4c3)cc2)cc1 ; c1ccc(-c2ccc(N(c3ccc(-c4cccc(-c5ccc6c(c5)nc(-c5ccccc5)n6-c5ccccc5)c4)cc3)c3ccc4ccccc4c3)cc2)cc1 ; c1ccc(-c2ccc(N(c3ccc(-c4cccc(-c5cccc6c5nc(-c5ccccc5)n6-c5ccccc5)c4)cc3)c3ccc4ccccc4c3)cc2)cc1]

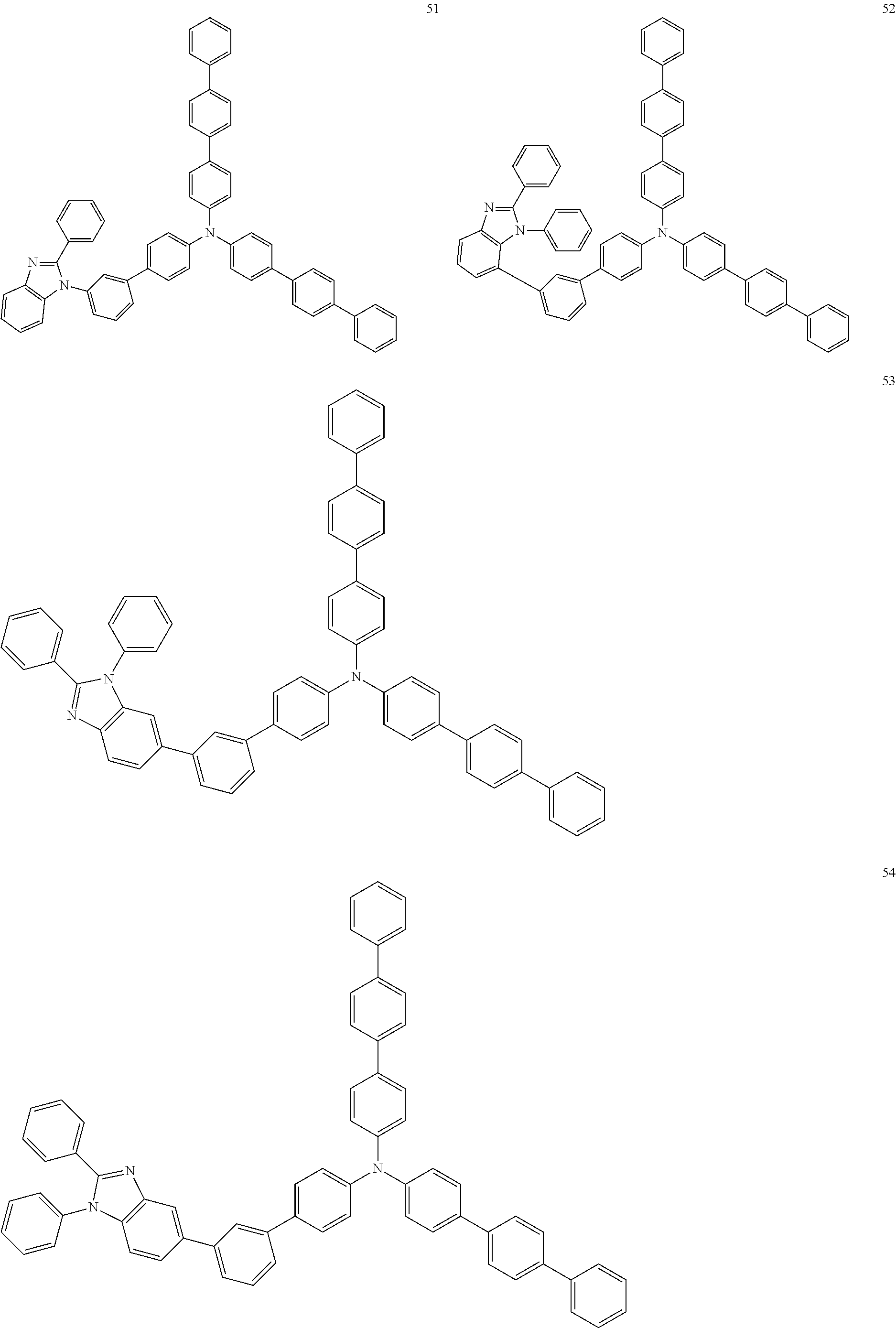

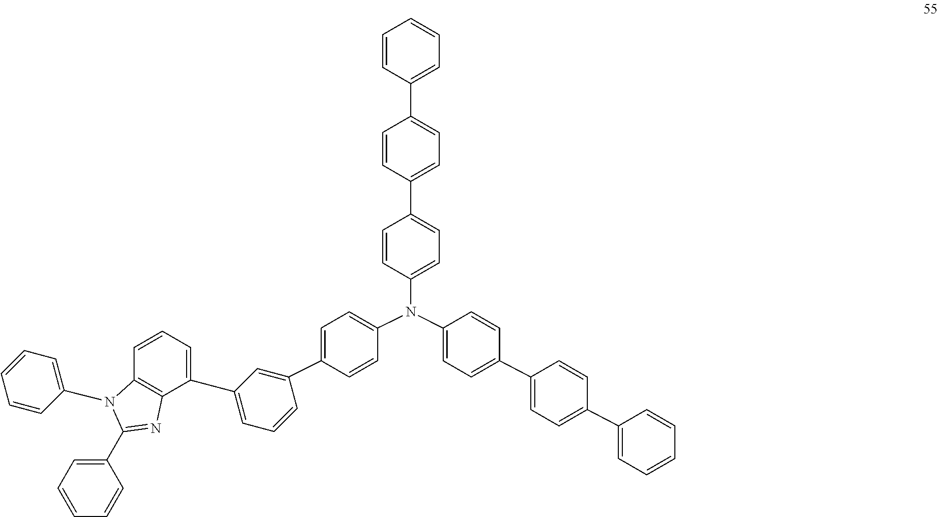 [SMILES: c1ccc(-c2ccc(-c3ccc(N(c4ccc(-c5ccc(-c6ccccc6)cc5)cc4)c4ccc(-c5cccc(-c6cccc7c6nc(-c6ccccc6)n7-c6ccccc6)c5)cc4)cc3)cc2)cc1]

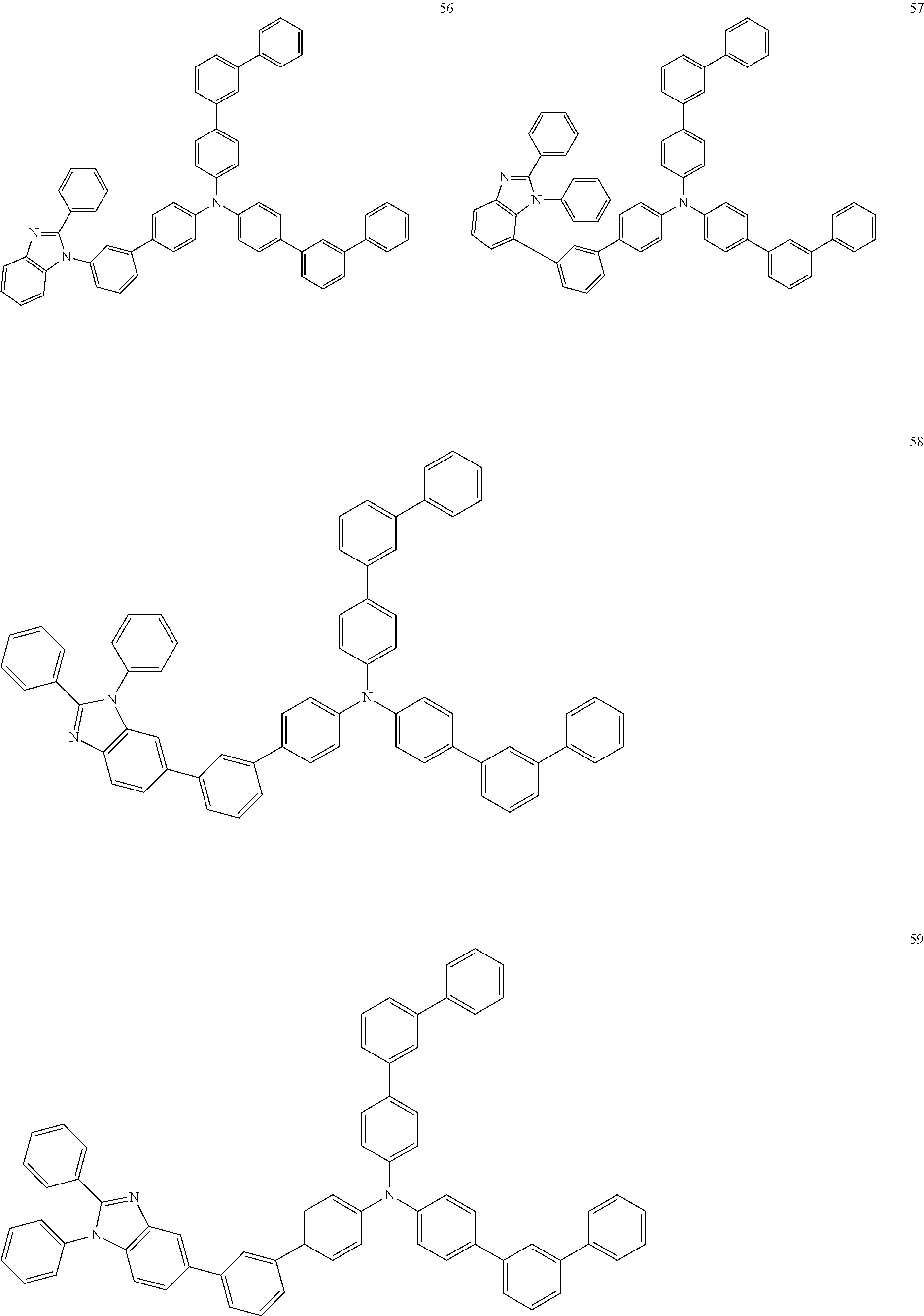 [SMILES: c1ccc(-c2cccc(-c3ccc(N(c4ccc(-c5cccc(-c6ccccc6)c5)cc4)c4ccc(-c5cccc(-n6c(-c7ccccc7)nc7ccccc76)c5)cc4)cc3)c2)cc1 ; c1ccc(-c2cccc(-c3ccc(N(c4ccc(-c5cccc(-c6ccccc6)c5)cc4)c4ccc(-c5cccc(-c6cccc7nc(-c8ccccc8)n(-c8ccccc8)c67)c5)cc4)cc3)c2)cc1 ; c1ccc(-c2cccc(-c3ccc(N(c4ccc(-c5cccc(-c6ccccc6)c5)cc4)c4ccc(-c5cccc(-c6ccc7nc(-c8ccccc8)n(-c8ccccc8)c7c6)c5)cc4)cc3)c2)cc1 ; c1ccc(-c2cccc(-c3ccc(N(c4ccc(-c5cccc(-c6ccccc6)c5)cc4)c4ccc(-c5cccc(-c6ccc7c(c6)nc(-c6ccccc6)n7-c6ccccc6)c5)cc4)cc3)c2)cc1]

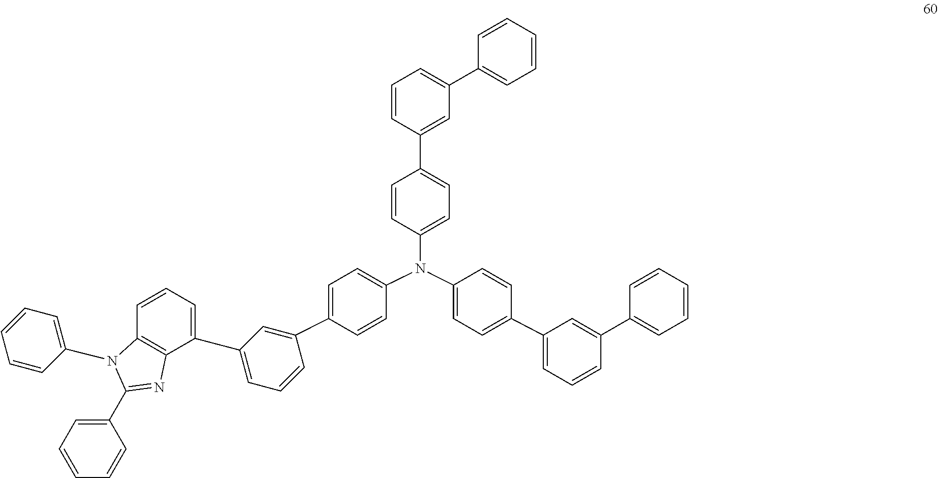 [SMILES: c1ccc(-c2cccc(-c3ccc(N(c4ccc(-c5cccc(-c6ccccc6)c5)cc4)c4ccc(-c5cccc(-c6cccc7c6nc(-c6ccccc6)n7-c6ccccc6)c5)cc4)cc3)c2)cc1]

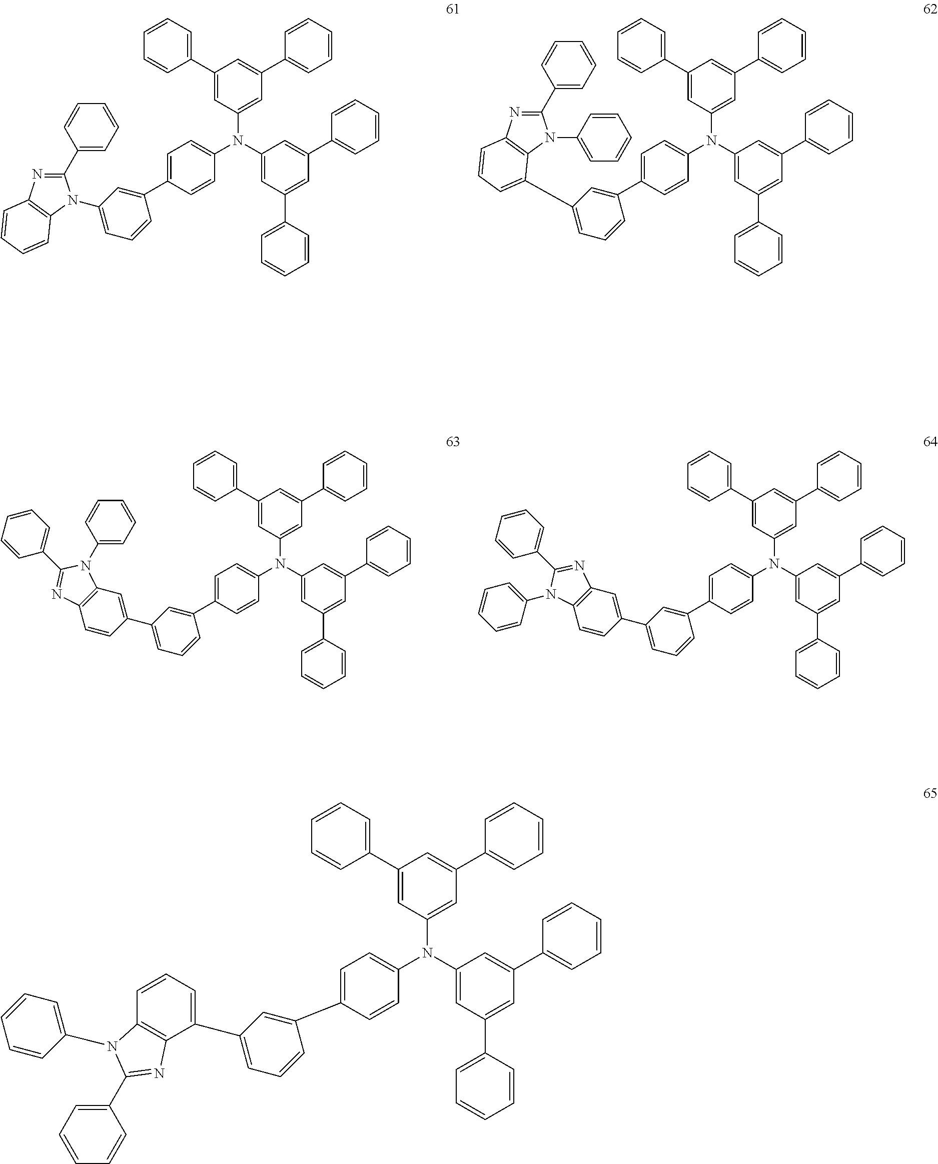

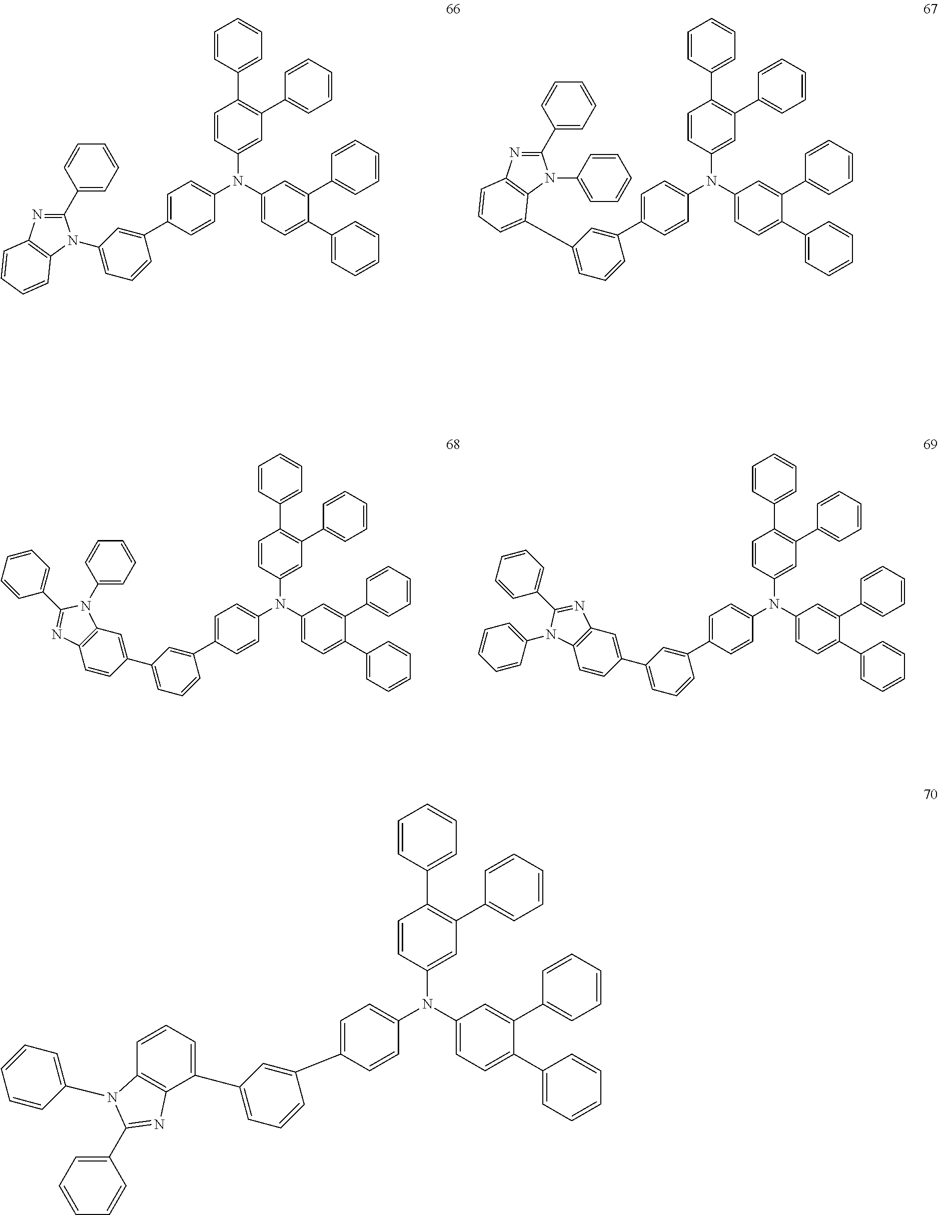

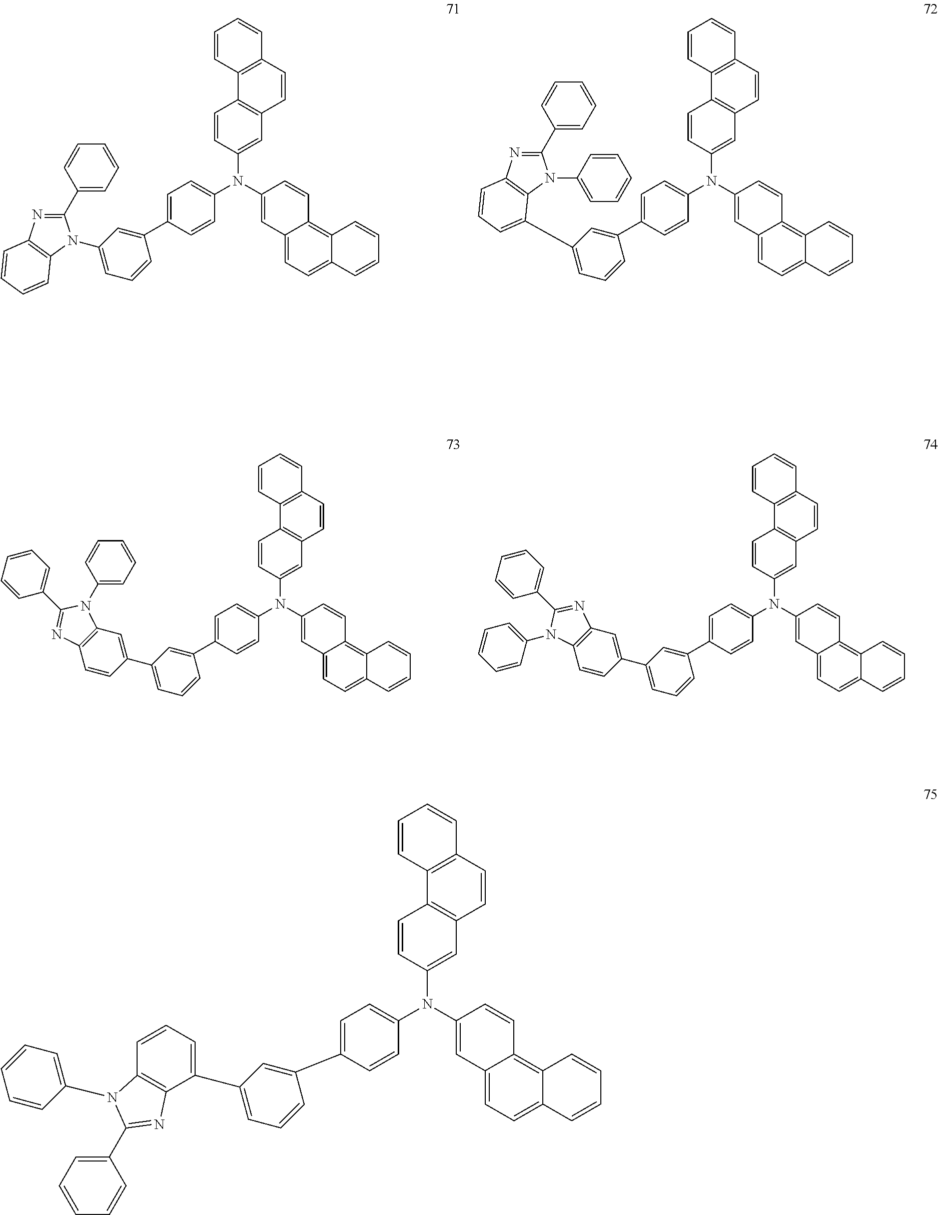

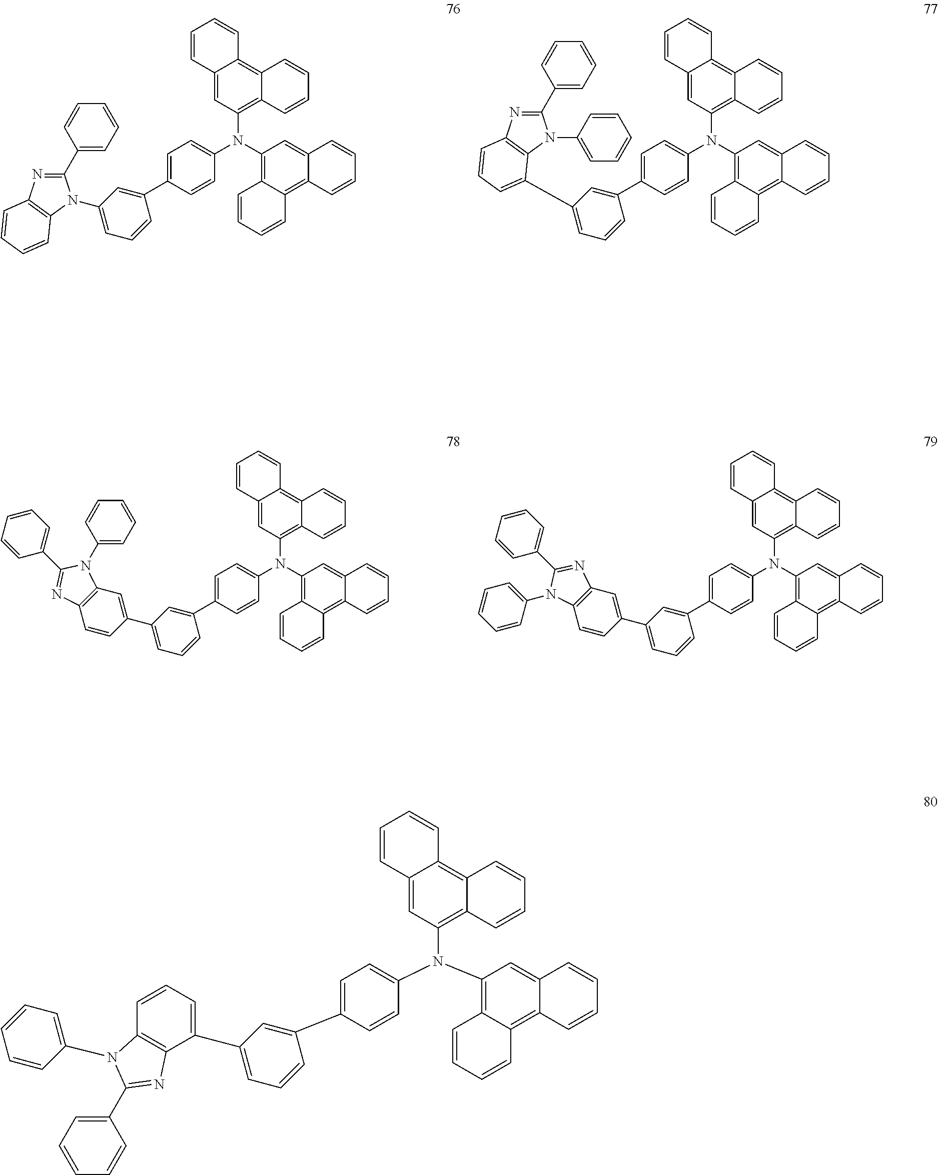

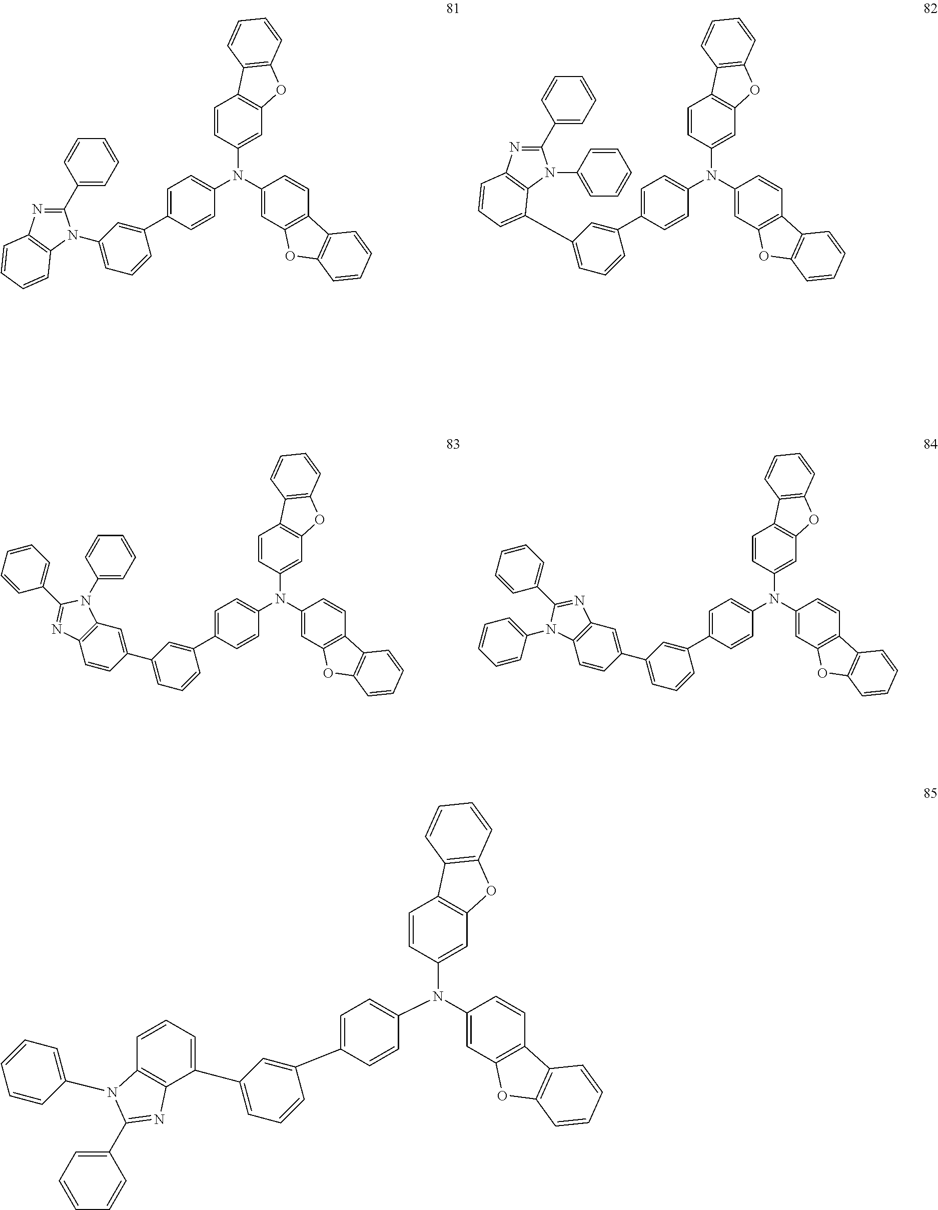

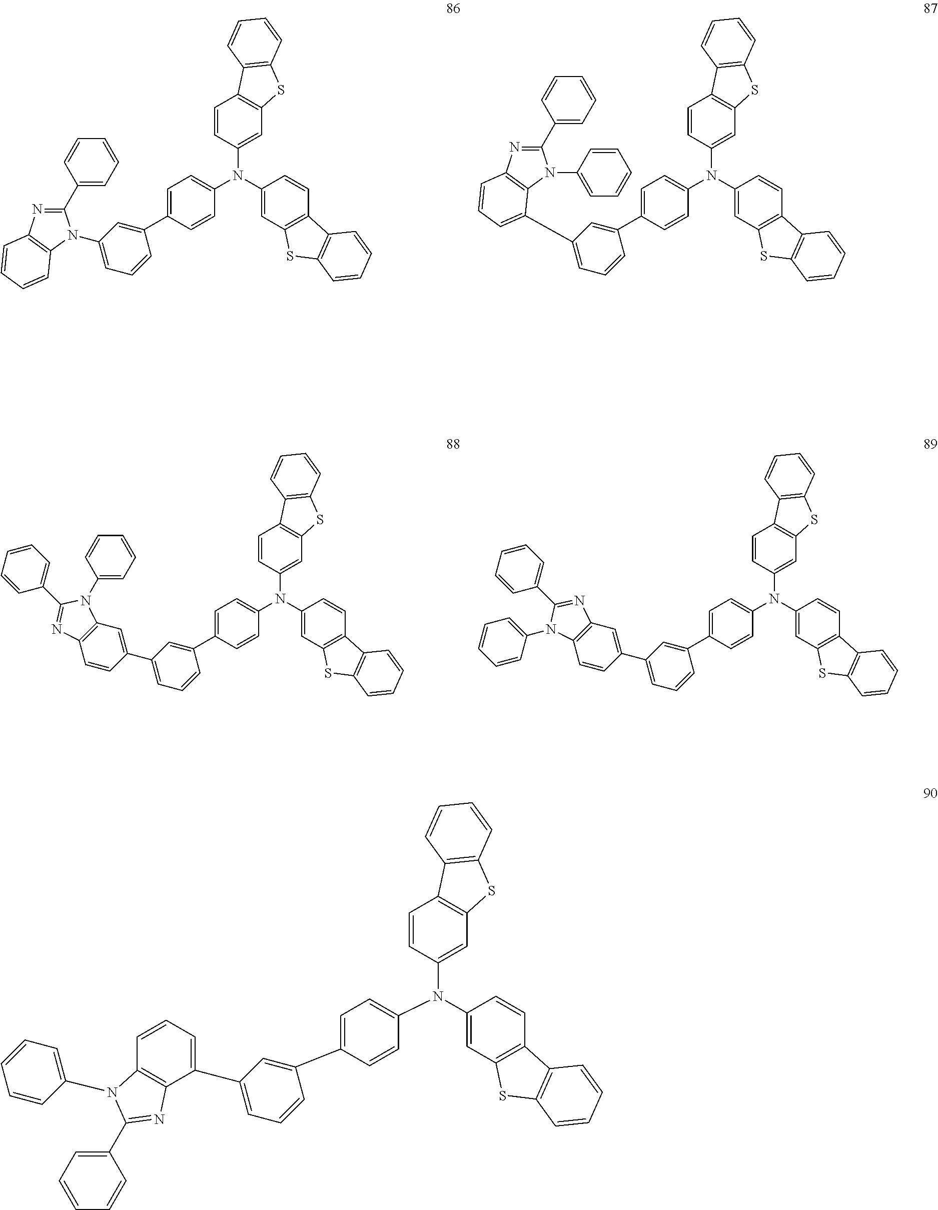

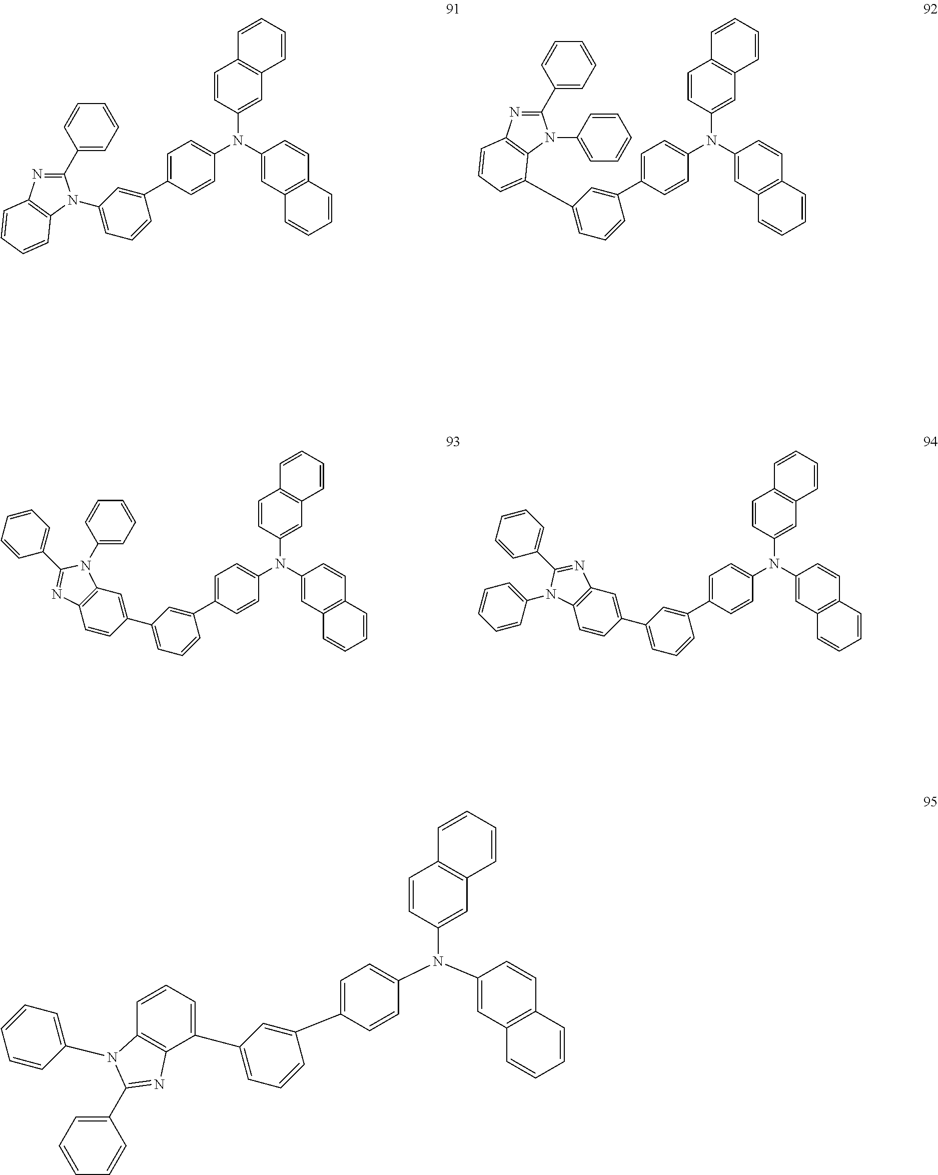 [SMILES: c1ccc(-c2nc3ccccc3n2-c2cccc(-c3ccc(N(c4ccc5ccccc5c4)c4ccc5ccccc5c4)cc3)c2)cc1 ; c1ccc(-c2nc3cccc(-c4cccc(-c5ccc(N(c6ccc7ccccc7c6)c6ccc7ccccc7c6)cc5)c4)c3n2-c2ccccc2)cc1 ; c1ccc(-c2nc3ccc(-c4cccc(-c5ccc(N(c6ccc7ccccc7c6)c6ccc7ccccc7c6)cc5)c4)cc3n2-c2ccccc2)cc1 ; c1ccc(-c2nc3cc(-c4cccc(-c5ccc(N(c6ccc7ccccc7c6)c6ccc7ccccc7c6)cc5)c4)ccc3n2-c2ccccc2)cc1 ; c1ccc(-c2nc3c(-c4cccc(-c5ccc(N(c6ccc7ccccc7c6)c6ccc7ccccc7c6)cc5)c4)cccc3n2-c2ccccc2)cc1]

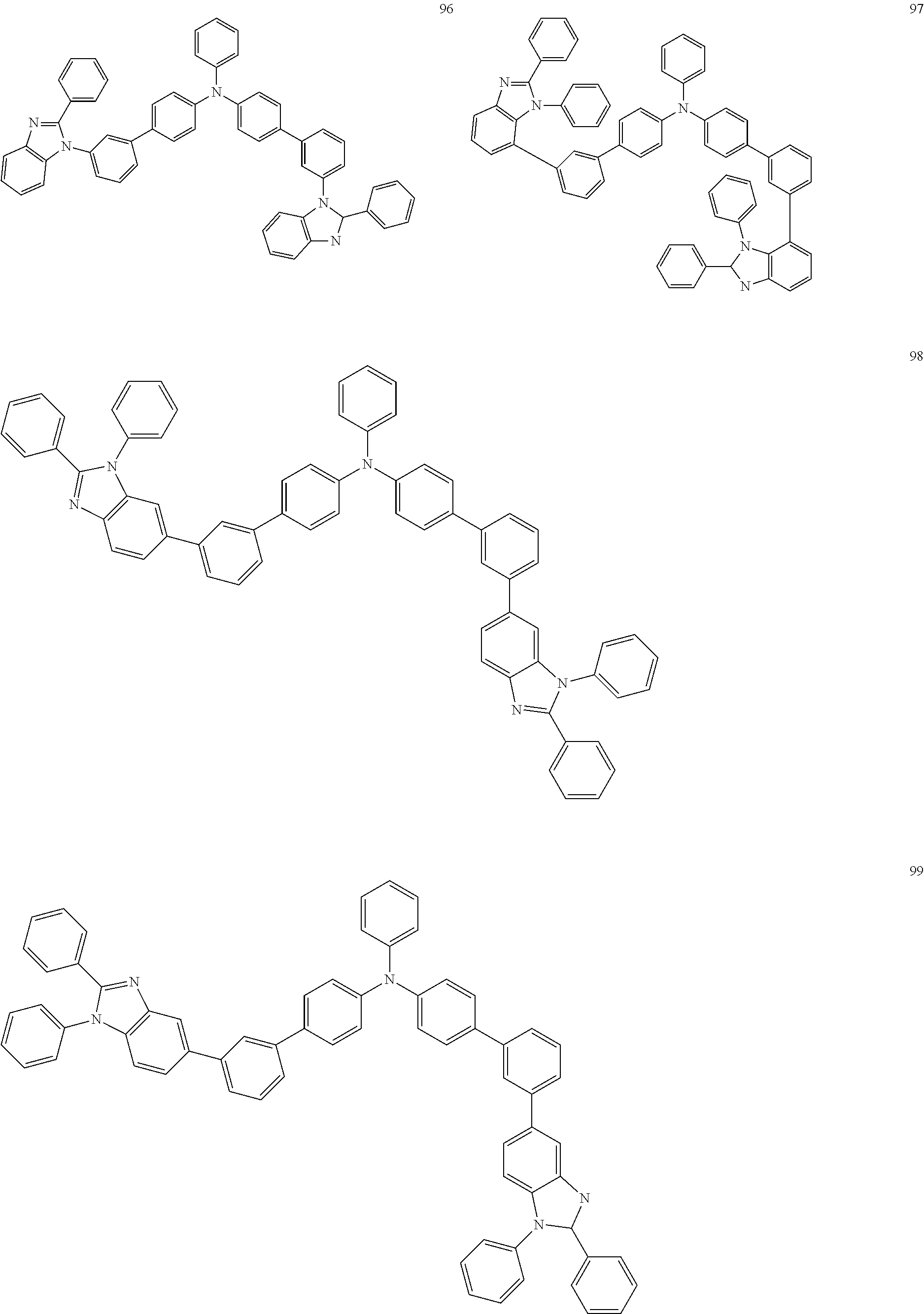 [SMILES: c1ccc(-c2nc3ccccc3n2-c2cccc(-c3ccc(N(c4ccccc4)c4ccc(-c5cccc(N6c7ccccc7NC6c6ccccc6)c5)cc4)cc3)c2)cc1 ; c1ccc(-c2nc3cccc(-c4cccc(-c5ccc(N(c6ccccc6)c6ccc(-c7cccc(-c8cccc9c8N(c8ccccc8)C(c8ccccc8)N9)c7)cc6)cc5)c4)c3n2-c2ccccc2)cc1 ; c1ccc(-c2nc3ccc(-c4cccc(-c5ccc(N(c6ccccc6)c6ccc(-c7cccc(-c8ccc9nc(-c%10ccccc%10)n(-c%10ccccc%10)c9c8)c7)cc6)cc5)c4)cc3n2-c2ccccc2)cc1 ; c1ccc(-c2nc3cc(-c4cccc(-c5ccc(N(c6ccccc6)c6ccc(-c7cccc(-c8ccc9c(c8)NC(c8ccccc8)N9c8ccccc8)c7)cc6)cc5)c4)ccc3n2-c2ccccc2)cc1]

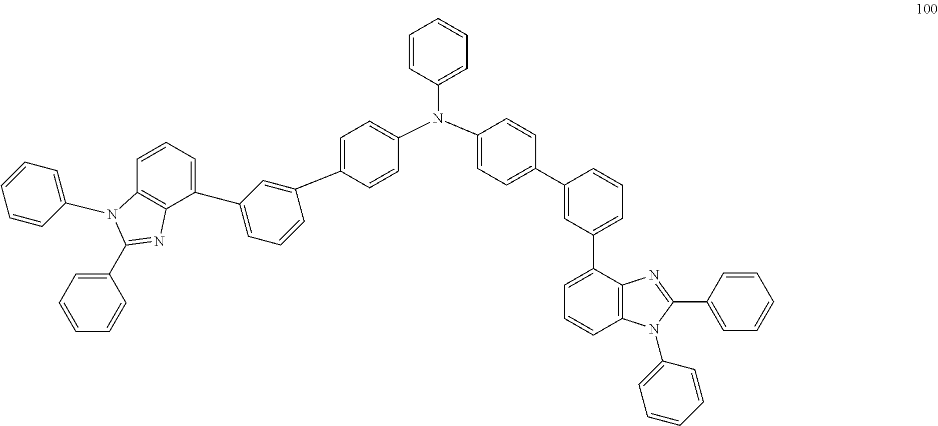 [SMILES: c1ccc(-c2nc3c(-c4cccc(-c5ccc(N(c6ccccc6)c6ccc(-c7cccc(-c8cccc9c8nc(-c8ccccc8)n9-c8ccccc8)c7)cc6)cc5)c4)cccc3n2-c2ccccc2)cc1]

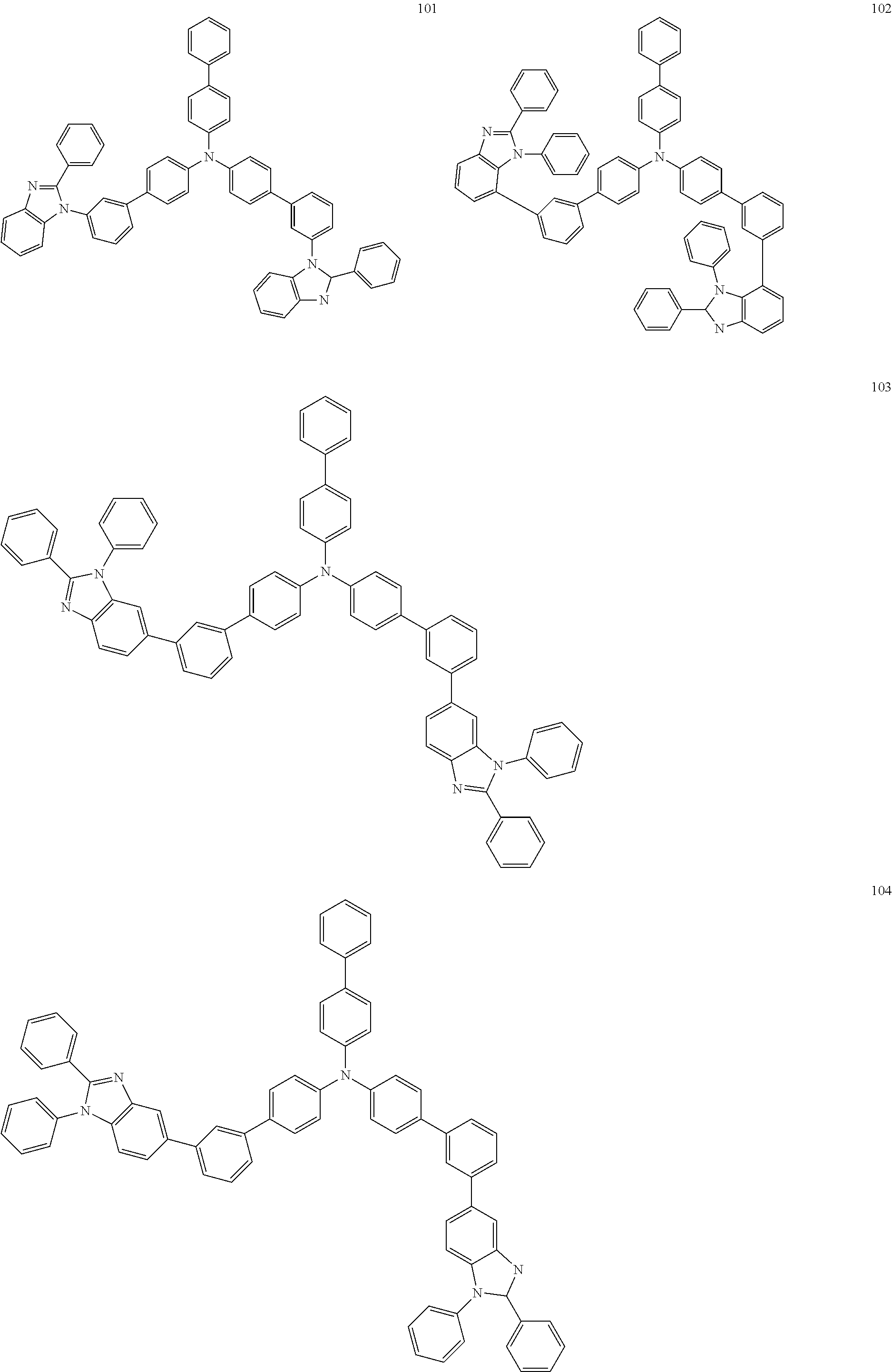

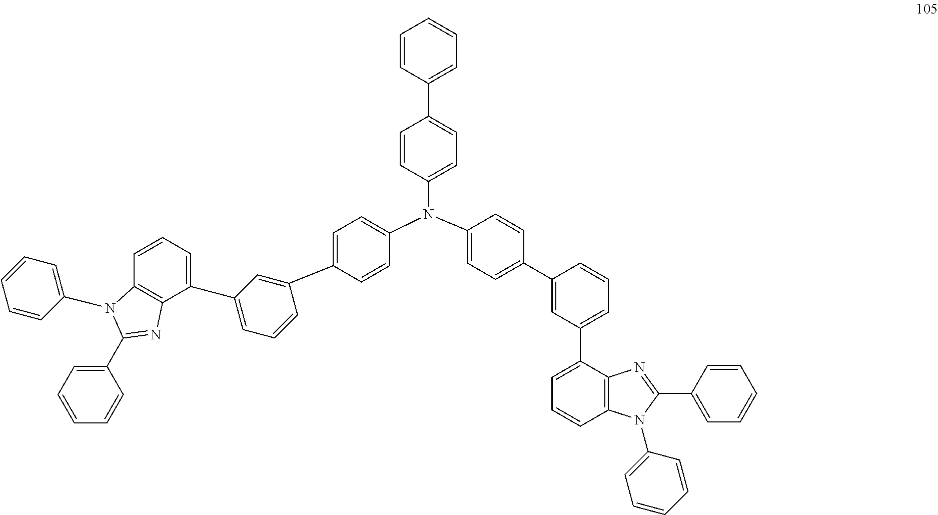 [SMILES: c1ccc(-c2ccc(N(c3ccc(-c4cccc(-c5cccc6c5nc(-c5ccccc5)n6-c5ccccc5)c4)cc3)c3ccc(-c4cccc(-c5cccc6c5nc(-c5ccccc5)n6-c5ccccc5)c4)cc3)cc2)cc1]

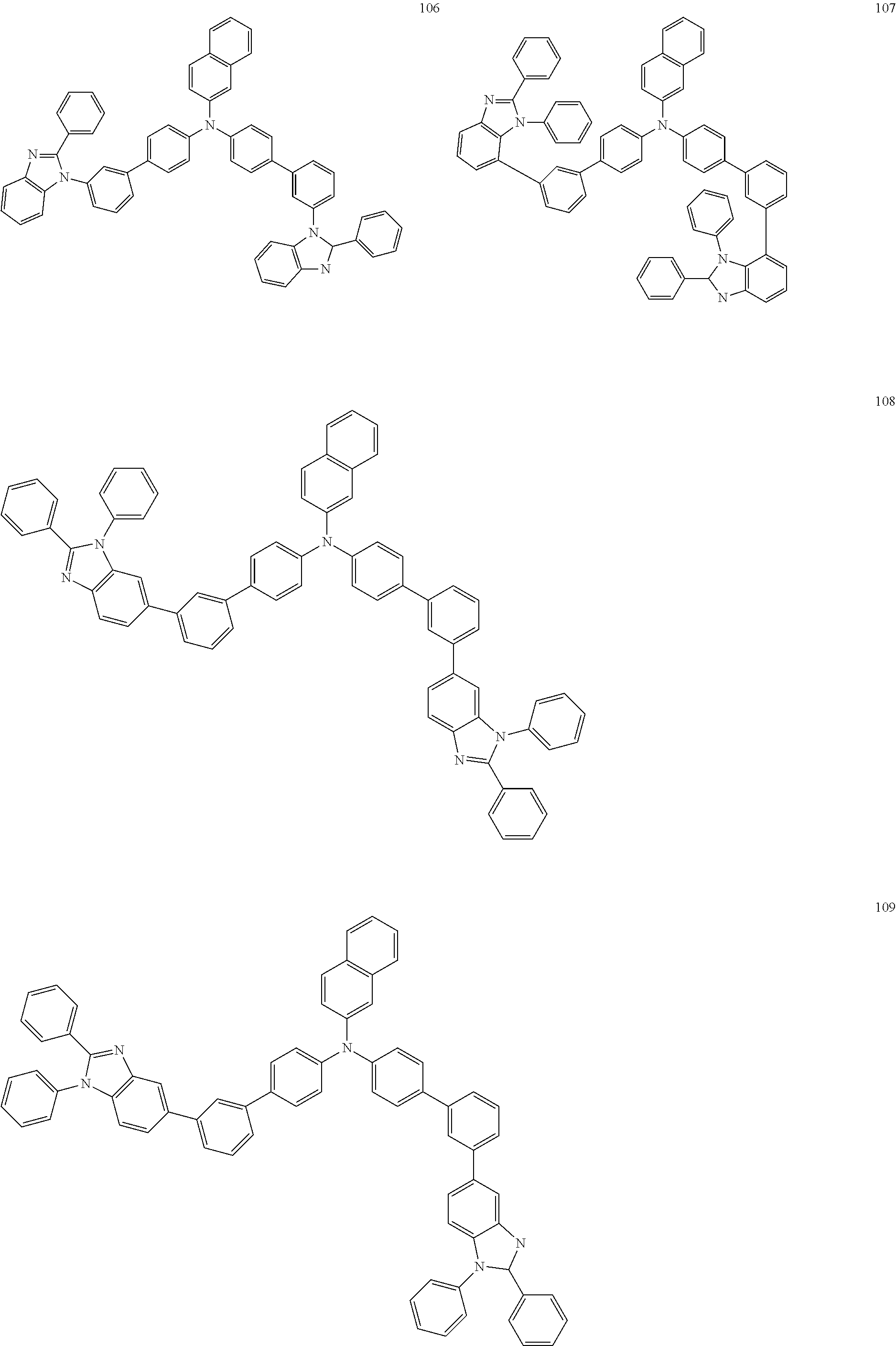

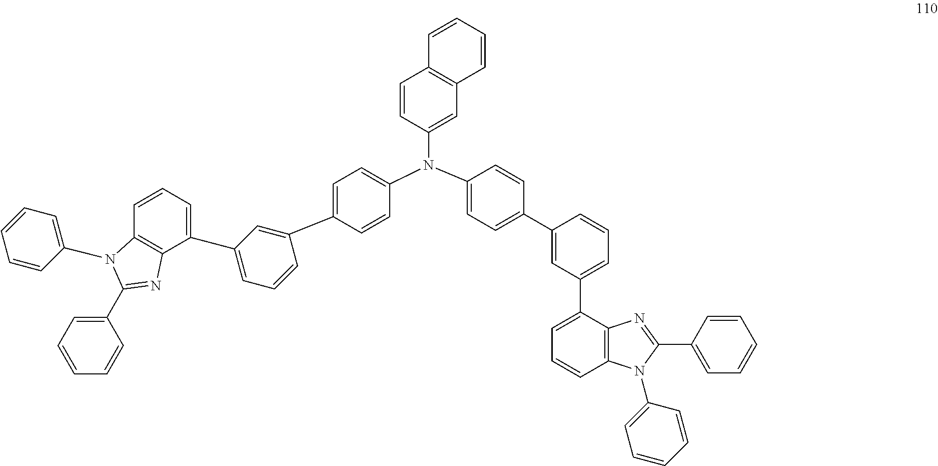 [SMILES: c1ccc(-c2nc3c(-c4cccc(-c5ccc(N(c6ccc(-c7cccc(-c8cccc9c8nc(-c8ccccc8)n9-c8ccccc8)c7)cc6)c6ccc7ccccc7c6)cc5)c4)cccc3n2-c2ccccc2)cc1]

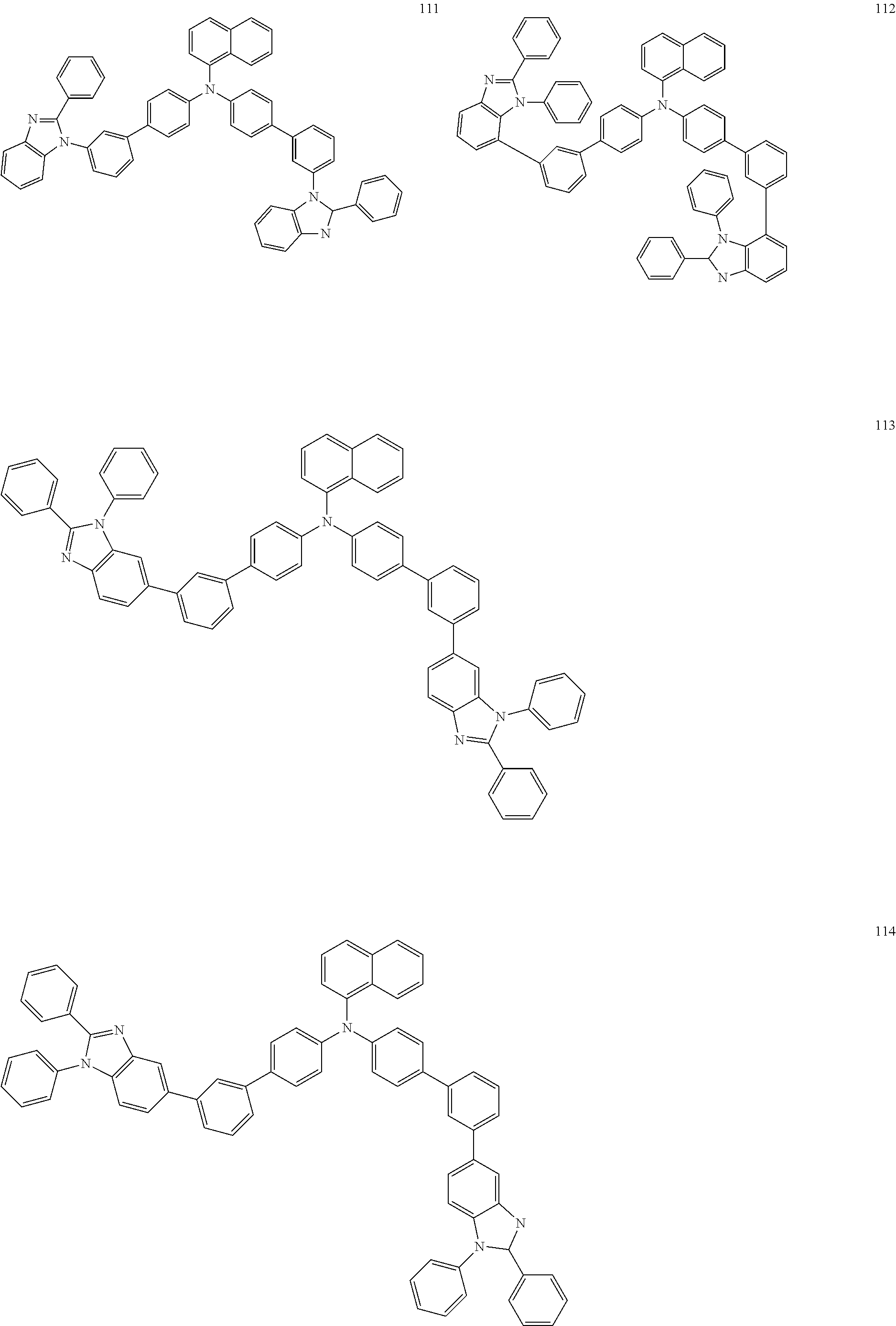

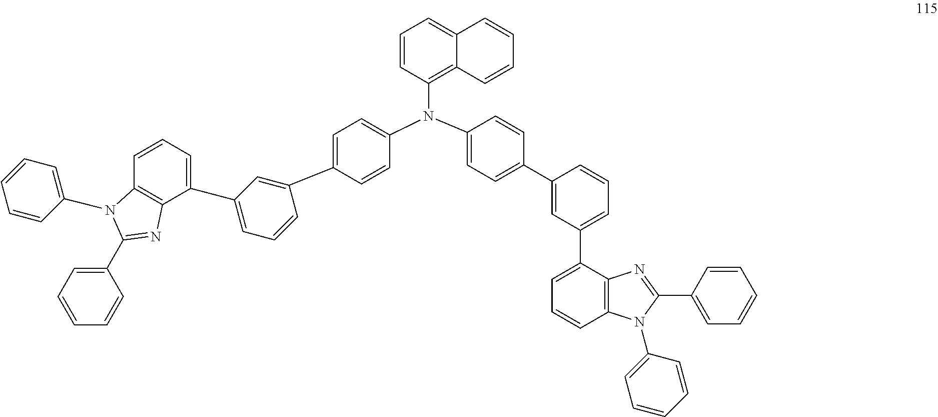 [SMILES: c1ccc(-c2nc3c(-c4cccc(-c5ccc(N(c6ccc(-c7cccc(-c8cccc9c8nc(-c8ccccc8)n9-c8ccccc8)c7)cc6)c6cccc7ccccc67)cc5)c4)cccc3n2-c2ccccc2)cc1]

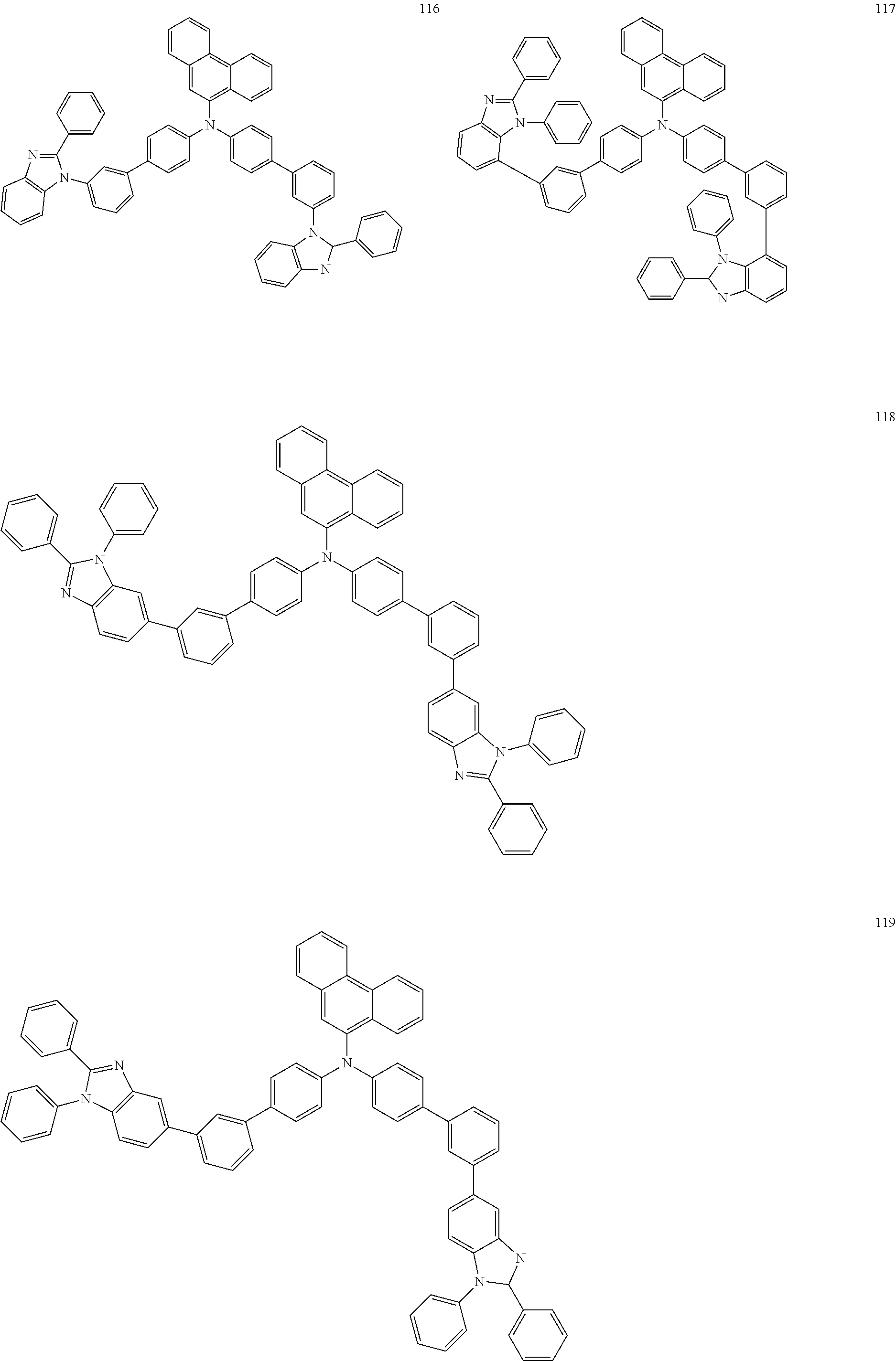

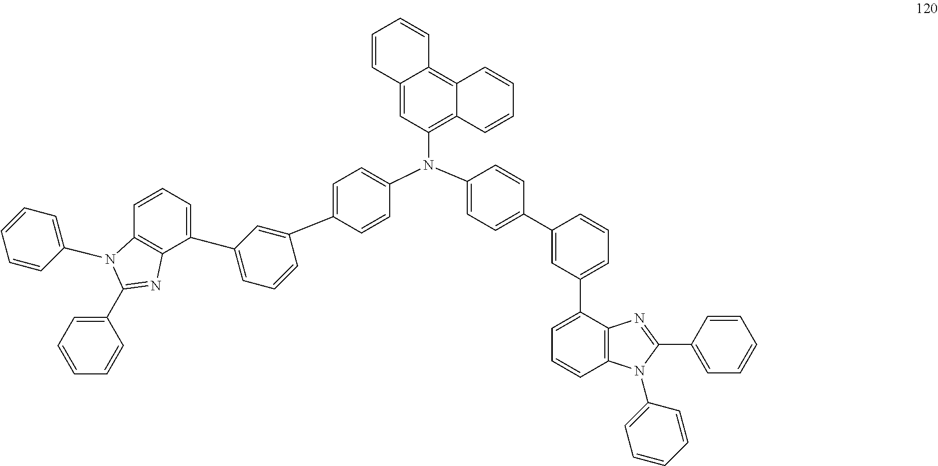 [SMILES: c1ccc(-c2nc3c(-c4cccc(-c5ccc(N(c6ccc(-c7cccc(-c8cccc9c8nc(-c8ccccc8)n9-c8ccccc8)c7)cc6)c6cc7ccccc7c7ccccc67)cc5)c4)cccc3n2-c2ccccc2)cc1]

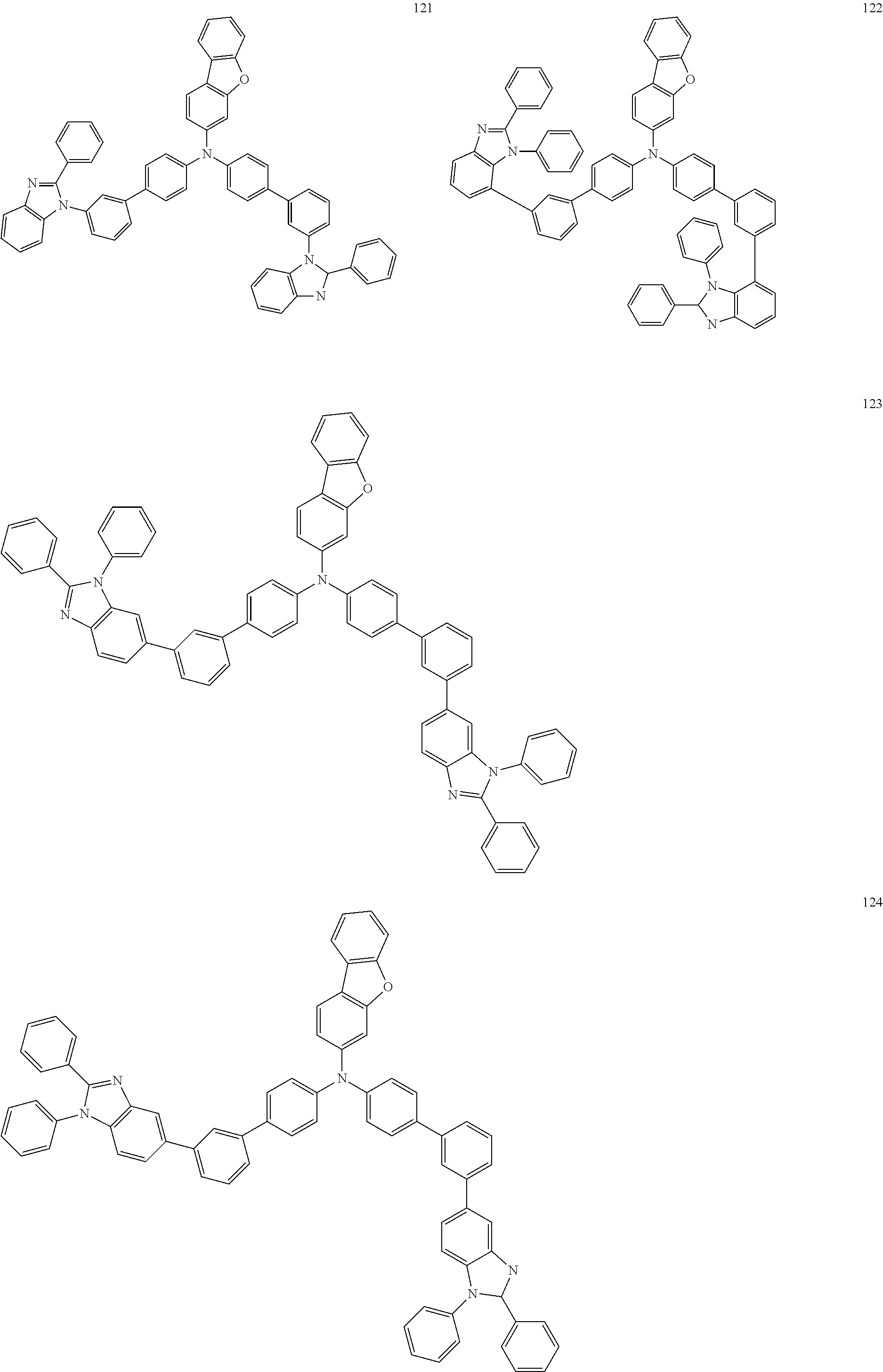

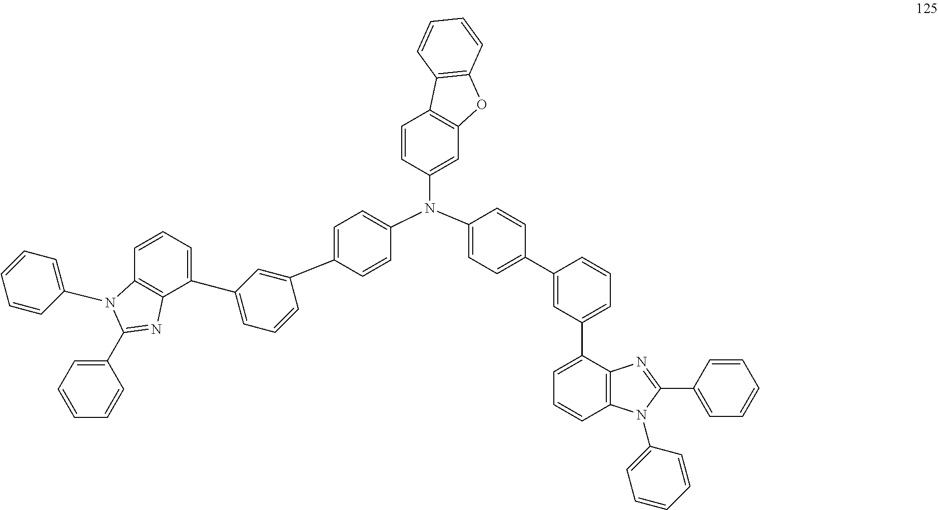 [SMILES: c1ccc(-c2nc3c(-c4cccc(-c5ccc(N(c6ccc(-c7cccc(-c8cccc9c8nc(-c8ccccc8)n9-c8ccccc8)c7)cc6)c6ccc7c(c6)oc6ccccc67)cc5)c4)cccc3n2-c2ccccc2)cc1]

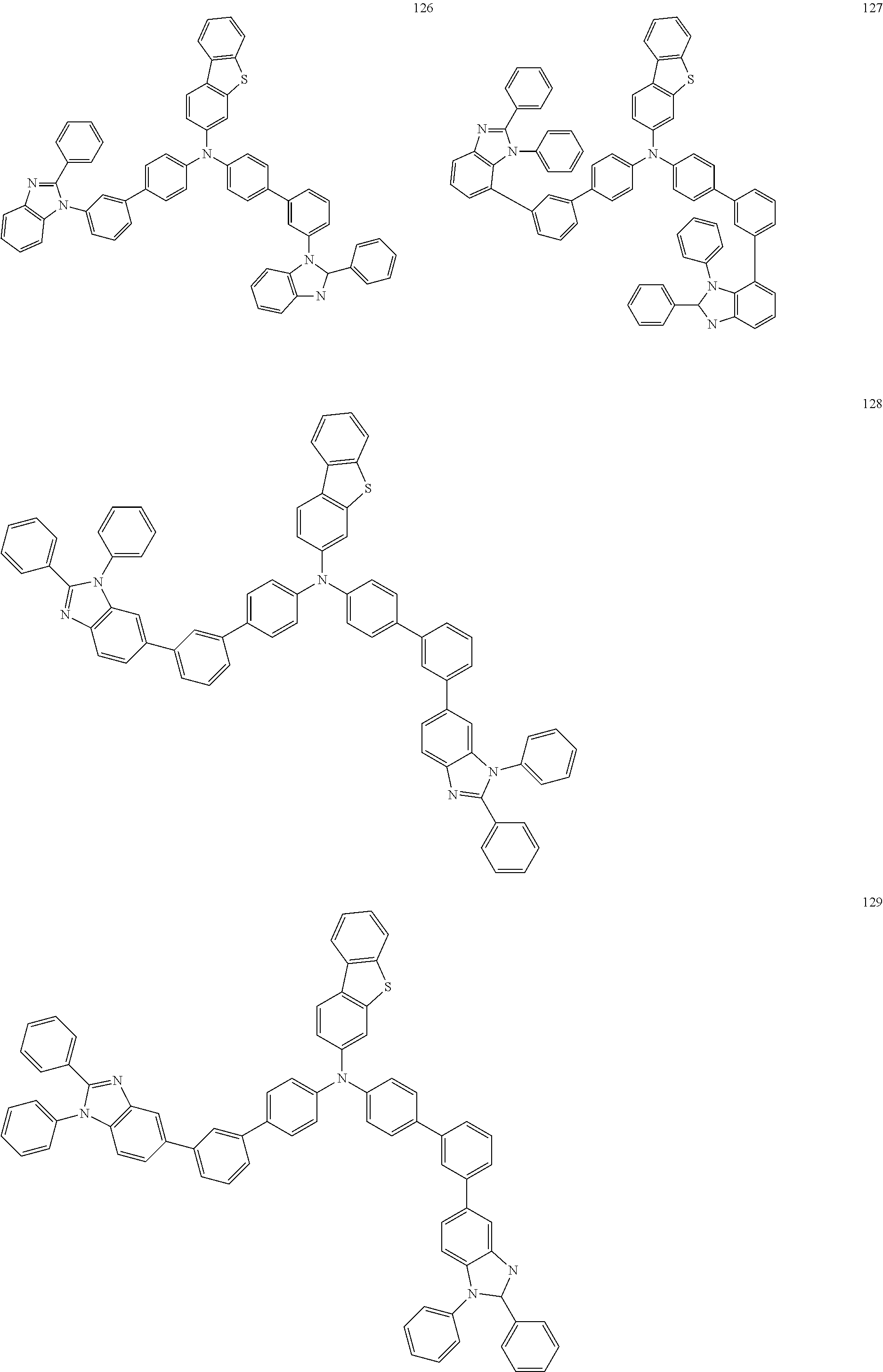

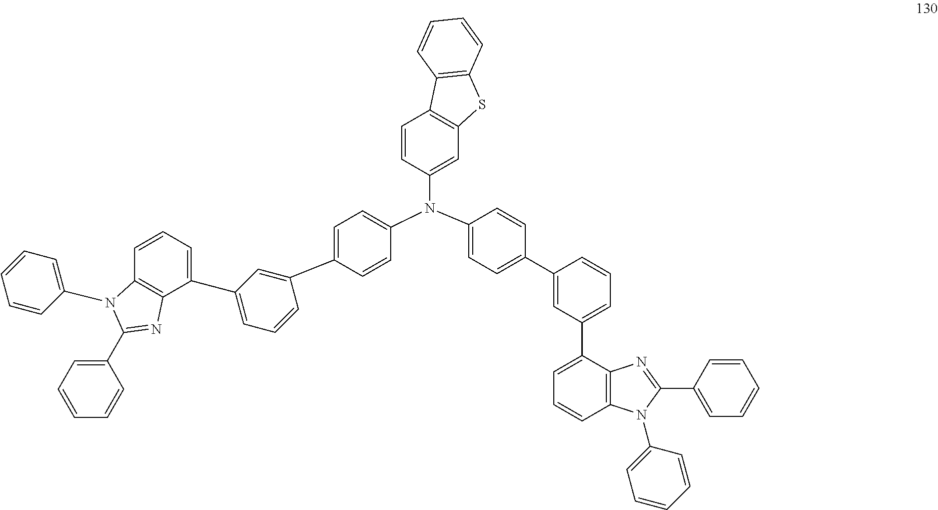 [SMILES: c1ccc(-c2nc3c(-c4cccc(-c5ccc(N(c6ccc(-c7cccc(-c8cccc9c8nc(-c8ccccc8)n9-c8ccccc8)c7)cc6)c6ccc7c(c6)sc6ccccc67)cc5)c4)cccc3n2-c2ccccc2)cc1]

The amine derivative according to an embodiment of the present disclosure may be synthesized according to the examples of Reaction Formulae (1) to (3).

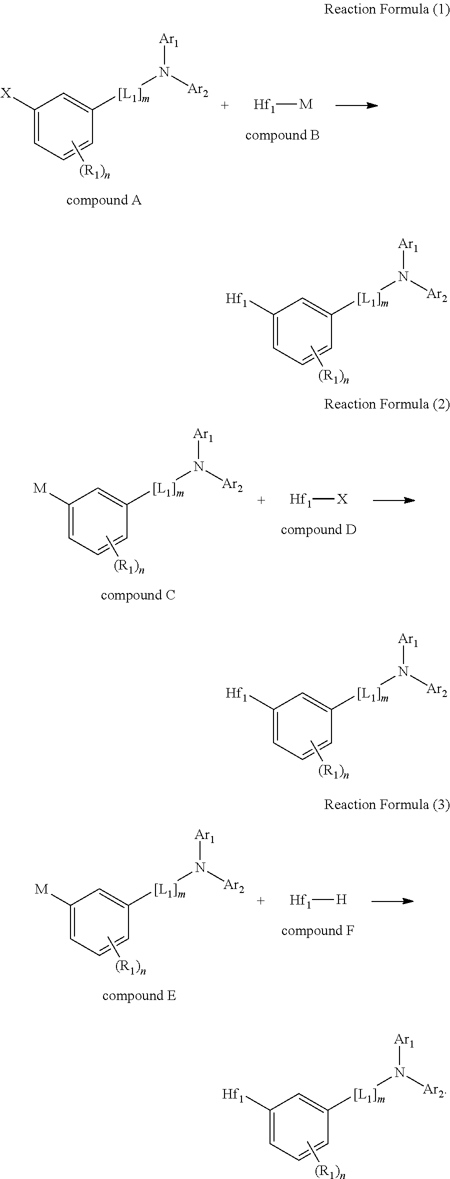

In Reaction Formula (1), when compound A is a compound having a leaving group such as a halogen (X) at the m position and compound B is a compound including a metal (M) such as boron (B), the amine derivative according to an embodiment of the present disclosure may be synthesized through a coupling reaction between compound A and compound B.

In Reaction Formula (2), when compound C is a compound having a leaving group such as a metal (M), e.g., B, at the m position, and compound D is a compound including X, the amine derivative according to an embodiment of the present disclosure may be synthesized through a coupling reaction between compound C and compound D.

In Reaction Formula (3), when compound E is a compound having a leaving group such as X at the m position and compound F is a compound including hydrogen (H), the amine derivative according to an embodiment of the present disclosure may be synthesized through a coupling reaction between compound E and compound F.

However, synthesis of the amine derivative according to an embodiment of the present disclosure is not limited to the synthetic examples in Reaction Formulae (1) to (3). For example, a pathway in which the coupling reactions in Reaction Formulae (1) to (3) are carried out at first, and then Ar₁ and Ar₂ are introduced may be selected.

<2. Organic Electroluminescent Device Including Material for Organic Electroluminescent Device Having Amine Derivative>

Referring to the drawing, an organic electroluminescent (EL) device including a material for an organic EL device according to an embodiment of the present disclosure will be described. The drawing is a schematic cross-sectional diagram showing an example of the organic EL device according to an embodiment of the present disclosure.

As shown in the drawing, the organic EL device 100 according to an embodiment of the present disclosure includes a substrate 110, a first electrode 120 disposed on the substrate 110, a hole injection layer 130 disposed on the first electrode 120, a hole transport layer 140 disposed on the hole injection layer 130, an emission layer 150 disposed on the hole transport layer 140, an electron transport layer 160 disposed on the emission layer 150, an electron injection layer 170 disposed on the electron transport layer 160, and a second electrode 180 disposed on the electron injection layer 170.

The material for an organic EL device according to an embodiment of the present disclosure may be included in the hole transport layer 140 and/or the emission layer 150. The material for an organic EL may be included in both of those layers. In one embodiment, the material for an organic EL device is included in the hole transport layer 140.

Each organic layer (e.g., thin layer) disposed between the first electrode 120 and the second electrode 180 of the organic EL device 100 may be provided through various suitable methods, e.g., various suitable deposition method, etc.

As the substrate 110, a substrate utilized in the general organic EL device may be utilized. For example, the substrate 110 may be a glass substrate, a semiconductor substrate, a transparent plastic substrate, etc.

The first electrode 120 is, for example, an anode, and may be provided on the substrate 110 utilizing a deposition method, a sputtering method, etc. For example, the first electrode 120 may be provided as a transmitting (e.g., a penetrating) electrode utilizing a conductive compound, an alloy, and/or a metal having high work function. The first electrode 120 may include, for example, indium tin oxide (ITO), indium zinc oxide (IZO), tin oxide (SnO₂), and/or zinc oxide (ZnO), which are transparent and have excellent conductivity. Also, the anode 120 may be provided as a reflective electrode utilizing magnesium (Mg), aluminum (Al), etc.

On the first electrode 120, the hole injection layer 130 is provided. The hole injection layer 130 has a function of facilitating the injection of holes from the first electrode 120, and is, for example, provided on the first electrode 120 in a thickness of about 10 nm to about 150 nm. The hole injection layer 130 may be provided utilizing any suitable materials. Examples of the suitable material may include triphenylamine-containing polyetherketone (TPAPEK), 4-isopropyl-4′-methyldiphenyl iodonium tetrakis (pentafluorophenyl) borate (PPBI), N,N′-diphenyl-N,N′-bis-[4-(phenyl-m-tolly-amino)-phenyl]-biphenyl-4,4′-diamine (DNTPD), a phthalocyanine compound (such as copper phthalocyanine), 4,4′,4″-tris(3-methylphenylamino)triphenylamine (m-MTDATA), N,N′-di(1-naphthyl)-N,N′-diphenyl benzidine (NPB), 4,4′,4″-tris{N,Ndiamino}triphenylamine (TDATA), 4,4′,4″-tris(N,N-2-naphthyl phenyl amino) triphenyl amine (2-TNATA), polyaniline/dodecyl benzene sulfonic acid (Pani/DBSA), poly(3,4-ethylenedioxythiophene)/poly(4-styrene sulfonate) (PEDOT/PSS), polyaniline/camphor sulfonic acid (Pani/CSA), polyaniline/poly(4-styrene sulfonate) (PANI/PSS), etc.

The hole transport layer 140 is provided on the hole injection layer 130. A plurality of hole transport layers 140 may be laminated. The hole transport layer 140 (including a hole transport material having a function of transporting holes) is provided, for example, on the hole injection layer 130 with a thickness of about 10 nm to about 150 nm. In one embodiment, the hole transport layer 140 includes the material for an organic EL device according to an embodiment of the present disclosure. In another embodiment, when the material for an organic EL device according to an embodiment of the present disclosure is utilized as a host material in the emission layer 150, the hole transport layer 140 may include an existing hole transport material. Examples of the existing hole transport material may include 1,1-bis[(di-4-tolylamino)phenyl]cyclohexane (TAPC), a carbazole derivative (such as N-phenyl carbazole, and/or polyvinylcarbazole), N,N′-bis(3-methylphenyl)-N,N′-diphenyl-[1,1-biphenyl]-4,4′-diamine (TPD), 4,4′,4″-tris(N-carbazolyl)triphenyl amine (TCTA), N,N′-di(1-naphthyl)-N,N′-diphenyl benzidine (NPB), etc.

The emission layer 150 is provided on the hole transport layer 140. The emission layer 150 (emitting light by fluorescence, phosphorescence, etc.) is provided with a thickness of about 10 nm to about 60 nm. A suitable emission material for the emission layer 150 may be, for example, selected from, but not limited to, a fluoranthene derivative, a pyrene derivative, an arylacetylene derivative, a fluorene derivative, a perylene derivative, and/or a chrysene derivative. In one embodiment, a pyrene derivative, a perylene derivative, and/or an anthracene derivative may be utilized. For example, as a material for the emission layer 150, an anthracene derivative represented by following Formula 5 may be utilized.

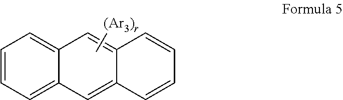

In Formula 5, Ar₃ is hydrogen, deuterium, a substituted or unsubstituted alkyl group having 1 or more to 50 or less carbon atoms, a substituted or unsubstituted cycloalkyl group having 3 or more to 50 or less carbon atoms for forming a ring, a substituted or unsubstituted alkoxy group having 1 or more to 50 or less carbon atoms, a substituted or unsubstituted aralkyl group having 7 or more to 50 or less carbon atoms, a substituted or unsubstituted aryloxy group having 6 or more to 50 or less carbon atoms for forming a ring, a substituted or unsubstituted arylthio group having 6 or more to 50 or less carbon atoms for forming a ring, a substituted or unsubstituted alkoxycarbonyl group having 2 or more to 50 or less carbon atoms, a substituted or unsubstituted aryl group having 6 or more to 50 or less carbon atoms for forming a ring, a substituted or unsubstituted heteroaryl group having 5 or more to 50 or less carbon atoms for forming a ring, a substituted or unsubstituted silly group, a carboxyl group, a halogen group, a cyano group, a nitro group, or a hydroxyl group; and r is an integer from 1 or more to 10 or less. When r is 2 or more, plurality of Ar₃s may be the same as or different from each other.

In Formula 5, for example, Ar₃ may be a phenyl group, a biphenyl group, a terphenyl group, a naphthyl group, a phenylnaphthyl group, a naphthylphenyl group, an anthryl group, a phenanthryl group, a fluorenyl group, an indenyl group, a pyrenyl group, an acetonaphthenyl group, a fluoranthenyl group, a triphenylenyl group, a pyridyl group, a furanyl group, a pyranyl group, a thienyl group, a kinoryl group, an isokinoryl group, a benzofuranyl group, a benzothienyl group, an indolyl group, a carbazolyl group, a benzoxazolyl group, a benzothiazolyl group, a quinoxalyl group, a benzimidazolyl group, a pyrazolyl group, a dibenzofuranyl group, a dibenzothienyl group, etc. In one embodiment, examples of Ar₃ may include a phenyl group, a biphenyl group, a terphenyl group, a fluorenyl group, a carbazolyl group, a dibenzofuranyl group, etc.

Examples of the compounds represented by Formula 5 may include compounds a-1 to a-12. However, the compound represented by Formula 5 is not limited to the following Compounds a-1 to a-12.

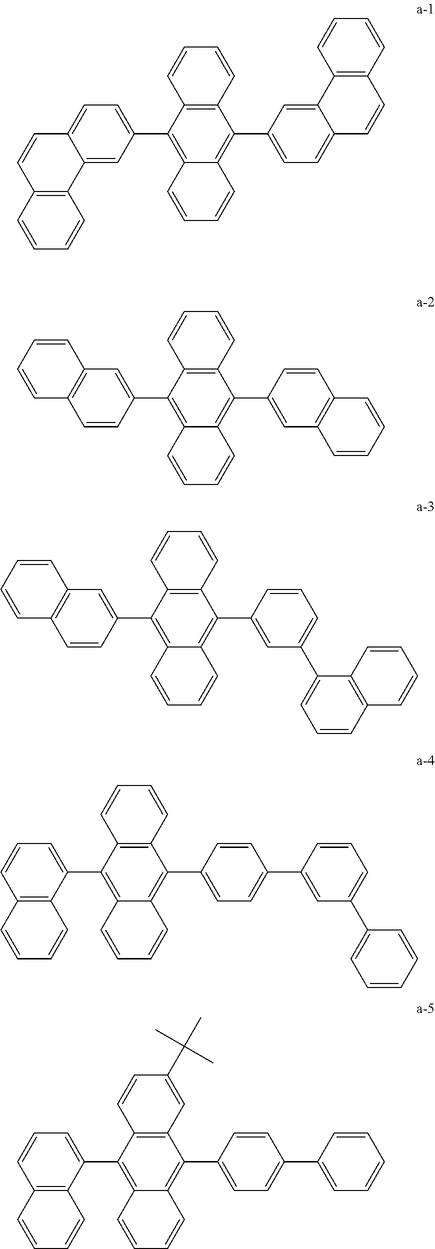

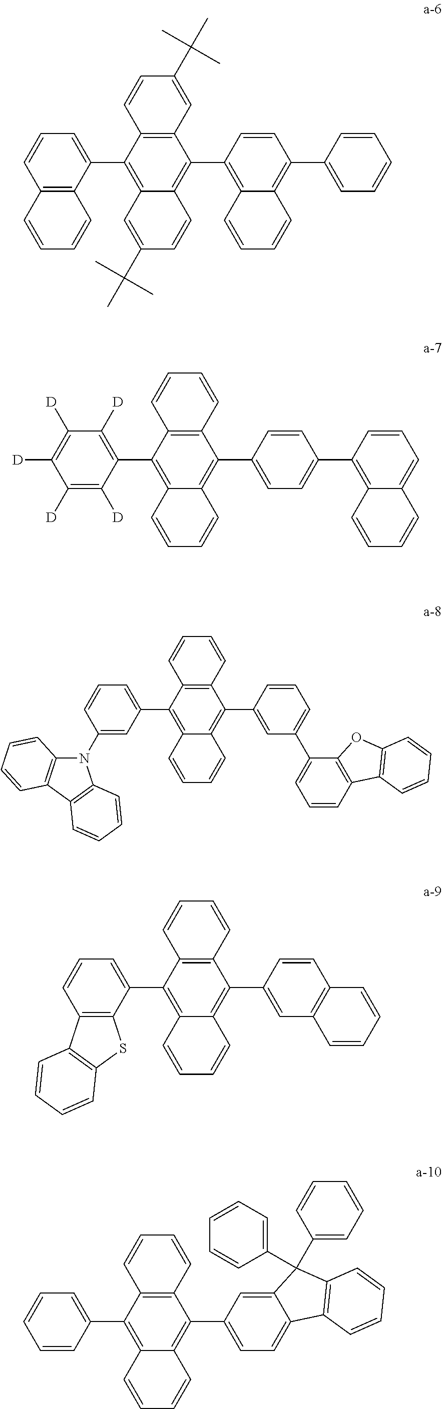

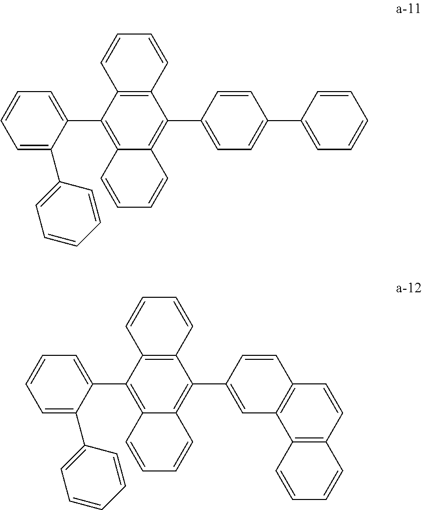

The emission layer 150 may include a dopant such as a styryl derivative (e.g., 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalene-2-yl)vinyl)phenyl)-N-phenylbenzenamine (N-BDAVBi), etc.), perylene and a derivative thereof (e.g., 2,5,8,11-tetra-t-butylperylene (TBPe)), and/or pyrene and a derivative thereof (e.g., 1,1-dipyrene, 1,4-dipyrenylbenzene, 1,4-Bis(N,N-diphenylamino)pyrene), but the dopant is not limited thereto throughout the embodiments of the present disclosure.

The electron transport layer 160 may be provided on the emission layer 150, wherein the electron transport layer 160 includes, for example, tris(8-hydroxyquinolinato)aluminium (Alq3), and/or a material having a nitrogen-containing aromatic ring (e.g., a material including a pyridine ring such as 1,3,5-tri[(3-pyridyl)-phen-3-yl]benzene, a material including a triazine ring such as 2,4,6-tris(3′-(pyridin-3-yl)biphenyl-3-yl)-1,3,5-triazine, and/or a material including an imidazole derivative such as 2-(4-(N-phenylbenzoimidazolyl-1-ylphenyl)-9,10-dinaphthylanthracene). The electron transport layer 160 including an electron transport material having a function of transporting electrons is, for example, provided on the emission layer 150 with a thickness of about 15 nm to about 50 nm.

On the electron transport layer 160, the electron injection layer 170 is provided utilizing, for example, a material including lithium fluoride (LiF), lithium-8-quinolinato (Liq), etc. The electron injection layer 170 having a function of facilitating the injection of electrons from the second electrode 180 is provided with a thickness of about 0.3 nm to about 9 nm.

In addition, the second electrode 180 is provided on the electron injection layer 170. The second electrode is, for example, a cathode. For example, the second electrode 180 may be provided as a reflective electrode with a conductive compound, an alloy, and/or a metal having low work function. The second electrode 180 may include, for example, Li, Mg, Al, Al—Li, calcium (Ca), Mg—In, Mg—Ag, etc. Also, the second electrode 180 may be formed as a transmitting (e.g., penetrating) electrode utilizing ITO, IZO, etc. Each layer may be provided by selecting an appropriate film-forming method such as deposition, sputtering, various suitable coating methods, etc., depending on the material for forming the film.

Hereby, an example of the configuration of the organic EL device 100 according to an embodiment of the present disclosure is described. In the organic EL device 100 including the material for an organic EL device according to an embodiment of the present disclosure, the driving voltage is lowered and the emission efficiency is improved.

Further, a configuration of the organic EL device 100 according to an embodiment of the present disclosure is not limited to the examples. The organic EL device 100 according to an embodiment of the present disclosure may be provided utilizing other various suitable configurations of organic EL devices. For example, the organic EL device 100 may not include (e.g., may exclude) at least one layer of the hole injection layer 130, the electron transport layer 160, and the electron injection layer 170. Also, each layer of the organic EL device 100 may be provided as a monolayer or multilayer.

Further, the organic EL device 100 may include a hole blocking layer between the hole transport layer 140 and the emission layer 150 to reduce or prevent triplet excitons or holes from diffusion into the electron transport layer 160. In addition, the hole blocking layer may include, for example, an oxadiazole derivative, a triazole derivative, a phenanthroline derivative, etc.

### EXAMPLE

Hereinafter, an organic electroluminescent (EL) device according to an embodiment of the present disclosure will be described in more detail with reference to Examples and Comparative Examples. Further, the following Examples are only an illustrative example of an organic EL device according to an embodiment of the present disclosure, and the organic EL device according to embodiments of the present disclosure is not limited to the following examples.

### Synthetic Example 1

### Synthesis of Example Compound 1

Example compound 1 was synthesized according to the following synthetic scheme.

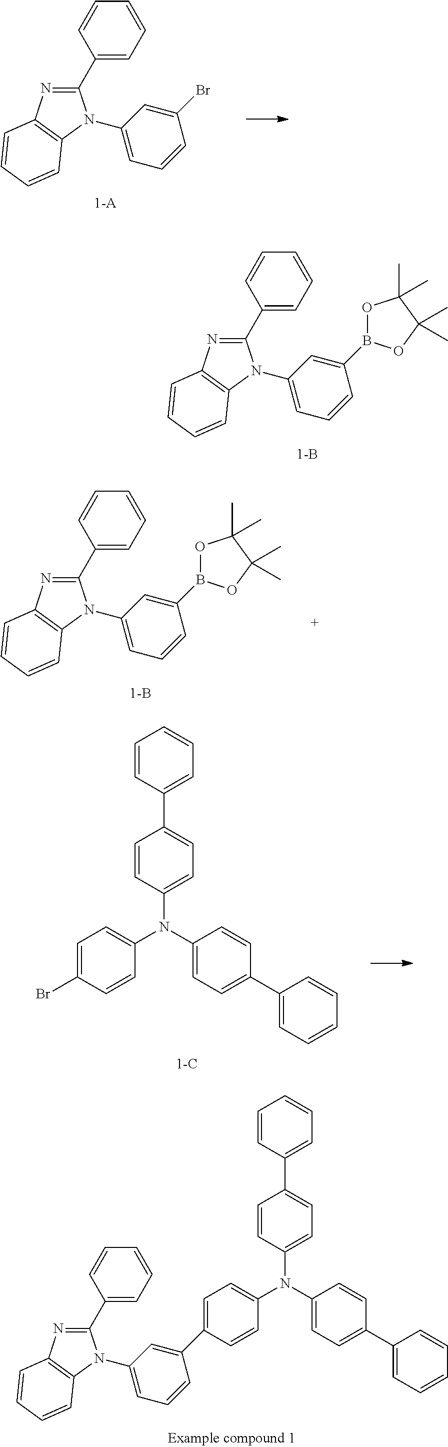 [SMILES: Brc1cccc(-n2c(-c3ccccc3)nc3ccccc32)c1 ; CC1(C)OB(c2cccc(-n3c(-c4ccccc4)nc4ccccc43)c2)OC1(C)C ; CC1(C)OB(c2cccc(-n3c(-c4ccccc4)nc4ccccc43)c2)OC1(C)C ; Brc1ccc(N(c2ccc(-c3ccccc3)cc2)c2ccc(-c3ccccc3)cc2)cc1 ; c1ccc(-c2ccc(N(c3ccc(-c4ccccc4)cc3)c3ccc(-c4cccc(-n5c(-c6ccccc6)nc6ccccc65)c4)cc3)cc2)cc1]

24.00 g of bromine 1-A was dissolved in 400 mL of degassed 1,4-dioxane. 19.25 g of bis(pinacolato)diboron, 1.188 g of tris(dibenzylideneacetone)dipalladium(0), and 10.13 g of potassium acetate (CH₃COOK) were then added, and the resultant was heated and stirred for about 8 hours under argon atmosphere under a reflux condition. After cooling to room temperature, methylene chloride and water were added to perform extraction. A methylene chloride layer was washed with water and saturated sodium chloride (NaCl) aqueous solution, and then anhydride magnesium sulfate (MgSO₄) was added to dehydrate the methylene chloride layer. The solvent was evaporated under reduced pressure, and the resulting product was purified with a silica gel column chromatograph to give 20.70 g of intermediate 1-B (yield: 76%).

3.788 g of intermediate 1-B, 4.788 g of bromine 1-C, and 2.646 g of potassium carbonate were added to 75 mL of degassed toluene, 6 mL of ethanol, and 12 mL of water, and the resultant was stirred at room temperature under argon atmosphere. In addition, 1.106 g of tetrakis(triphenylphosphine)palladium(0) was added, and the resultant was stirred for about 6 hours under heat reflux. After cooling to room temperature, methylene chloride and water were added to perform extraction. A methylene chloride layer was washed with water and a saturated NaCl aqueous solution, and then anhydride MgSO₄ was added to dehydrate the methylene chloride layer. The solvent was evaporated under reduced pressure, and the resulting product was purified with a silica gel column chromatograph to give 4.397 g of Example compound 1 (yield: 69%). The compound was identified by detecting a molecular ion peak utilizing Fast atom bombardment mass spectrometry (FAB-MS) which resulted in the value of 665.28 (C₄₉H₃₅N₃).

### Synthetic Example 2

### Synthesis of Example Compound 4

Example compound 4 was synthesized according to the following synthetic scheme.

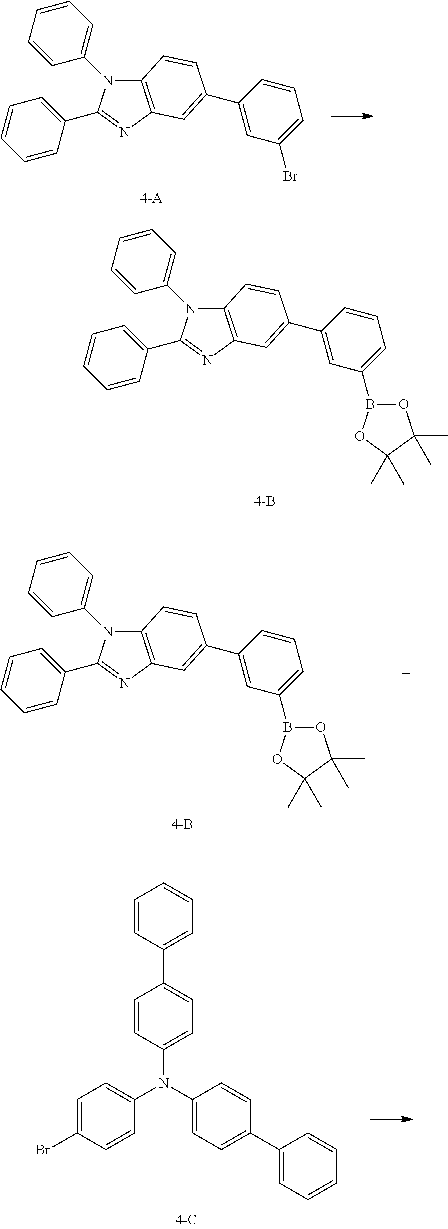 [SMILES: Brc1cccc(-c2ccc3c(c2)nc(-c2ccccc2)n3-c2ccccc2)c1 ; CC1(C)OB(c2cccc(-c3ccc4c(c3)nc(-c3ccccc3)n4-c3ccccc3)c2)OC1(C)C ; CC1(C)OB(c2cccc(-c3ccc4c(c3)nc(-c3ccccc3)n4-c3ccccc3)c2)OC1(C)C ; Brc1ccc(N(c2ccc(-c3ccccc3)cc2)c2ccc(-c3ccccc3)cc2)cc1]

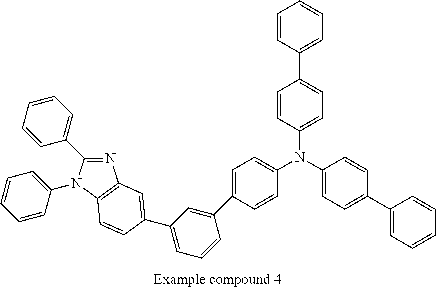 [SMILES: c1ccc(-c2ccc(N(c3ccc(-c4ccccc4)cc3)c3ccc(-c4cccc(-c5ccc6c(c5)nc(-c5ccccc5)n6-c5ccccc5)c4)cc3)cc2)cc1]

6.481 g of bromine 4-A was dissolved in 180 mL of degassed dimethyl sulfoxide (DMSO). 4.257 g of bis(pinacolato)diboron, 0.374 g of [1,1′-bis(diphenylphosphino)ferrocene]dichloropalladium(2), and 4.489 g of CH₃COOK were added, and the resultant was heated and stirred for about 3 hours under argon atmosphere at about 100° C. After cooling to room temperature, methylene chloride and water were added to perform extraction. A methylene chloride layer was washed with water and a saturated NaCl aqueous solution, and then anhydride MgSO₄ was added to dehydrate the methylene chloride layer. The solvent was evaporated under reduced pressure, and the resulting product was purified with a silica gel column chromatograph to give 5.687 g of intermediate 4-B (yield: 79%).

3.010 g of intermediate 4-B, 3.192 g of bromine 4-C, and 1.764 g of potassium carbonate (K₂CO₃) were added to 50 mL of degassed toluene, 4 mL of ethanol, and 8 mL of water, and the resultant was stirred at room temperature under argon atmosphere. In addition, 0.737 g of tetrakis(triphenylphosphine)palladium(0) was added, and the resultant was stirred for about 6 hours under heat reflux. After cooling to room temperature, methylene chloride and water were added to perform extraction. A methylene chloride layer was washed with water and a saturated NaCl aqueous solution, and then anhydride MgSO₄ was added to dehydrate the methylene chloride layer. The solvent was evaporated under reduced pressure, and the resulting product was purified with a silica gel column chromatograph to give 3.124 g of Example compound 4 (yield: 66%). The compound was identified by detecting a molecular ion peak utilizing FAB-MS which resulted in the value of 741.31 (C₅₅H₃₉N₃).

### Synthetic Example 3

### Synthesis of Example Compound 81

Example compound 81 was synthesized according to the following synthetic scheme.

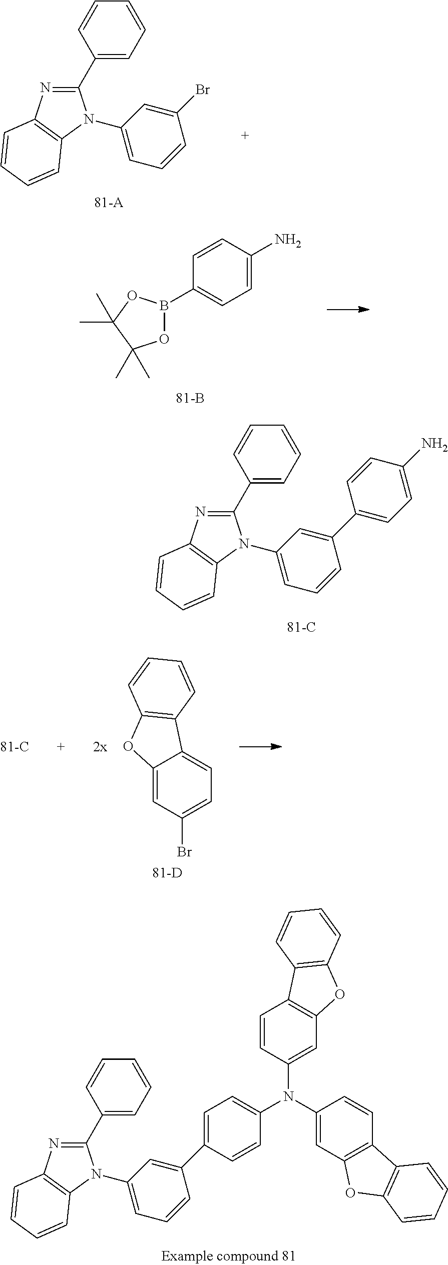 [SMILES: Brc1cccc(-n2c(-c3ccccc3)nc3ccccc32)c1 ; CC1(C)OB(c2ccc(N)cc2)OC1(C)C ; Nc1ccc(-c2cccc(-n3c(-c4ccccc4)nc4ccccc43)c2)cc1 ; Brc1ccc2c(c1)oc1ccccc12 ; c1ccc(-c2nc3ccccc3n2-c2cccc(-c3ccc(N(c4ccc5c(c4)oc4ccccc45)c4ccc5c(c4)oc4ccccc45)cc3)c2)cc1]

Into a 500 mL 3-neck flask, 2.32 g of 4-(4,4,5,5-tetramethyl-1,3,2-dioxaborolan-2-yl)aniline (compound 81-B), 3.52 g of bromine 81-A, 0.54 g of Pd(PPh₃)₄, and 2.79 g of K₂CO₃ were added, and the resultant was heated and stirred in a mixed solvent of 200 mL of toluene, 32 mL of water, and 12 mL of ethanol for about 12 hours at about 90° C. After air cooling, water was added to separate an organic layer, and a solvent was evaporated. The resulting crude product was purified with a silica gel column chromatograph (utilizing a mixed solvent of dichloromethane and hexane), and then recrystallization was performed with a mixed solvent of ethyl acetate/hexane to give 1.93 g of intermediate 81-C as a white solid (yield: 53%). The compound was identified by detecting a molecular ion peak utilizing FAB-MS which resulted in the value of 361.16 (C₂₅H₁₉N₃).

Under argon atmosphere, into a 200 mL 3-neck flask, 1.92 g of intermediate 81-C, 3.15 g of bromodibenzofuran (compound 81-D), 0.384 g of Pd₂(dba)₃.CHCl₃, 2.06 g of sodium tert-butoxide (t-BuONa), 65 mL of dehydrated toluene, and 0.56 mL of 2 M tert-butylphosphine/dehydrated toluene ((t-Bu)3P/dehydrated toluene) solution were added, and the resultant was stirred for about 7 hours under heat reflux. After air cooling, water was added to separate an organic layer, and a solvent was evaporated. The resulting crude product was purified with a silica gel column chromatograph (utilizing a mixed solvent of dichloromethane and hexane), and then recrystallization was performed with a mixed solvent of methylene chloride/ethanol to give 3.30 g Example compound 81 as a white solid (yield: 89%). The compound was identified by detecting a molecular ion peak utilizing FAB-MS which resulted in the value of 693.24 (C₄₉H₃₁N₃O₂).

### Synthetic Example 4

### Synthesis of Example Compound 84

Example compound 84 was synthesized according to the following synthetic scheme.

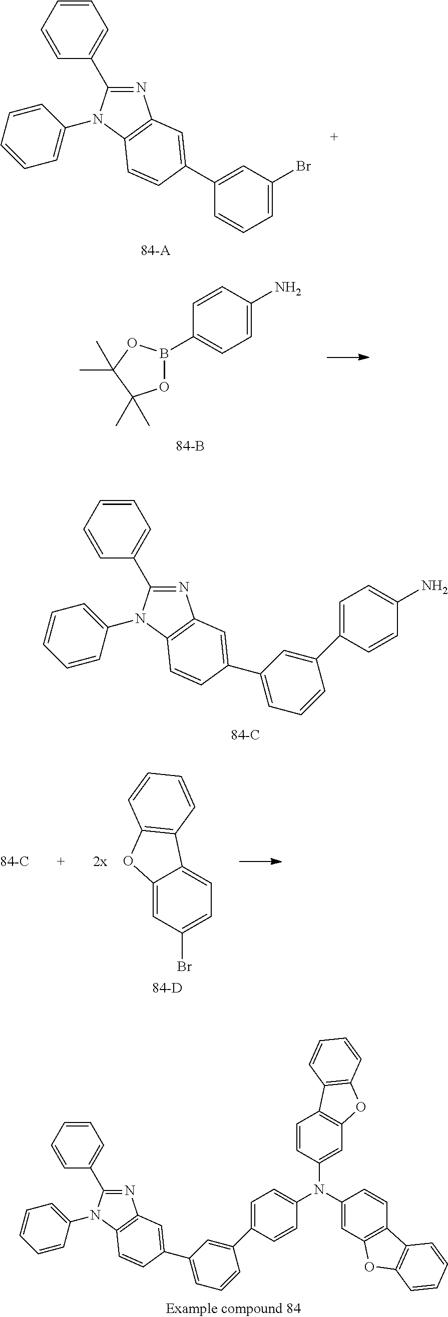 [SMILES: Brc1cccc(-c2ccc3c(c2)nc(-c2ccccc2)n3-c2ccccc2)c1 ; CC1(C)OB(c2ccc(N)cc2)OC1(C)C ; Nc1ccc(-c2cccc(-c3ccc4c(c3)nc(-c3ccccc3)n4-c3ccccc3)c2)cc1 ; Brc1ccc2c(c1)oc1ccccc12 ; c1ccc(-c2nc3cc(-c4cccc(-c5ccc(N(c6ccc7c(c6)oc6ccccc67)c6ccc7c(c6)oc6ccccc67)cc5)c4)ccc3n2-c2ccccc2)cc1]

Into a 1000 mL 3-neck flask, 8.57 g of bromine 84-A, 4.64 g of 4-(4,4,5,5-tetramethyl-1,3,2-dioxaborolan-2-yl)aniline (compound 84-B), 1.04 g of Pd(PPh₃)₄, and 5.59 g of K₂CO₃ were added, and the resultant was heated and stirred in a mixed solvent of 400 mL of toluene, 64 mL of water, and 25 mL of ethanol for about 12 hours at about 90° C. After air cooling, water was added to separate an organic layer, and a solvent was evaporated. The resulting crude product was purified with a silica gel column chromatograph (utilizing a mixed solvent of dichloromethane and hexane), and then recrystallization was performed with a mixed solvent of ethyl acetate/hexane to give 5.90 g of intermediate 84-C as a white solid (yield: 67%). The compound was identified by detecting a molecular ion peak utilizing FAB-MS which resulted in the value of 437.19 (C₃₁H₂₃N₃).

Under argon atmosphere, into a 300 mL 3-neck flask, 4.78 g of intermediate 84-C, 8.16 g of 3-bromodibenzofuran (compound 84-D), 0.791 g of Pd₂(dba)₃.CHCl₃, and 4.20 g of t-BuONa were added, and the resultant was stirred in a 70 mL of dehydrated toluene solvent for about 8 hours under heat reflux. After air cooling, water was added to separate an organic layer, and a solvent was evaporated. The resulting crude product was purified with a silica gel column chromatograph (utilizing a mixed solvent of dichloromethane and hexane), and then recrystallization was performed with a mixed solvent of methylene chloride/ethanol to give 5.30 g of Example compound 84 as a white solid (yield: 63%). The compound was identified by detecting a molecular ion peak utilizing FAB-MS which resulted in the value of 769.27 (C₅₅H₃₅N₃O₂).

### Synthetic Example 5

### Synthesis of Example Compound 104

Example compound 104 was synthesized according to the following synthetic scheme.

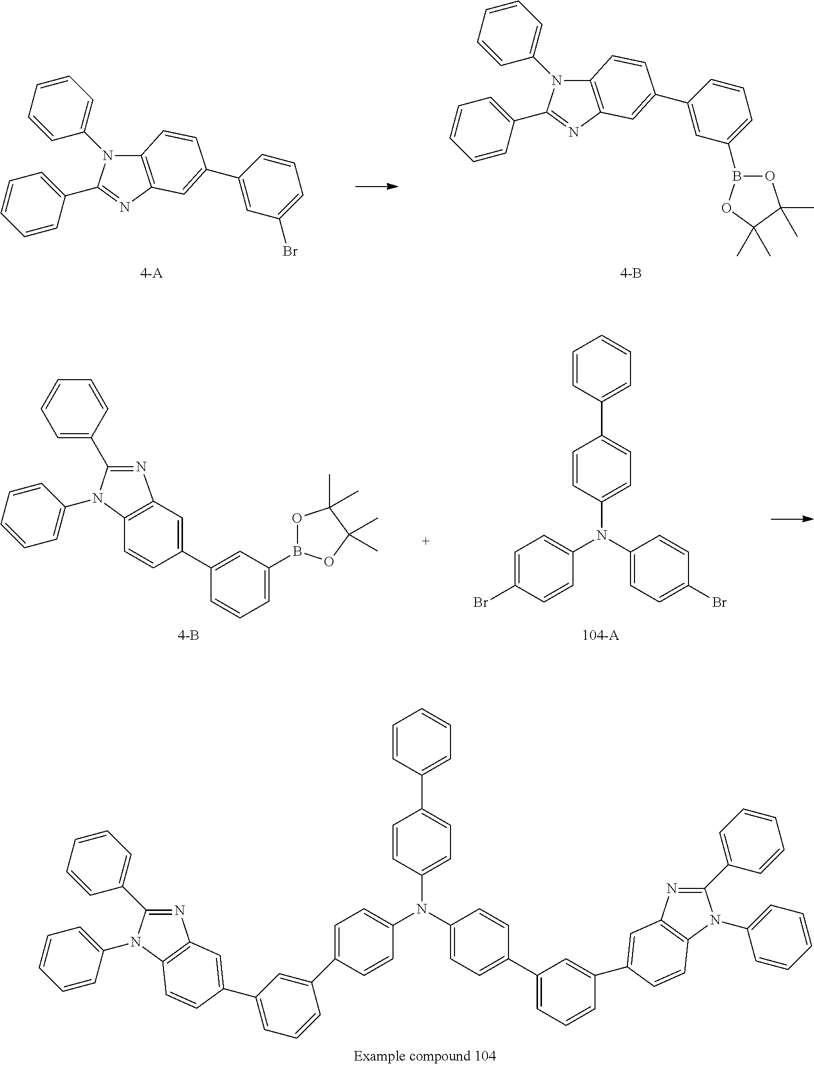 [SMILES: Brc1cccc(-c2ccc3c(c2)nc(-c2ccccc2)n3-c2ccccc2)c1 ; CC1(C)OB(c2cccc(-c3ccc4c(c3)nc(-c3ccccc3)n4-c3ccccc3)c2)OC1(C)C ; CC1(C)OB(c2cccc(-c3ccc4c(c3)nc(-c3ccccc3)n4-c3ccccc3)c2)OC1(C)C ; Brc1ccc(N(c2ccc(Br)cc2)c2ccc(-c3ccccc3)cc2)cc1 ; c1ccc(-c2ccc(N(c3ccc(-c4cccc(-c5ccc6c(c5)nc(-c5ccccc5)n6-c5ccccc5)c4)cc3)c3ccc(-c4cccc(-c5ccc6c(c5)nc(-c5ccccc5)n6-c5ccccc5)c4)cc3)cc2)cc1]

As described in Synthesis Example 2, intermediate 4-B was synthesized. Into a 1000 mL 3-neck flask, 18.90 g of intermediate 4-B, 9.57 g of dibromine 104-A, 4.52 g of Pd(PPh₃)₄, and 11.06 g of sodium carbonate (Na₂Co₃) were added, and the resultant was heated and stirred in a mixed solvent of 400 mL of toluene, 80 mL of water, and 40 mL of ethanol for about 9 hours at about 90° C. After air cooling, water was added to separate an organic layer, and a solvent was evaporated. The resulting crude product was purified with a silica gel column chromatograph (utilizing a mixed solvent of dichloromethane and hexane), and then recrystallization was performed with a mixed solvent of methylene chloride/ethanol to give 9.90 g of Example compound 104 as a white solid (yield: 49%). The compound was identified by detecting a molecular ion peak utilizing FAB-MS which resulted in the value of 1009.41 (C₇₄H₅₁N₅).

(Manufacture of Organic Electroluminescent Device)

Thereafter, an organic electroluminescent (EL) device was manufactured according to the manufacturing method below. First, an ITO-glass substrate, which has previously passed through patterning and washing treatment, was surface treated with ultraviolet ozone (O₃). In addition, the thickness of the ITO film (i.e. a first electrode) was about 150 nm. After ozone treatment, the substrate was washed. After the washing was completed, the substrate was set in a glass bell jar type deposition apparatus for the formation of the organic layers. The deposition was performed under a vacuum level of about 10⁻⁴ to about 10⁻⁵ Pa in the order of a hole injection layer, a hole transport layer (HTL), an emission layer, and an electron transport layer. As a material for the hole injection layer, 2-TNATA was utilized, and the thickness of the hole injection layer was about 60 nm. As a material for the HTL, those indicated in Table 1 were utilized, and the thickness of the HTL was about 30 nm.

In addition, the thickness of the emission layer was about 25 nm. A host of an emission material was 9,10-di(2-naphthyl)anthracene (ADN). As a dopant, 2,5,8,11-tetera-t-butylphenylene (TBP) was utilized. An amount of the dopant was 3 wt % with respect to a weight of the host (e.g., based on 100 wt % of the host). As a material for the electron transport layer, Alq3 was utilized, and the thickness of the electron transport layer was about 25 nm.

In succession (e.g., subsequently), the substrate was moved to a glass bell jar type deposition apparatus for the formation of the metal films, where an electron injection layer and a cathode material were deposited (e.g., deposited sequentially) under a vacuum level of about 10⁻⁴ to about 10⁻⁵ Pa. As a material for the electron injection layer, LiF was utilized, and the thickness of the electron injection layer was about 1.0 nm. As a material for a second electrode, Al was utilized, and the thickness of the second electrode was about 100 nm.

TABLE 1

| | | Current | | Emission |
|---|---|---|---|---|
| | | density | Voltage | efficiency |
| | HTL | (mA/cm²) | (V) | (cd/A) |
| Example 1 | Example | 10 | 6.5 | 7.2 |
| | compound 1 | | | |
| Example 2 | Example | 10 | 6.6 | 7.1 |
| | compound 4 | | | |
| Comparative | Comparative | 10 | 7.9 | 5.5 |
| Example 1 | Example | | | |
| | compound C1 | | | |
| Example 3 | Example | 10 | 6.6 | 7.2 |
| | compound 81 | | | |
| Example 4 | Example | 10 | 6.7 | 7.1 |
| | compound 84 | | | |
| Comparative | Comparative | 10 | 8.1 | 5.3 |
| Example 2 | Example | | | |
| | compound C2 | | | |
| Example 5 | Example | 10 | 7.0 | 6.7 |
| | compound 104 | | | |
| Comparative | Comparative | 10 | 8.1 | 5.5 |
| Example 3 | Example | | | |
| | compound C3 | | | |

In Table 1, Comparative Example compounds C1 to C3 are represented by the following Formulae. Comparative Example compounds C1 to C3 are amine derivatives having a benzimidazole structure, and the carbon located at the 2 position of the benzimidazole structure binds to a nitrogen atom of an amine (e.g., amine group) through an m-phenylene group.

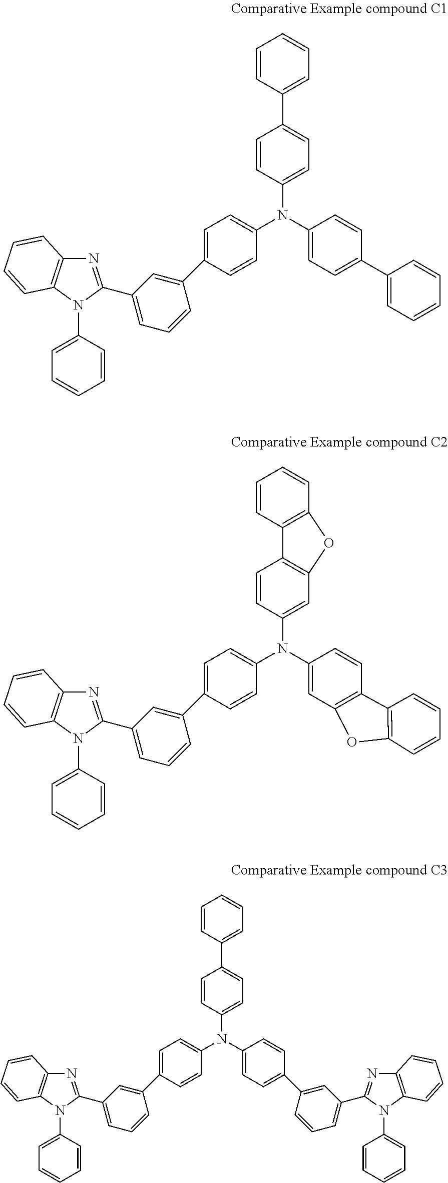 [SMILES: c1ccc(-c2ccc(N(c3ccc(-c4ccccc4)cc3)c3ccc(-c4cccc(-c5nc6ccccc6n5-c5ccccc5)c4)cc3)cc2)cc1 ; c1ccc(-n2c(-c3cccc(-c4ccc(N(c5ccc6c(c5)oc5ccccc56)c5ccc6c(c5)oc5ccccc56)cc4)c3)nc3ccccc32)cc1 ; c1ccc(-c2ccc(N(c3ccc(-c4cccc(-c5nc6ccccc6n5-c5ccccc5)c4)cc3)c3ccc(-c4cccc(-c5nc6ccccc6n5-c5ccccc5)c4)cc3)cc2)cc1]

### Comparative Example Compound C1

The Comparative Example compound C1 was synthesized according to the following synthetic scheme.

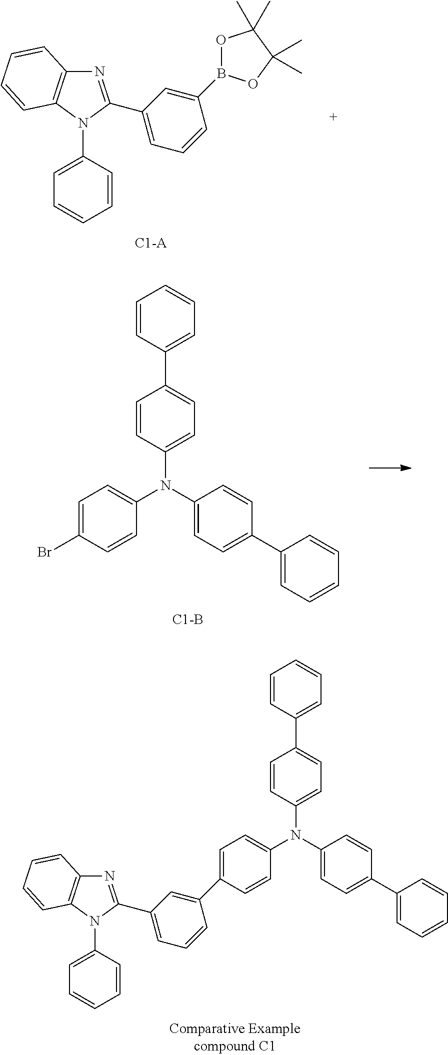 [SMILES: CC1(C)OB(c2cccc(-c3nc4ccccc4n3-c3ccccc3)c2)OC1(C)C ; Brc1ccc(N(c2ccc(-c3ccccc3)cc2)c2ccc(-c3ccccc3)cc2)cc1 ; "C1-B" ; c1ccc(-c2ccc(N(c3ccc(-c4ccccc4)cc3)c3ccc(-c4cccc(-c5nc6ccccc6n5-c5ccccc5)c4)cc3)cc2)cc1]

Into a 100 mL flask, 4.36 g of benzimidazole compound C1-A (which is synthesized according to the synthetic method disclosed in Japanese Patent Publication No. 2007-269772, the entire content of which is incorporated herein by reference), 4.76 g of arylamine compound C1-B, 2.76 g of K₂CO₃, and 1.16 g of tetrakis(triphenylphosphine)palladium were added, and 60 mL of degassed toluene, 10.5 mL of ethanol, and 4.5 mL of water were added, and the resultant was heated for about 6 hours at about 100° C. under argon atmosphere. After the reaction was completed, methylene chloride was added to the reactant solution, and extraction was performed. Thereafter, an organic layer was dehydrated with MgSO₄, and then the solvent was evaporated under a reduced pressure. The residual (debris) was purified with a silica gel column chromatograph to give 4.25 g of Comparative Example compound C1 (yield: 58%). The compound was identified by detecting a molecular ion peak utilizing FAB-MS which resulted in the value of 665.28 (C₄₉H₃₅N₃).

Comparative Example compound C2 was synthesized according to the following synthetic scheme.

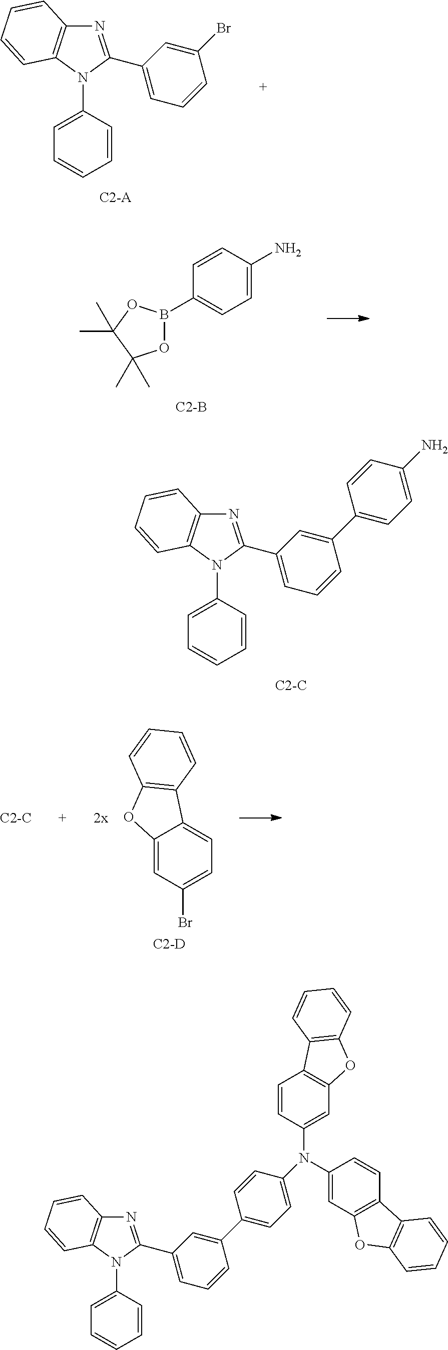 [SMILES: Brc1cccc(-c2nc3ccccc3n2-c2ccccc2)c1 ; CC1(C)OB(c2ccc(N)cc2)OC1(C)C ; Nc1ccc(-c2cccc(-c3nc4ccccc4n3-c3ccccc3)c2)cc1 ; Brc1ccc2c(c1)oc1ccccc12 ; c1ccc(-n2c(-c3cccc(-c4ccc(N(c5ccc6c(c5)oc5ccccc56)c5ccc6c(c5)oc5ccccc56)cc4)c3)nc3ccccc32)cc1]

### Comparative Example Compound C2

Into a 1000 mL 3-neck flask, 3.71 g of 4-(4,4,5,5-tetramethyl-1,3,2-dioxaborolan-2-yl)aniline (compound C2-B), 5.63 g of bromine C2-A, 0.87 g of Pd(PPh₃)₄, and 4.49 g of K₂CO₃ were added, and the resultant was heated and stirred in a mixed solvent of 300 mL of toluene, 51 mL of water, and 19 mL of ethanol for about 10 hours at about 90° C. After air cooling, water was added to separate an organic layer, and the solvent was evaporated. The resulting crude product was purified with a silica gel column chromatograph (utilizing a mixed solvent of dichloromethane and hexane), and then recrystallization was performed with a mixed solvent of ethyl acetate/hexane to give 2.97 g of intermediate C2-C as a white solid (yield: 51%). The compound was identified by detecting a molecular ion peak utilizing FAB-MS which resulted in the value of 361.16 (C₂₅H₁₉N₃).

Under Ar atmosphere, into a 200 mL 3-neck flask, 3.07 g of intermediate C2-C, 5.04 g of 3-bromodibenzofuran (compound C2-D), 0.576 g of Pd₂(dba)₃.CHCl₃, 3.30 g of t-BuONa, 105 mL of dehydrated toluene, and 0.90 mL of 2 M (t-Bu)3P/dehydrated toluene solution were added, and the resultant was stirred for about 8 hours under heat reflux. After air cooling, water was added to separate an organic layer, and the solvent was evaporated. The resulting crude product was purified with a silica gel column chromatograph (utilizing a mixed solvent of dichloromethane and hexane), and then recrystallization was performed with a mixed solvent of methylene chloride/ethanol to give 3.31 g of Comparative Example compound C2 as a white solid (yield: 56%). The compound was identified by detecting a molecular ion peak utilizing FAB-MS which resulted in the value of 693.24 (C₄₉H₃₁N₃O₂).

### Comparative Example Compound C3

Comparative Example compound C3 was synthesized according to the following synthetic scheme.

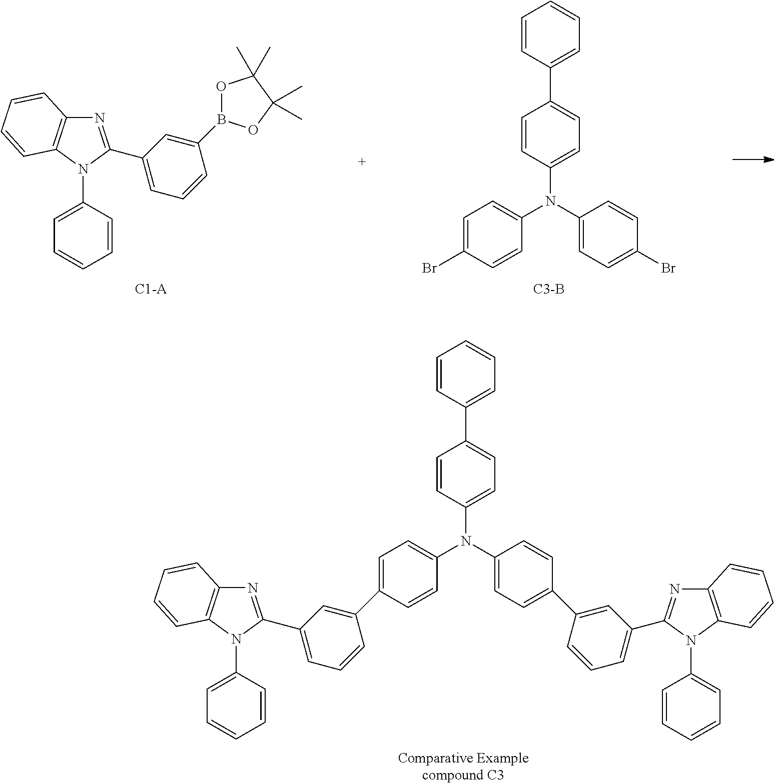

Into a 500 mL 3-neck flask, 9.45 g of benzimidazole compound C1-A (which is synthesized according to the synthetic method disclosed in Japanese Patent Publication No. 2007-269772, the entire content of which is incorporated herein by reference), 11.43 g of dibromine C3-B, 2.32 g of Pd(PPh₃)₄, and 5.50 g of sodium carbonate were added, and then the resultant was heated and stirred in a mixed solvent of 200 mL of toluene, 40 mL of water, and 20 mL of ethanol for about 8 hours at about 90° C. After air cooling, water was added to separate an organic layer, and the solvent was evaporated. The resulting crude product was purified with a silica gel column chromatograph (utilizing a mixed solvent of dichloromethane and hexane), and the recrystallization was performed with a mixed solvent of methylene chloride/ethanol to give 9.82 g of Comparative Example compound C3 as a white solid (yield: 51%). The compound was identified by detecting a molecular ion peak utilizing FAB-MS which resulted in the value of 857.35 (C₆₂H₄₃N₅).

(Characterization)

Emission life expectancy and driving voltage of the manufactured organic EL device were measured. In addition, to test the electroluminescent characteristics of the manufactured organic EL device 100, brightness distribution characteristic measurement system No. C9920-11 from HAMAMATSU Photonics co. was utilized. Additionally, current density was measured at about 10 mA/cm², and half-life expectancy was measured at about 1000 cd/m². The results are shown in Table 1.

Referring to Table 1, it has been found that the driving voltage is lowered and the emission efficiency is enhanced in Examples 1 to 5 with respect to (e.g., compared to) Comparative Examples 1 to 3, wherein, in Examples 1 to 5, a hole transport layer (HTL) was provided to include the amine derivative according to an embodiment of the present disclosure.

For example, it has been found that Examples 1 to 5 each show a lowered driving voltage and an enhanced emission efficiency with respect to Comparative Examples 1 to 3, wherein, in Examples 1 to 5, the HTL was provided to include the amine derivative according to an embodiment of the present disclosure, and in Comparative Examples 1 to 3, an amine derivative was utilized in which a nitrogen atom of an amine (e.g., amine group) binds to a carbon at the position 2 of a benzimidazole structure through an m-phenylene group. Also, when comparing amine derivatives having the same functional groups, although both Example compound 1 and Comparative Example compound C1 have a benzimidazole structure and a biphenylene group, Example 1 utilizing Example compound 1 shows a lowered driving voltage and an enhanced emission efficiency when compared with Comparative Example 1, which utilizes Comparative Example compound C1. Although both Example compound 81 and Comparative Example compound C2 have a benzimidazole structure and a dibenzofuranyl group, when comparing Example 3 and Comparative Example 2, which respectively utilizes Example compound 81 and Comparative Example compound C2, Example 3 utilizing Example compound 81 shows a lowered driving voltage and an enhanced emission efficiency. In addition, although both Example compound 104 and Comparative Example compound C3 have two benzimidazole structures, when comparing Example 5 and Comparative Example 3, which respectively utilizes Example compound 104 and Comparative Example compound C3, Example 5 utilizing Example compound 104 shows a lowered driving voltage and an enhanced emission efficiency.

In Example compounds 1, 4, 81, 84, and 104, a nitrogen atom in an amine (e.g., amine group) binds to a carbon atom located at positions 4 to 7, or binds to a nitrogen atom located at the 1 position of a benzimidazole structure through an m-phenylene group. Meanwhile, in Comparative Example compounds C1 to C3, a nitrogen atom of an amine (e.g., amine group) binds to a carbon located at the position 2 of a benzimidazole structure through an m-phenylene group. Due to the difference in structures, spreadability of π electron conjugation between nitrogen atoms of Example compounds 1, 4, 81, 84, and 104 becomes narrower than that of Comparative Example compounds C1 to C3, and thus transportation of electrons from the emission layer to the hole transport layer may be reduced or inhibited. Thus, in Examples 1 to 5 utilizing Example compounds 1, 4, 81, 84, and 104 respectively, it has been shown that the driving voltage is lowered and the emission efficiency is enhanced.

As such, in the Examples, the driving voltage of an organic EL device was lowered and the emission efficiency was significantly improved in blue to green regions.

Hereby, in an embodiment of the present disclosure, because the material for an organic EL device includes the amine derivative represented by Formula 1, in the organic EL device utilizing the same, the driving voltage is lowered and the emission efficiency is significantly improved. Therefore, the material for an organic EL device according to an embodiment of the present disclosure is useful for commercialization for various usages.

According to the present disclosure as described above, an organic EL device may have a lowered driving voltage and an enhanced emission efficiency.

Expressions such as “at least one of,” when preceding a list of elements, modify the entire list of elements and do not modify the individual elements of the list. Further, the use of “may” when describing embodiments of the present invention refers to “one or more embodiments of the present invention.” Also, the term “exemplary” is intended to refer to an example or illustration. It will be understood that when an element or layer is referred to as being “on”, “connected to”, “coupled to”, or “adjacent to” another element or layer, it can be directly on, connected to, coupled to, or adjacent to the other element or layer, or one or more intervening elements or layers may be present. In contrast, when an element or layer is referred to as being “directly on,” “directly connected to”, “directly coupled to”, or “immediately adjacent to” another element or layer, there are no intervening elements or layers present.

As used herein, the term “substantially,” “about,” and similar terms are used as terms of approximation and not as terms of degree, and are intended to account for the inherent deviations in measured or calculated values that would be recognized by those of ordinary skill in the art. Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein. All such ranges are intended to be inherently described in this specification such that amending to expressly recite any such subranges would comply with the requirements of 35 U.S.C. §112, first paragraph, or 35 U.S.C. §112(a), and 35 U.S.C. §132(a).

The above-disclosed subject matter is to be considered illustrative and not restrictive, and the appended claims (and equivalents thereof) are intended to cover all such modifications, enhancements, and other embodiments, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. An amine derivative, represented by Formula 1: 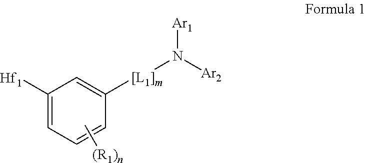
wherein in Formula 1, Ar₁ and Ar₂ are each independently a substituted or unsubstituted aryl group having 6 to 30 carbon atoms for forming a ring, or a substituted or unsubstituted heteroaryl group having 2 to 30 carbon atoms for forming a ring;
L₁ is a divalent linking group;
R₁ is a substituted or unsubstituted alkyl group having 1 to 10 carbon atoms in a straight chain, branched chain, or ring form; a substituted or unsubstituted aryl group having 6 to 20 carbon atoms for forming a ring; a substituted or unsubstituted heteroaryl group having 1 to 20 carbon atoms for forming a ring; or an alkyl group, an aryl group, or a heteroaryl group formed through condensation of adjacent substituents;
n and m are each independently an integer from 0 to 4; and
Hf₁ is represented by Formula 2-1 or Formula 2-2; 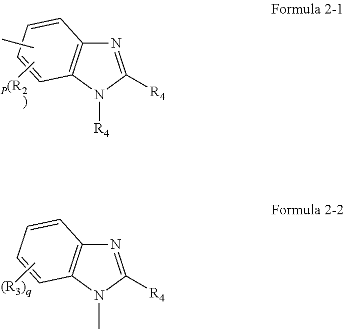
wherein in Formula 2-1 and 2-2, R₂, R₃, and R₄ are each independently a substituted or unsubstituted alkyl group having 1 to 10 carbon atoms in a straight chain, branched chain, or ring form; a substituted or unsubstituted aryl group having 6 to 20 carbon atoms for forming a ring; a substituted or unsubstituted heteroaryl group having 1 to 20 carbon atoms for forming a ring; or an aryl group or a heteroaryl group formed through condensation of adjacent substituents;
p is an integer from 0 to 3; and
q is an integer from 0 to 4.

2. The amine derivative of claim 1, wherein L₁ is a substituted or unsubstituted arylene group having 6 to 30 carbon atoms for forming a ring, or a substituted or unsubstituted heteroarylene group having 2 to 30 carbon atoms for forming a ring.

3. The amine derivative of claim 1, wherein Ar₁ and Ar₂ are each independently a non-nitrogen atom-containing aryl group or heteroaryl group which is unsubstituted or substituted with a substituent which does not include a nitrogen atom.

4. The amine derivative of claim 1, wherein Ar₁ is a non-nitrogen atom-containing aryl group or heteroaryl group which is unsubstituted or substituted with a substituent which does not include a nitrogen atom, and Ar₂ is represented by Formula 3: 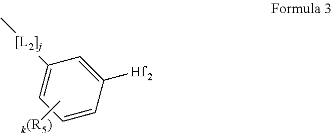
wherein in Formula 3,
L₂ is a divalent linking group;
R₅ is a substituted or unsubstituted alkyl group having 1 to 10 carbon atoms in a straight chain, branched chain, or ring form; a substituted or unsubstituted aryl group having 6 to 20 carbon atoms for forming a ring; a substituted or unsubstituted heteroaryl group having 1 to 20 carbon atoms for forming a ring; or an alkyl group, an aryl group, or a heteroaryl group formed through condensation of adjacent substituents;
j and k are each independently an integer from 0 to 4; and
Hf₂ is represented by Formula 4-1 or Formula 4-2; 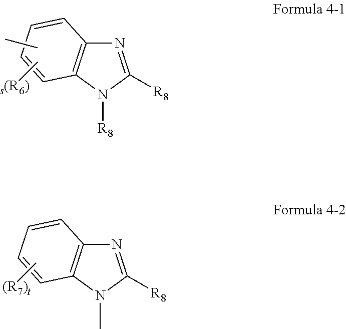
wherein in Formula 4-1 and 4-2,
R₆, R₇, and R₈ are each independently a substituted or unsubstituted alkyl group having 1 to 10 carbon atoms in a straight chain, branched chain, or ring form; a substituted or unsubstituted aryl group having 6 to 20 carbon atoms for forming a ring; a substituted or unsubstituted heteroaryl group having 1 to 20 carbon atoms for forming a ring; or an aryl group or a heteroaryl group formed through condensation of adjacent substituents;
s is an integer from 0 to 3; and
t is an integer from 0 to 4.

5. The amine derivative of claim 1, wherein the amine derivative represented by Formula 1 is at least one of compounds 1 to 130:

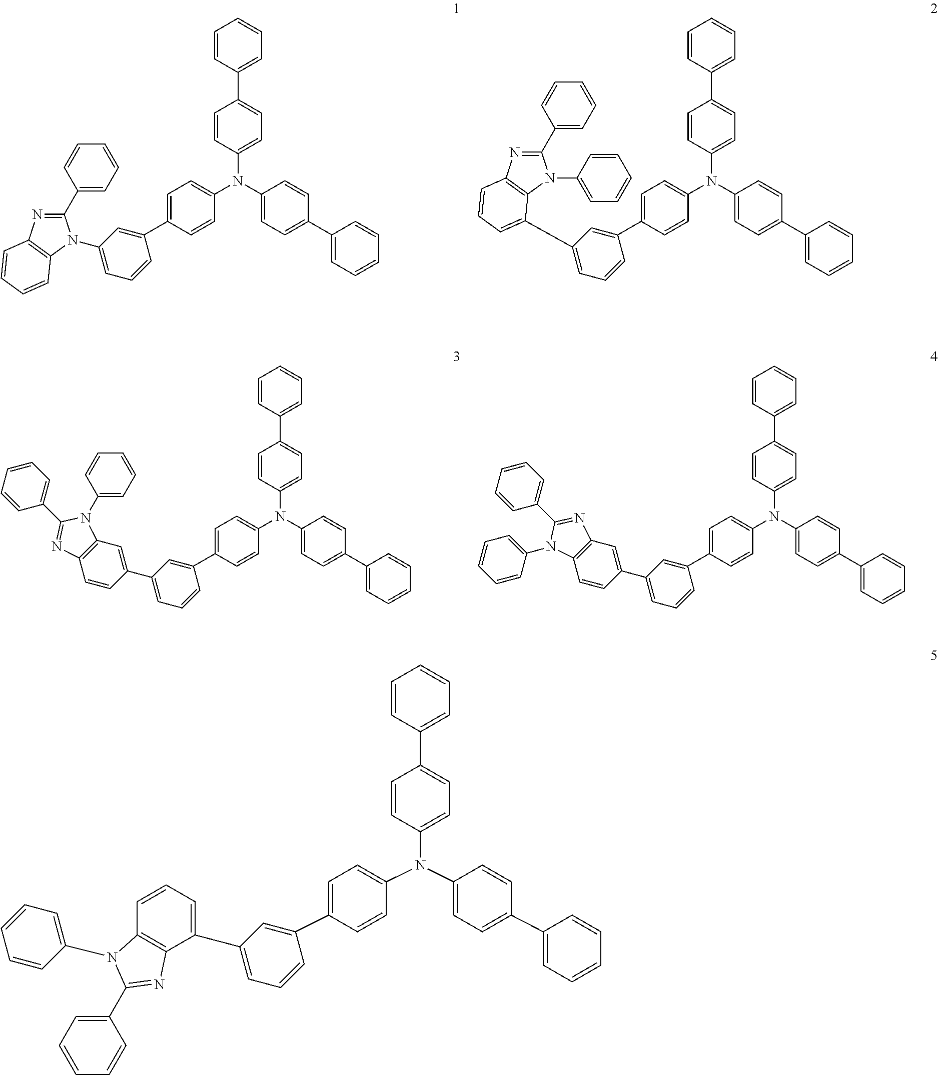 [SMILES: c1ccc(-c2ccc(N(c3ccc(-c4ccccc4)cc3)c3ccc(-c4cccc(-n5c(-c6ccccc6)nc6ccccc65)c4)cc3)cc2)cc1 ; c1ccc(-c2ccc(N(c3ccc(-c4ccccc4)cc3)c3ccc(-c4cccc(-c5cccc6nc(-c7ccccc7)n(-c7ccccc7)c56)c4)cc3)cc2)cc1 ; c1ccc(-c2ccc(N(c3ccc(-c4ccccc4)cc3)c3ccc(-c4cccc(-c5ccc6nc(-c7ccccc7)n(-c7ccccc7)c6c5)c4)cc3)cc2)cc1 ; c1ccc(-c2ccc(N(c3ccc(-c4ccccc4)cc3)c3ccc(-c4cccc(-c5ccc6c(c5)nc(-c5ccccc5)n6-c5ccccc5)c4)cc3)cc2)cc1 ; c1ccc(-c2ccc(N(c3ccc(-c4ccccc4)cc3)c3ccc(-c4cccc(-c5cccc6c5nc(-c5ccccc5)n6-c5ccccc5)c4)cc3)cc2)cc1]

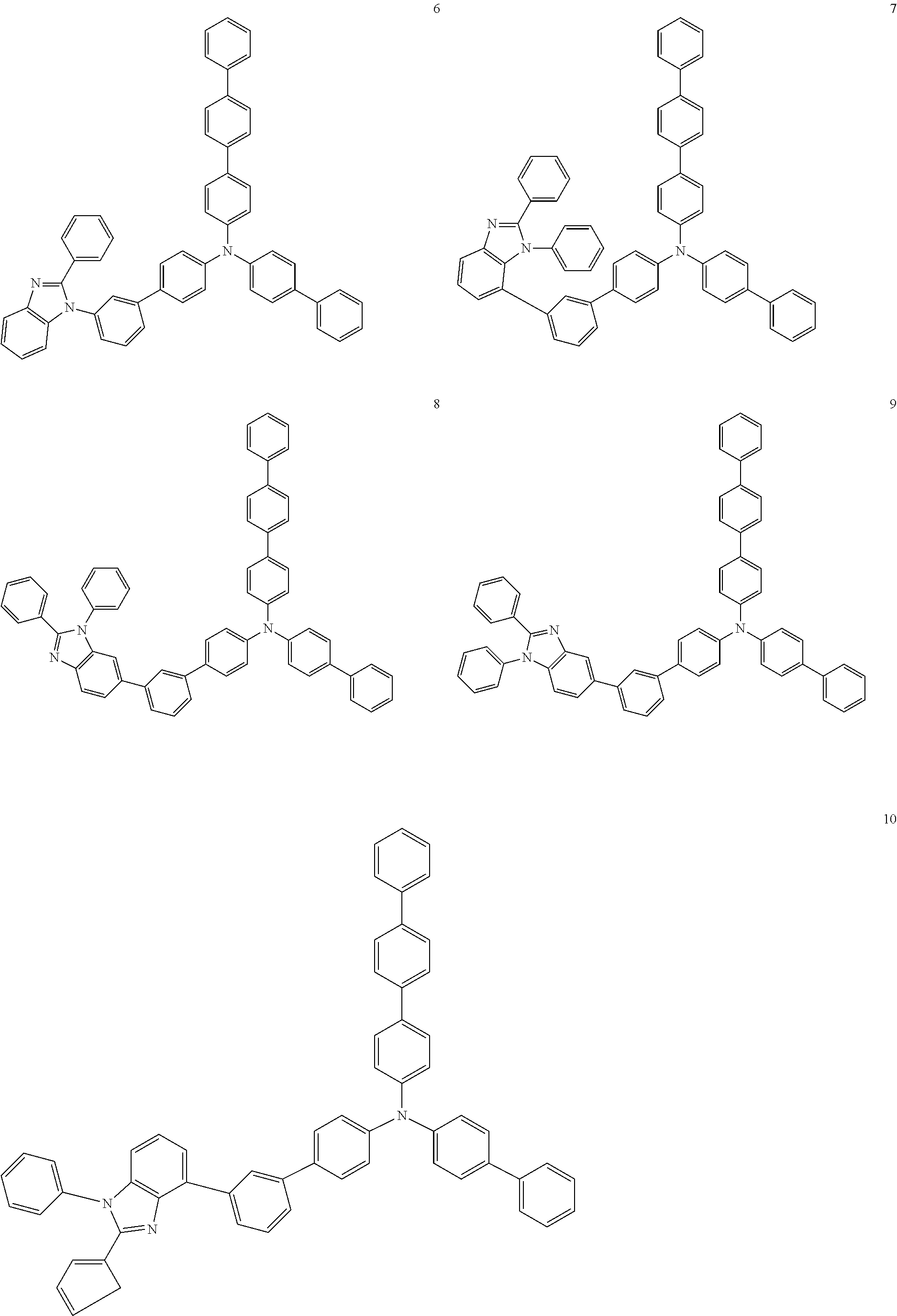

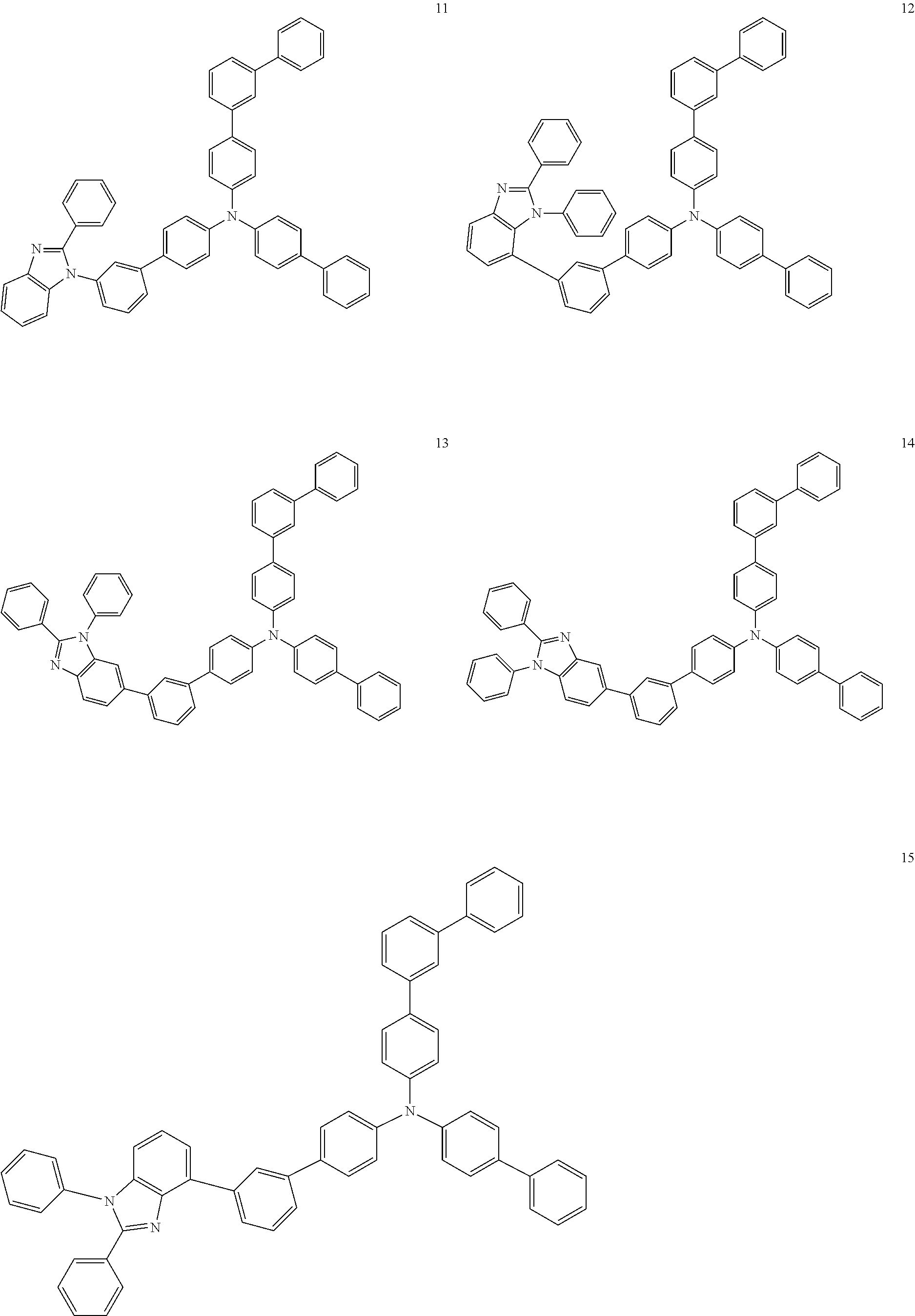

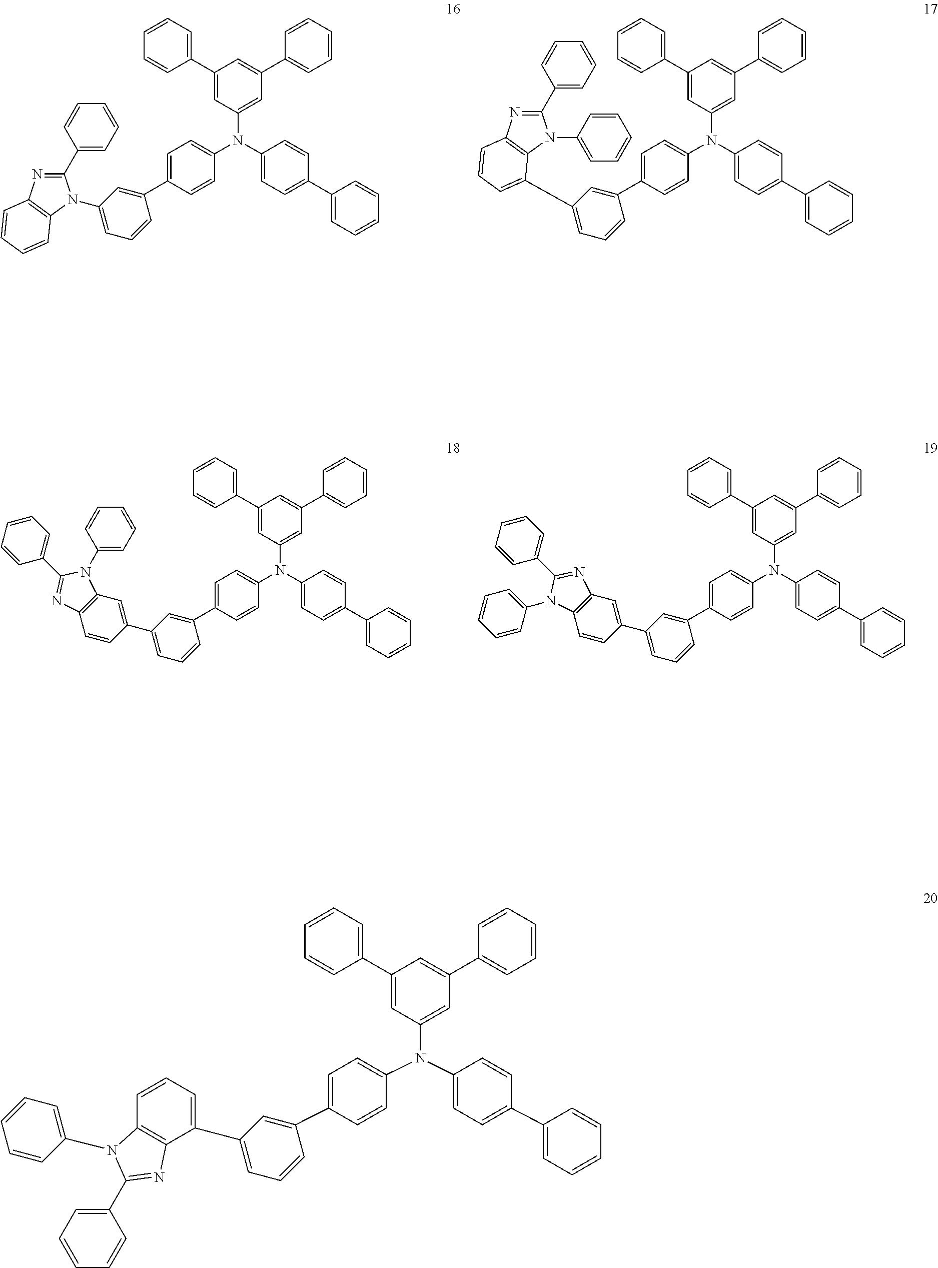

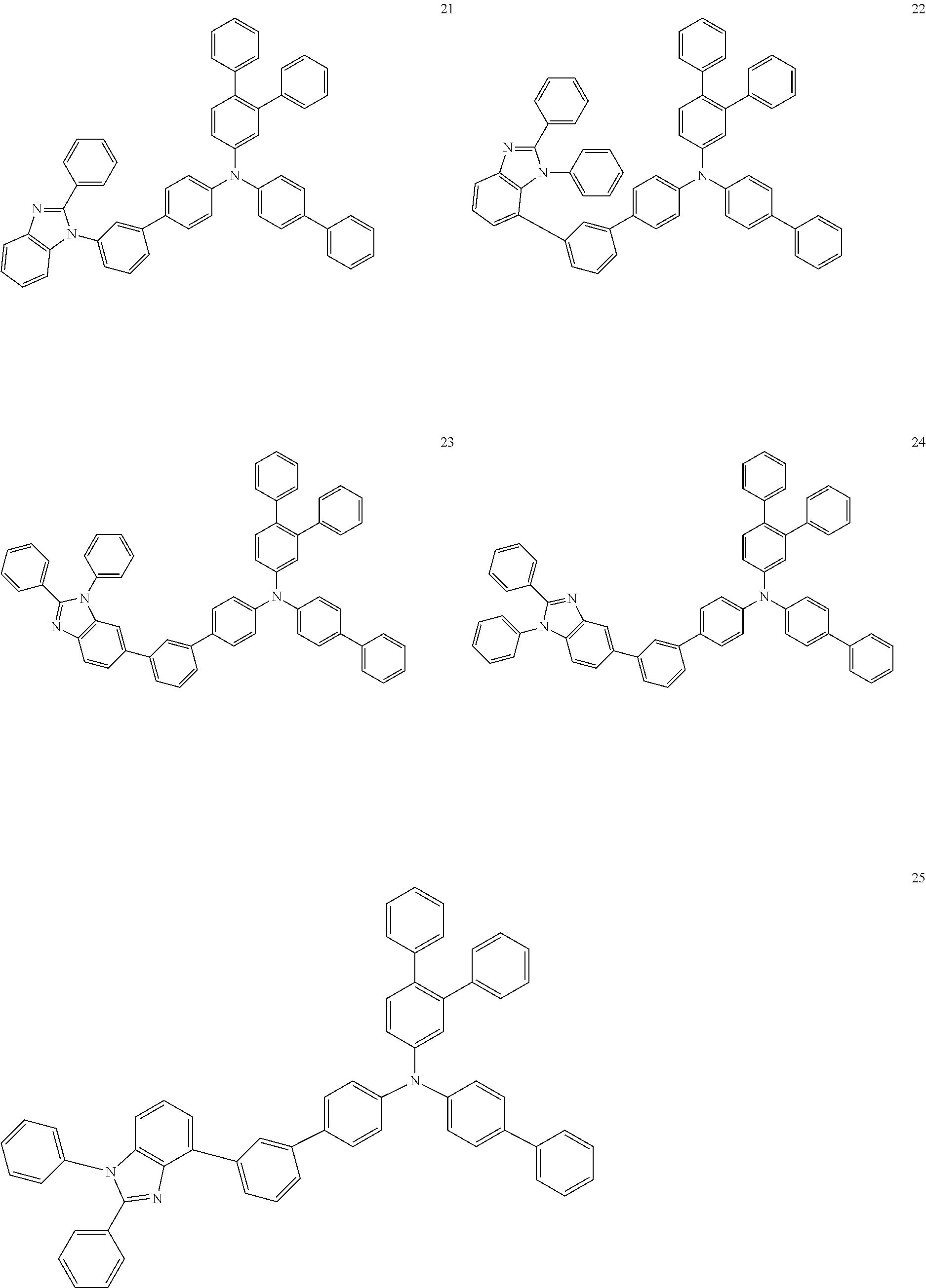

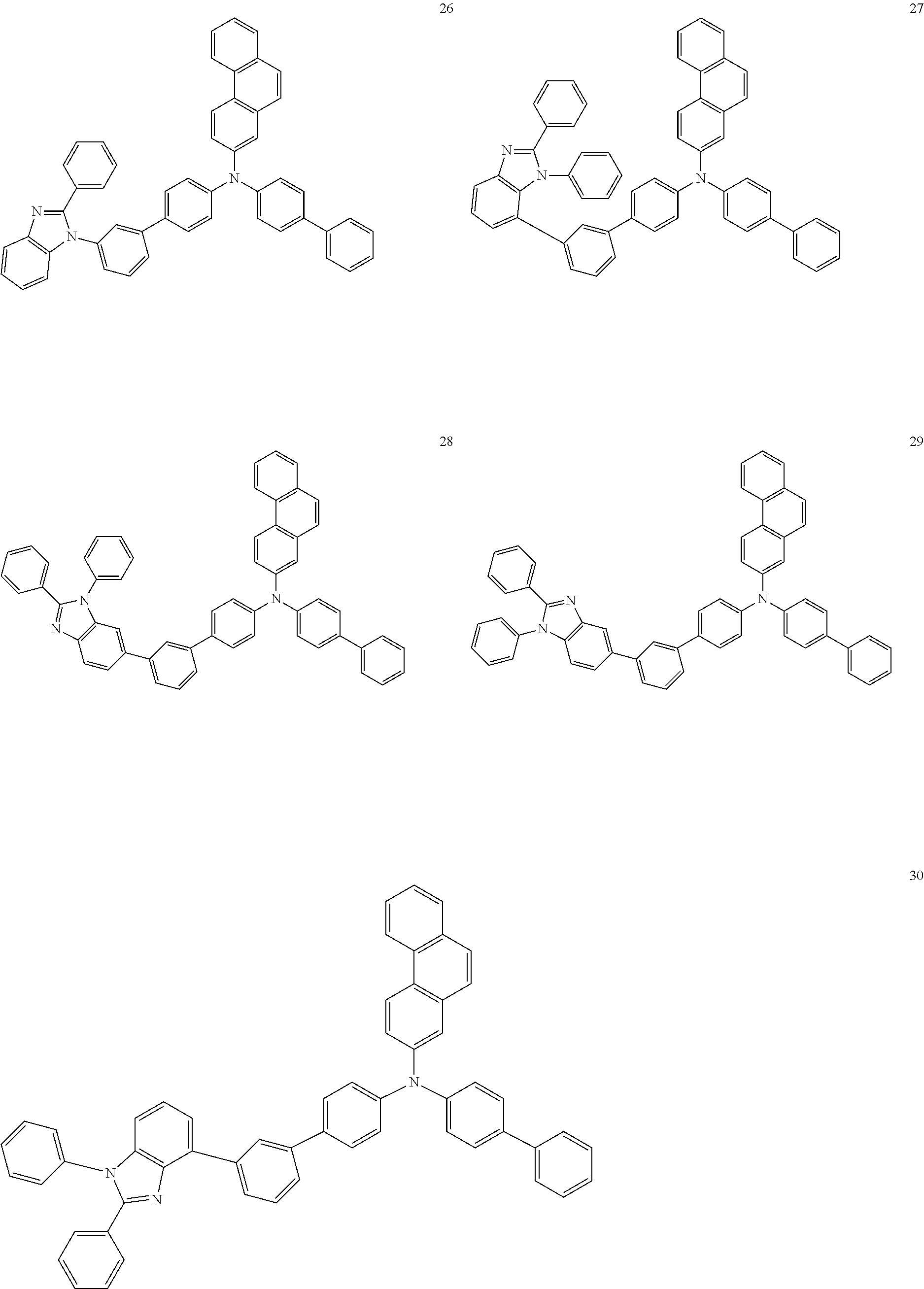

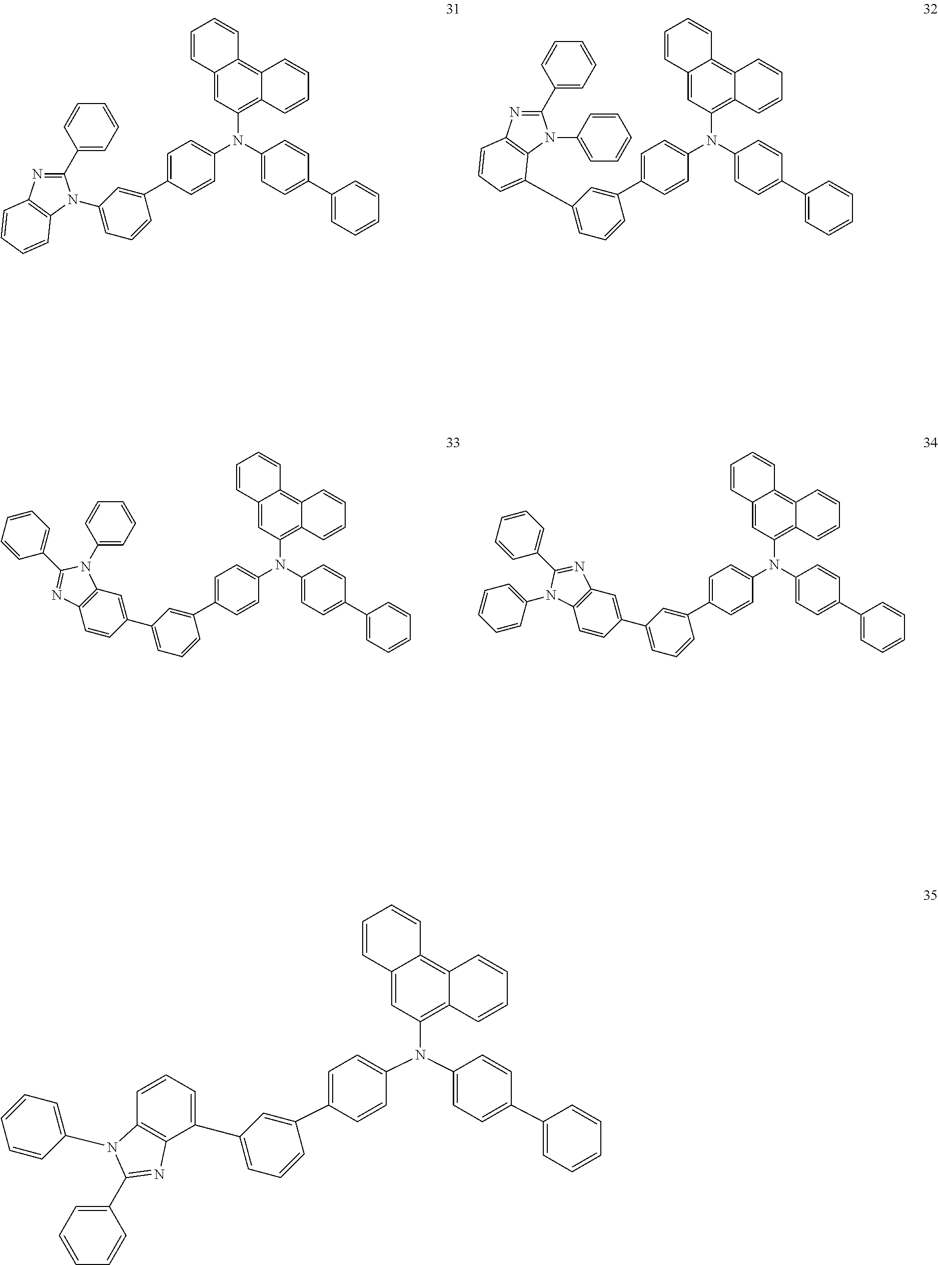

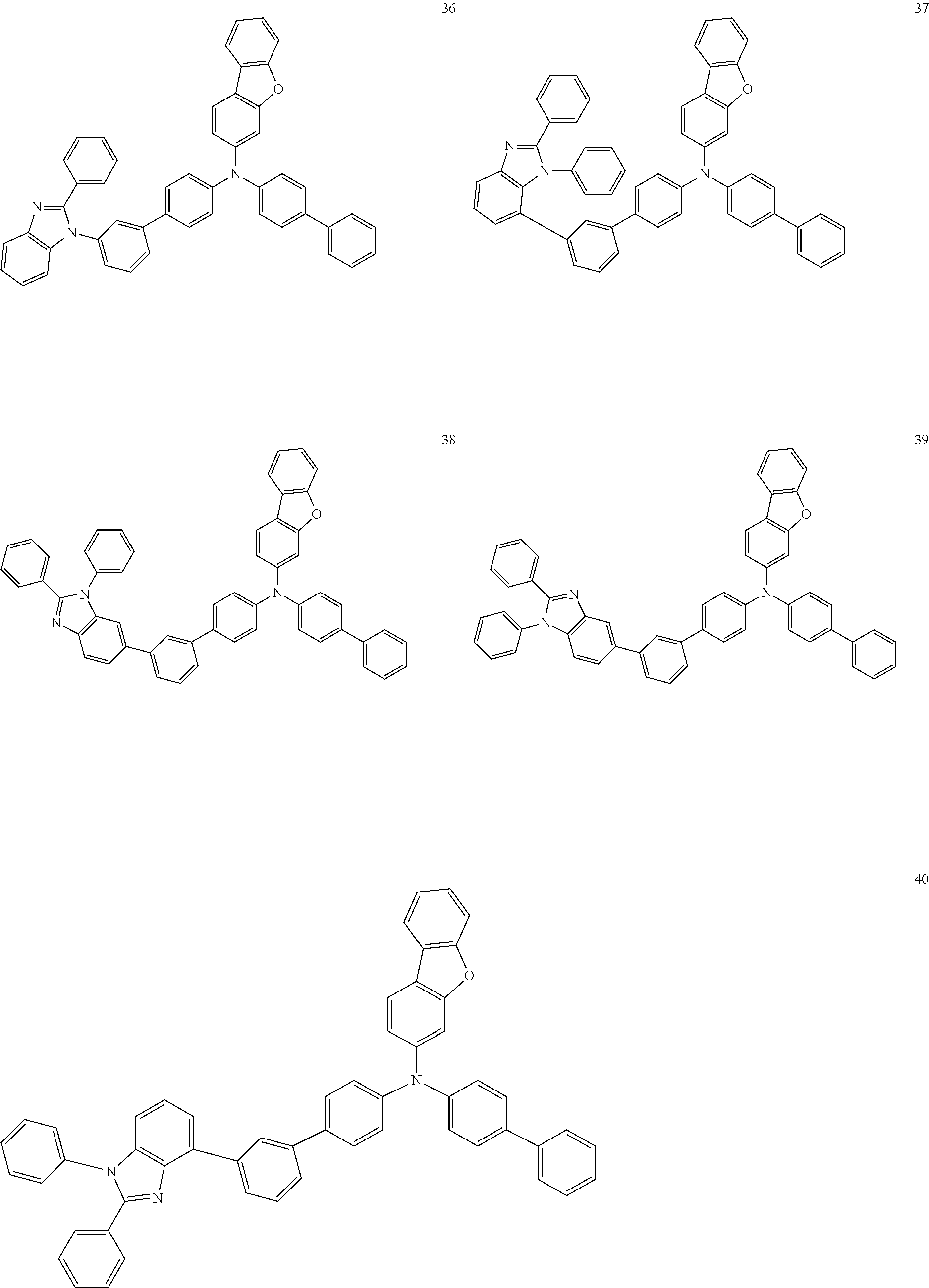

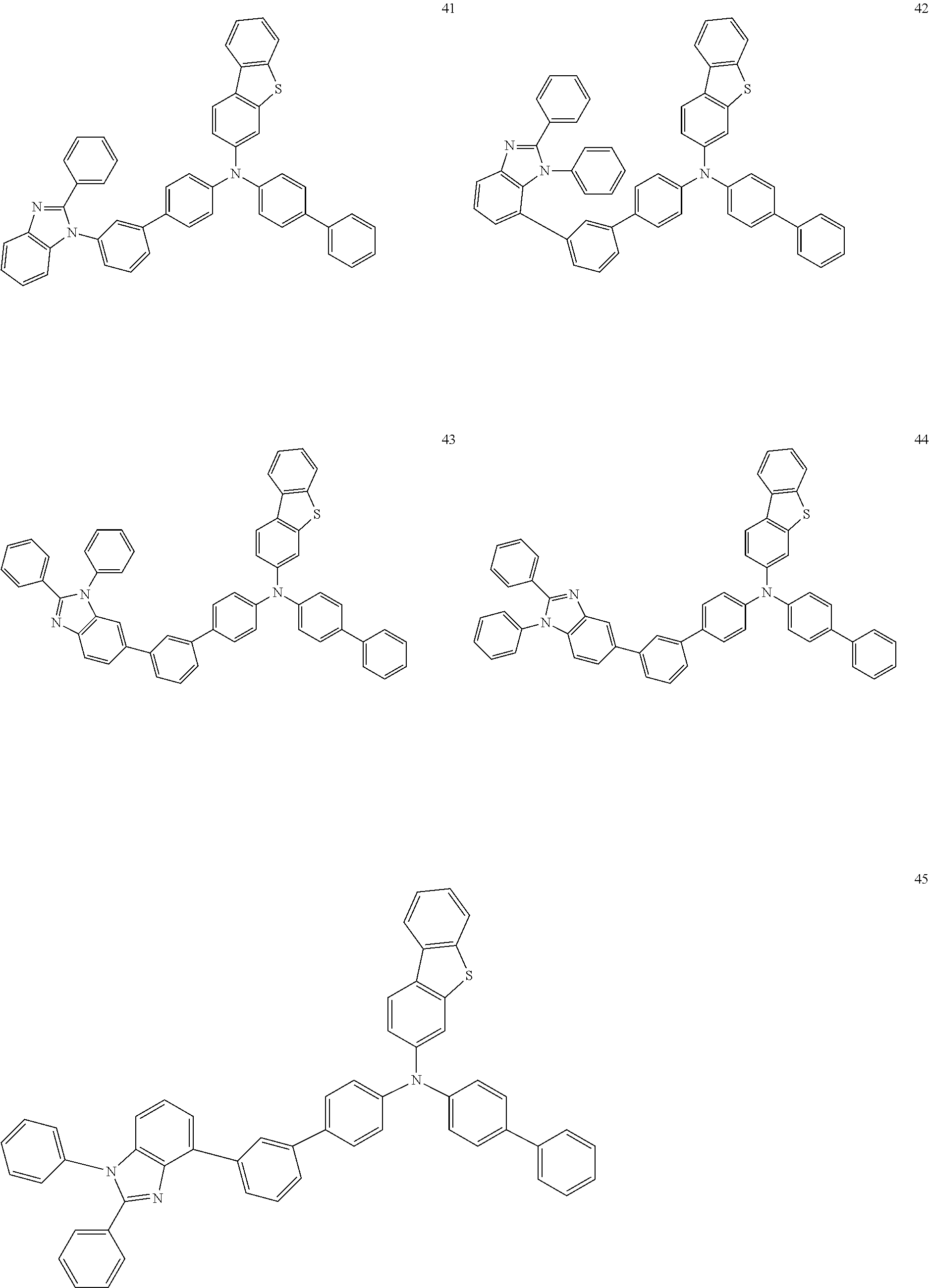

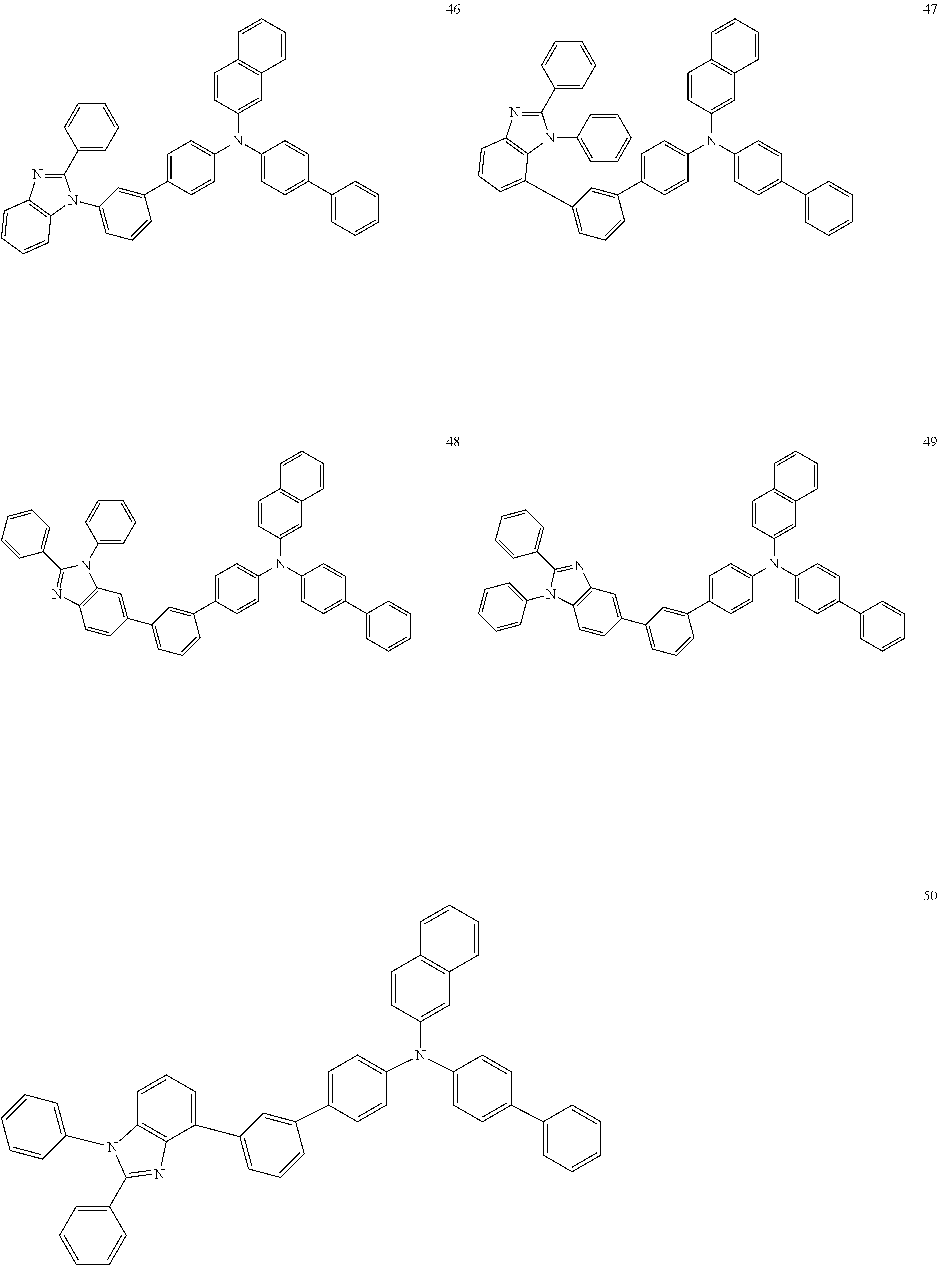 [SMILES: c1ccc(-c2ccc(N(c3ccc(-c4cccc(-n5c(-c6ccccc6)nc6ccccc65)c4)cc3)c3ccc4ccccc4c3)cc2)cc1 ; c1ccc(-c2ccc(N(c3ccc(-c4cccc(-c5cccc6nc(-c7ccccc7)n(-c7ccccc7)c56)c4)cc3)c3ccc4ccccc4c3)cc2)cc1 ; c1ccc(-c2ccc(N(c3ccc(-c4cccc(-c5ccc6nc(-c7ccccc7)n(-c7ccccc7)c6c5)c4)cc3)c3ccc4ccccc4c3)cc2)cc1 ; c1ccc(-c2ccc(N(c3ccc(-c4cccc(-c5ccc6c(c5)nc(-c5ccccc5)n6-c5ccccc5)c4)cc3)c3ccc4ccccc4c3)cc2)cc1 ; c1ccc(-c2ccc(N(c3ccc(-c4cccc(-c5cccc6c5nc(-c5ccccc5)n6-c5ccccc5)c4)cc3)c3ccc4ccccc4c3)cc2)cc1]

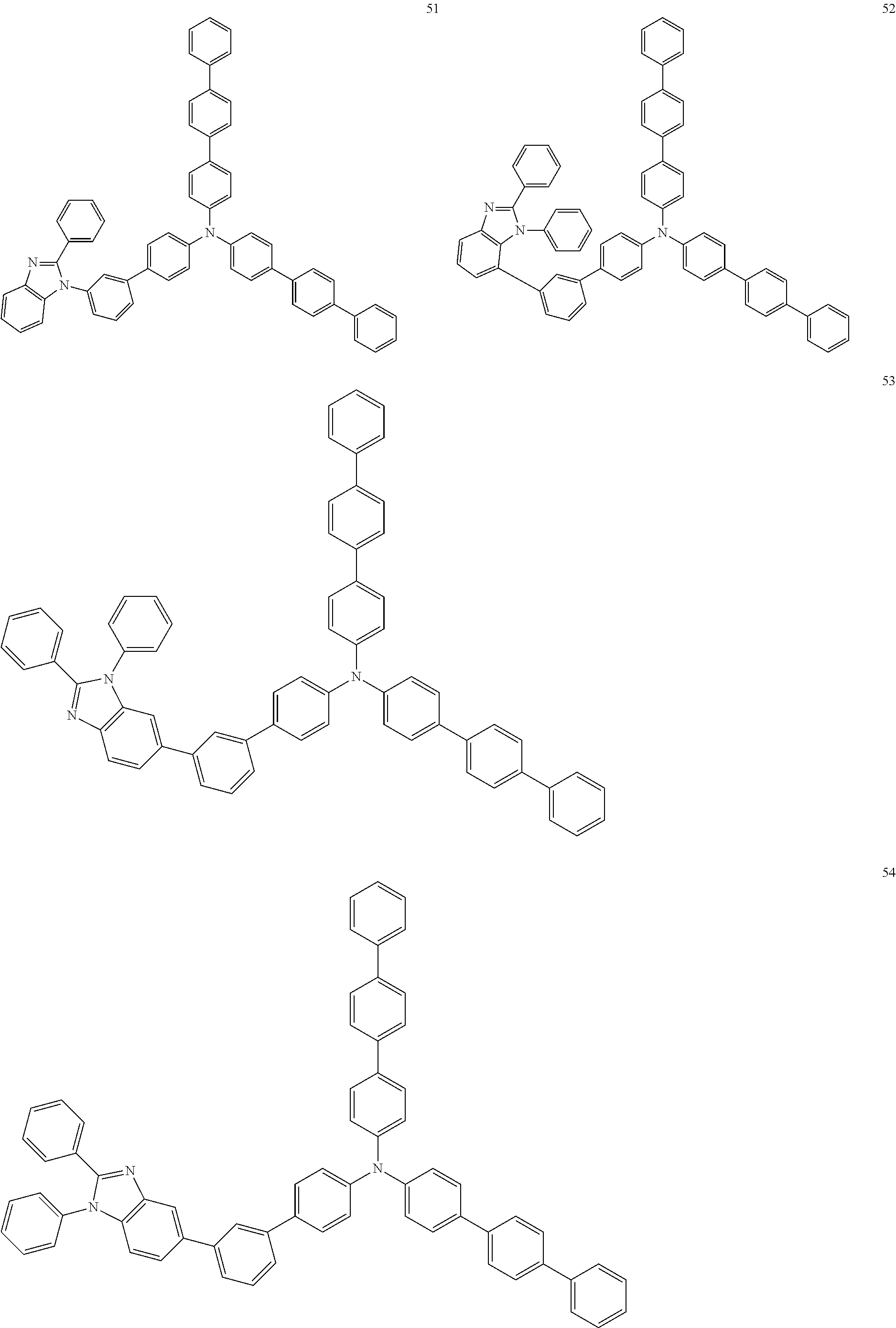

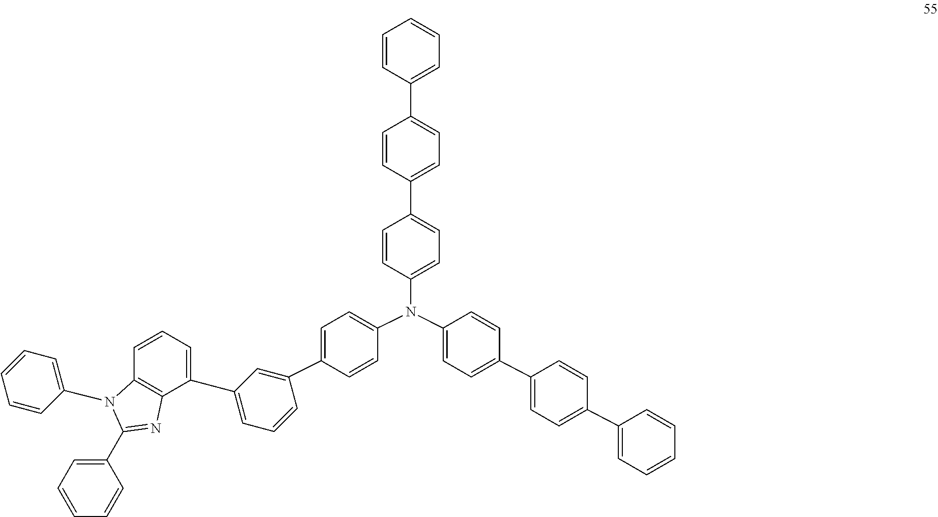 [SMILES: c1ccc(-c2ccc(-c3ccc(N(c4ccc(-c5ccc(-c6ccccc6)cc5)cc4)c4ccc(-c5cccc(-c6cccc7c6nc(-c6ccccc6)n7-c6ccccc6)c5)cc4)cc3)cc2)cc1]

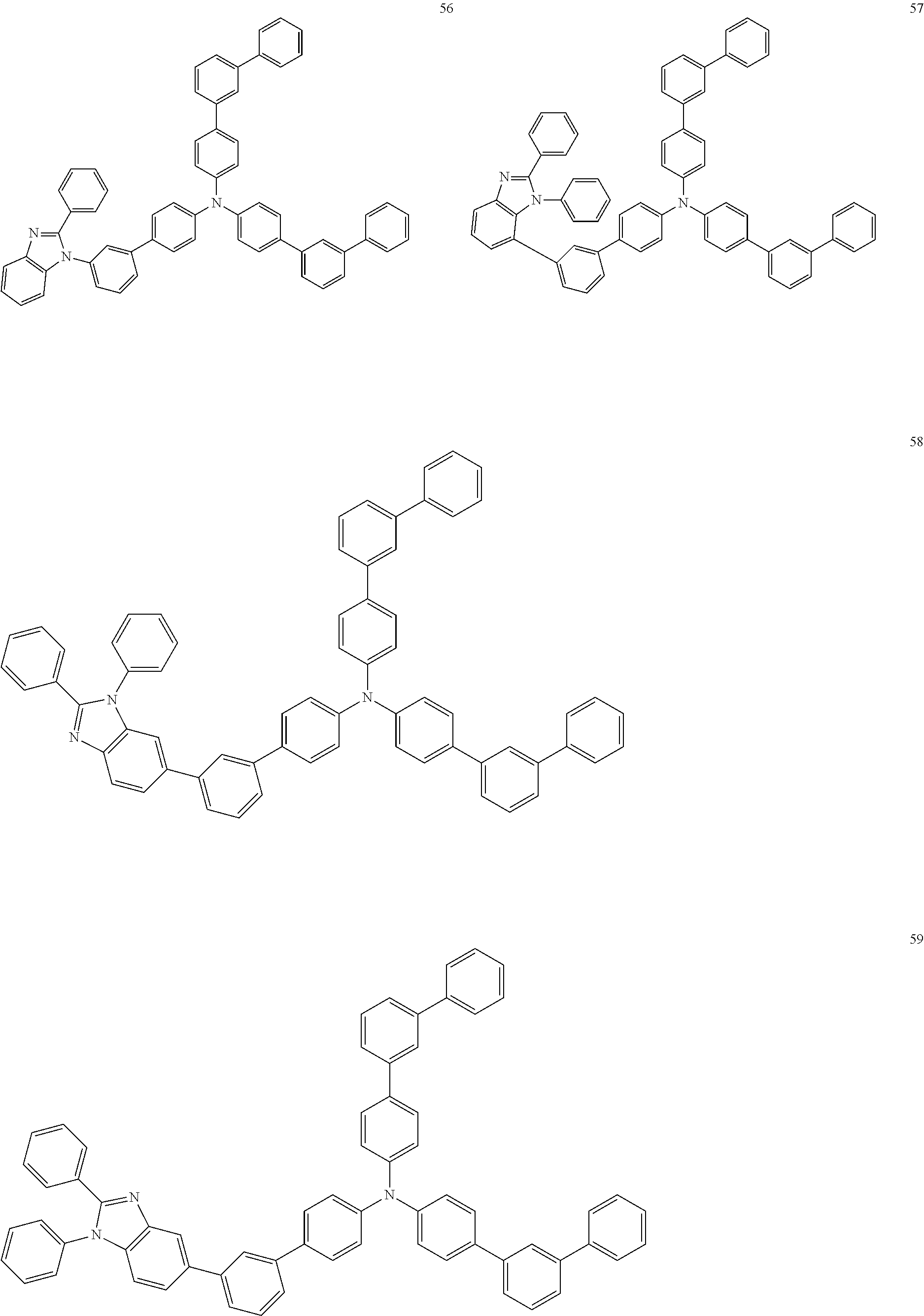 [SMILES: c1ccc(-c2cccc(-c3ccc(N(c4ccc(-c5cccc(-c6ccccc6)c5)cc4)c4ccc(-c5cccc(-n6c(-c7ccccc7)nc7ccccc76)c5)cc4)cc3)c2)cc1 ; c1ccc(-c2cccc(-c3ccc(N(c4ccc(-c5cccc(-c6ccccc6)c5)cc4)c4ccc(-c5cccc(-c6cccc7nc(-c8ccccc8)n(-c8ccccc8)c67)c5)cc4)cc3)c2)cc1 ; c1ccc(-c2cccc(-c3ccc(N(c4ccc(-c5cccc(-c6ccccc6)c5)cc4)c4ccc(-c5cccc(-c6ccc7nc(-c8ccccc8)n(-c8ccccc8)c7c6)c5)cc4)cc3)c2)cc1 ; c1ccc(-c2cccc(-c3ccc(N(c4ccc(-c5cccc(-c6ccccc6)c5)cc4)c4ccc(-c5cccc(-c6ccc7c(c6)nc(-c6ccccc6)n7-c6ccccc6)c5)cc4)cc3)c2)cc1]

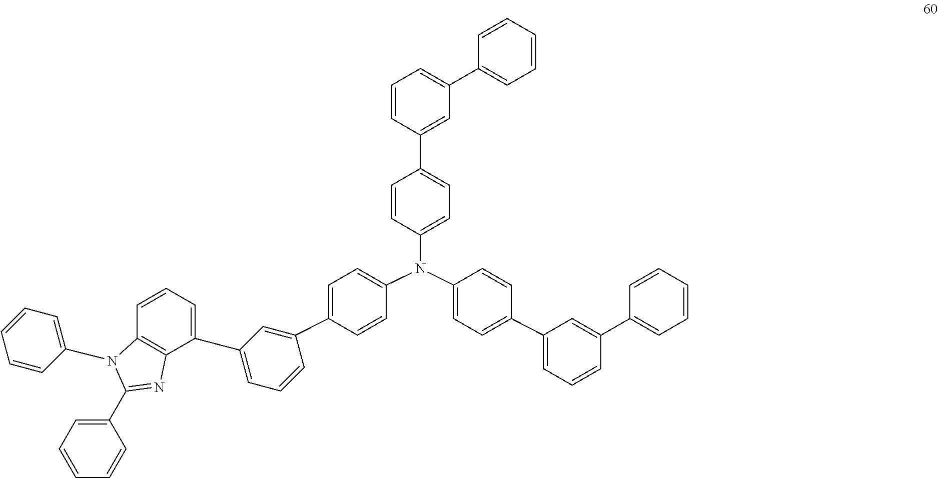 [SMILES: c1ccc(-c2cccc(-c3ccc(N(c4ccc(-c5cccc(-c6ccccc6)c5)cc4)c4ccc(-c5cccc(-c6cccc7c6nc(-c6ccccc6)n7-c6ccccc6)c5)cc4)cc3)c2)cc1]

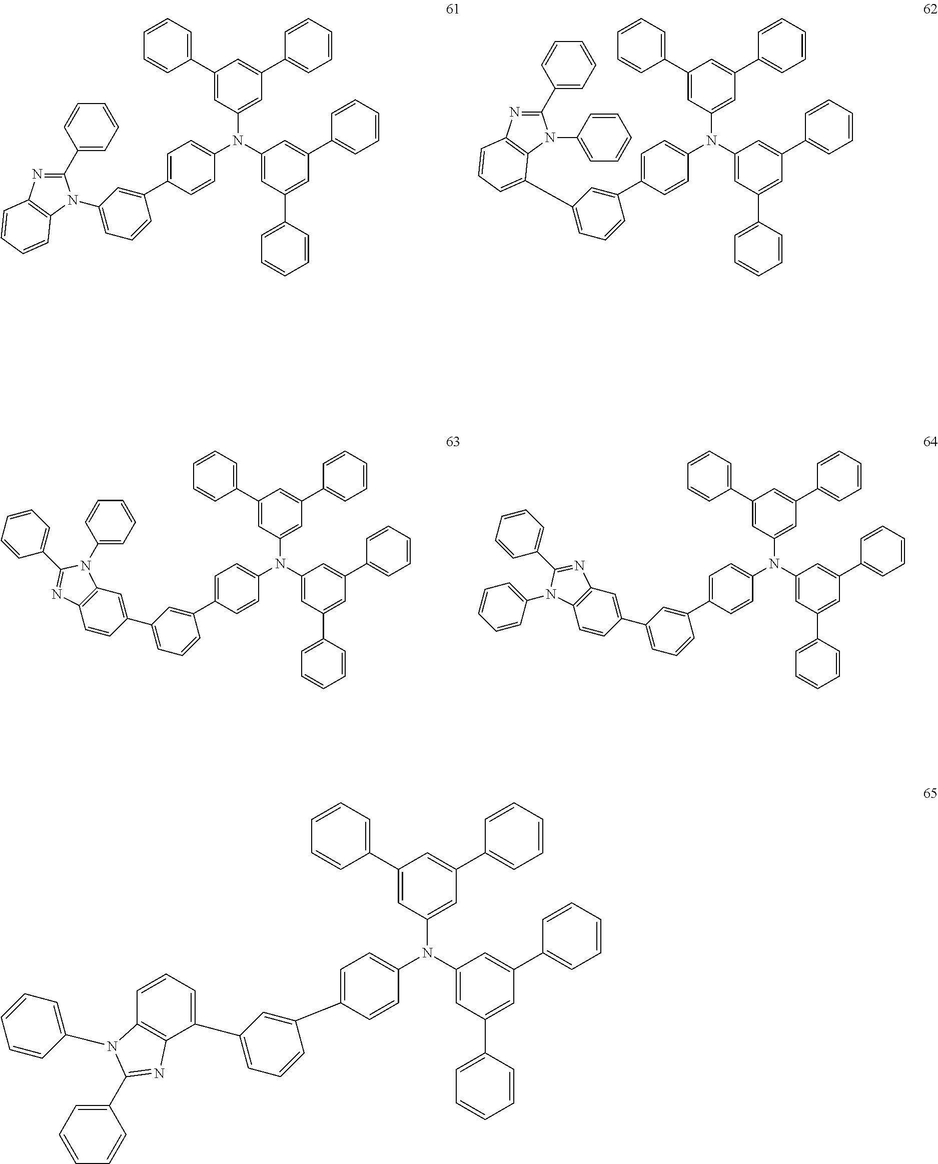

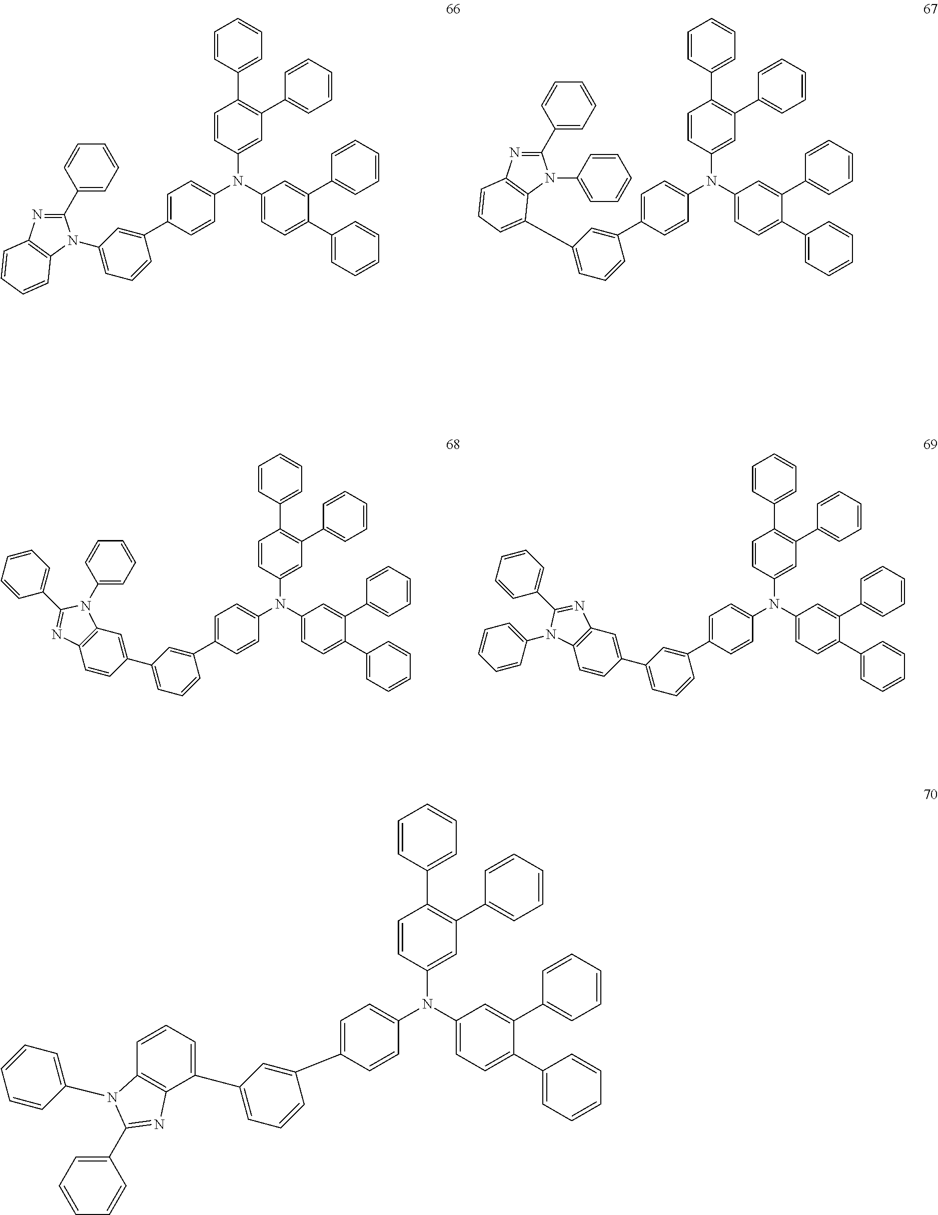

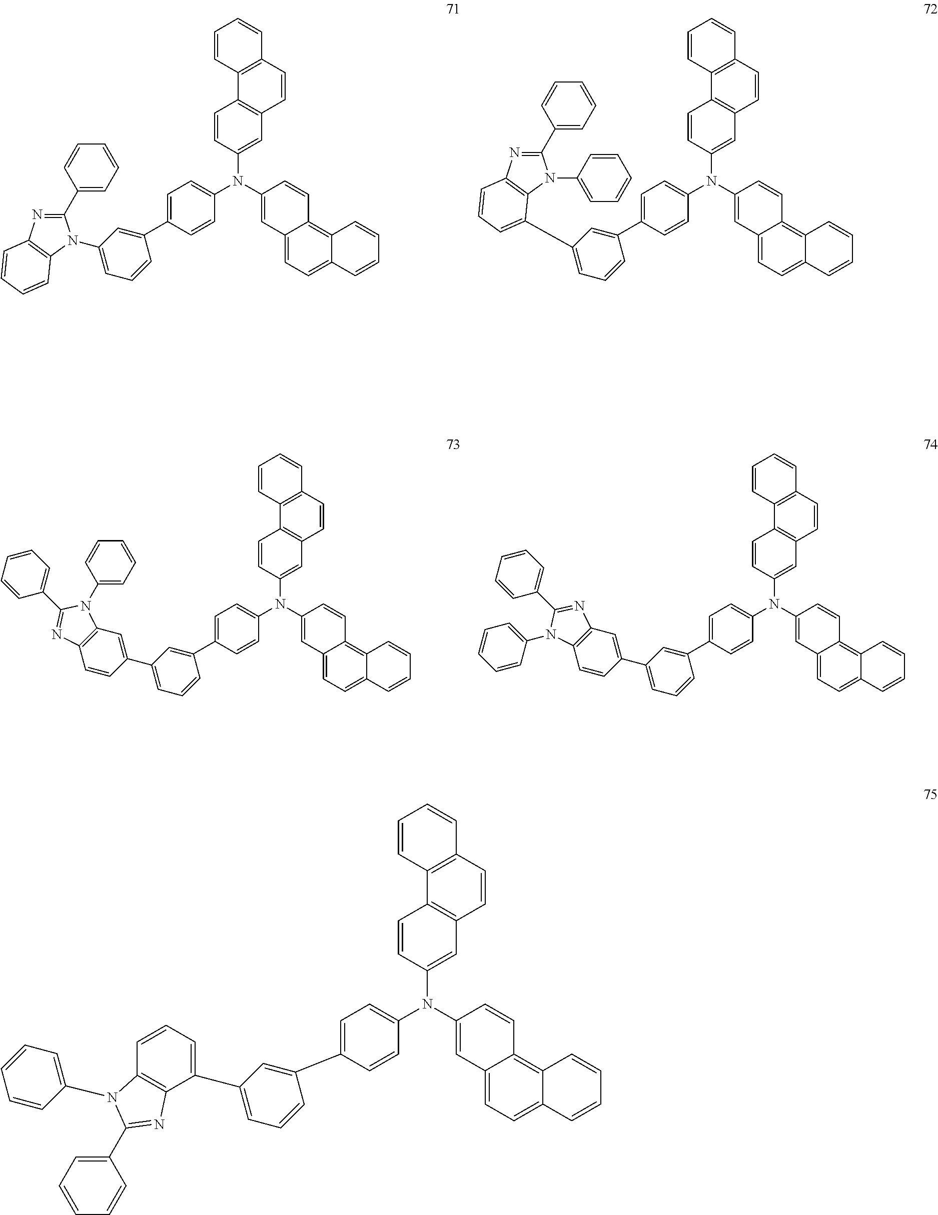

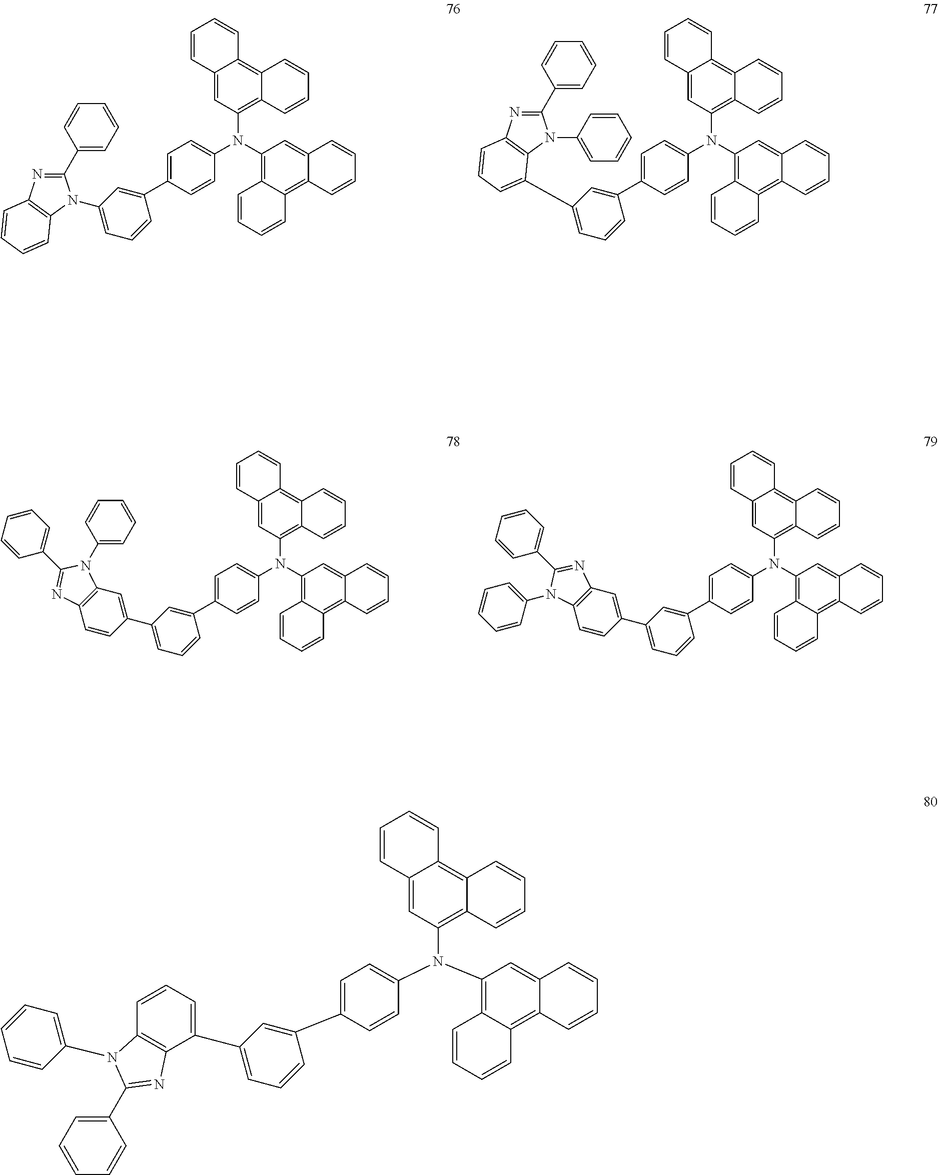

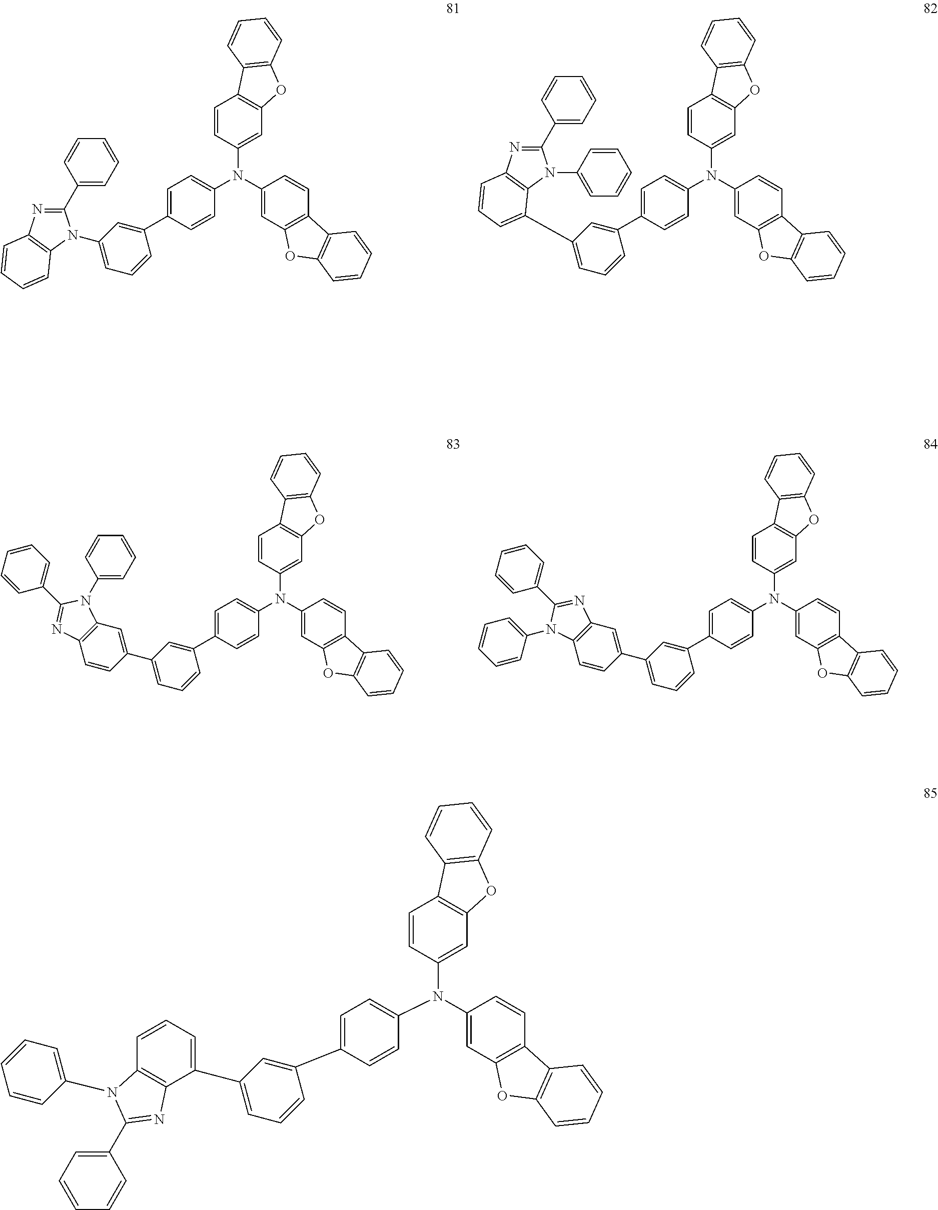

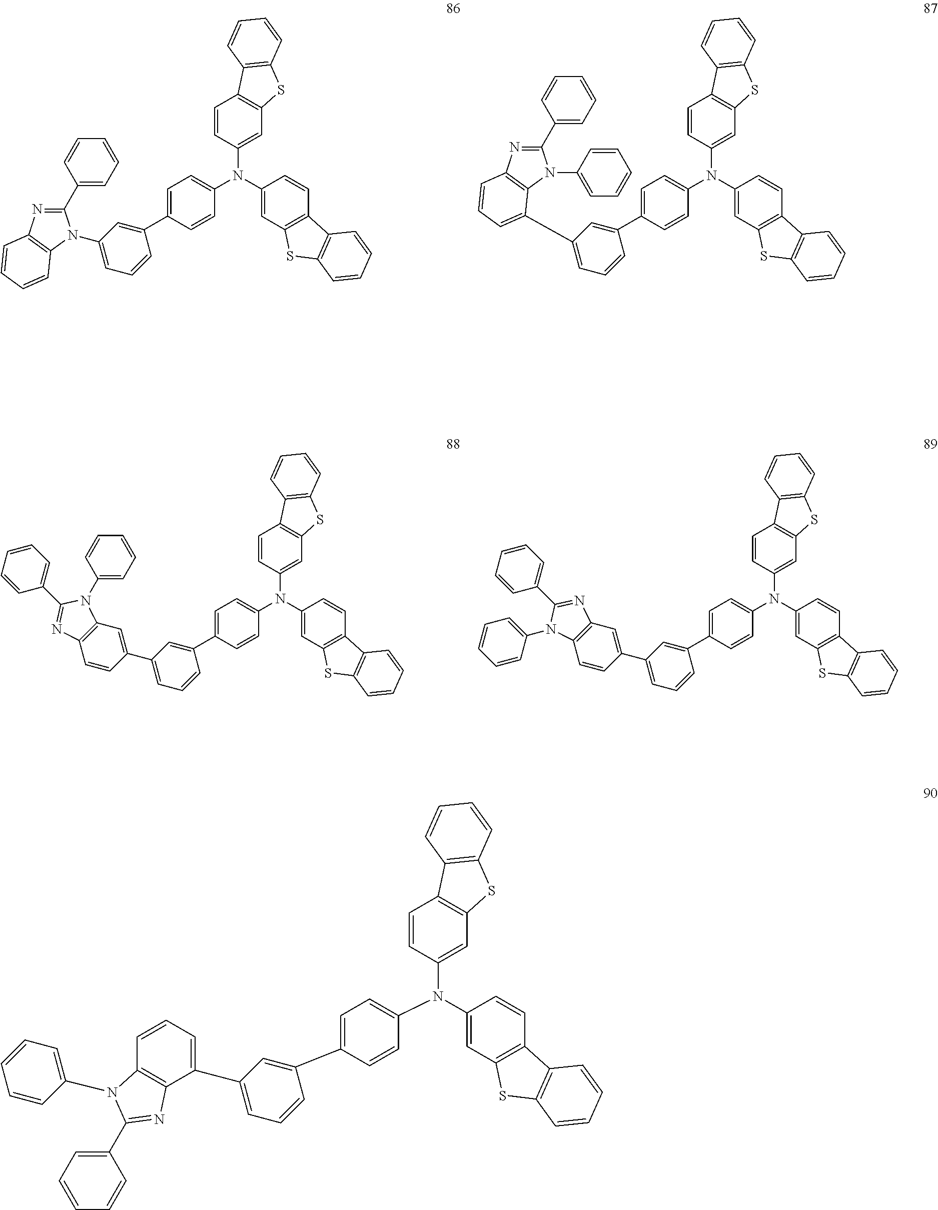

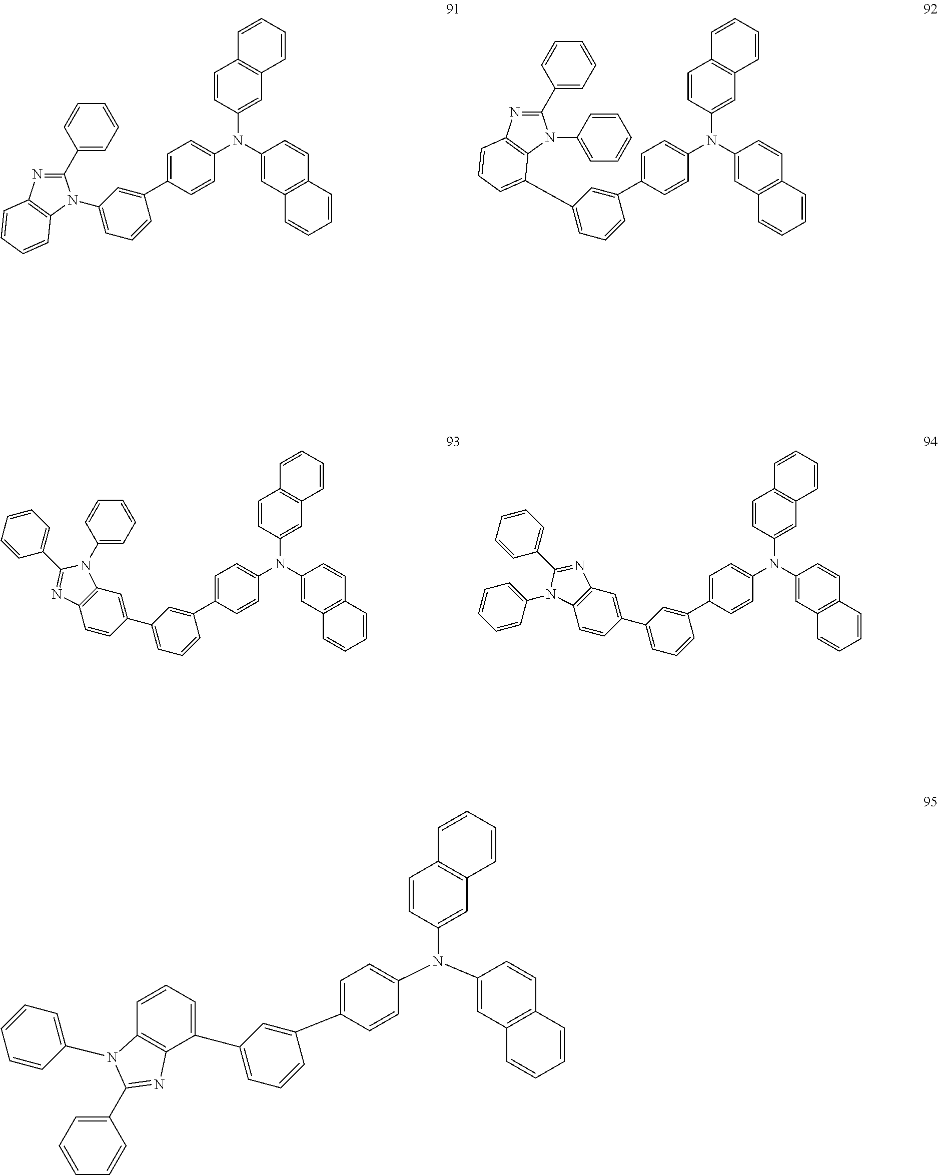 [SMILES: c1ccc(-c2nc3ccccc3n2-c2cccc(-c3ccc(N(c4ccc5ccccc5c4)c4ccc5ccccc5c4)cc3)c2)cc1 ; c1ccc(-c2nc3cccc(-c4cccc(-c5ccc(N(c6ccc7ccccc7c6)c6ccc7ccccc7c6)cc5)c4)c3n2-c2ccccc2)cc1 ; c1ccc(-c2nc3ccc(-c4cccc(-c5ccc(N(c6ccc7ccccc7c6)c6ccc7ccccc7c6)cc5)c4)cc3n2-c2ccccc2)cc1 ; c1ccc(-c2nc3cc(-c4cccc(-c5ccc(N(c6ccc7ccccc7c6)c6ccc7ccccc7c6)cc5)c4)ccc3n2-c2ccccc2)cc1 ; c1ccc(-c2nc3c(-c4cccc(-c5ccc(N(c6ccc7ccccc7c6)c6ccc7ccccc7c6)cc5)c4)cccc3n2-c2ccccc2)cc1]

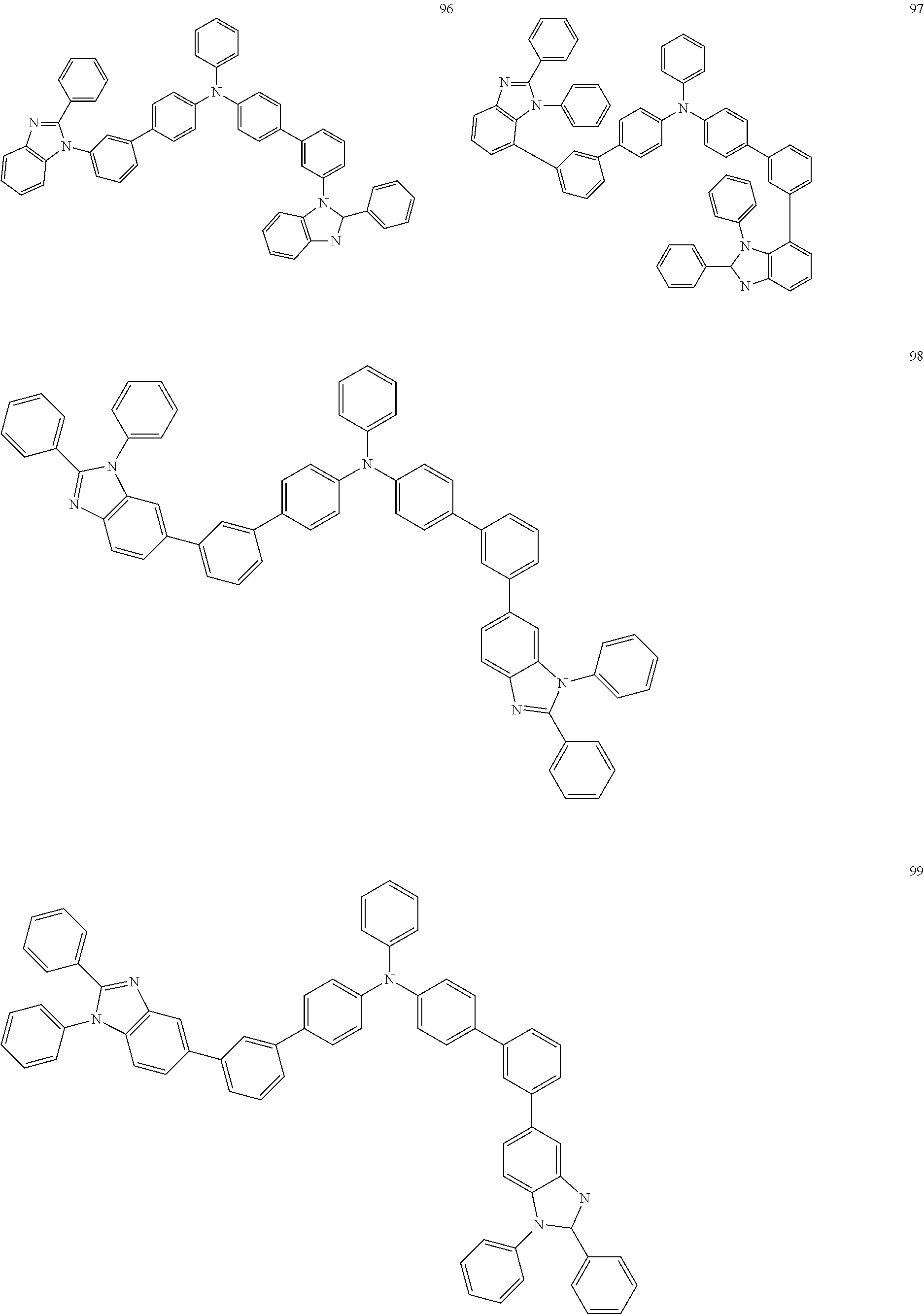 [SMILES: c1ccc(-c2nc3ccccc3n2-c2cccc(-c3ccc(N(c4ccccc4)c4ccc(-c5cccc(N6c7ccccc7NC6c6ccccc6)c5)cc4)cc3)c2)cc1 ; c1ccc(-c2nc3cccc(-c4cccc(-c5ccc(N(c6ccccc6)c6ccc(-c7cccc(-c8cccc9c8N(c8ccccc8)C(c8ccccc8)N9)c7)cc6)cc5)c4)c3n2-c2ccccc2)cc1 ; c1ccc(-c2nc3ccc(-c4cccc(-c5ccc(N(c6ccccc6)c6ccc(-c7cccc(-c8ccc9nc(-c%10ccccc%10)n(-c%10ccccc%10)c9c8)c7)cc6)cc5)c4)cc3n2-c2ccccc2)cc1 ; c1ccc(-c2nc3cc(-c4cccc(-c5ccc(N(c6ccccc6)c6ccc(-c7cccc(-c8ccc9c(c8)NC(c8ccccc8)N9c8ccccc8)c7)cc6)cc5)c4)ccc3n2-c2ccccc2)cc1]

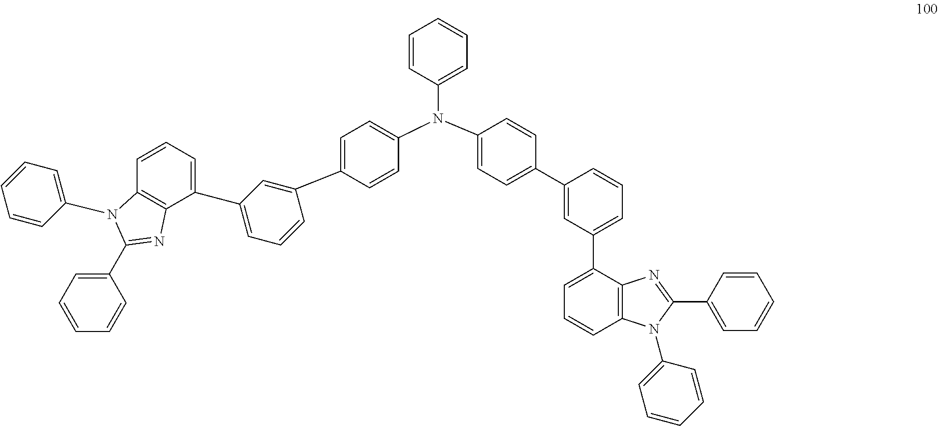 [SMILES: c1ccc(-c2nc3c(-c4cccc(-c5ccc(N(c6ccccc6)c6ccc(-c7cccc(-c8cccc9c8nc(-c8ccccc8)n9-c8ccccc8)c7)cc6)cc5)c4)cccc3n2-c2ccccc2)cc1]

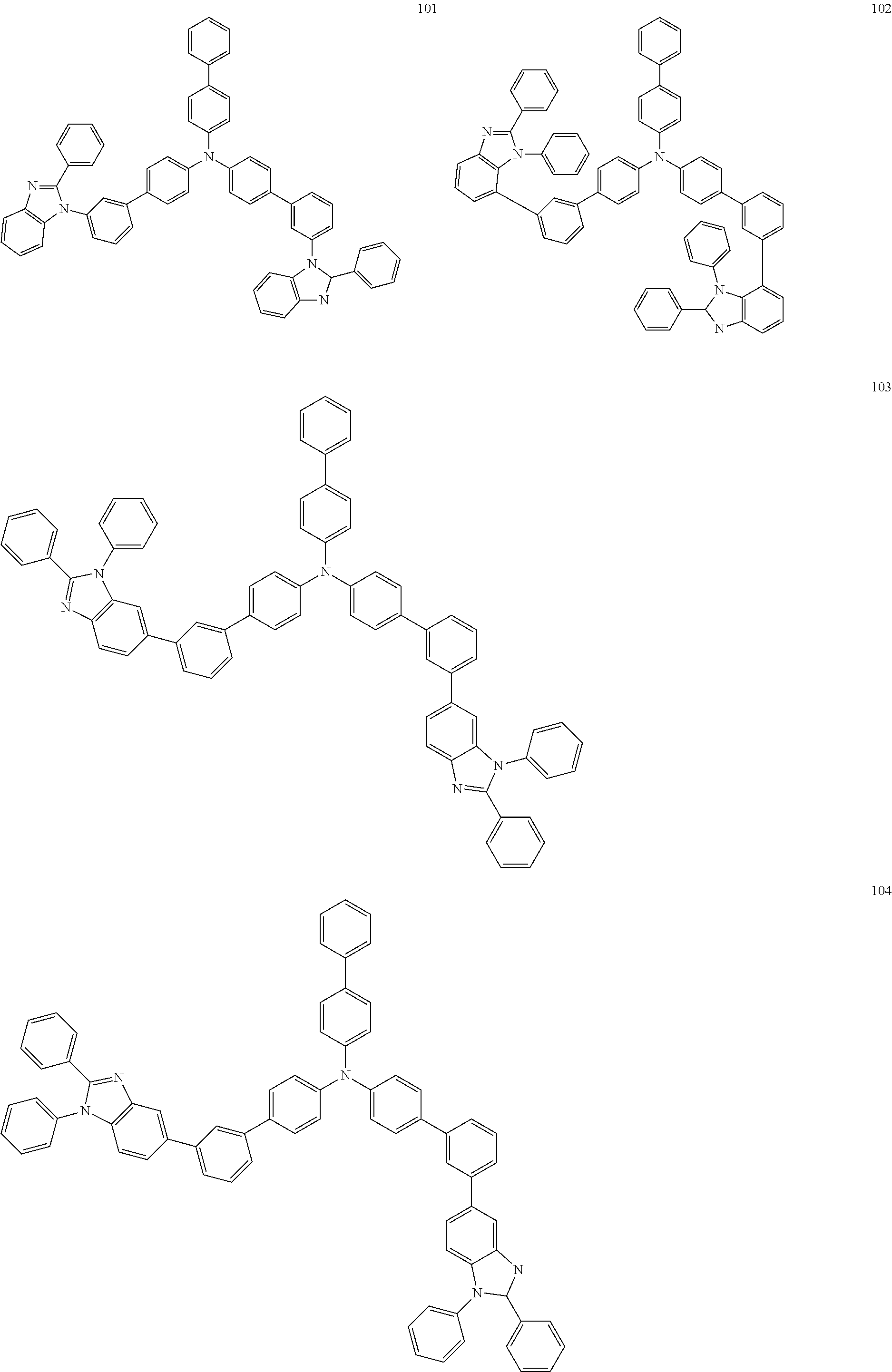

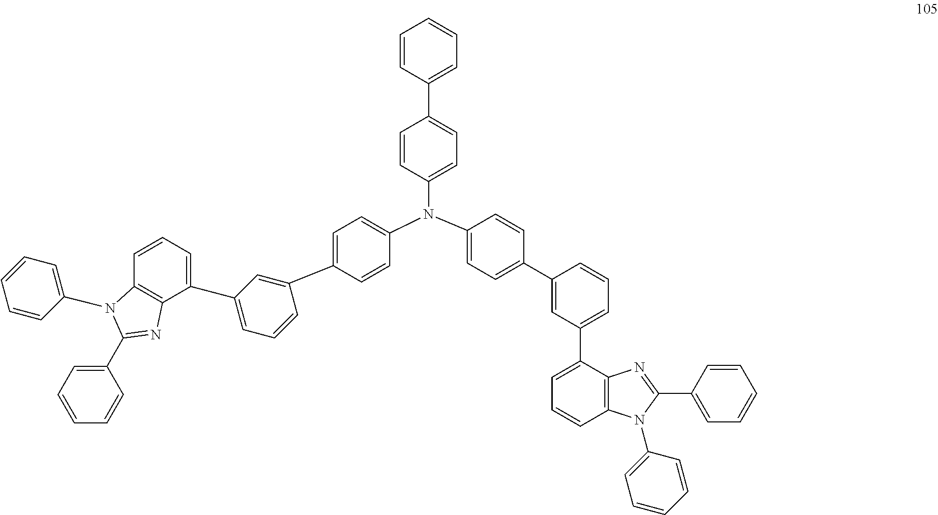 [SMILES: c1ccc(-c2ccc(N(c3ccc(-c4cccc(-c5cccc6c5nc(-c5ccccc5)n6-c5ccccc5)c4)cc3)c3ccc(-c4cccc(-c5cccc6c5nc(-c5ccccc5)n6-c5ccccc5)c4)cc3)cc2)cc1]

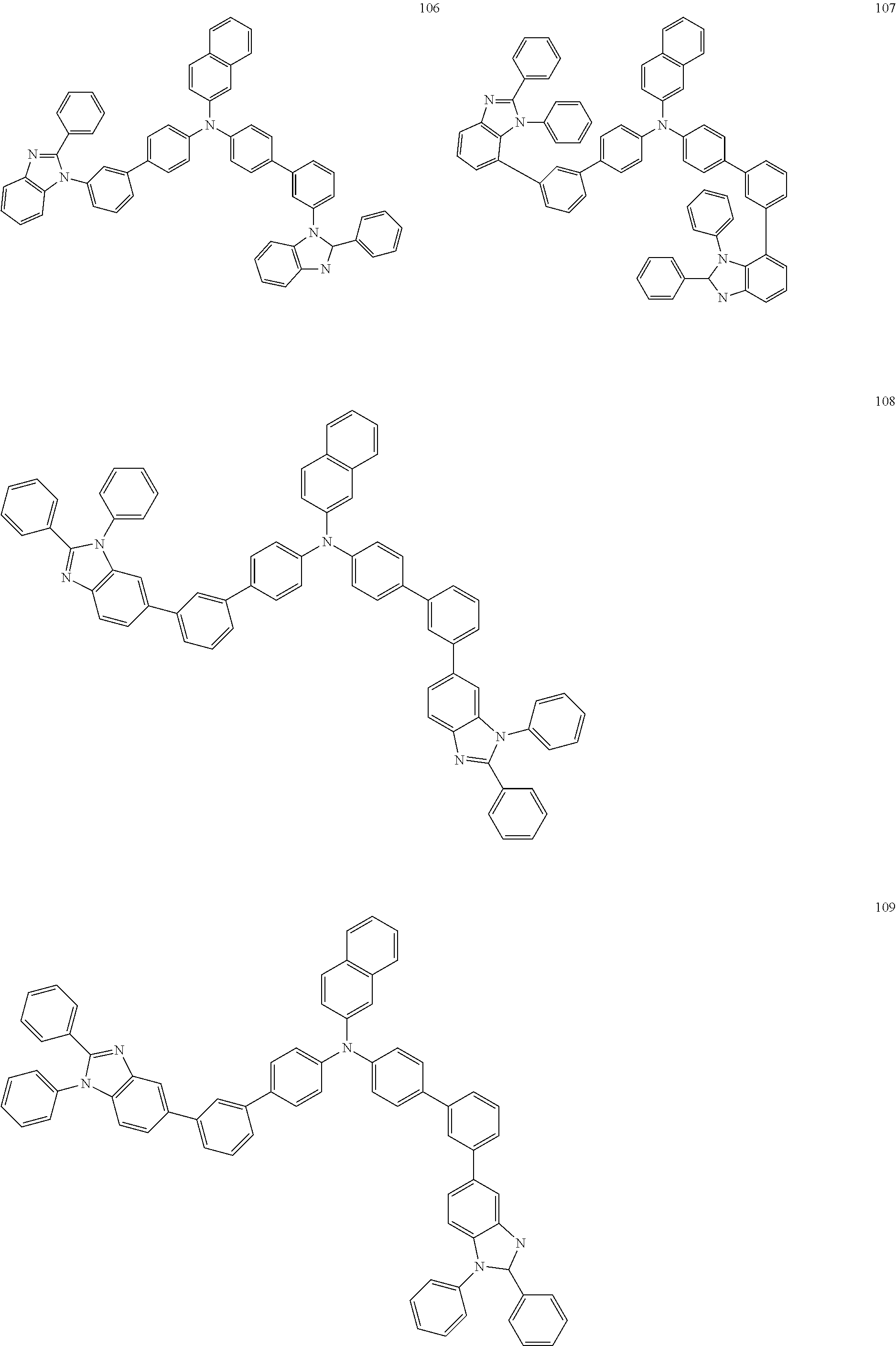

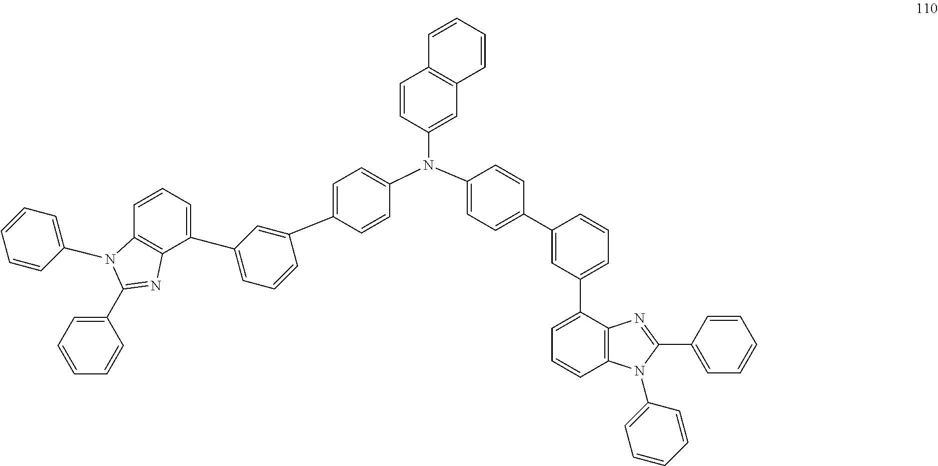 [SMILES: c1ccc(-c2nc3c(-c4cccc(-c5ccc(N(c6ccc(-c7cccc(-c8cccc9c8nc(-c8ccccc8)n9-c8ccccc8)c7)cc6)c6ccc7ccccc7c6)cc5)c4)cccc3n2-c2ccccc2)cc1]

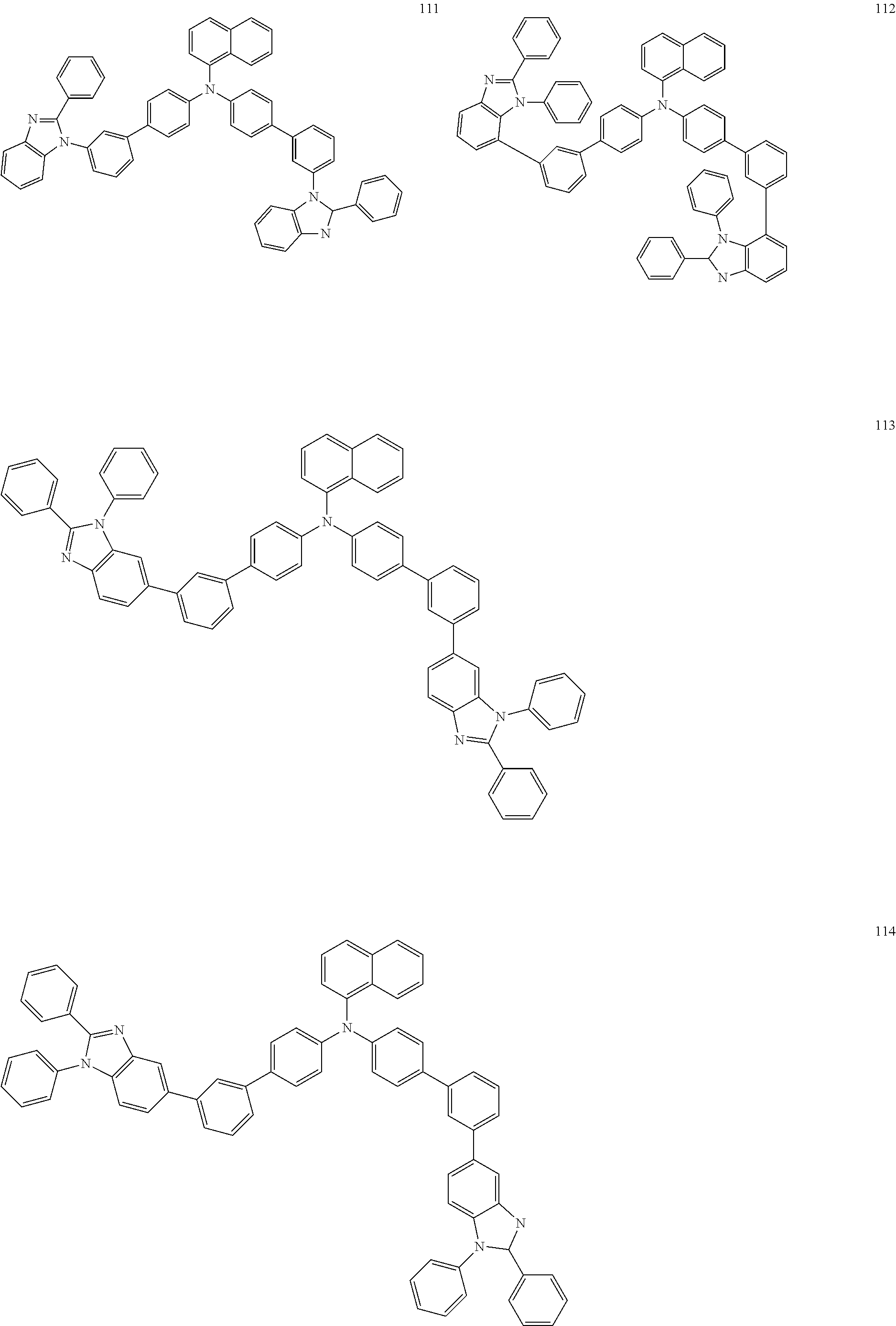

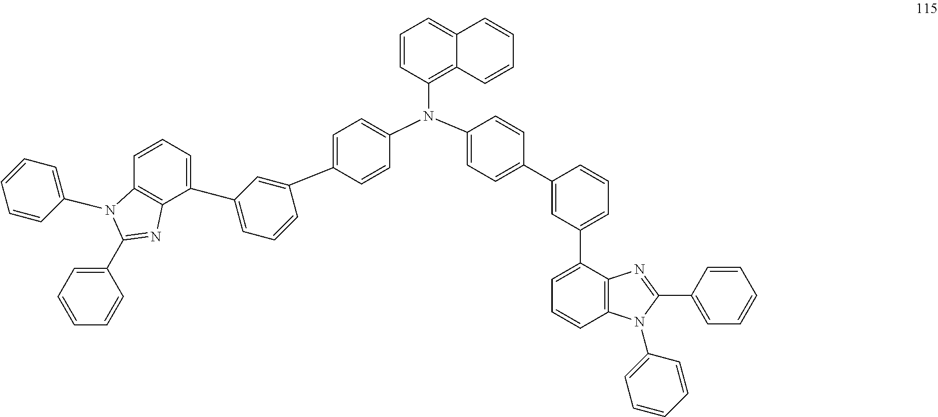 [SMILES: c1ccc(-c2nc3c(-c4cccc(-c5ccc(N(c6ccc(-c7cccc(-c8cccc9c8nc(-c8ccccc8)n9-c8ccccc8)c7)cc6)c6cccc7ccccc67)cc5)c4)cccc3n2-c2ccccc2)cc1]

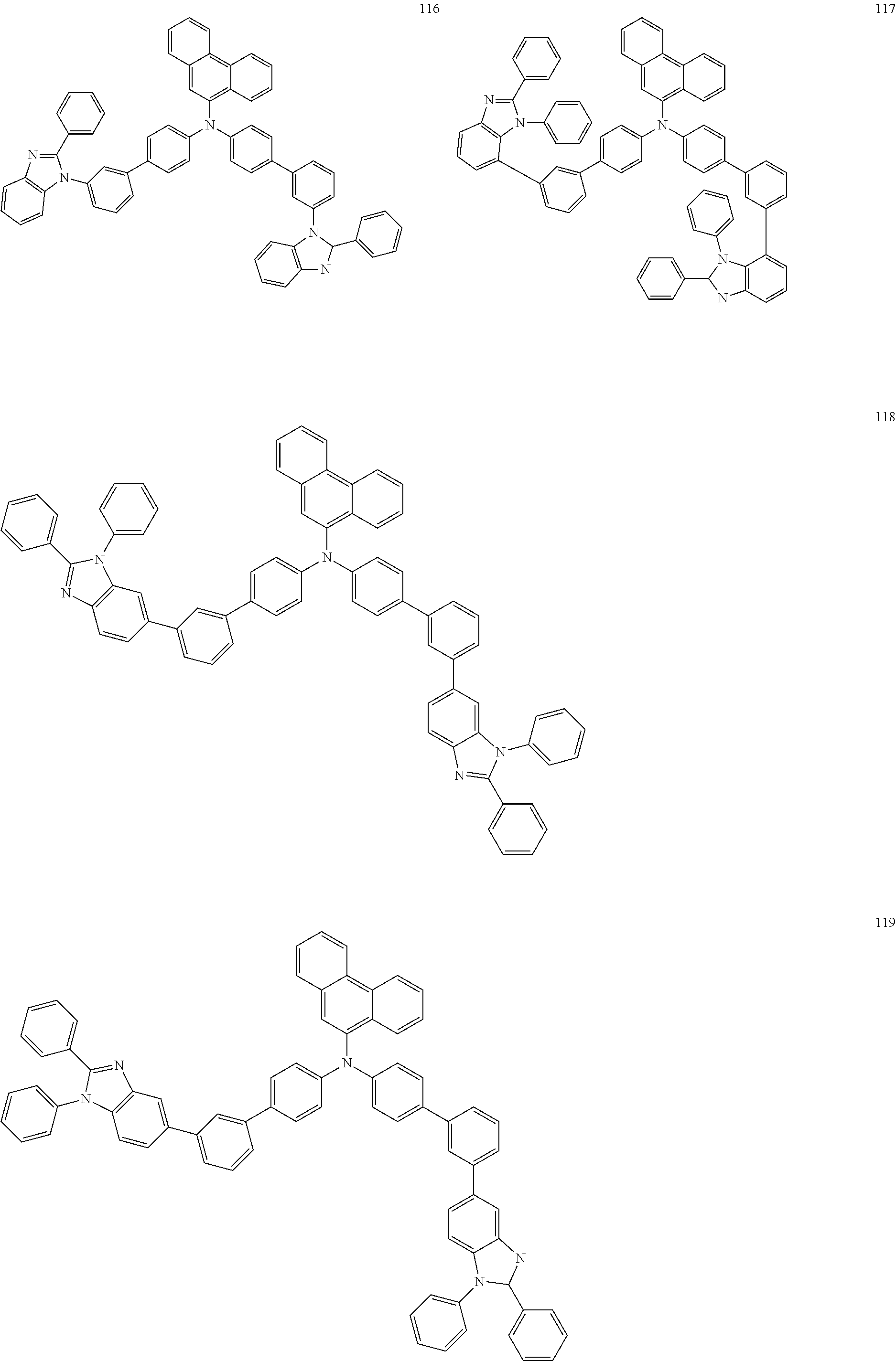

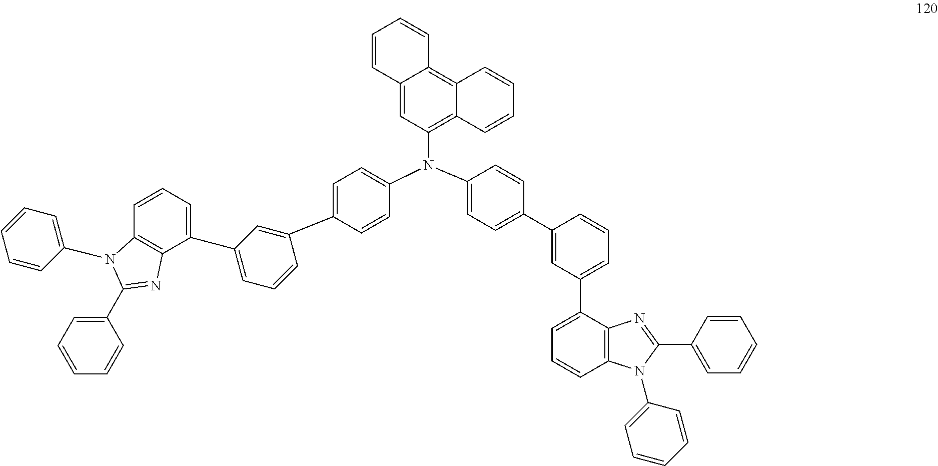 [SMILES: c1ccc(-c2nc3c(-c4cccc(-c5ccc(N(c6ccc(-c7cccc(-c8cccc9c8nc(-c8ccccc8)n9-c8ccccc8)c7)cc6)c6cc7ccccc7c7ccccc67)cc5)c4)cccc3n2-c2ccccc2)cc1]

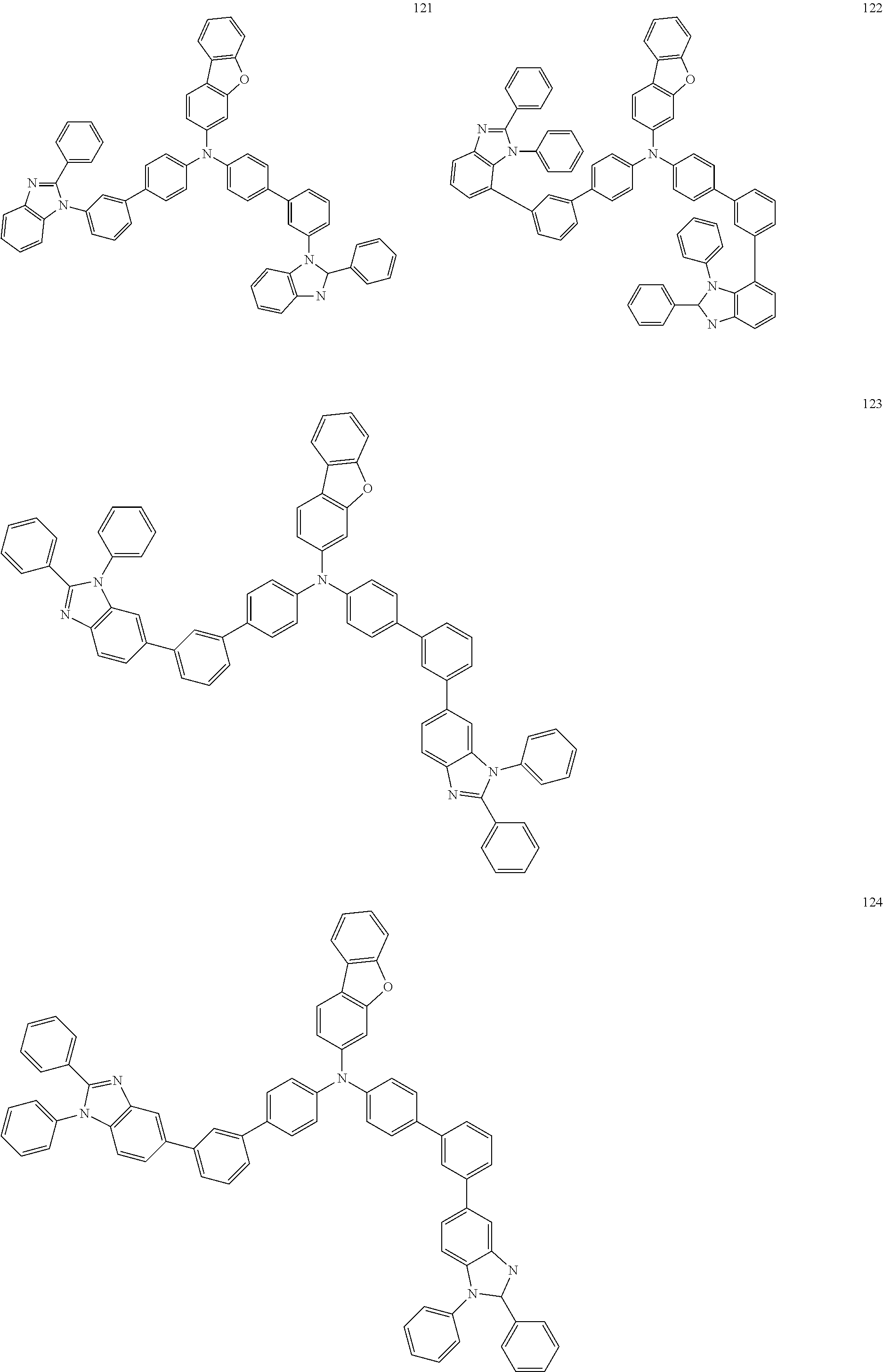

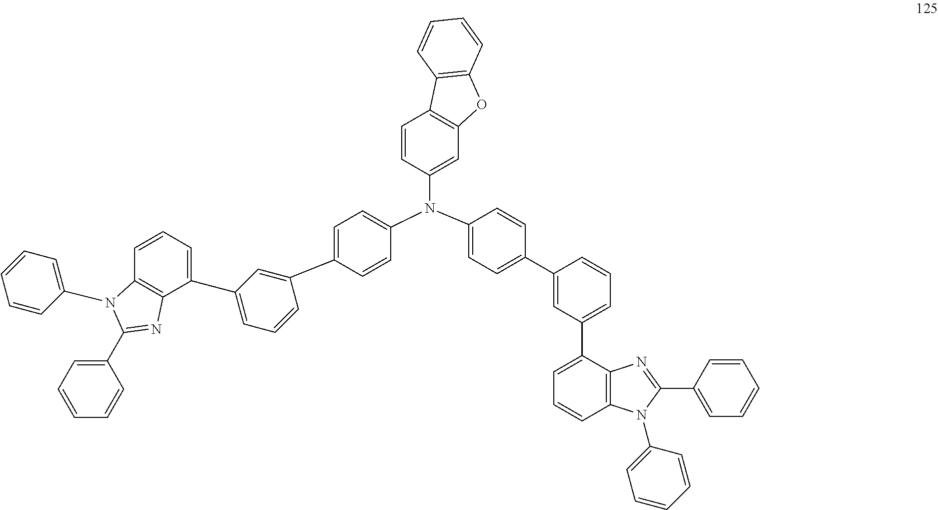 [SMILES: c1ccc(-c2nc3c(-c4cccc(-c5ccc(N(c6ccc(-c7cccc(-c8cccc9c8nc(-c8ccccc8)n9-c8ccccc8)c7)cc6)c6ccc7c(c6)oc6ccccc67)cc5)c4)cccc3n2-c2ccccc2)cc1]

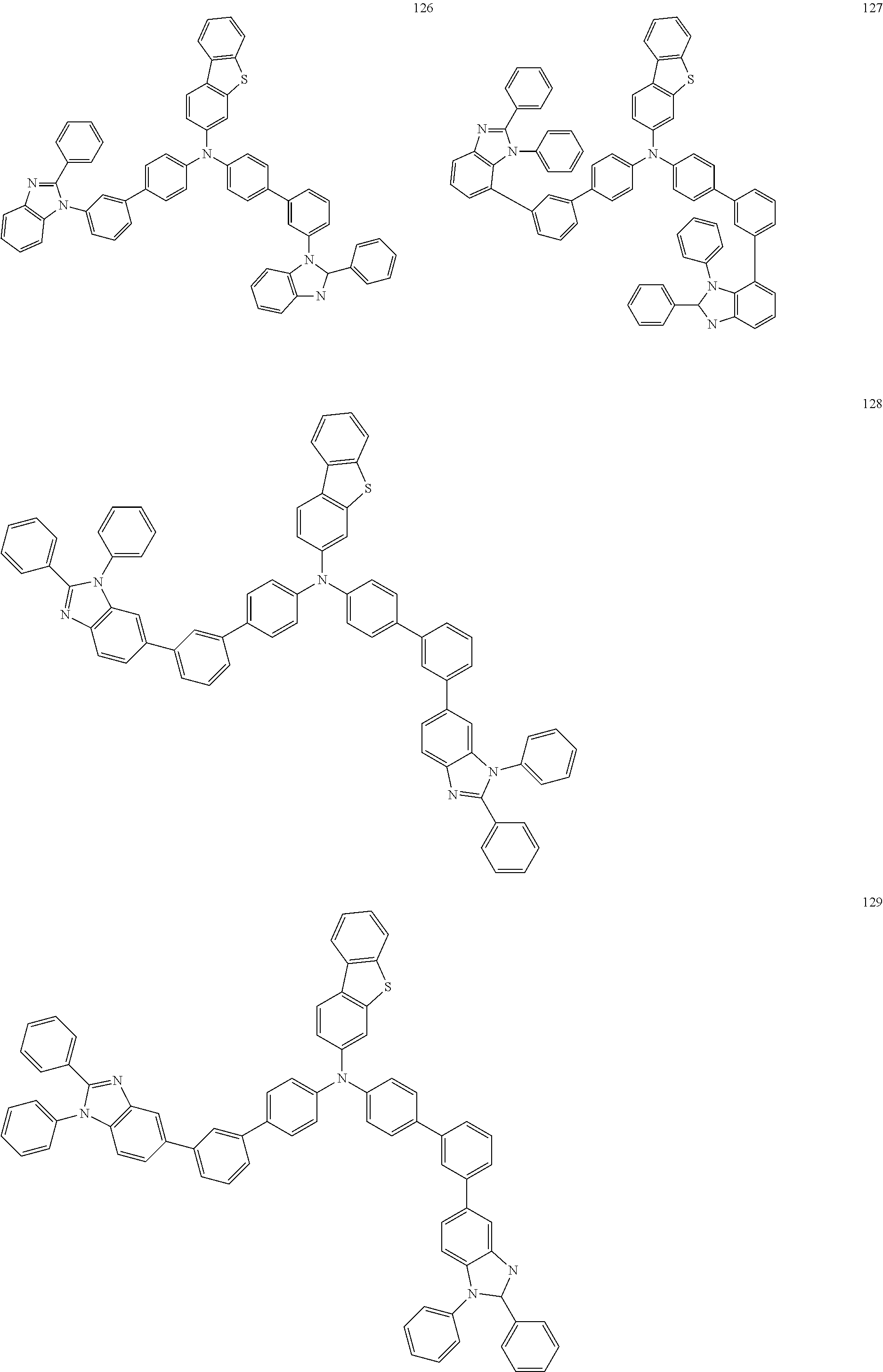

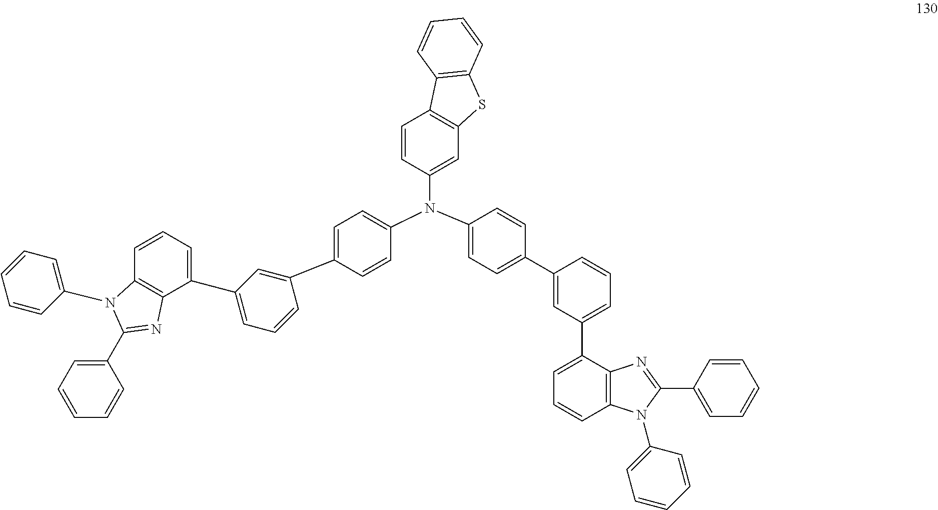 [SMILES: c1ccc(-c2nc3c(-c4cccc(-c5ccc(N(c6ccc(-c7cccc(-c8cccc9c8nc(-c8ccccc8)n9-c8ccccc8)c7)cc6)c6ccc7c(c6)sc6ccccc67)cc5)c4)cccc3n2-c2ccccc2)cc1]

6. A material for an organic electroluminescent device comprising an amine derivative represented by Formula 1: 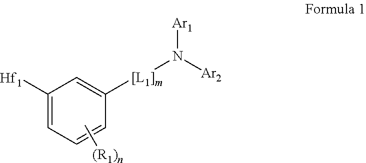
wherein in Formula 1,
Ar₁ and Ar₂ are each independently a substituted or unsubstituted aryl group having 6 to 30 carbon atoms for forming a ring, or a substituted or unsubstituted heteroaryl group having 2 to 30 carbon atoms for forming a ring;
L₁ is a divalent linking group;
R₁ is a substituted or unsubstituted alkyl group having 1 to 10 carbon atoms in a straight chain, branched chain, or ring form; a substituted or unsubstituted aryl group having 6 to 20 carbon atoms for forming a ring; a substituted or unsubstituted heteroaryl group having 1 to 20 carbon atoms for forming a ring; or an alkyl group, an aryl group, or a heteroaryl group formed through condensation of adjacent substituents;
n and m are each independently an integer from 0 to 4; and
Hf₁ is represented by Formula 2-1 or Formula 2-2; 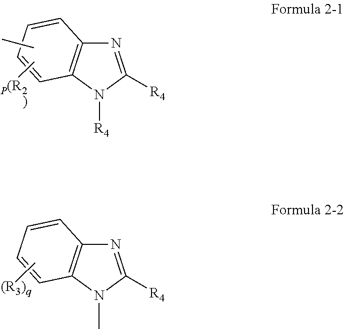
wherein in Formula 2-1 and 2-2,
R₂, R₃, and R₄ are each independently a substituted or unsubstituted alkyl group having 1 to 10 carbon atoms in a straight chain, branched chain, or ring form; a substituted or unsubstituted aryl group having 6 to 20 carbon atoms for forming a ring; a substituted or unsubstituted heteroaryl group having 1 to 20 carbon atoms for forming a ring; or an aryl group or a heteroaryl group formed through condensation of adjacent substituents;
p is an integer from 0 to 3; and
q is an integer from 0 to 4.

7. The material for an organic electroluminescent device of claim 6, wherein L₁ is a substituted or unsubstituted arylene group having 6 to 30 carbon atoms for forming a ring, or a substituted or unsubstituted heteroarylene group having 2 to 30 carbon atoms for forming a ring.

8. The material for an organic electroluminescent device of claim 6, wherein Ar₁ and Ar₂ are each independently a non-nitrogen atom-containing aryl group or heteroaryl group which is unsubstituted or substituted with a substituent which does not include a nitrogen atom.

9. The material for an organic electroluminescent device of claim 6, wherein Ar₁ is a non-nitrogen atom-containing aryl group or heteroaryl group which is unsubstituted or substituted with a substituent which does not include a nitrogen atom, and Ar₂ is represented by Formula 3: 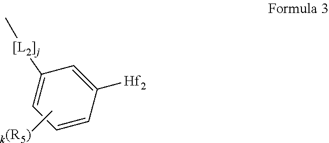
wherein in Formula 3,
L₂ is a divalent linking group;
R₅ is a substituted or unsubstituted alkyl group having 1 to 10 carbon atoms in a straight chain, branched chain, or ring form; a substituted or unsubstituted aryl group having 6 to 20 carbon atoms for forming a ring; a substituted or unsubstituted heteroaryl group having 1 to 20 carbon atoms for forming a ring; or an alkyl group, an aryl group, or a heteroaryl group formed through condensation of adjacent substituents;
j and k are each independently an integer from 0 to 4; and
Hf₂ is represented by Formula 4-1 or Formula 4-2; 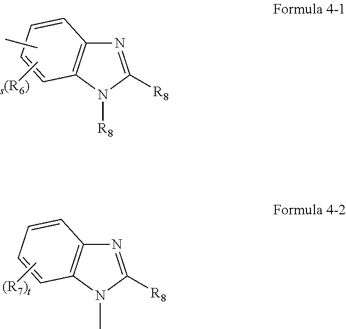
wherein in Formula 4-1 and 4-2,
R₆, R₇, and R₈ are each independently a substituted or unsubstituted alkyl group having 1 to 10 carbon atoms in a straight chain, branched chain, or ring form; a substituted or unsubstituted aryl group having 6 to 20 carbon atoms for forming a ring; a substituted or unsubstituted heteroaryl group having 1 to 20 carbon atoms for forming a ring; or an aryl group or a heteroaryl group formed through condensation of adjacent substituents;
s is an integer from 0 to 3; and
t is an integer from 0 to 4.

10. The material for an organic electroluminescent device of claim 6, wherein the amine derivative represented by Formula 1 is at least one of compounds 1 to 130:

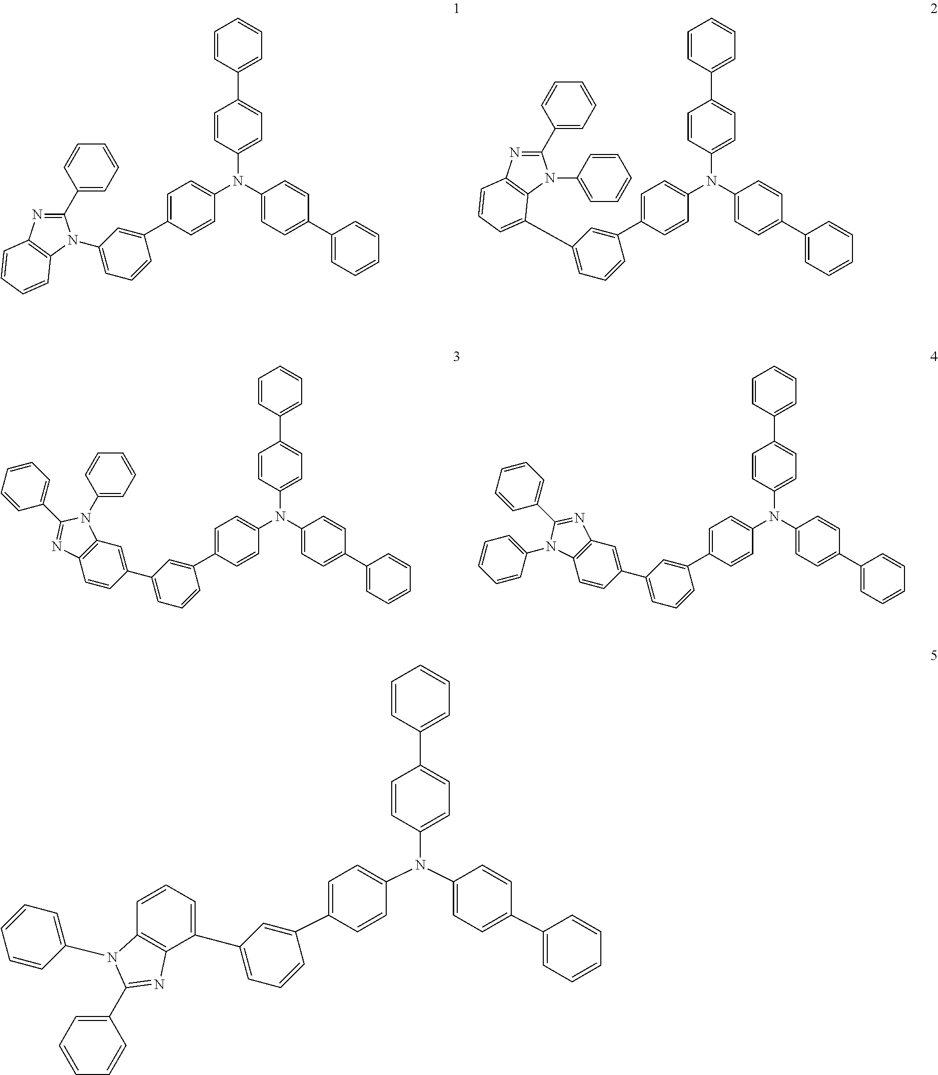 [SMILES: c1ccc(-c2ccc(N(c3ccc(-c4ccccc4)cc3)c3ccc(-c4cccc(-n5c(-c6ccccc6)nc6ccccc65)c4)cc3)cc2)cc1 ; c1ccc(-c2ccc(N(c3ccc(-c4ccccc4)cc3)c3ccc(-c4cccc(-c5cccc6nc(-c7ccccc7)n(-c7ccccc7)c56)c4)cc3)cc2)cc1 ; c1ccc(-c2ccc(N(c3ccc(-c4ccccc4)cc3)c3ccc(-c4cccc(-c5ccc6nc(-c7ccccc7)n(-c7ccccc7)c6c5)c4)cc3)cc2)cc1 ; c1ccc(-c2ccc(N(c3ccc(-c4ccccc4)cc3)c3ccc(-c4cccc(-c5ccc6c(c5)nc(-c5ccccc5)n6-c5ccccc5)c4)cc3)cc2)cc1 ; c1ccc(-c2ccc(N(c3ccc(-c4ccccc4)cc3)c3ccc(-c4cccc(-c5cccc6c5nc(-c5ccccc5)n6-c5ccccc5)c4)cc3)cc2)cc1]

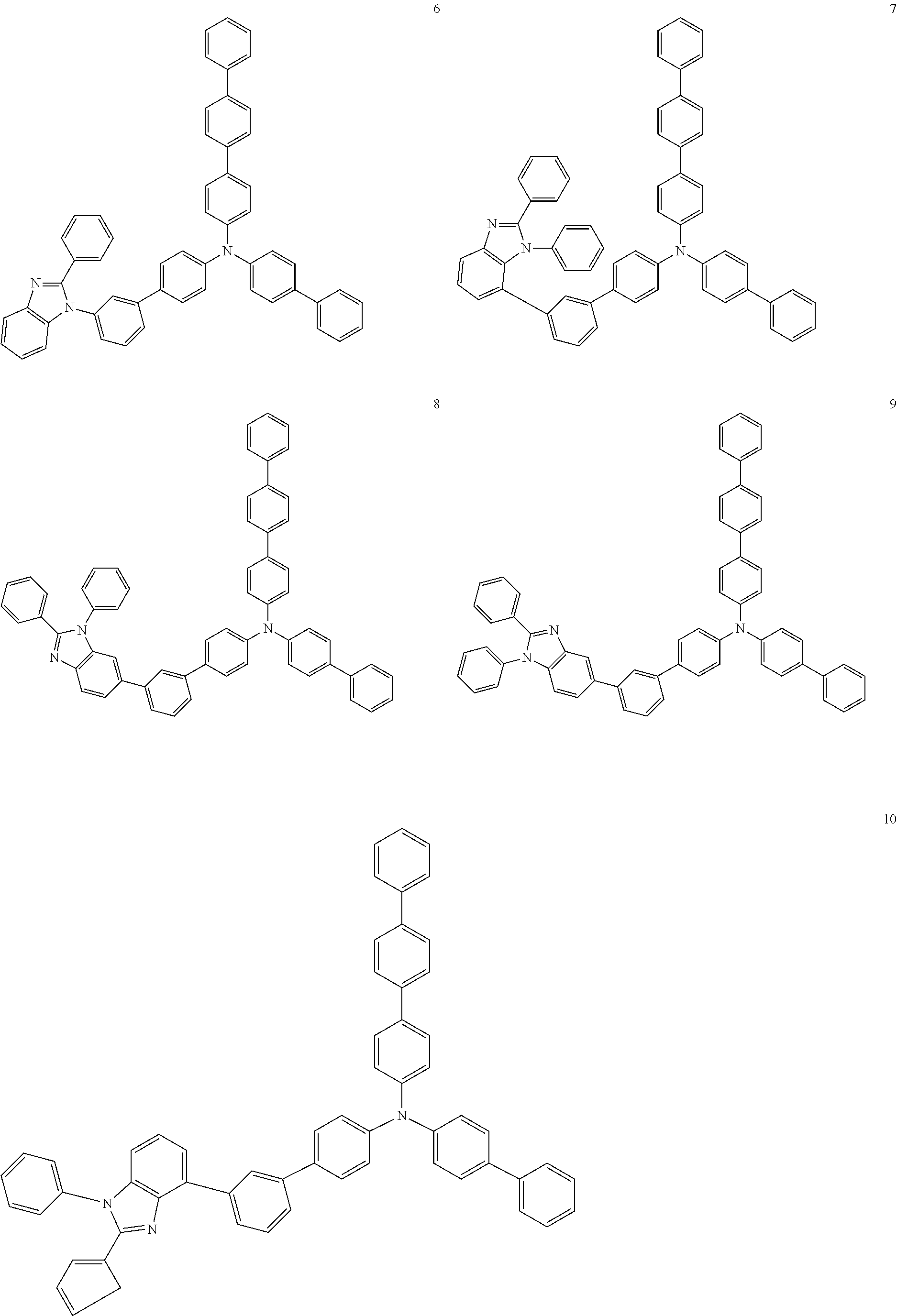

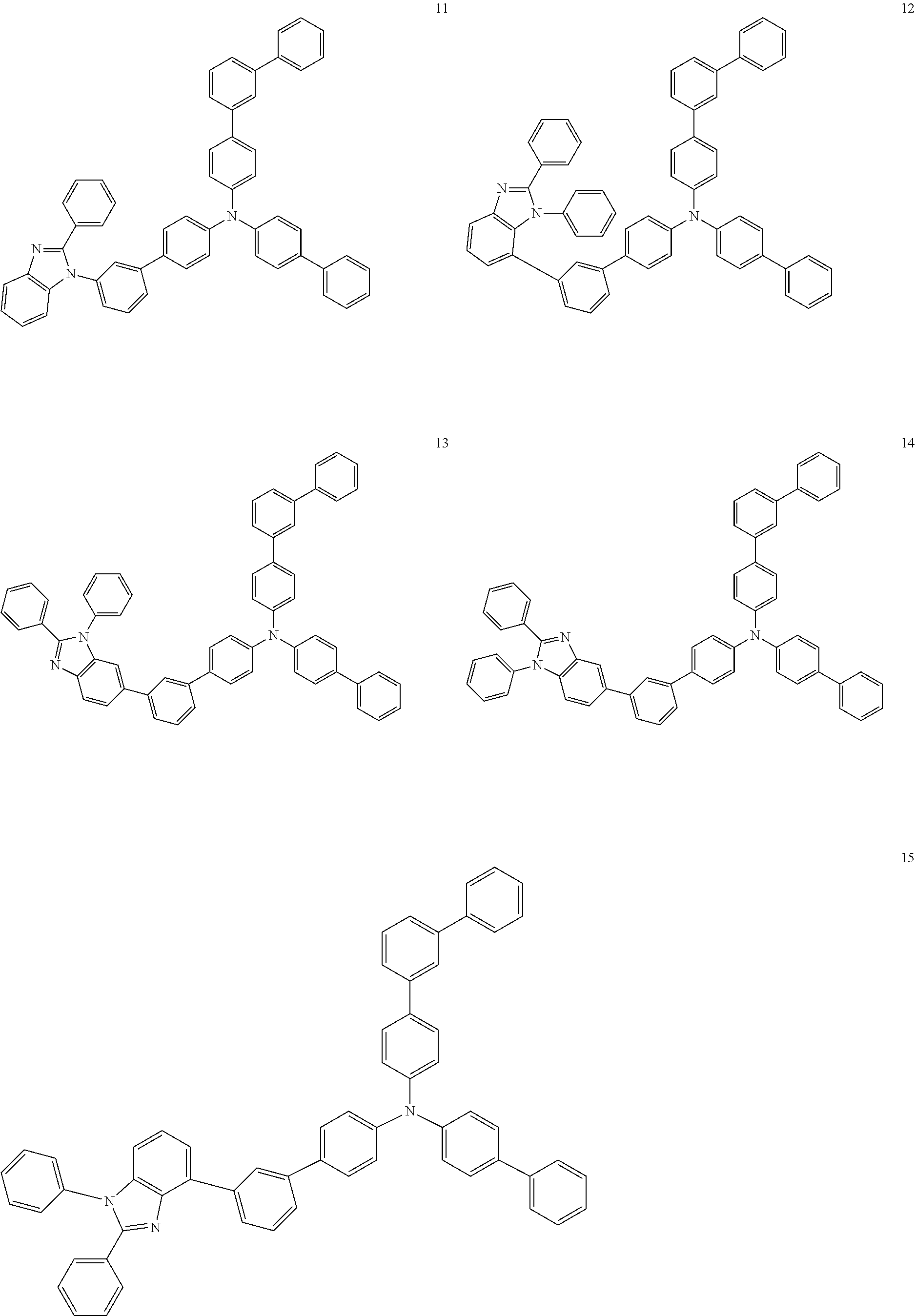

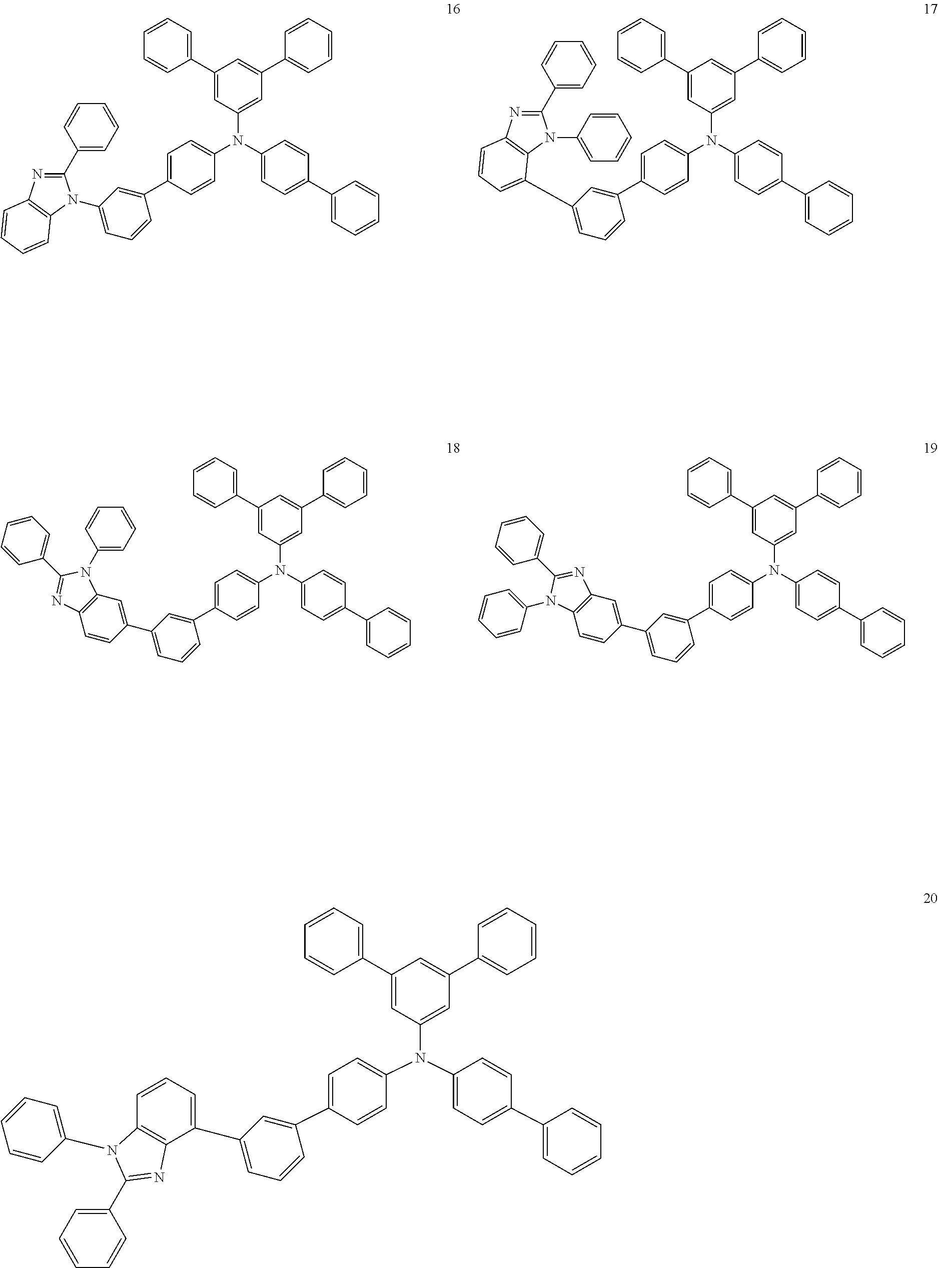

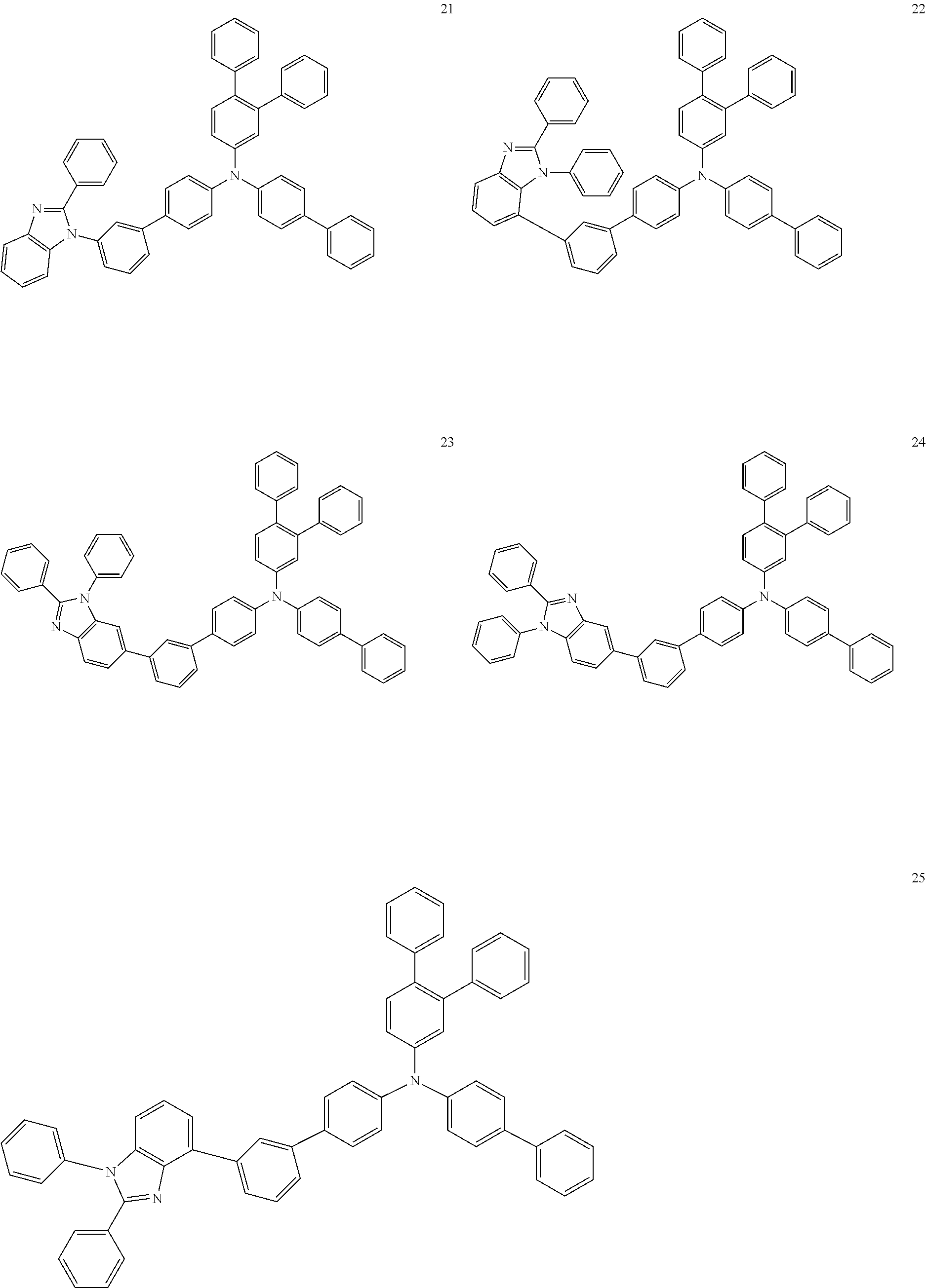

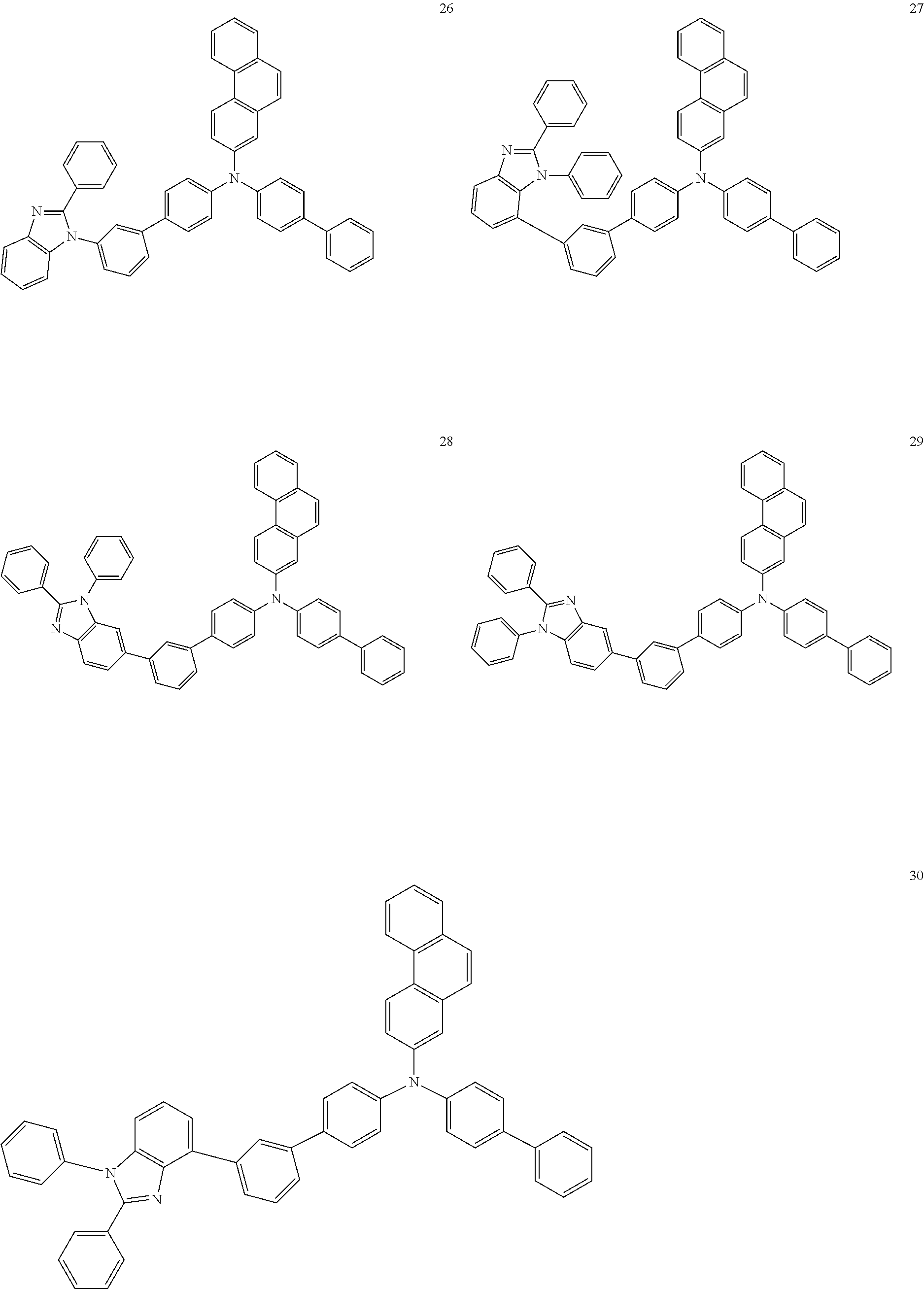

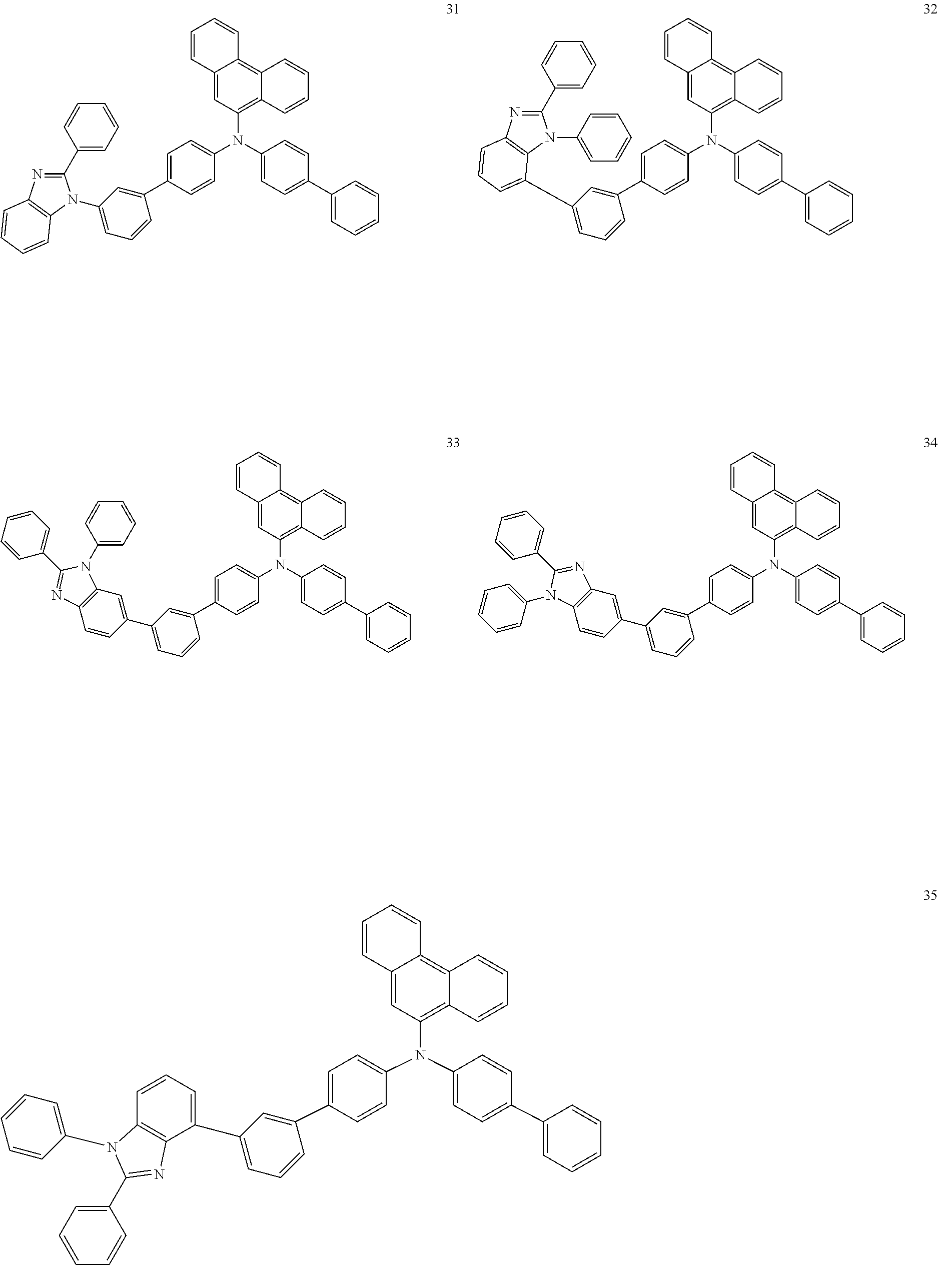

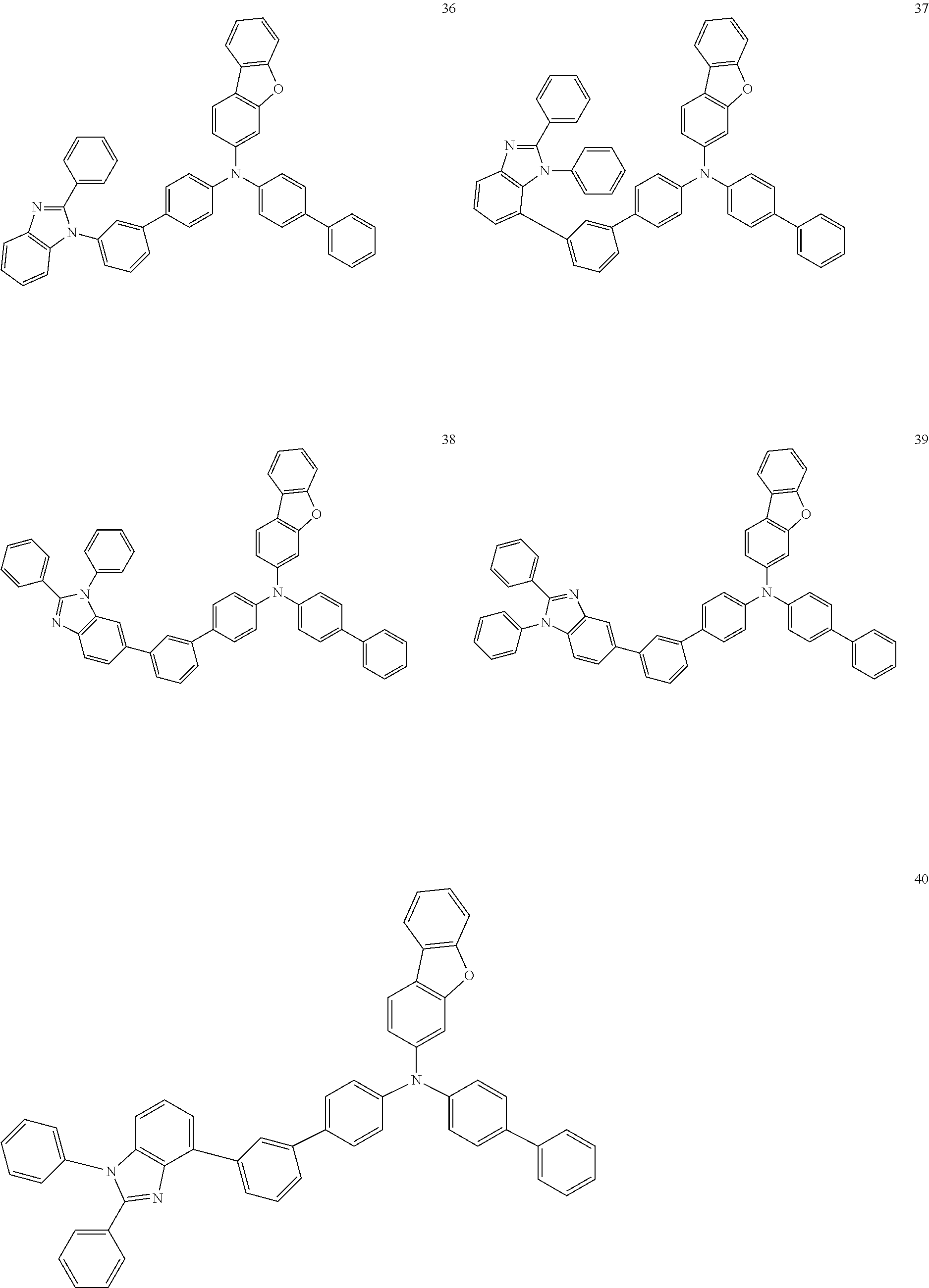

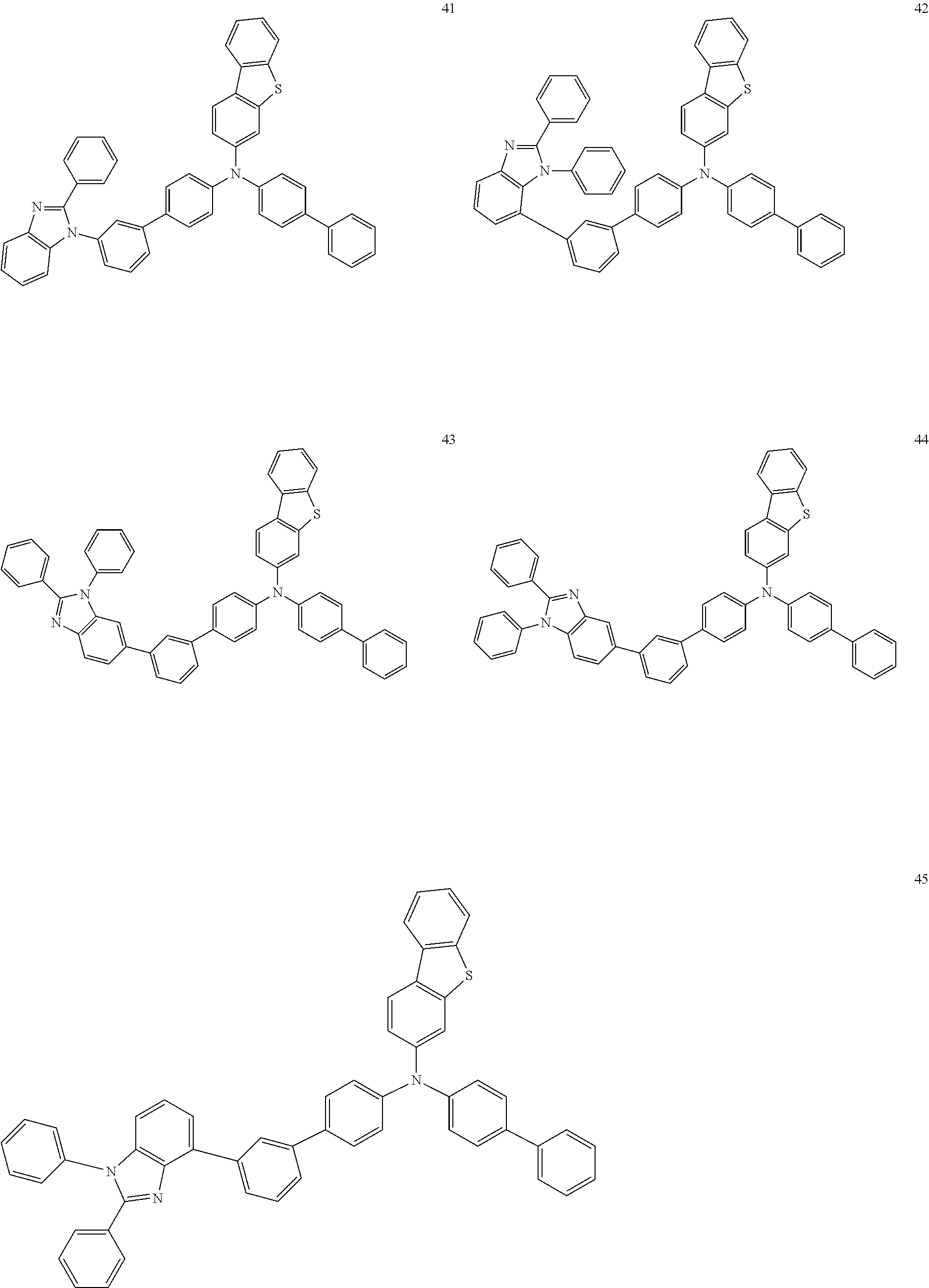

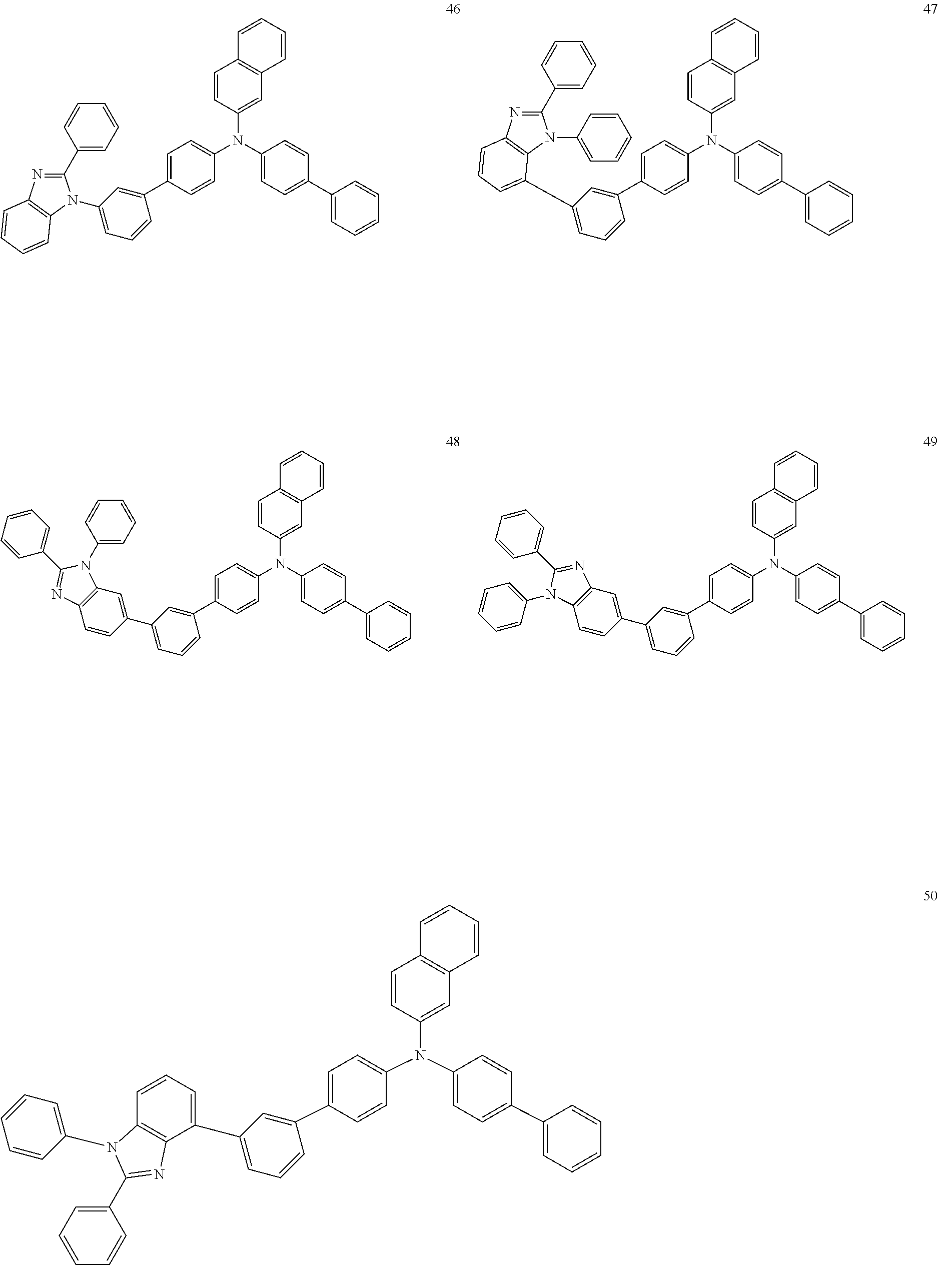 [SMILES: c1ccc(-c2ccc(N(c3ccc(-c4cccc(-n5c(-c6ccccc6)nc6ccccc65)c4)cc3)c3ccc4ccccc4c3)cc2)cc1 ; c1ccc(-c2ccc(N(c3ccc(-c4cccc(-c5cccc6nc(-c7ccccc7)n(-c7ccccc7)c56)c4)cc3)c3ccc4ccccc4c3)cc2)cc1 ; c1ccc(-c2ccc(N(c3ccc(-c4cccc(-c5ccc6nc(-c7ccccc7)n(-c7ccccc7)c6c5)c4)cc3)c3ccc4ccccc4c3)cc2)cc1 ; c1ccc(-c2ccc(N(c3ccc(-c4cccc(-c5ccc6c(c5)nc(-c5ccccc5)n6-c5ccccc5)c4)cc3)c3ccc4ccccc4c3)cc2)cc1 ; c1ccc(-c2ccc(N(c3ccc(-c4cccc(-c5cccc6c5nc(-c5ccccc5)n6-c5ccccc5)c4)cc3)c3ccc4ccccc4c3)cc2)cc1]

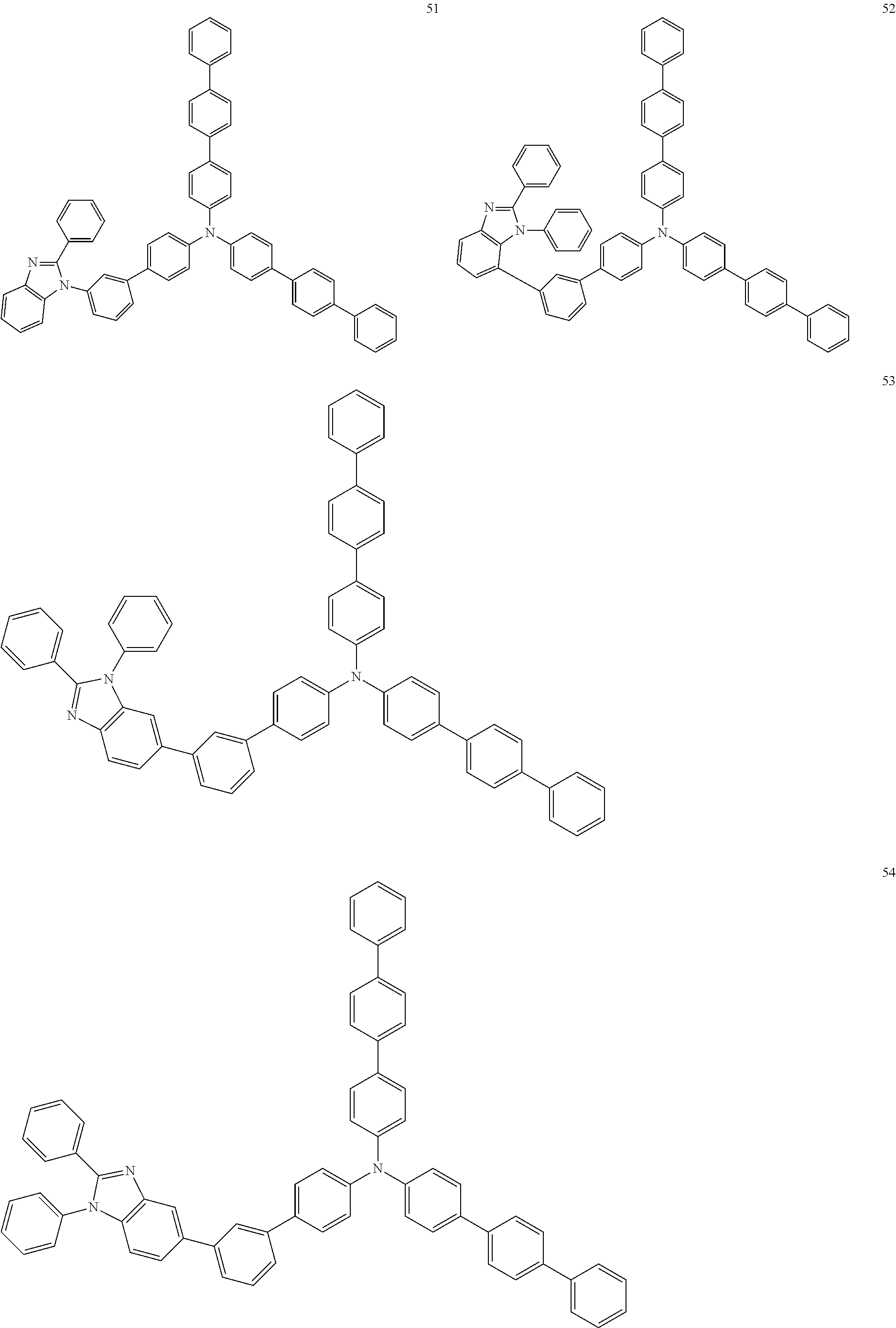

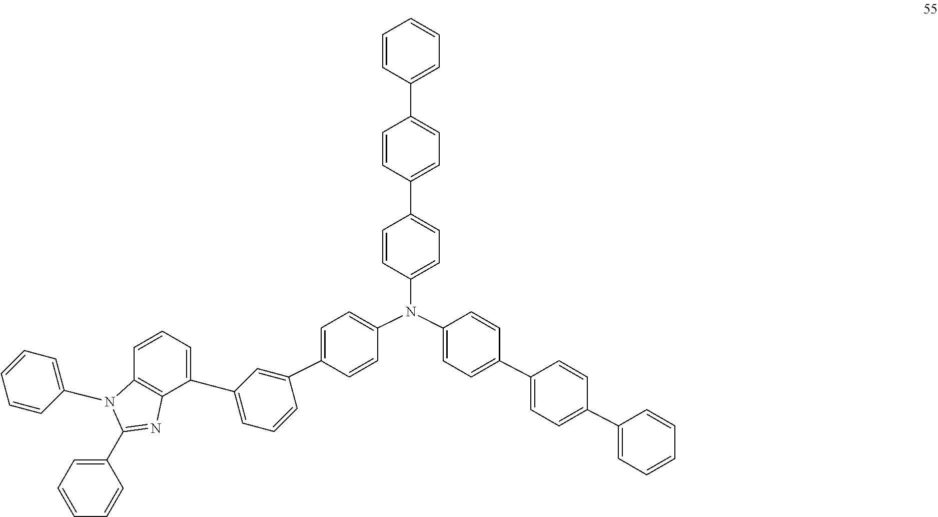 [SMILES: c1ccc(-c2ccc(-c3ccc(N(c4ccc(-c5ccc(-c6ccccc6)cc5)cc4)c4ccc(-c5cccc(-c6cccc7c6nc(-c6ccccc6)n7-c6ccccc6)c5)cc4)cc3)cc2)cc1]

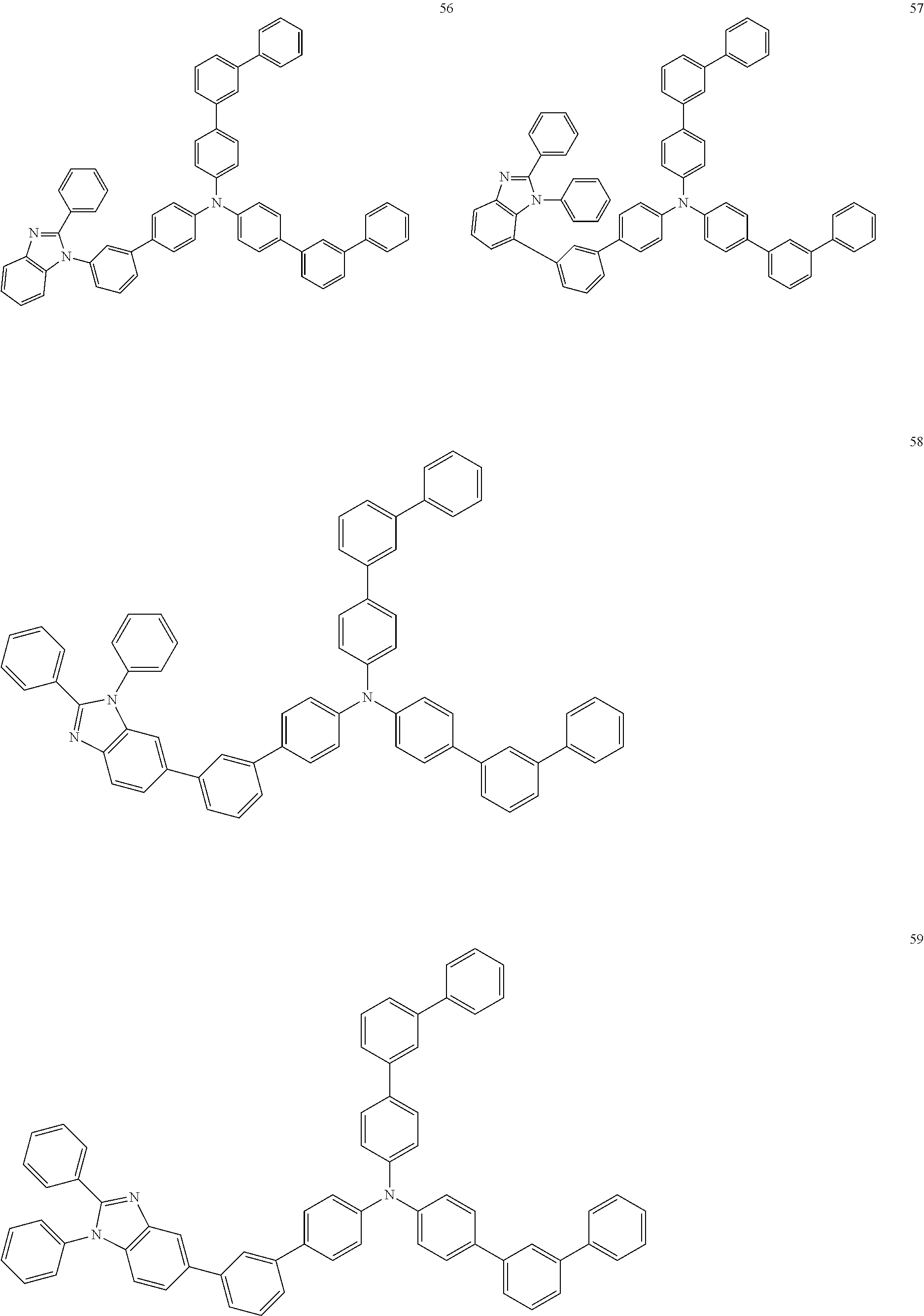 [SMILES: c1ccc(-c2cccc(-c3ccc(N(c4ccc(-c5cccc(-c6ccccc6)c5)cc4)c4ccc(-c5cccc(-n6c(-c7ccccc7)nc7ccccc76)c5)cc4)cc3)c2)cc1 ; c1ccc(-c2cccc(-c3ccc(N(c4ccc(-c5cccc(-c6ccccc6)c5)cc4)c4ccc(-c5cccc(-c6cccc7nc(-c8ccccc8)n(-c8ccccc8)c67)c5)cc4)cc3)c2)cc1 ; c1ccc(-c2cccc(-c3ccc(N(c4ccc(-c5cccc(-c6ccccc6)c5)cc4)c4ccc(-c5cccc(-c6ccc7nc(-c8ccccc8)n(-c8ccccc8)c7c6)c5)cc4)cc3)c2)cc1 ; c1ccc(-c2cccc(-c3ccc(N(c4ccc(-c5cccc(-c6ccccc6)c5)cc4)c4ccc(-c5cccc(-c6ccc7c(c6)nc(-c6ccccc6)n7-c6ccccc6)c5)cc4)cc3)c2)cc1]

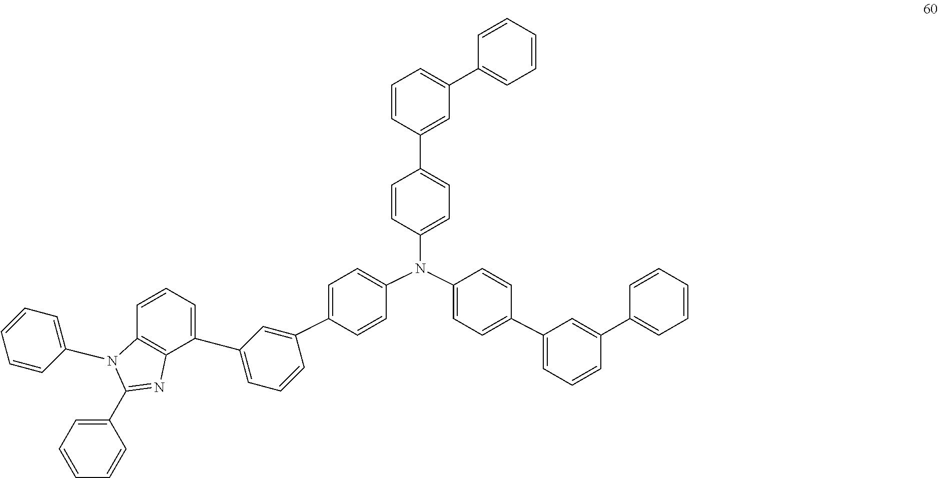 [SMILES: c1ccc(-c2cccc(-c3ccc(N(c4ccc(-c5cccc(-c6ccccc6)c5)cc4)c4ccc(-c5cccc(-c6cccc7c6nc(-c6ccccc6)n7-c6ccccc6)c5)cc4)cc3)c2)cc1]

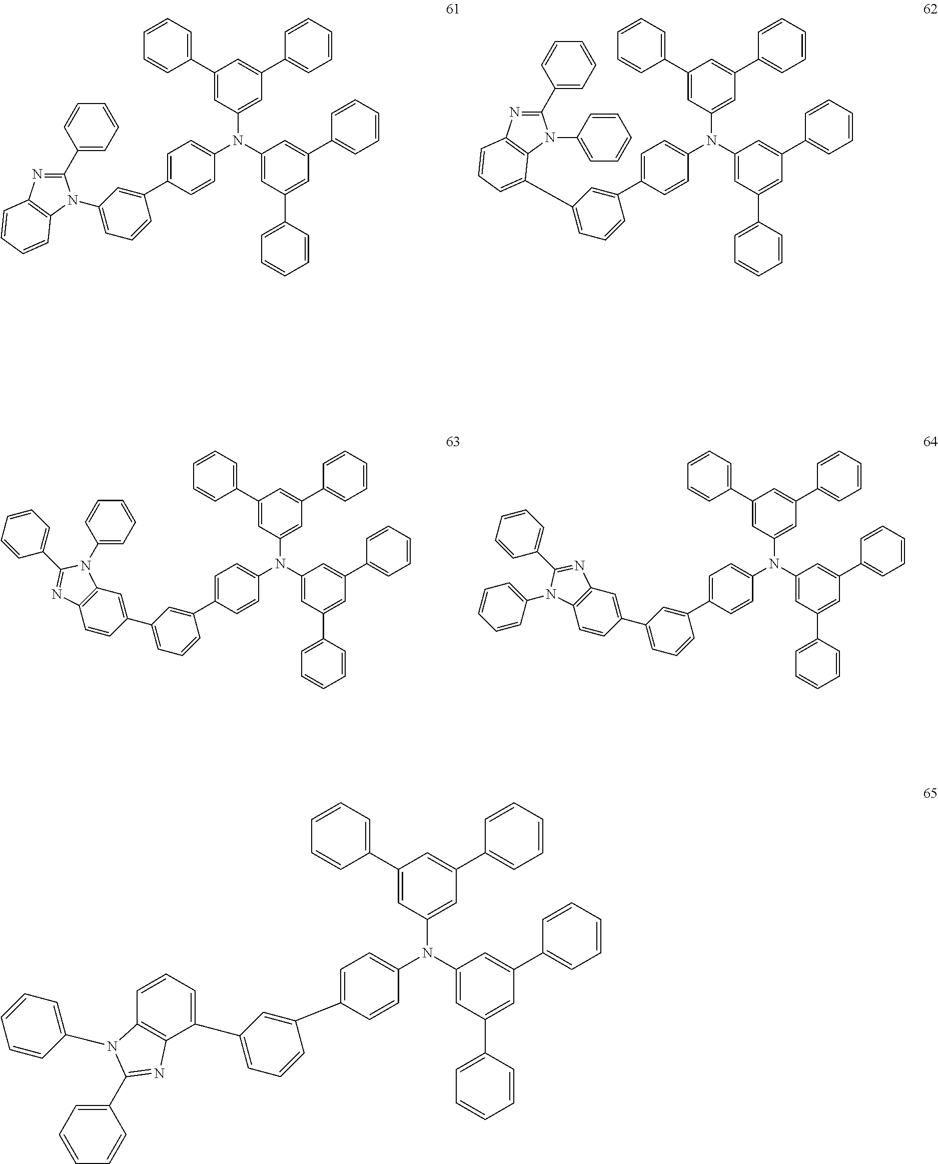

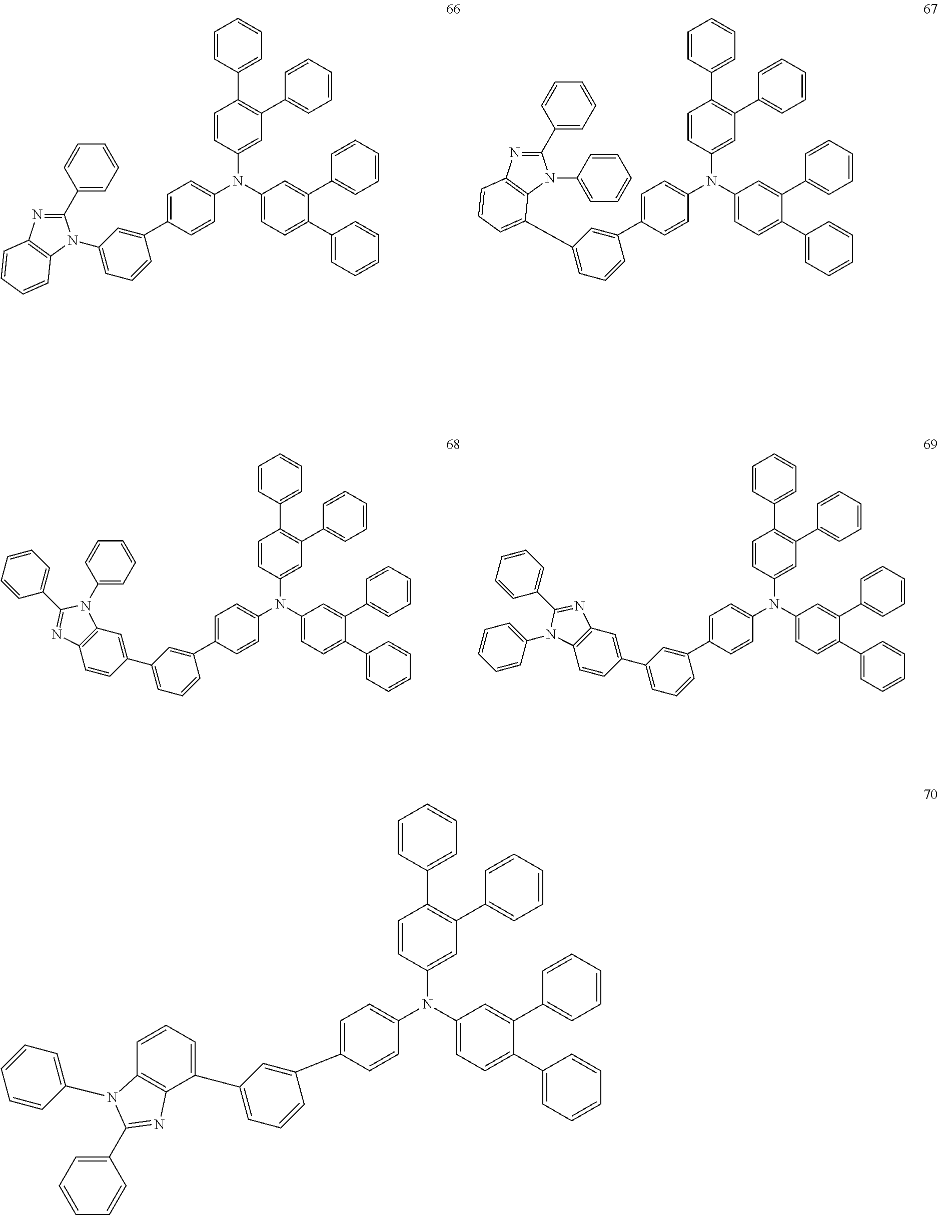

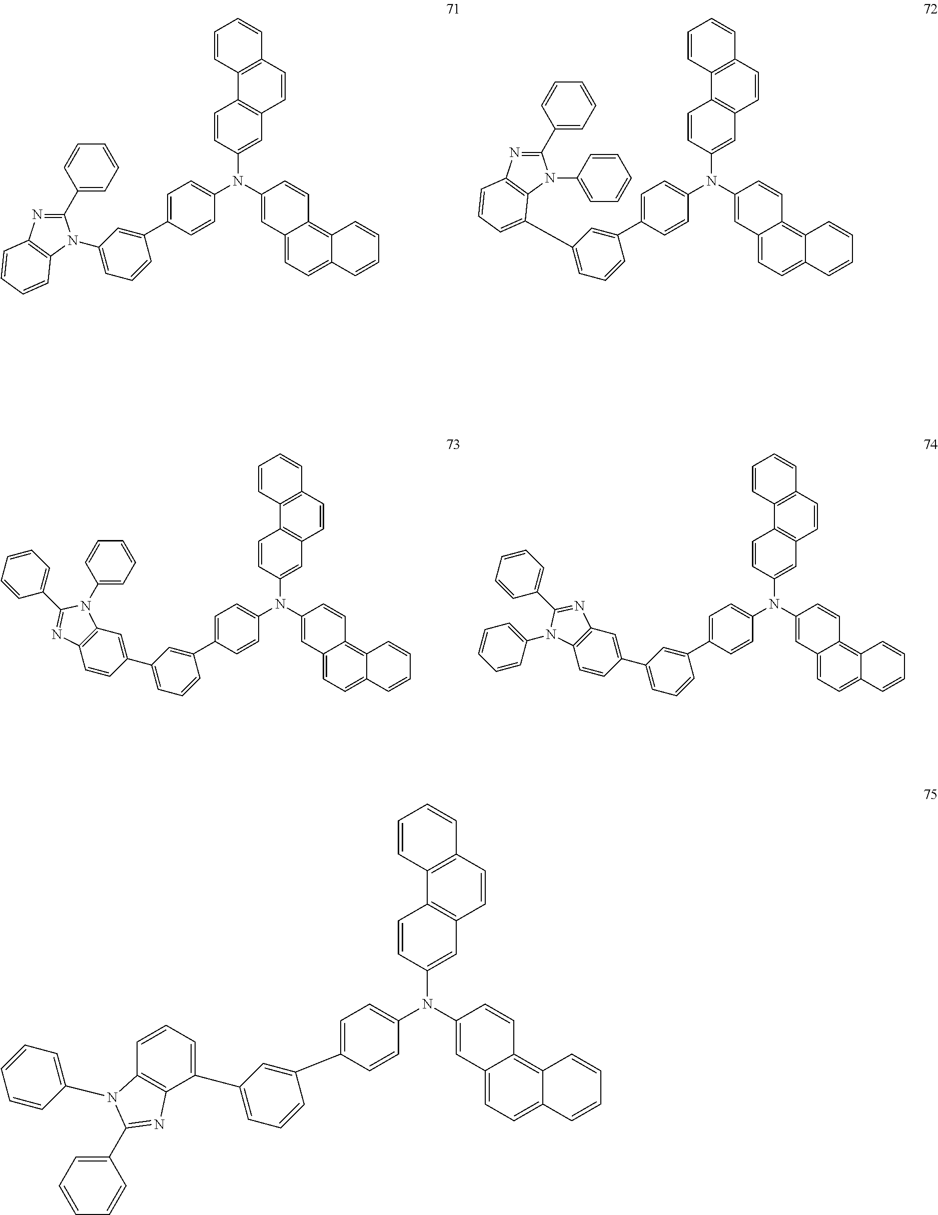

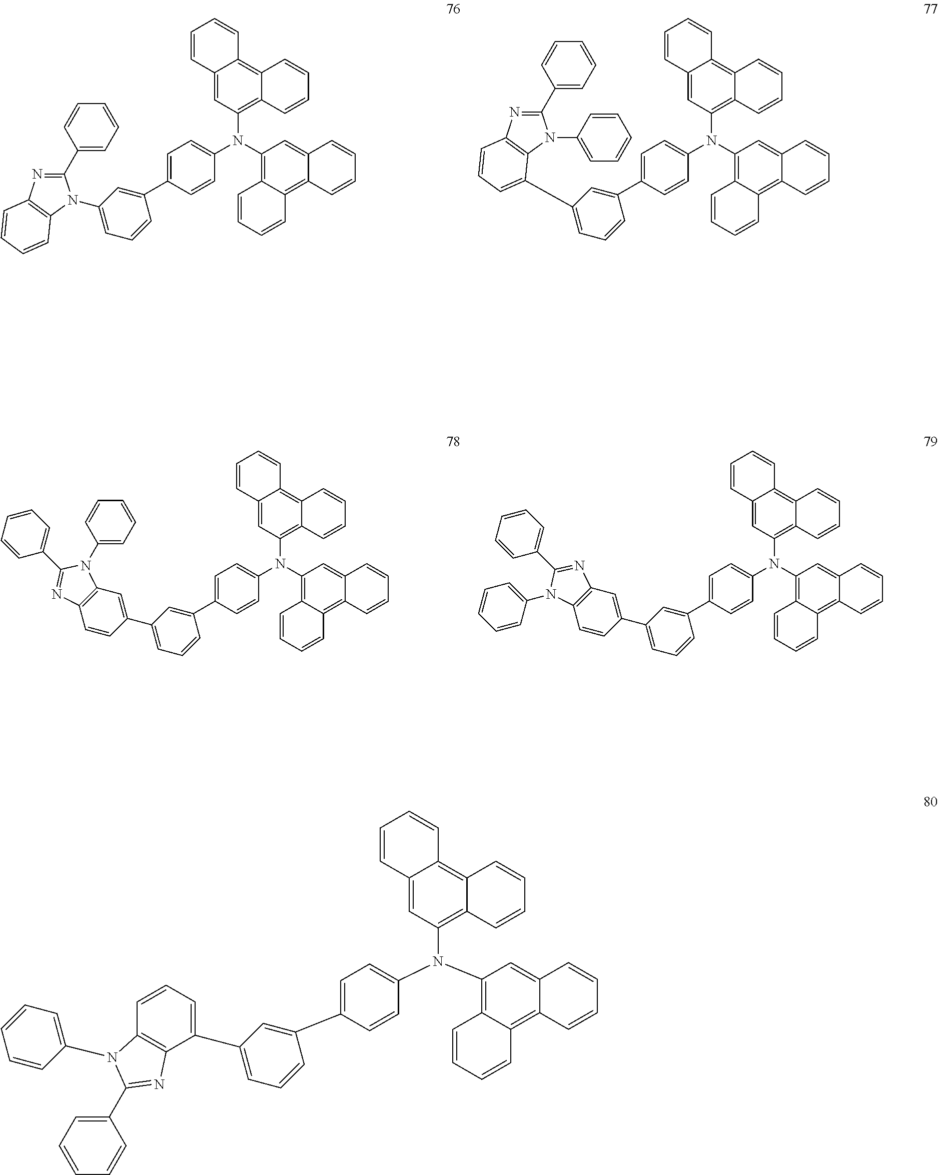

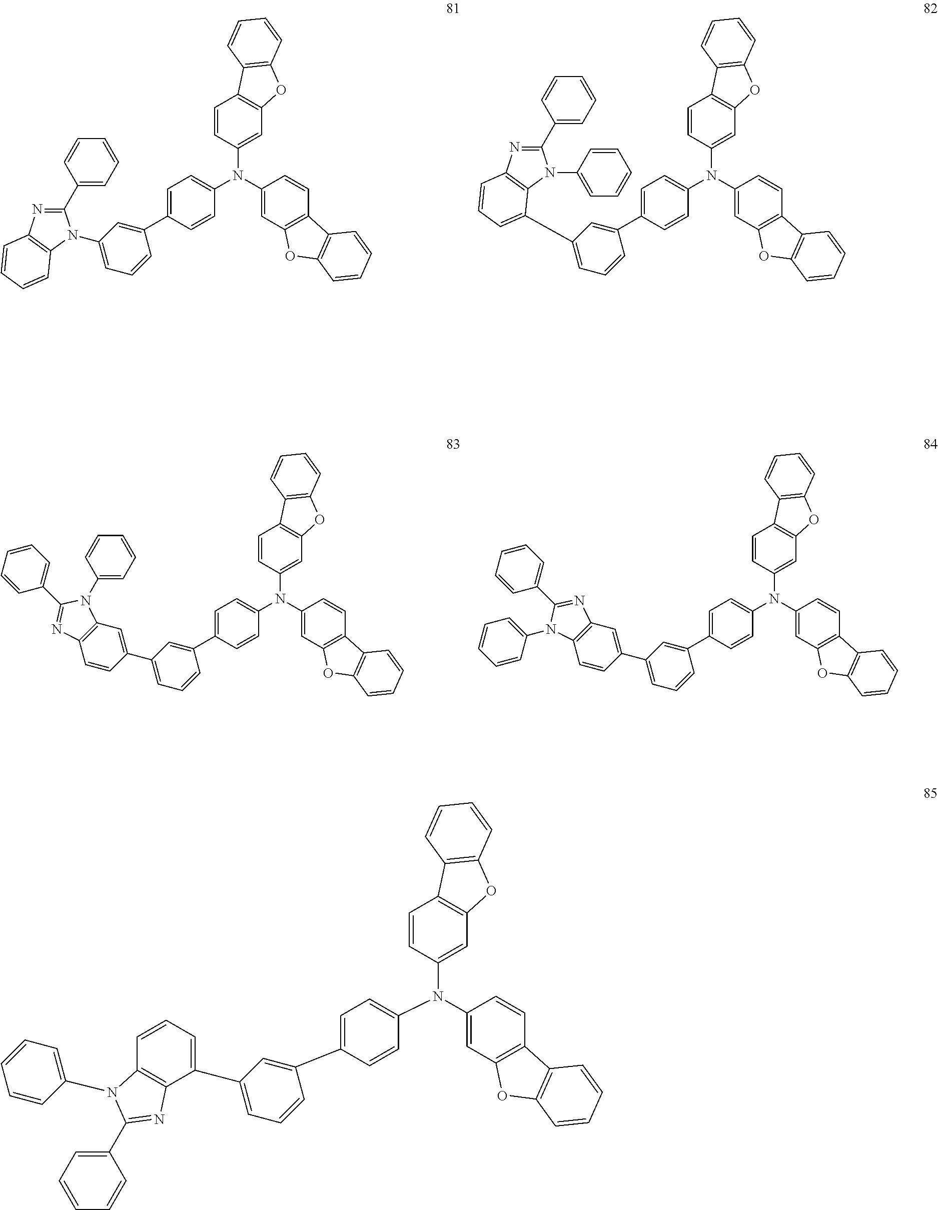

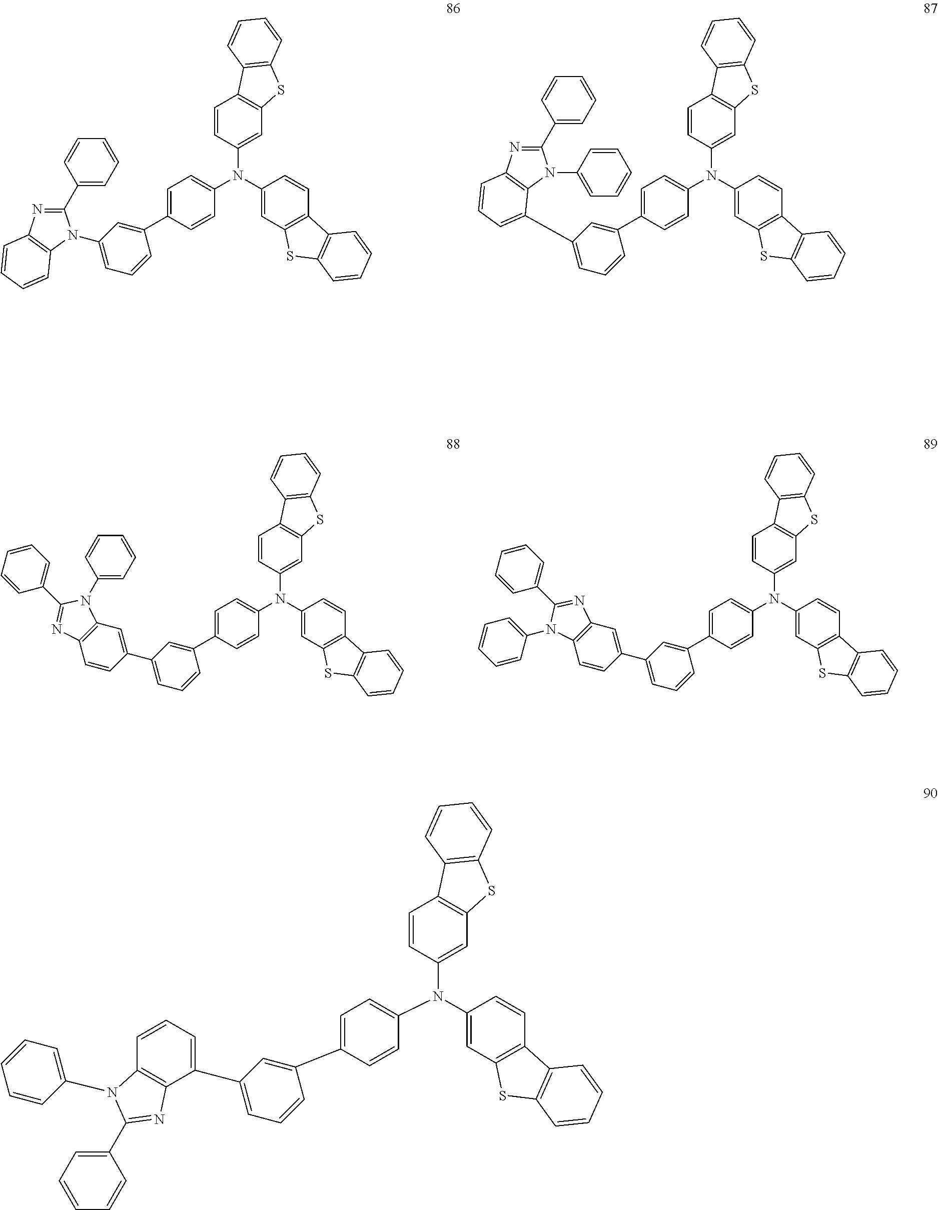

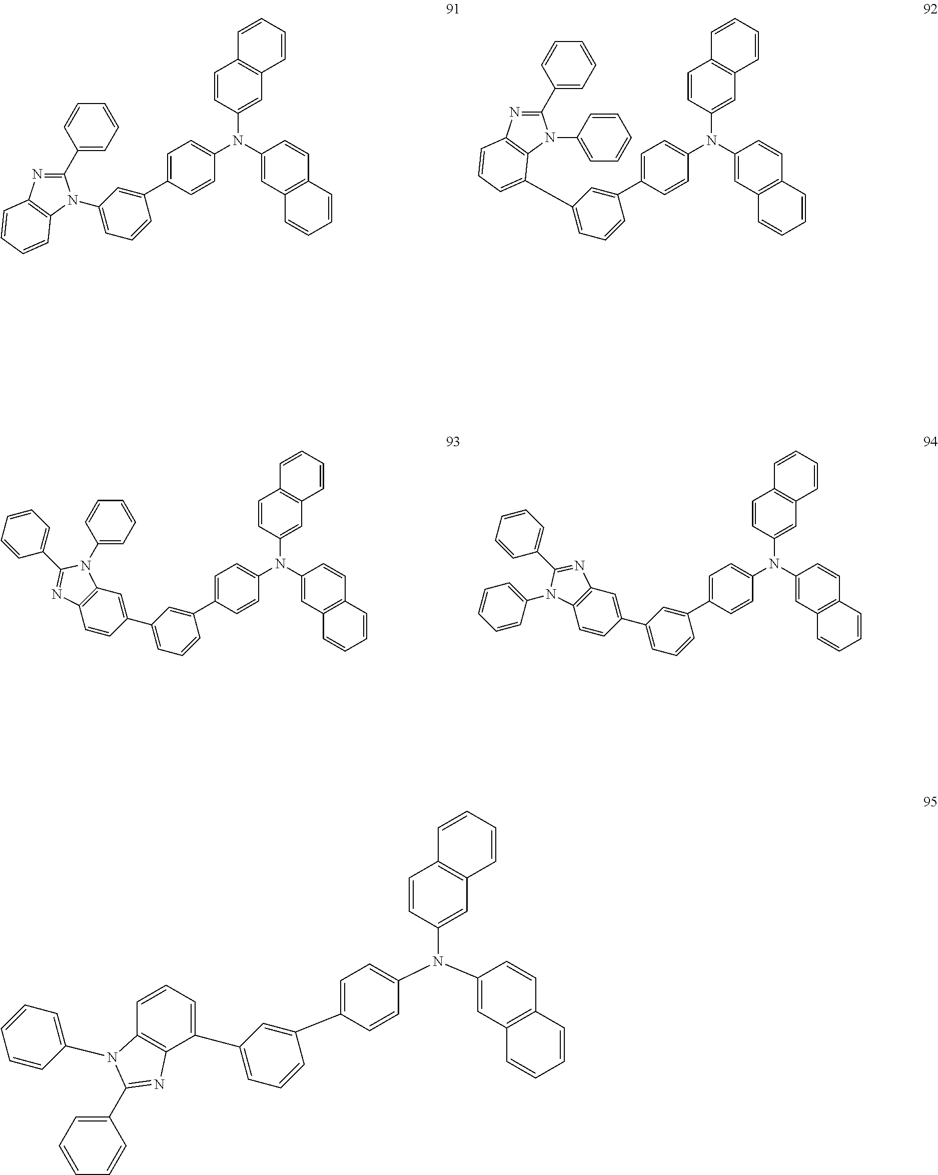 [SMILES: c1ccc(-c2nc3ccccc3n2-c2cccc(-c3ccc(N(c4ccc5ccccc5c4)c4ccc5ccccc5c4)cc3)c2)cc1 ; c1ccc(-c2nc3cccc(-c4cccc(-c5ccc(N(c6ccc7ccccc7c6)c6ccc7ccccc7c6)cc5)c4)c3n2-c2ccccc2)cc1 ; c1ccc(-c2nc3ccc(-c4cccc(-c5ccc(N(c6ccc7ccccc7c6)c6ccc7ccccc7c6)cc5)c4)cc3n2-c2ccccc2)cc1 ; c1ccc(-c2nc3cc(-c4cccc(-c5ccc(N(c6ccc7ccccc7c6)c6ccc7ccccc7c6)cc5)c4)ccc3n2-c2ccccc2)cc1 ; c1ccc(-c2nc3c(-c4cccc(-c5ccc(N(c6ccc7ccccc7c6)c6ccc7ccccc7c6)cc5)c4)cccc3n2-c2ccccc2)cc1]

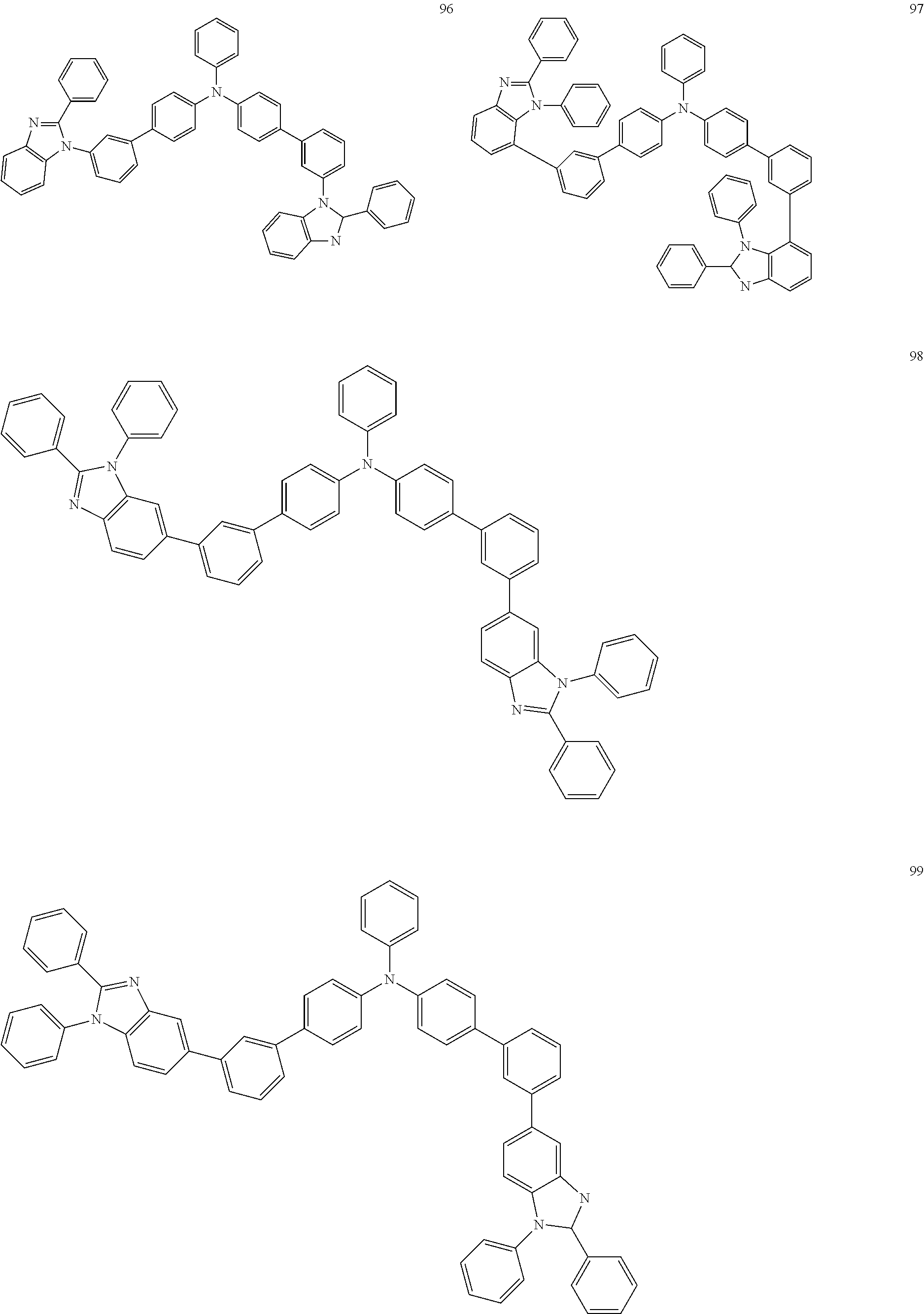 [SMILES: c1ccc(-c2nc3ccccc3n2-c2cccc(-c3ccc(N(c4ccccc4)c4ccc(-c5cccc(N6c7ccccc7NC6c6ccccc6)c5)cc4)cc3)c2)cc1 ; c1ccc(-c2nc3cccc(-c4cccc(-c5ccc(N(c6ccccc6)c6ccc(-c7cccc(-c8cccc9c8N(c8ccccc8)C(c8ccccc8)N9)c7)cc6)cc5)c4)c3n2-c2ccccc2)cc1 ; c1ccc(-c2nc3ccc(-c4cccc(-c5ccc(N(c6ccccc6)c6ccc(-c7cccc(-c8ccc9nc(-c%10ccccc%10)n(-c%10ccccc%10)c9c8)c7)cc6)cc5)c4)cc3n2-c2ccccc2)cc1 ; c1ccc(-c2nc3cc(-c4cccc(-c5ccc(N(c6ccccc6)c6ccc(-c7cccc(-c8ccc9c(c8)NC(c8ccccc8)N9c8ccccc8)c7)cc6)cc5)c4)ccc3n2-c2ccccc2)cc1]

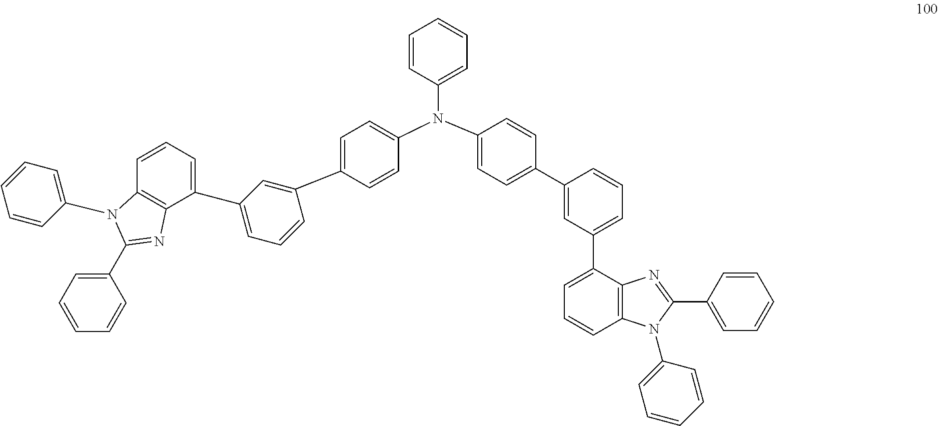 [SMILES: c1ccc(-c2nc3c(-c4cccc(-c5ccc(N(c6ccccc6)c6ccc(-c7cccc(-c8cccc9c8nc(-c8ccccc8)n9-c8ccccc8)c7)cc6)cc5)c4)cccc3n2-c2ccccc2)cc1]

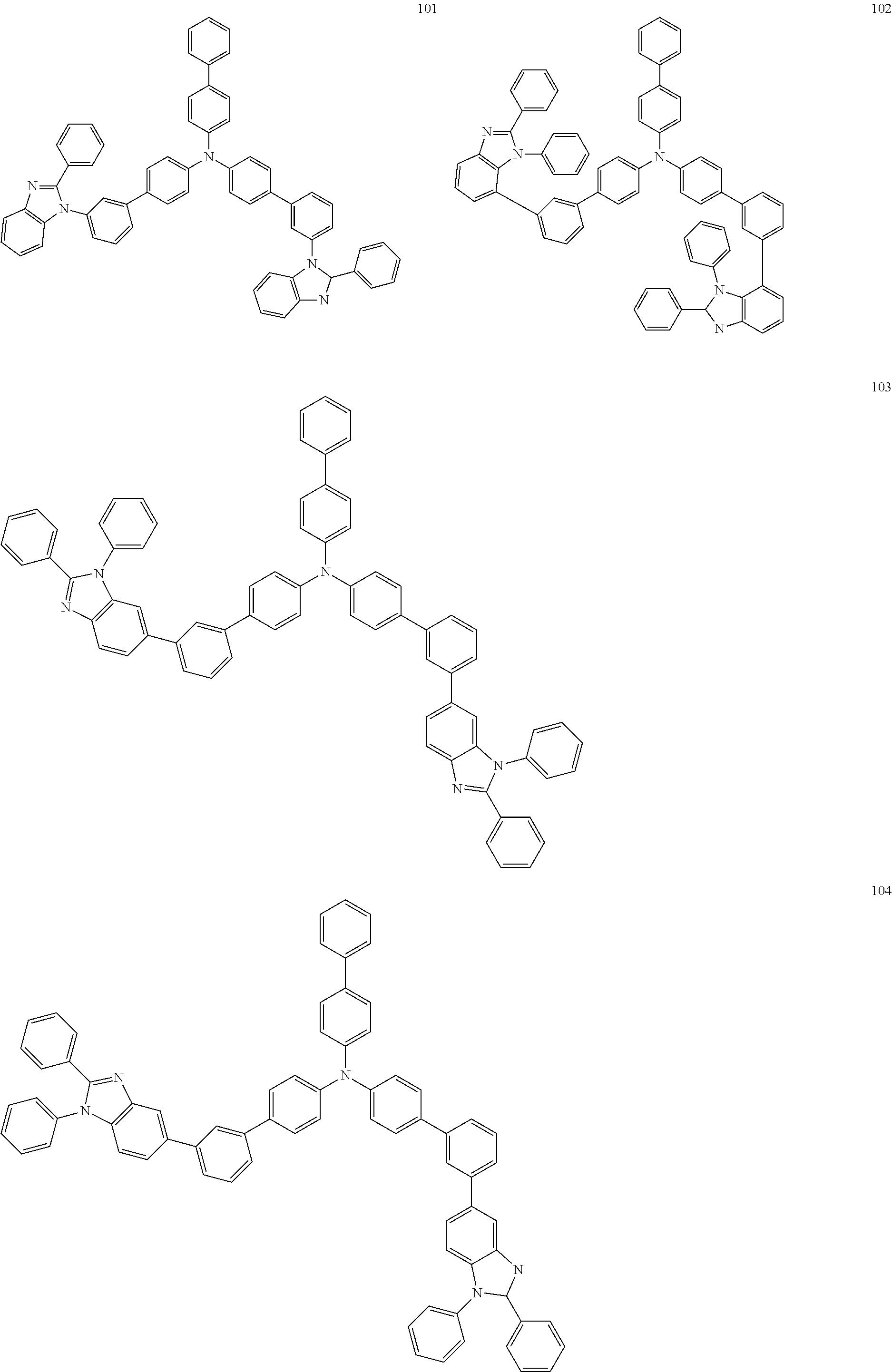

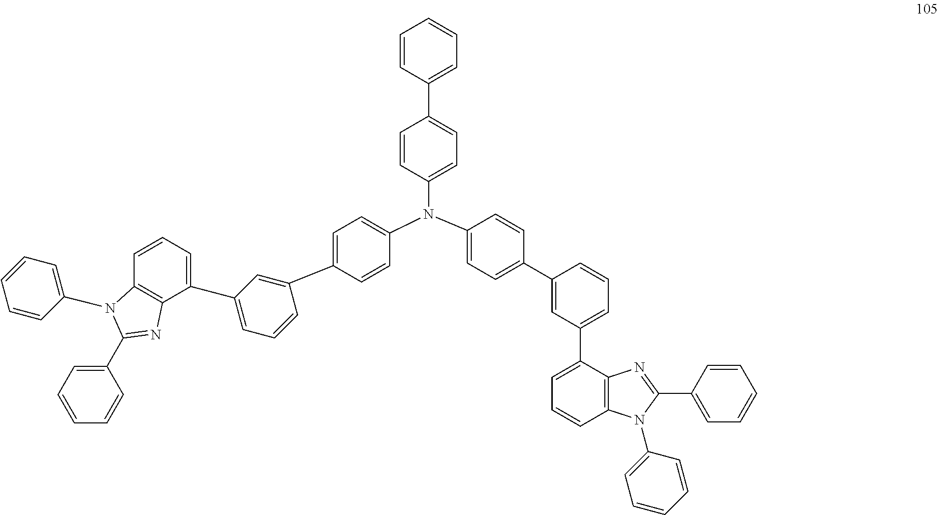 [SMILES: c1ccc(-c2ccc(N(c3ccc(-c4cccc(-c5cccc6c5nc(-c5ccccc5)n6-c5ccccc5)c4)cc3)c3ccc(-c4cccc(-c5cccc6c5nc(-c5ccccc5)n6-c5ccccc5)c4)cc3)cc2)cc1]

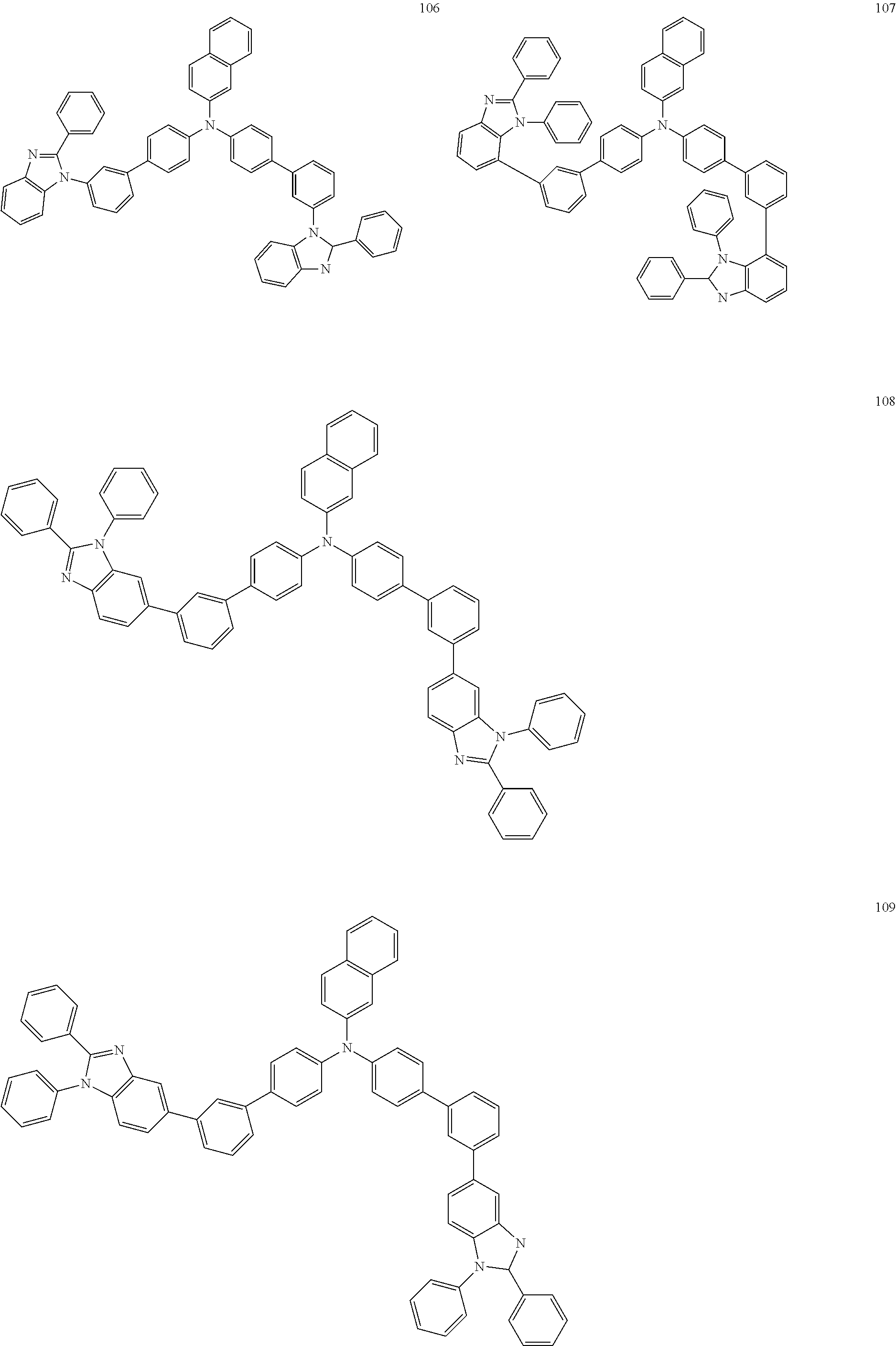

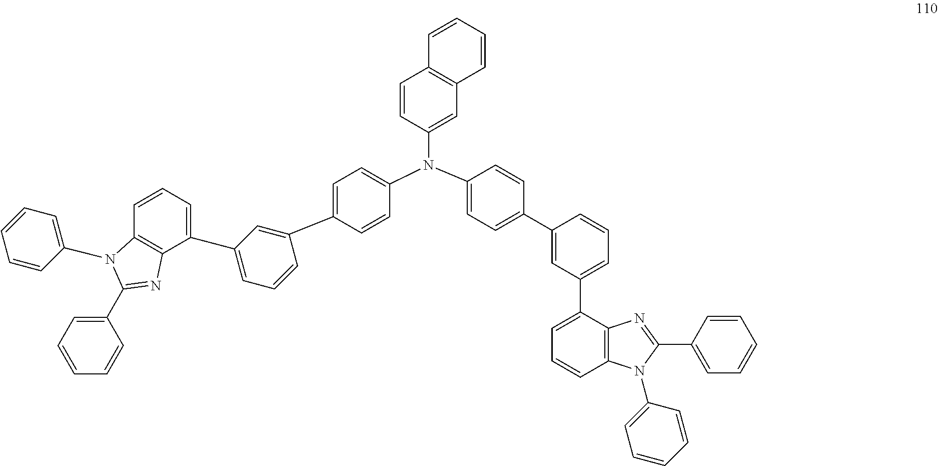 [SMILES: c1ccc(-c2nc3c(-c4cccc(-c5ccc(N(c6ccc(-c7cccc(-c8cccc9c8nc(-c8ccccc8)n9-c8ccccc8)c7)cc6)c6ccc7ccccc7c6)cc5)c4)cccc3n2-c2ccccc2)cc1]

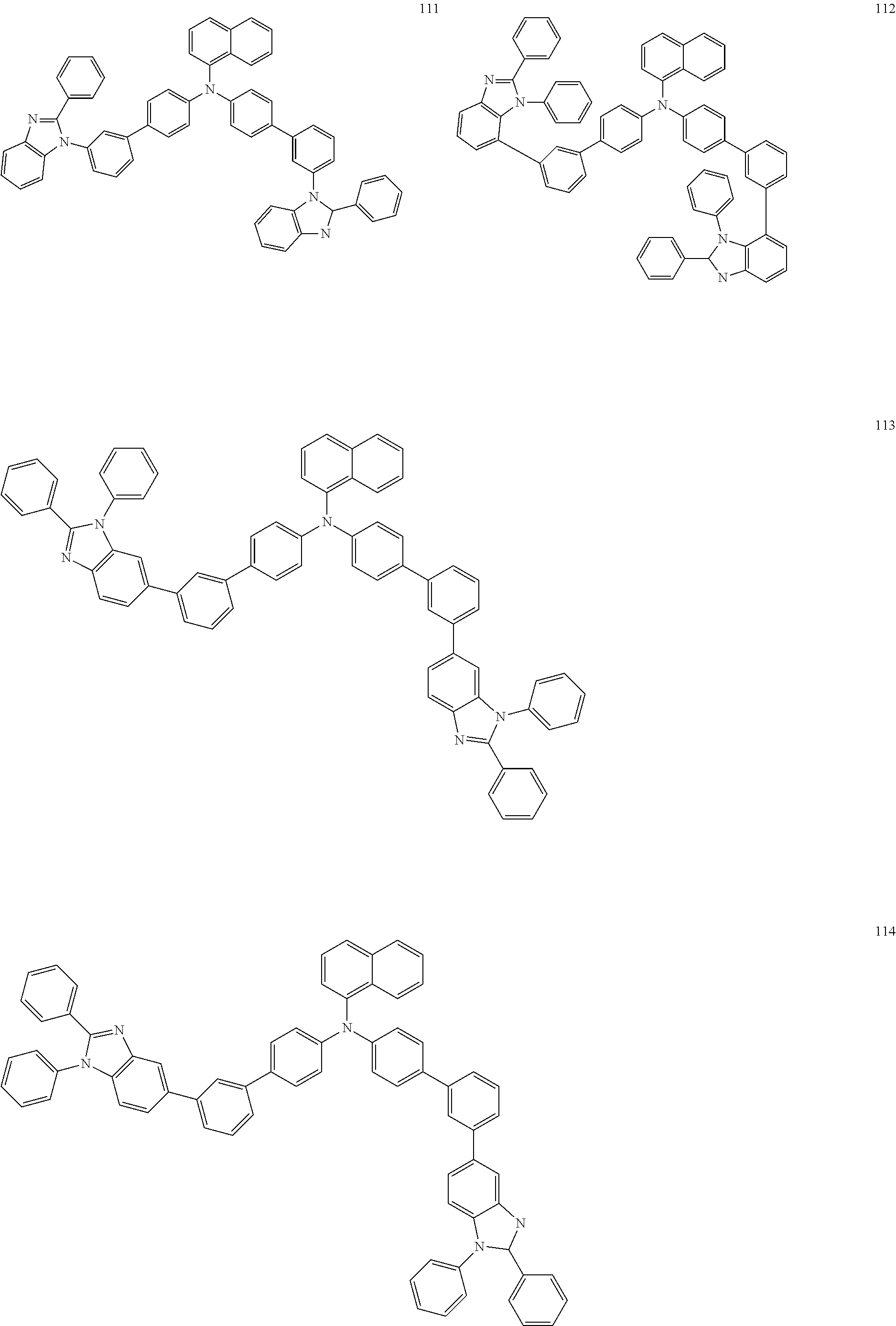

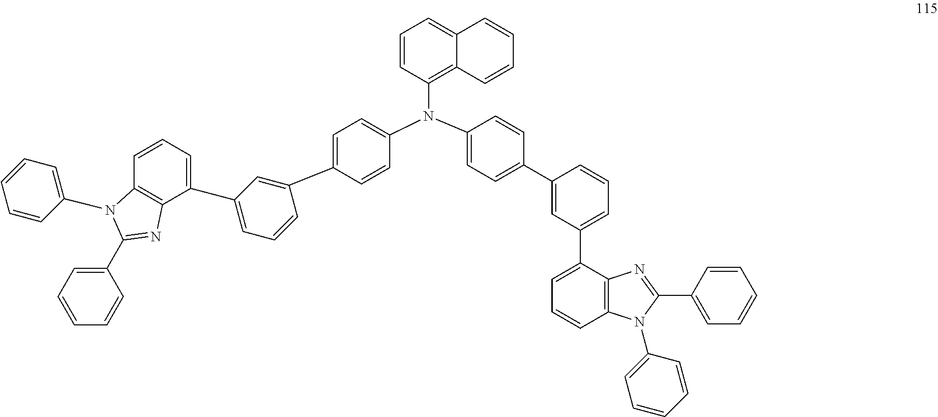 [SMILES: c1ccc(-c2nc3c(-c4cccc(-c5ccc(N(c6ccc(-c7cccc(-c8cccc9c8nc(-c8ccccc8)n9-c8ccccc8)c7)cc6)c6cccc7ccccc67)cc5)c4)cccc3n2-c2ccccc2)cc1]

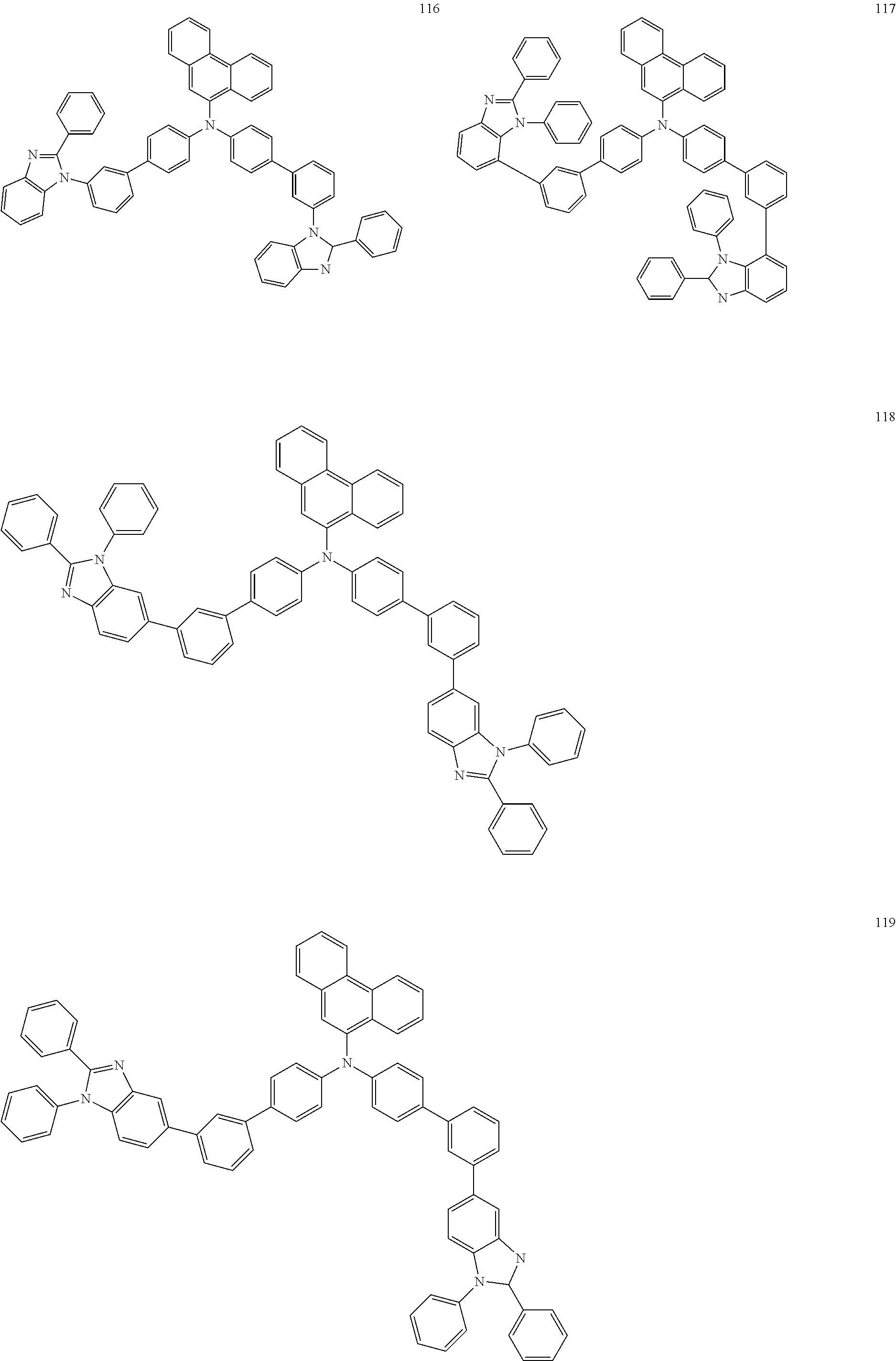

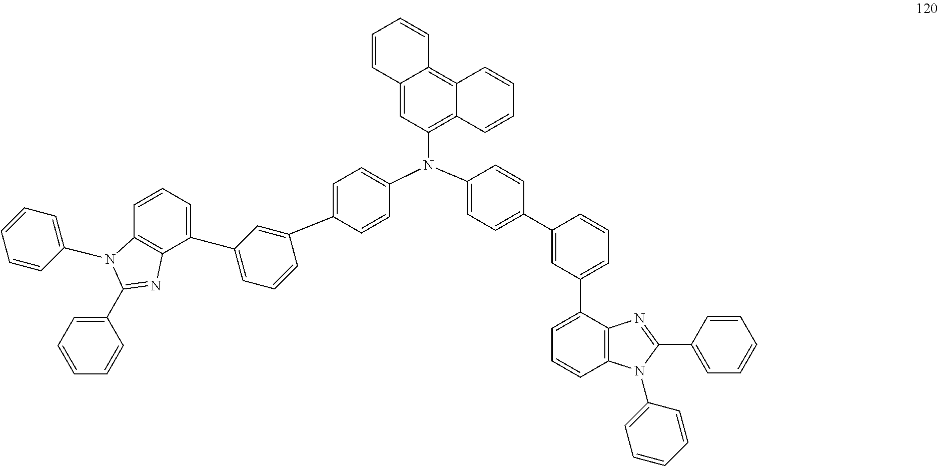 [SMILES: c1ccc(-c2nc3c(-c4cccc(-c5ccc(N(c6ccc(-c7cccc(-c8cccc9c8nc(-c8ccccc8)n9-c8ccccc8)c7)cc6)c6cc7ccccc7c7ccccc67)cc5)c4)cccc3n2-c2ccccc2)cc1]

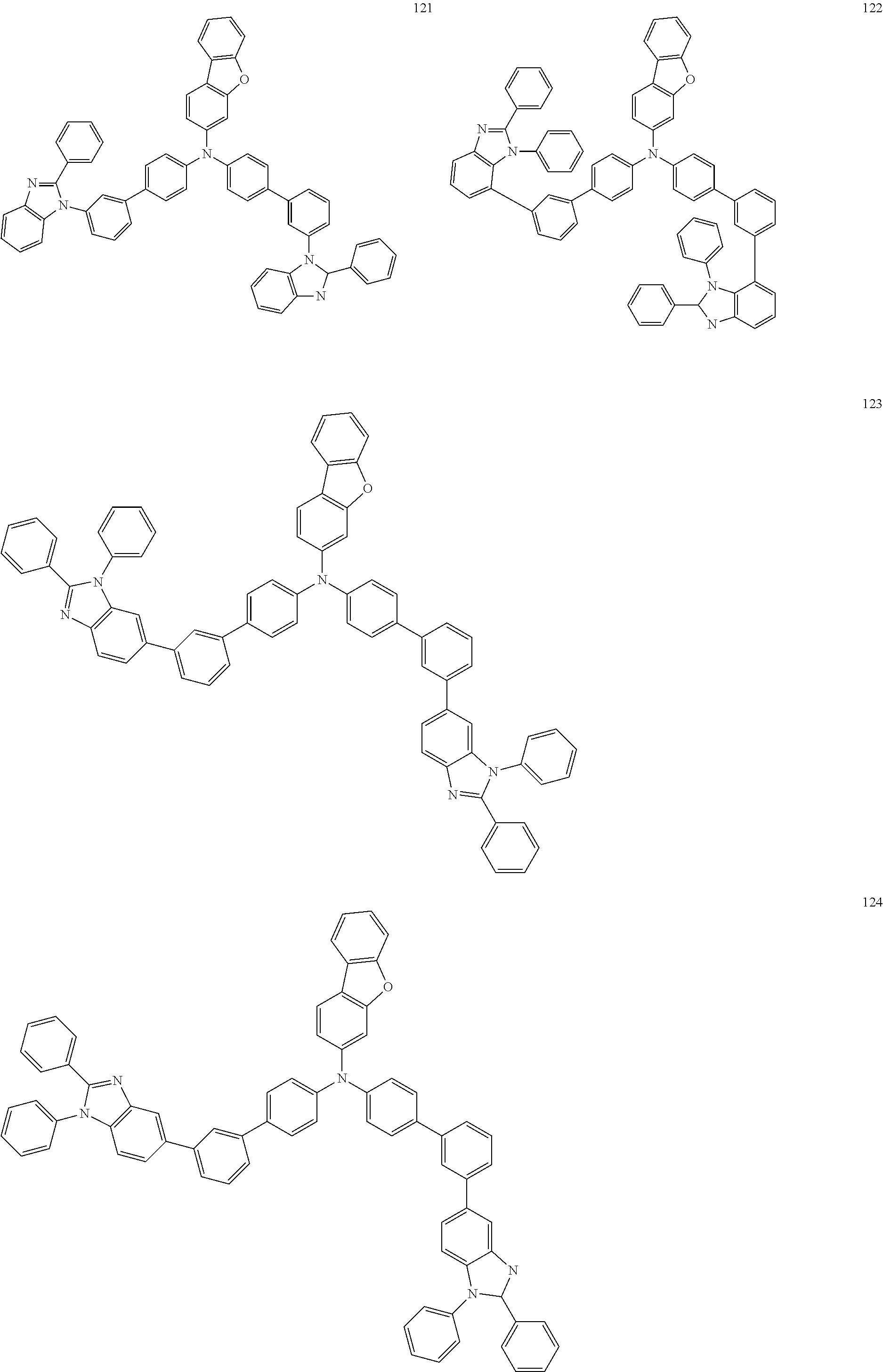

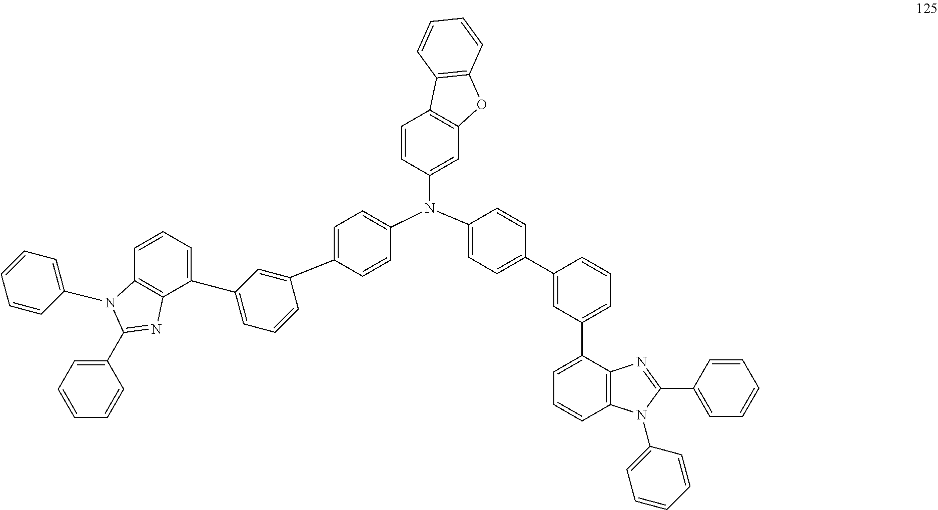 [SMILES: c1ccc(-c2nc3c(-c4cccc(-c5ccc(N(c6ccc(-c7cccc(-c8cccc9c8nc(-c8ccccc8)n9-c8ccccc8)c7)cc6)c6ccc7c(c6)oc6ccccc67)cc5)c4)cccc3n2-c2ccccc2)cc1]

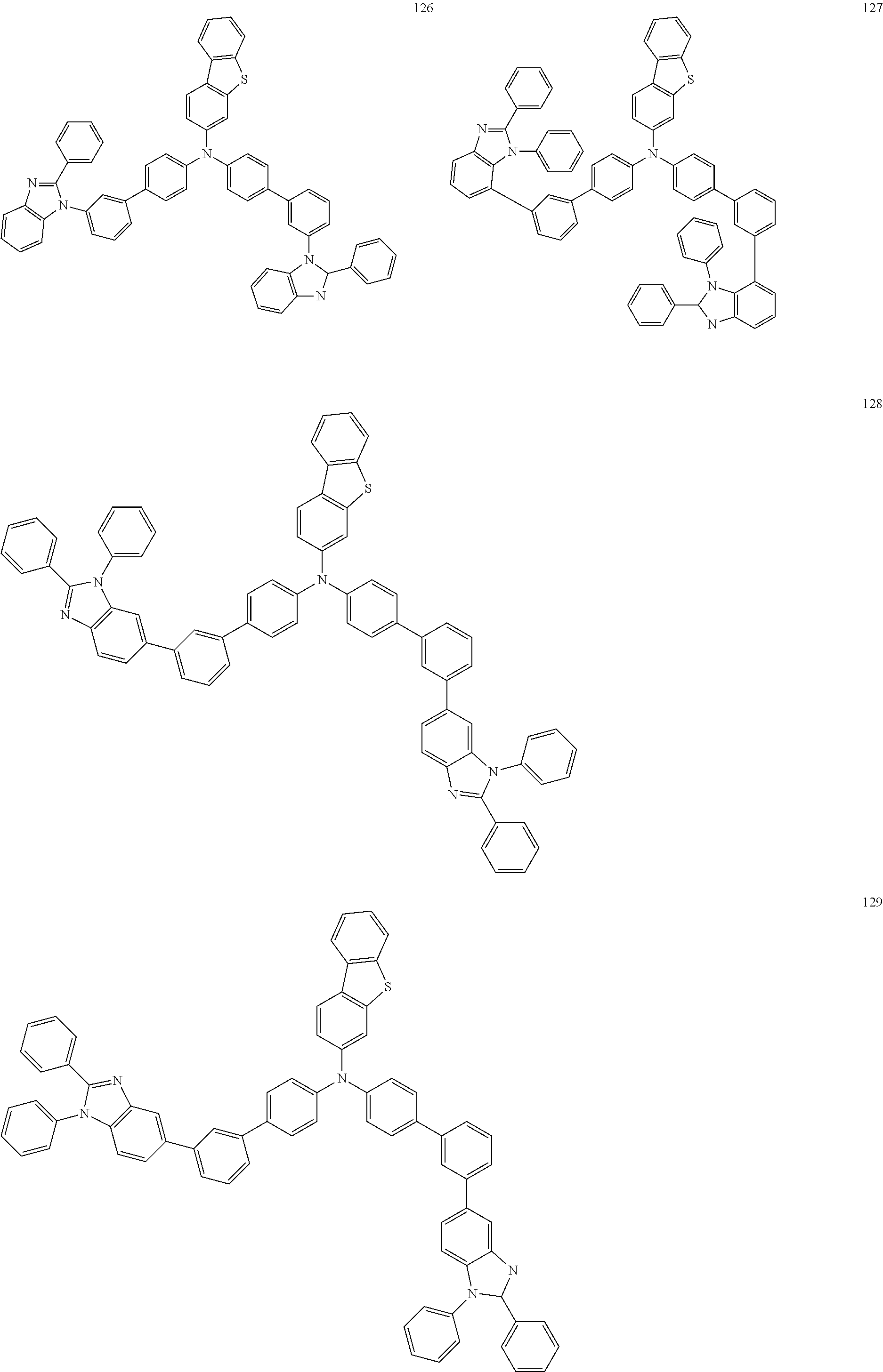

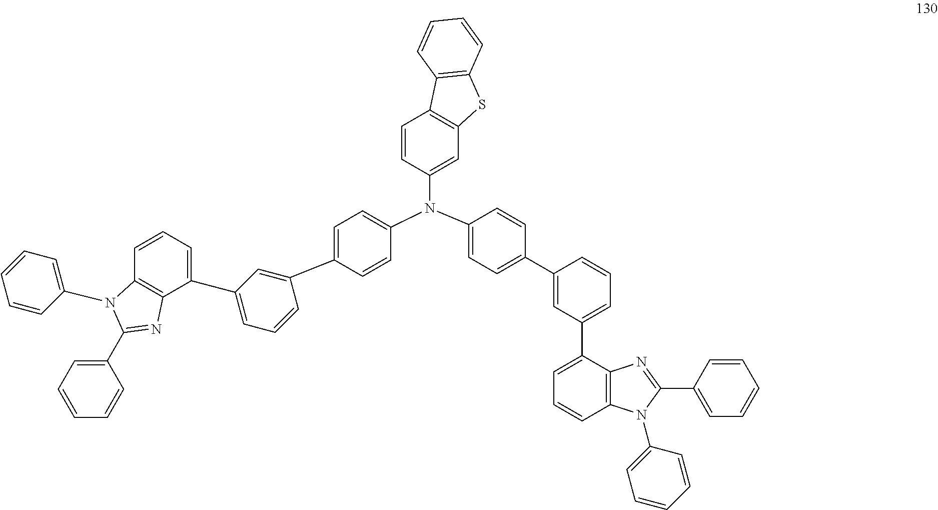 [SMILES: c1ccc(-c2nc3c(-c4cccc(-c5ccc(N(c6ccc(-c7cccc(-c8cccc9c8nc(-c8ccccc8)n9-c8ccccc8)c7)cc6)c6ccc7c(c6)sc6ccccc67)cc5)c4)cccc3n2-c2ccccc2)cc1]

11. An organic electroluminescent device comprising:
an anode,
an emission layer; and
at least one layer between the anode and the emission layer, the at least one layer comprising an amine derivative represented by Formula 1: 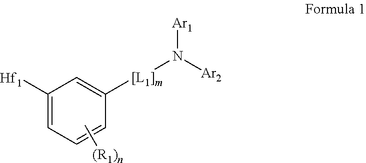
wherein in Formula 1,
Ar₁ and Ar₂ are each independently a substituted or unsubstituted aryl group having 6 to 30 carbon atoms for forming a ring, or a substituted or unsubstituted heteroaryl group having 2 to 30 carbon atoms for forming a ring;
L₁ is a divalent linking group;
R₁ is a substituted or unsubstituted alkyl group having 1 to 10 carbon atoms in a straight chain, branched chain, or ring form; a substituted or unsubstituted aryl group having 6 to 20 carbon atoms for forming a ring; a substituted or unsubstituted heteroaryl group having 1 to 20 carbon atoms for forming a ring; or an alkyl group, an aryl group, or a heteroaryl group formed through condensation of adjacent substituents;
n and m are each independently an integer from 0 to 4; and
Hf₁ is represented by Formula 2-1 or Formula 2-2; 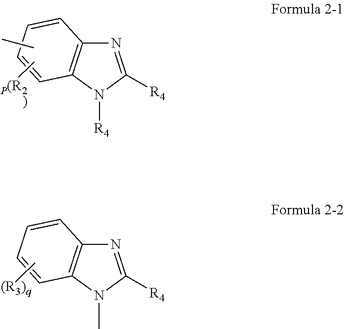
wherein in Formulae 2-1 and 2-2,
R₂, R₃, and R₄ are each independently a substituted or unsubstituted alkyl group having 1 to 10 carbon atoms in a straight chain, branched chain, or ring form; a substituted or unsubstituted aryl group having 6 to 20 carbon atoms for forming a ring; a substituted or unsubstituted heteroaryl group having 1 to 20 carbon atoms for forming a ring; or an aryl group or a heteroaryl group formed through condensation of adjacent substituents;
p is an integer from 0 to 3; and
q is an integer from 0 to 4.

12. The organic electroluminescent device of claim 11, wherein L₁ is a substituted or unsubstituted arylene group having 6 to 30 carbon atoms for forming a ring, or a substituted or unsubstituted heteroarylene group having 2 to 30 carbon atoms for forming a ring.

13. The organic electroluminescent device of claim 11, wherein Ar₁ and Ar₂ are each independently a non-nitrogen atom-containing aryl group or heteroaryl group which is unsubstituted or substituted with a substituent which does not include a nitrogen atom.

14. The organic electroluminescent device of claim 11, wherein Ar₁ is a non-nitrogen atom-containing aryl group or heteroaryl group which is unsubstituted or substituted with a substituent which does not include a nitrogen atom, and Ar₂ is represented by Formula 3: 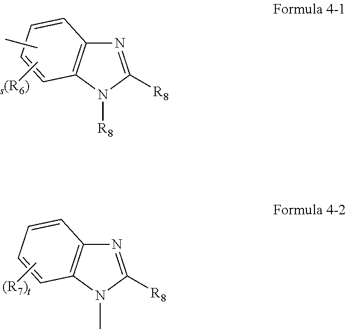
wherein in Formula 3,
L₂ is a divalent linking group;
R₅ is a substituted or unsubstituted alkyl group having 1 to 10 carbon atoms in a straight chain, branched chain, or ring form; a substituted or unsubstituted aryl group having 6 to 20 carbon atoms for forming a ring; a substituted or unsubstituted heteroaryl group having 1 to 20 carbon atoms for forming a ring; or an alkyl group, an aryl group, or a heteroaryl group formed through condensation of adjacent substituents;
j and k are each independently an integer from 0 to 4; and
Hf₂ is represented by Formula 4-1 or Formula 4-2; 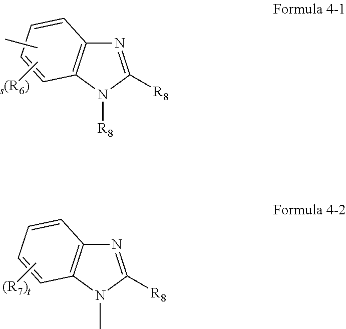
wherein in Formulae 4-1 and 4-2,
R₆, R₇, and R₈ are each independently a substituted or unsubstituted alkyl group having 1 to 10 carbon atoms in a straight chain, branched chain, or ring form; a substituted or unsubstituted aryl group having 6 to 20 carbon atoms for forming a ring; a substituted or unsubstituted heteroaryl group having 1 to 20 carbon atoms for forming a ring; or an aryl group or a heteroaryl group formed through condensation of adjacent substituents;
s is an integer from 0 to 3; and
t is an integer from 0 to 4.

15. The organic electroluminescent device of claim 11, wherein the amine derivative represented by Formula 1 is at least one of compounds 1 to 130: 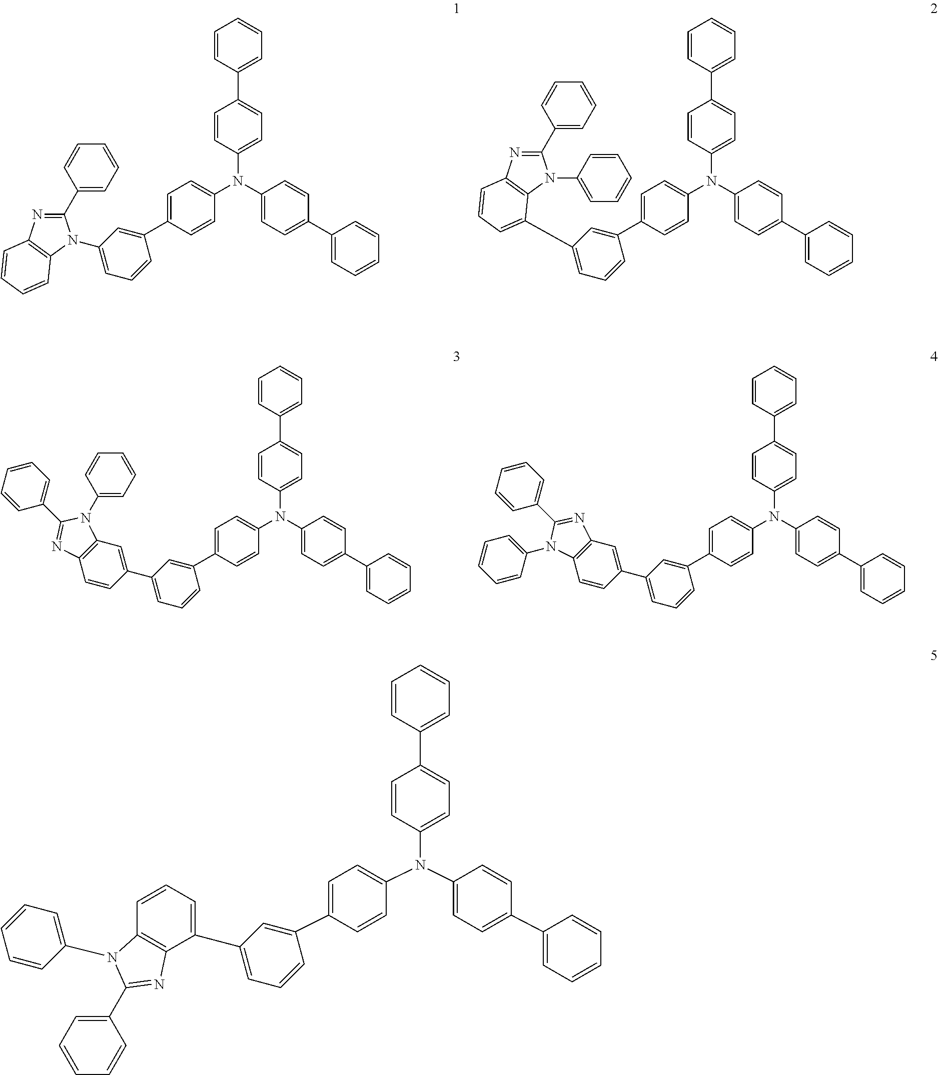 [SMILES: c1ccc(-c2ccc(N(c3ccc(-c4ccccc4)cc3)c3ccc(-c4cccc(-n5c(-c6ccccc6)nc6ccccc65)c4)cc3)cc2)cc1 ; c1ccc(-c2ccc(N(c3ccc(-c4ccccc4)cc3)c3ccc(-c4cccc(-c5cccc6nc(-c7ccccc7)n(-c7ccccc7)c56)c4)cc3)cc2)cc1 ; c1ccc(-c2ccc(N(c3ccc(-c4ccccc4)cc3)c3ccc(-c4cccc(-c5ccc6nc(-c7ccccc7)n(-c7ccccc7)c6c5)c4)cc3)cc2)cc1 ; c1ccc(-c2ccc(N(c3ccc(-c4ccccc4)cc3)c3ccc(-c4cccc(-c5ccc6c(c5)nc(-c5ccccc5)n6-c5ccccc5)c4)cc3)cc2)cc1 ; c1ccc(-c2ccc(N(c3ccc(-c4ccccc4)cc3)c3ccc(-c4cccc(-c5cccc6c5nc(-c5ccccc5)n6-c5ccccc5)c4)cc3)cc2)cc1] 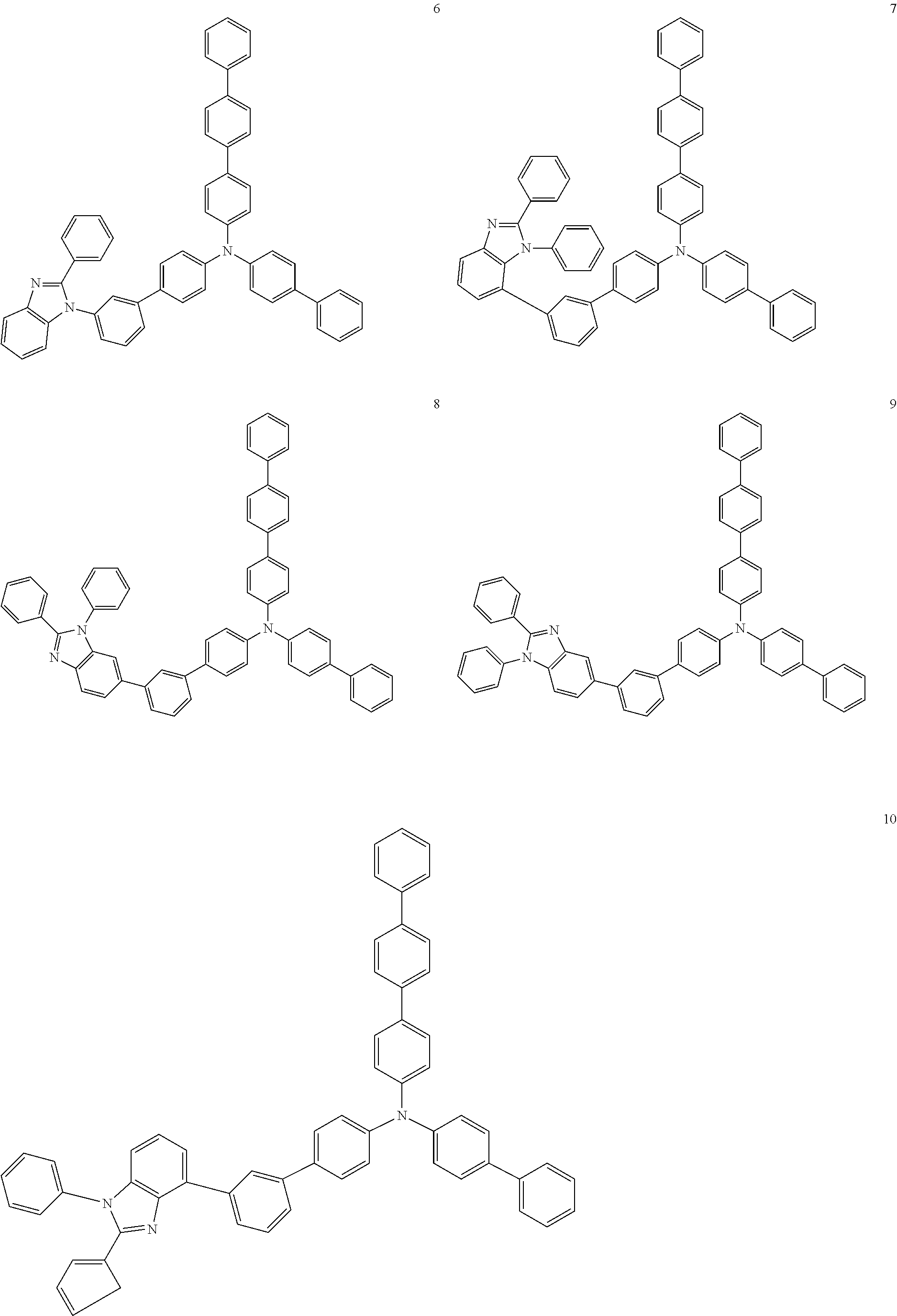 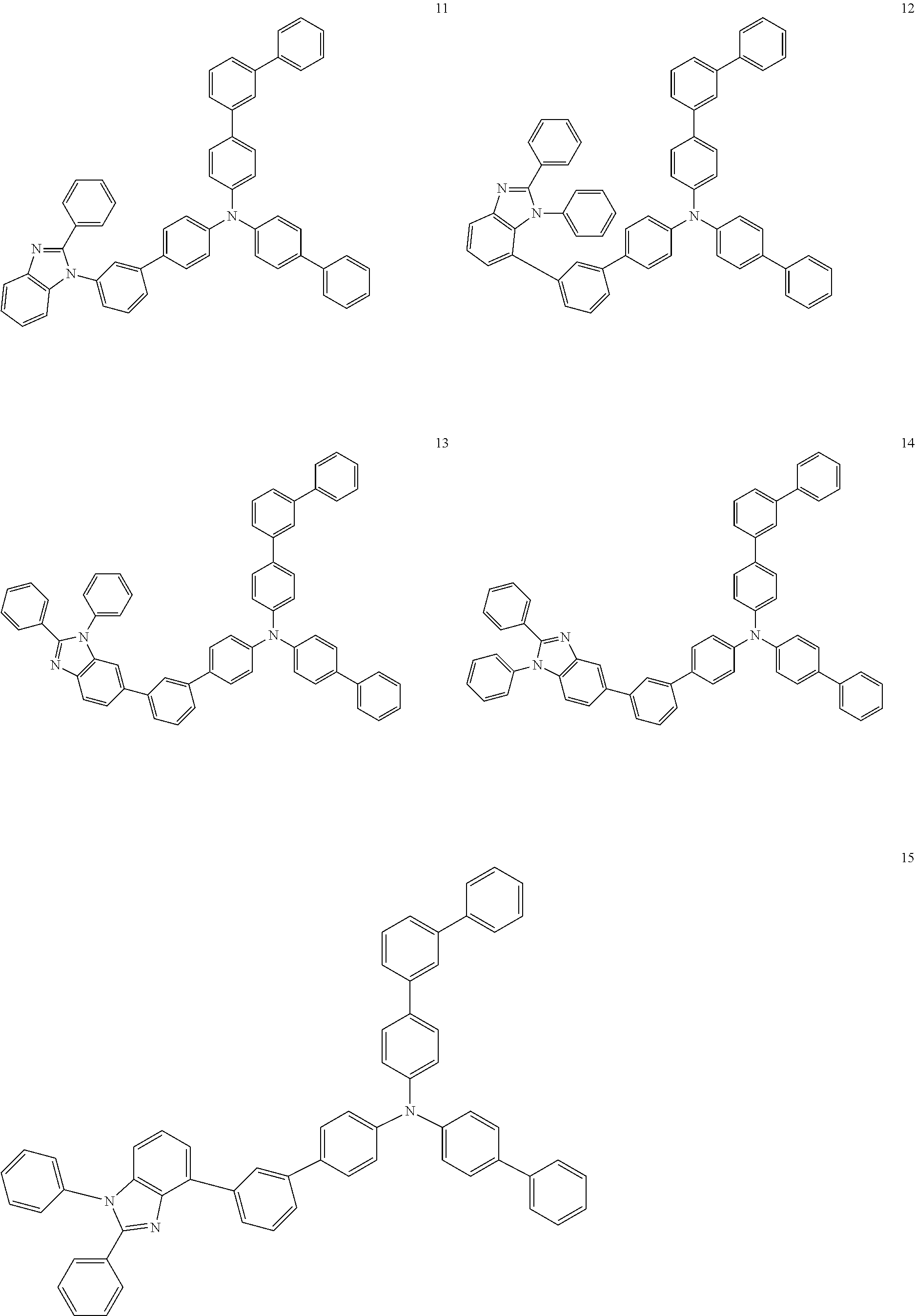 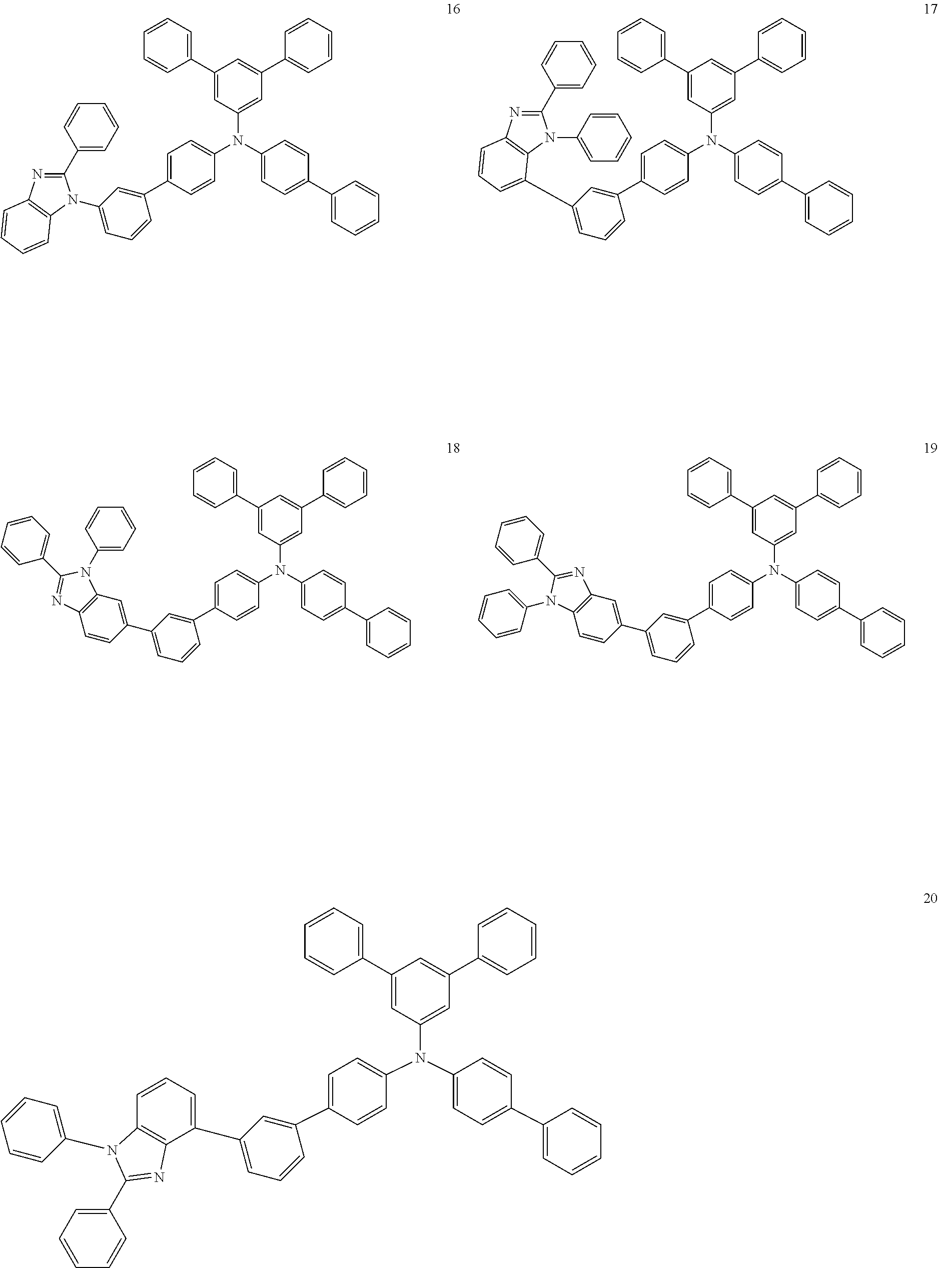 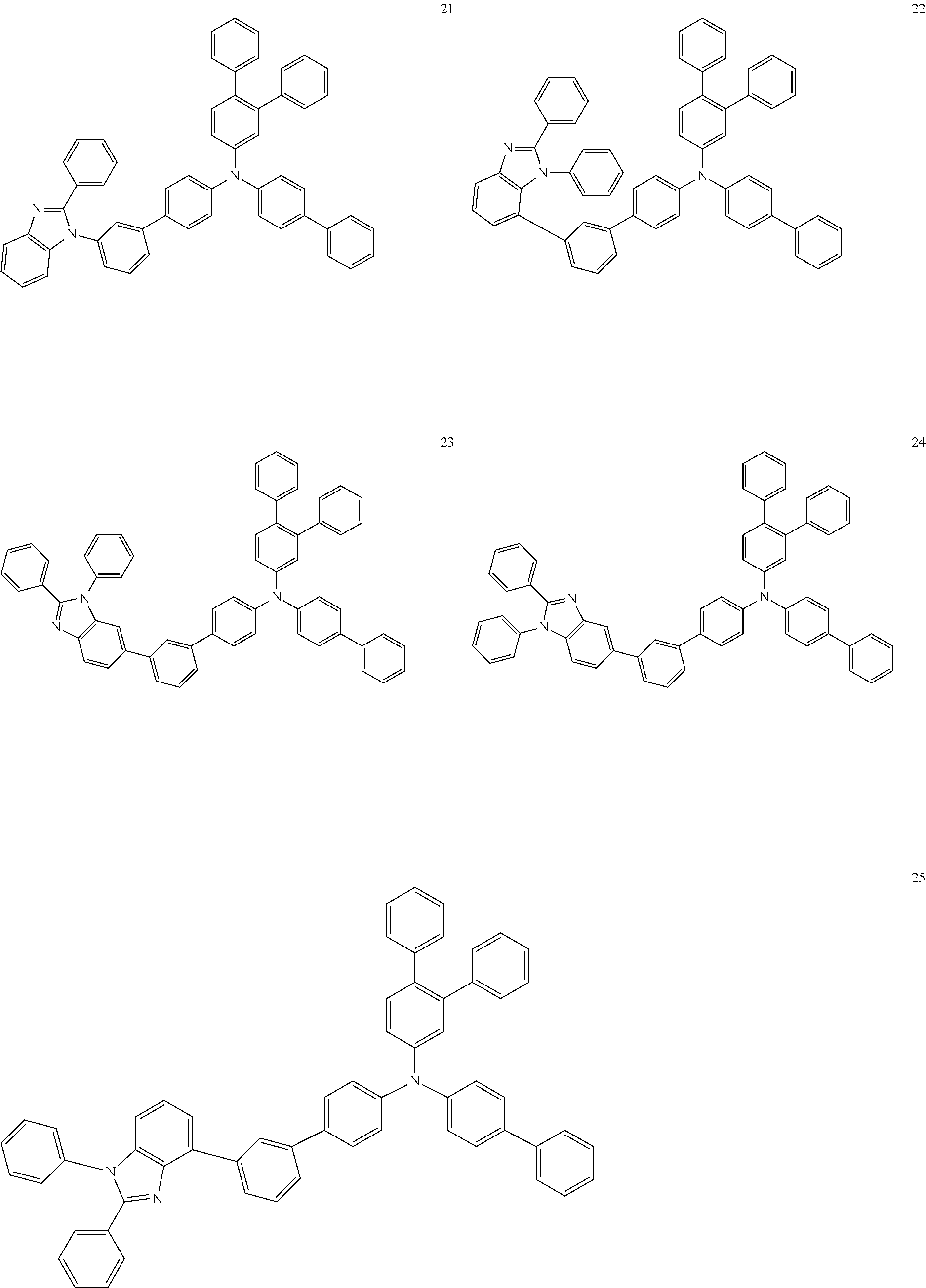 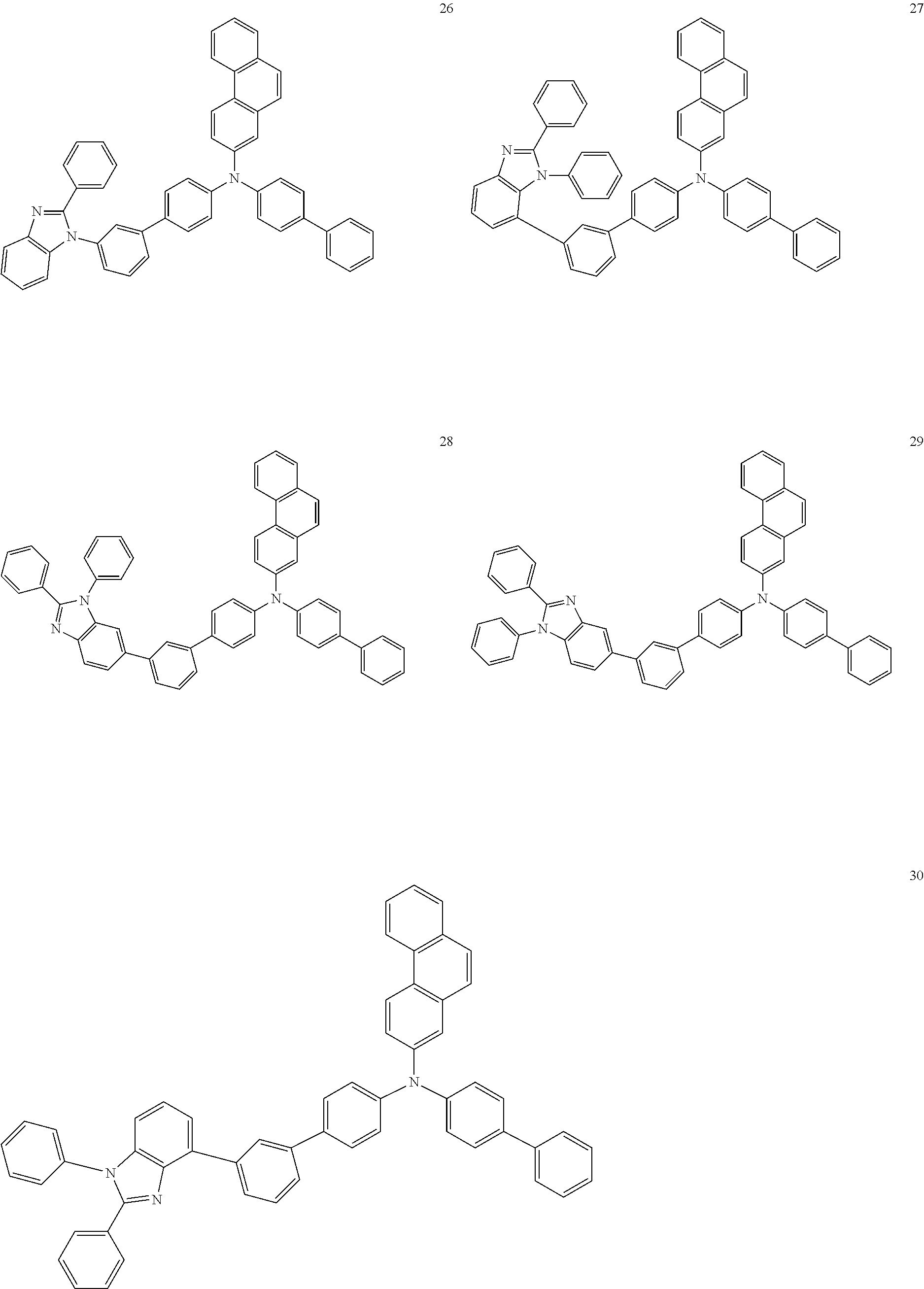 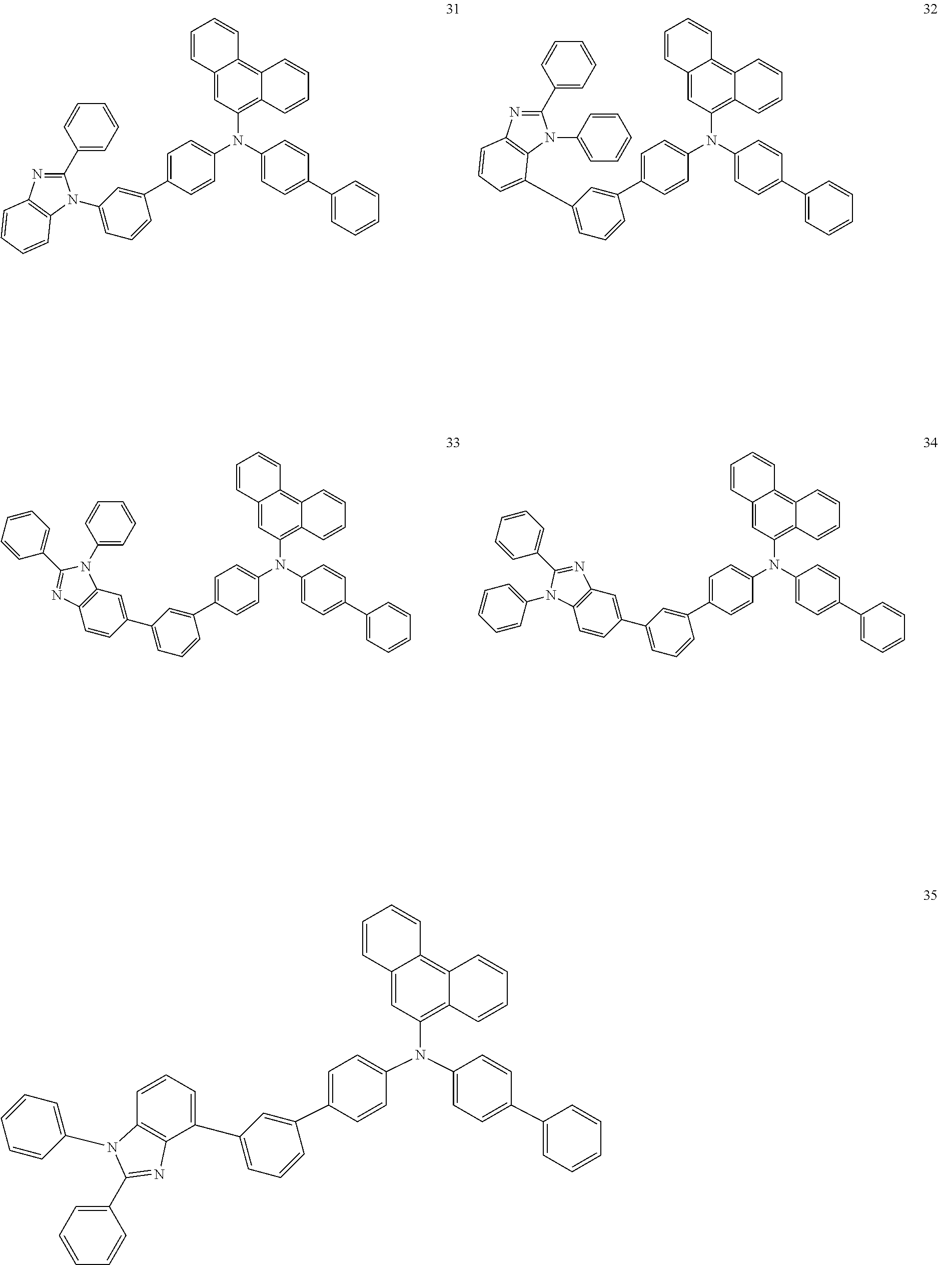 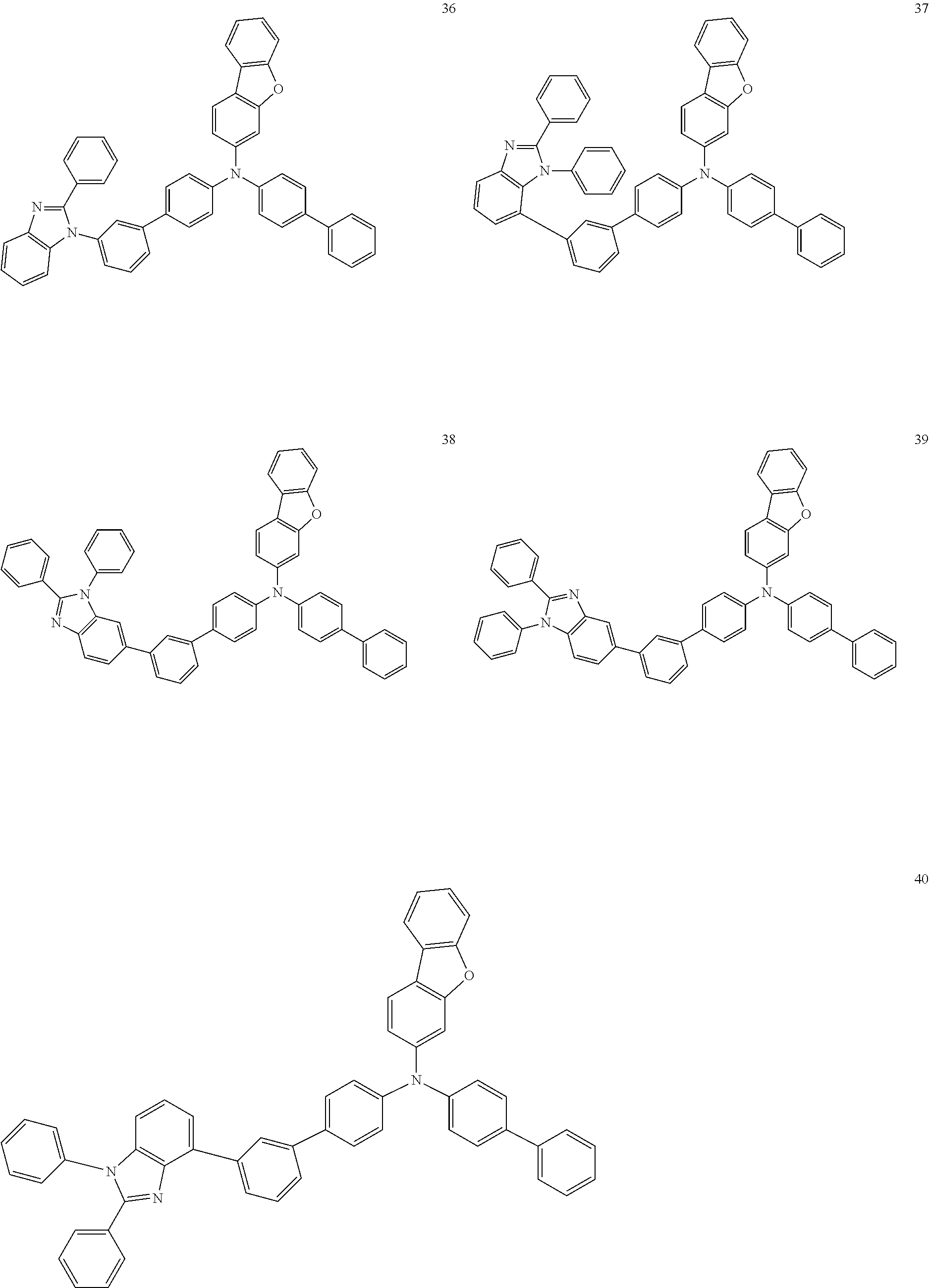 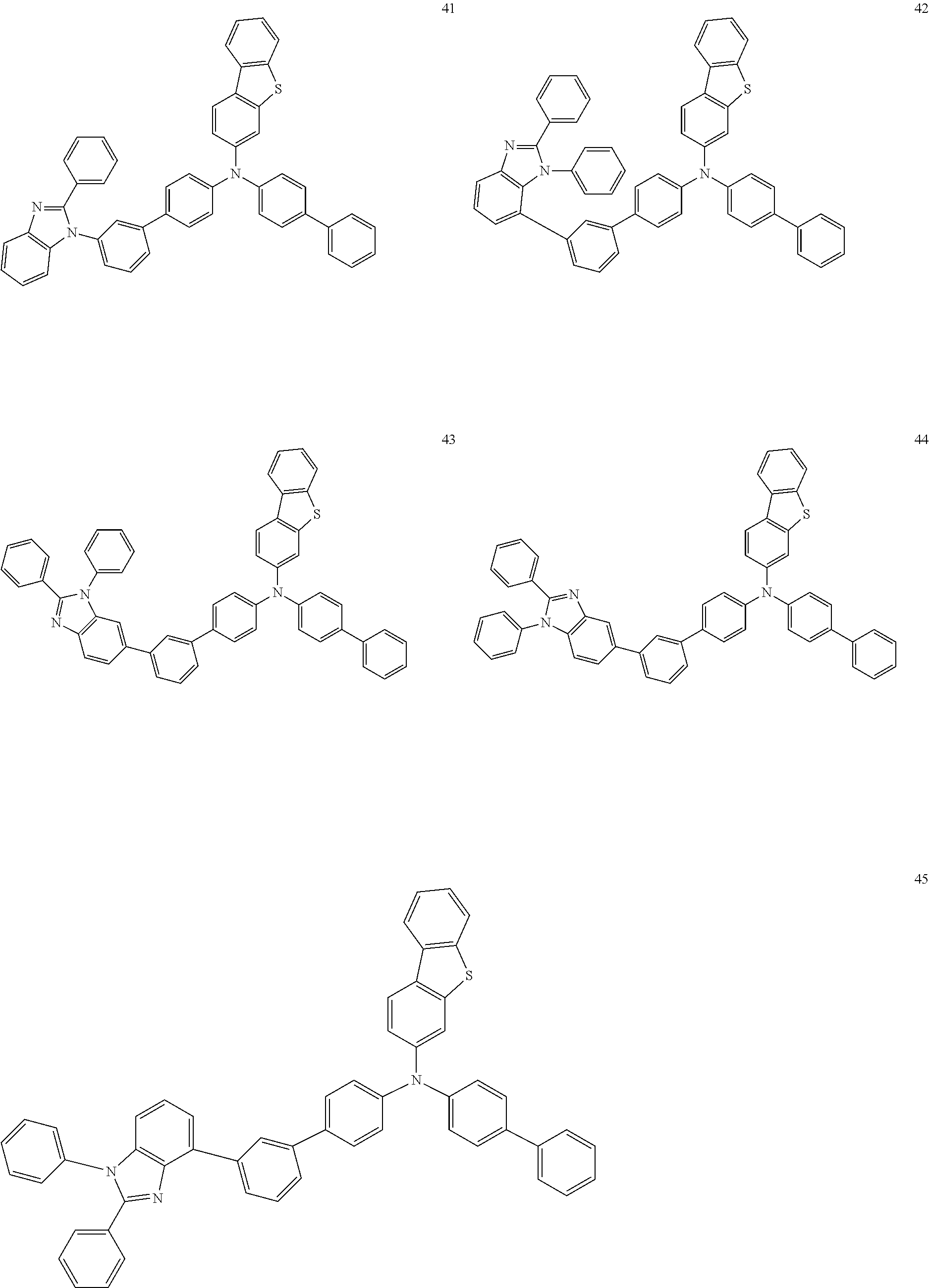 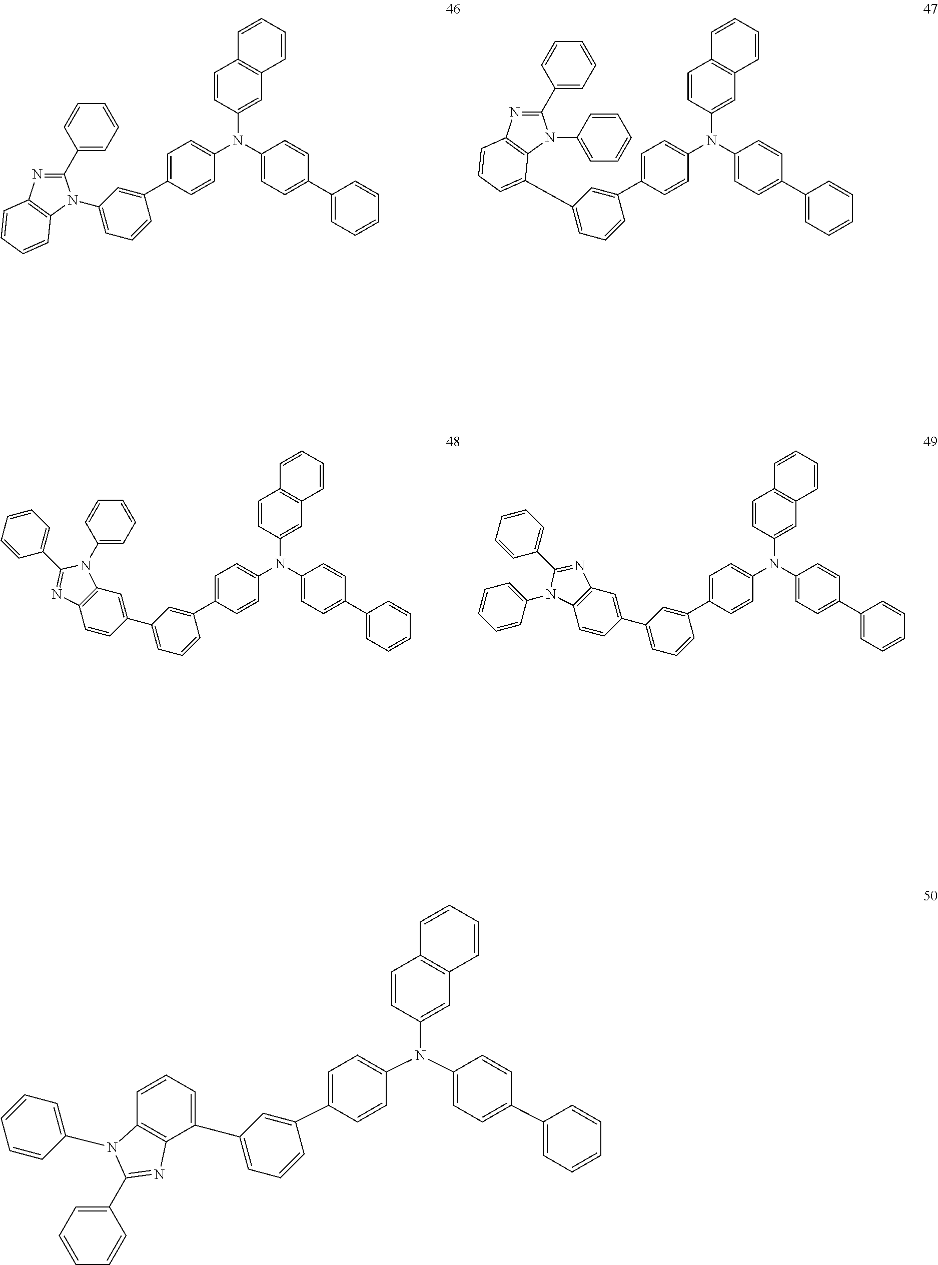 [SMILES: c1ccc(-c2ccc(N(c3ccc(-c4cccc(-n5c(-c6ccccc6)nc6ccccc65)c4)cc3)c3ccc4ccccc4c3)cc2)cc1 ; c1ccc(-c2ccc(N(c3ccc(-c4cccc(-c5cccc6nc(-c7ccccc7)n(-c7ccccc7)c56)c4)cc3)c3ccc4ccccc4c3)cc2)cc1 ; c1ccc(-c2ccc(N(c3ccc(-c4cccc(-c5ccc6nc(-c7ccccc7)n(-c7ccccc7)c6c5)c4)cc3)c3ccc4ccccc4c3)cc2)cc1 ; c1ccc(-c2ccc(N(c3ccc(-c4cccc(-c5ccc6c(c5)nc(-c5ccccc5)n6-c5ccccc5)c4)cc3)c3ccc4ccccc4c3)cc2)cc1 ; c1ccc(-c2ccc(N(c3ccc(-c4cccc(-c5cccc6c5nc(-c5ccccc5)n6-c5ccccc5)c4)cc3)c3ccc4ccccc4c3)cc2)cc1] 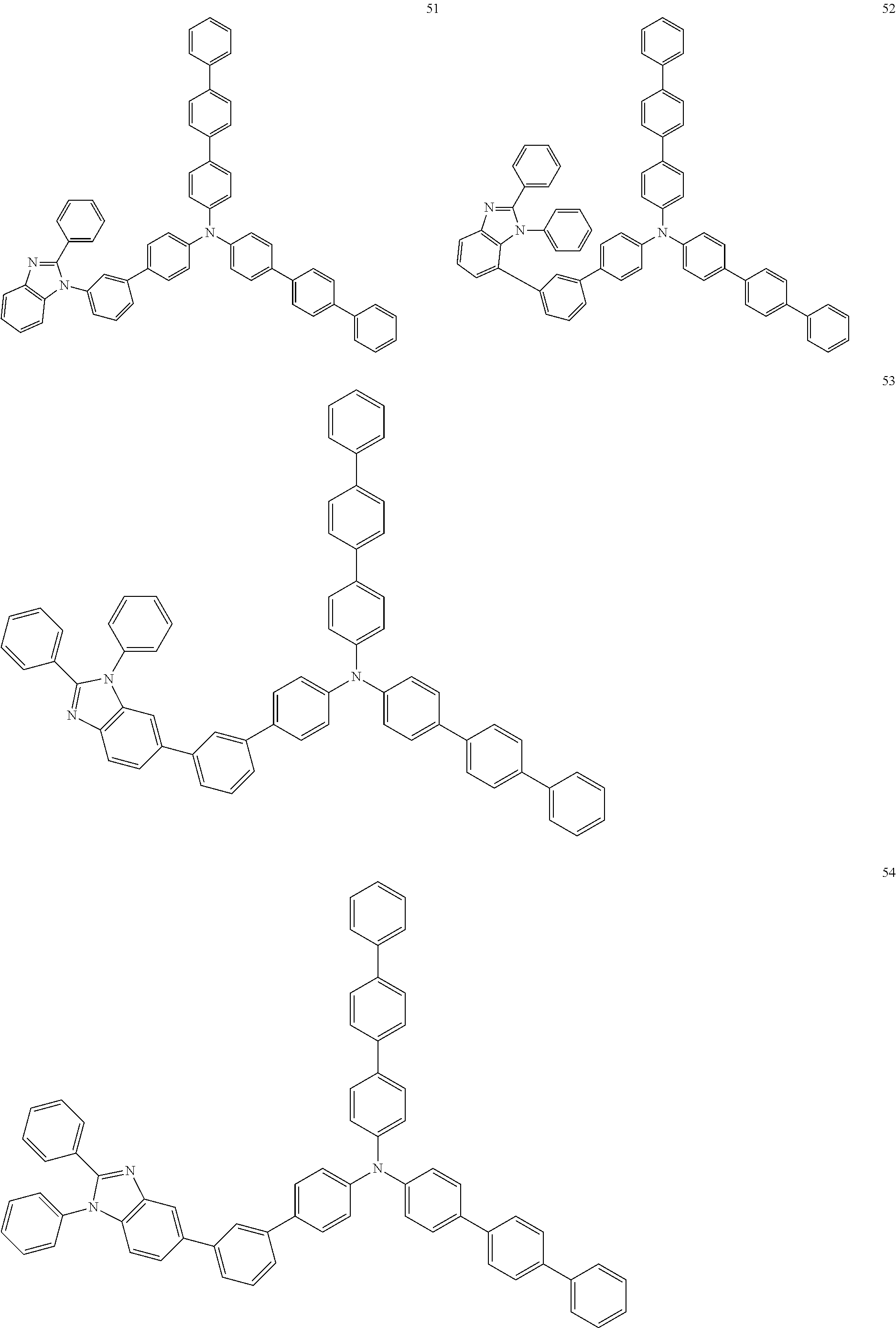 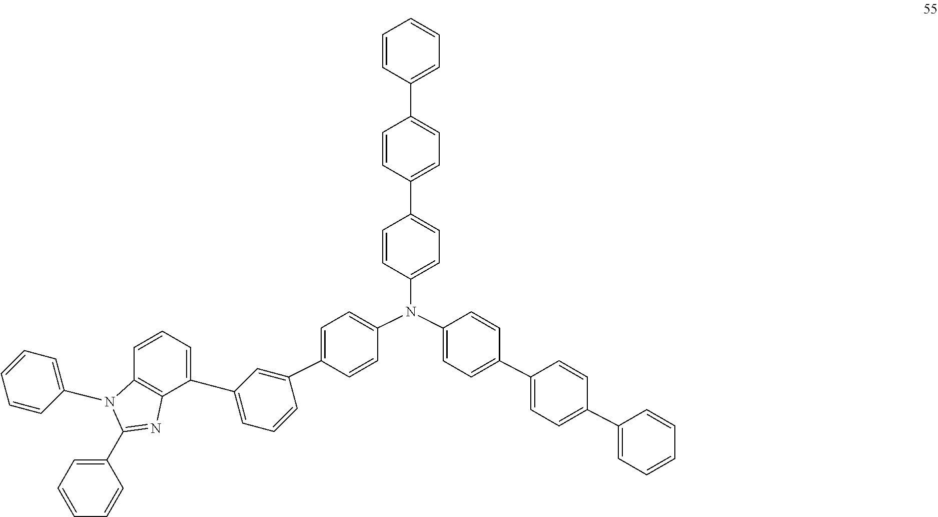 [SMILES: c1ccc(-c2ccc(-c3ccc(N(c4ccc(-c5ccc(-c6ccccc6)cc5)cc4)c4ccc(-c5cccc(-c6cccc7c6nc(-c6ccccc6)n7-c6ccccc6)c5)cc4)cc3)cc2)cc1] 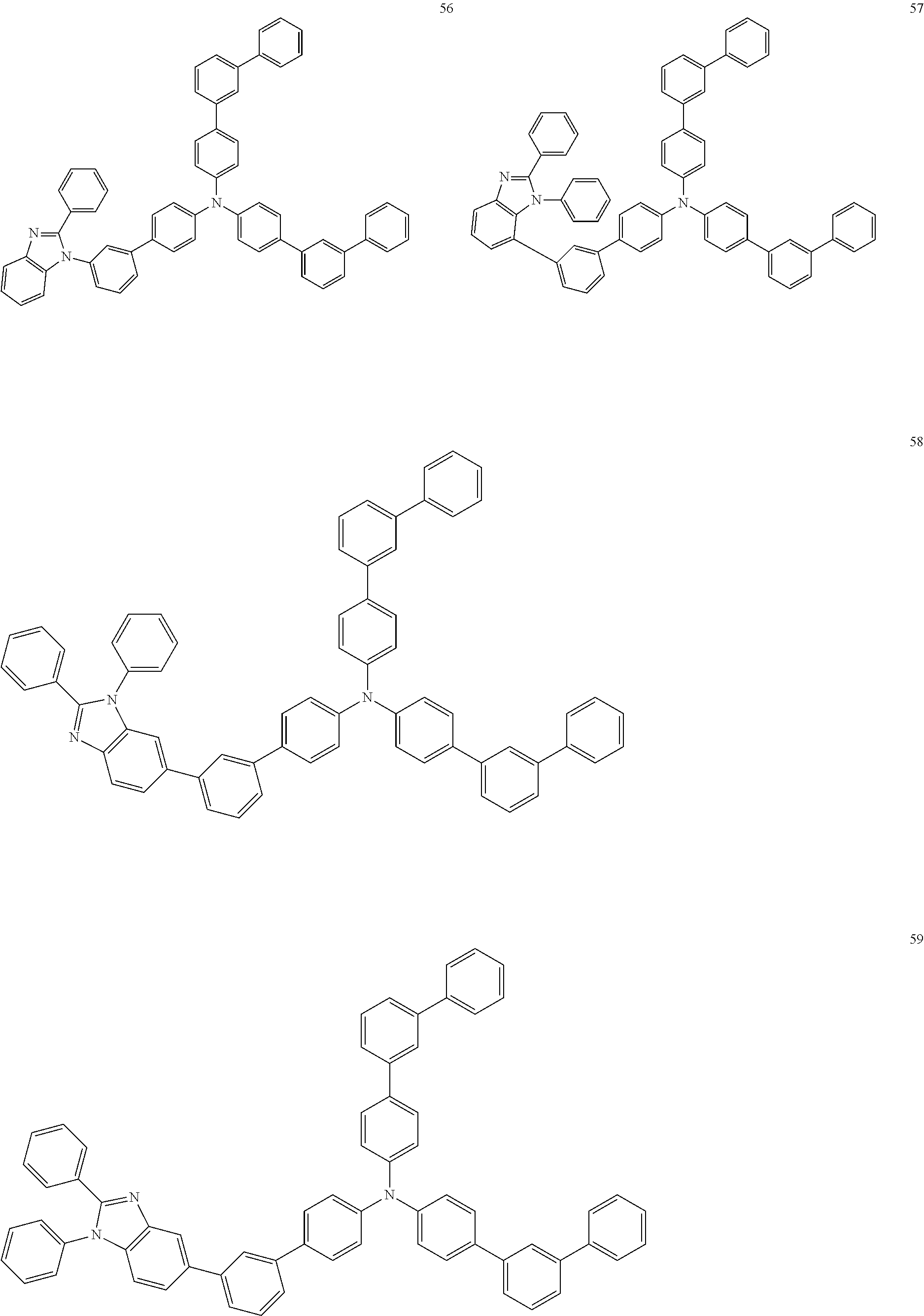 [SMILES: c1ccc(-c2cccc(-c3ccc(N(c4ccc(-c5cccc(-c6ccccc6)c5)cc4)c4ccc(-c5cccc(-n6c(-c7ccccc7)nc7ccccc76)c5)cc4)cc3)c2)cc1 ; c1ccc(-c2cccc(-c3ccc(N(c4ccc(-c5cccc(-c6ccccc6)c5)cc4)c4ccc(-c5cccc(-c6cccc7nc(-c8ccccc8)n(-c8ccccc8)c67)c5)cc4)cc3)c2)cc1 ; c1ccc(-c2cccc(-c3ccc(N(c4ccc(-c5cccc(-c6ccccc6)c5)cc4)c4ccc(-c5cccc(-c6ccc7nc(-c8ccccc8)n(-c8ccccc8)c7c6)c5)cc4)cc3)c2)cc1 ; c1ccc(-c2cccc(-c3ccc(N(c4ccc(-c5cccc(-c6ccccc6)c5)cc4)c4ccc(-c5cccc(-c6ccc7c(c6)nc(-c6ccccc6)n7-c6ccccc6)c5)cc4)cc3)c2)cc1] 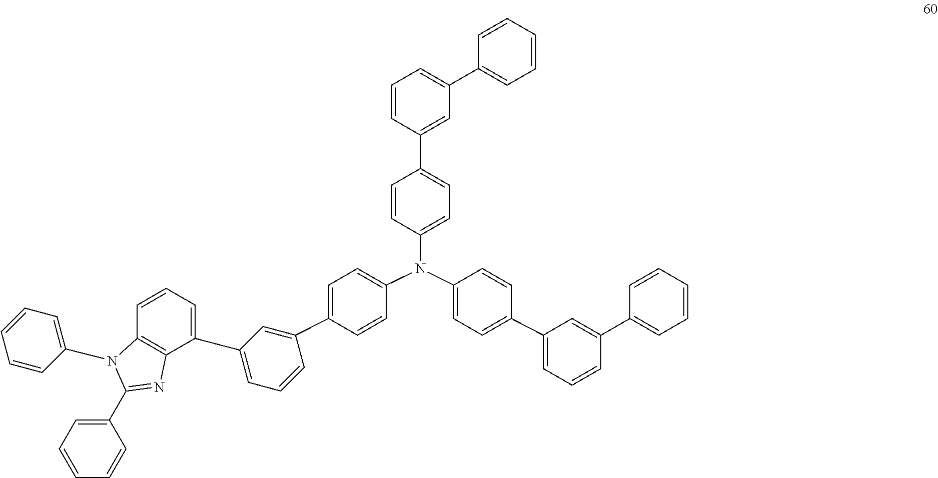 [SMILES: c1ccc(-c2cccc(-c3ccc(N(c4ccc(-c5cccc(-c6ccccc6)c5)cc4)c4ccc(-c5cccc(-c6cccc7c6nc(-c6ccccc6)n7-c6ccccc6)c5)cc4)cc3)c2)cc1] 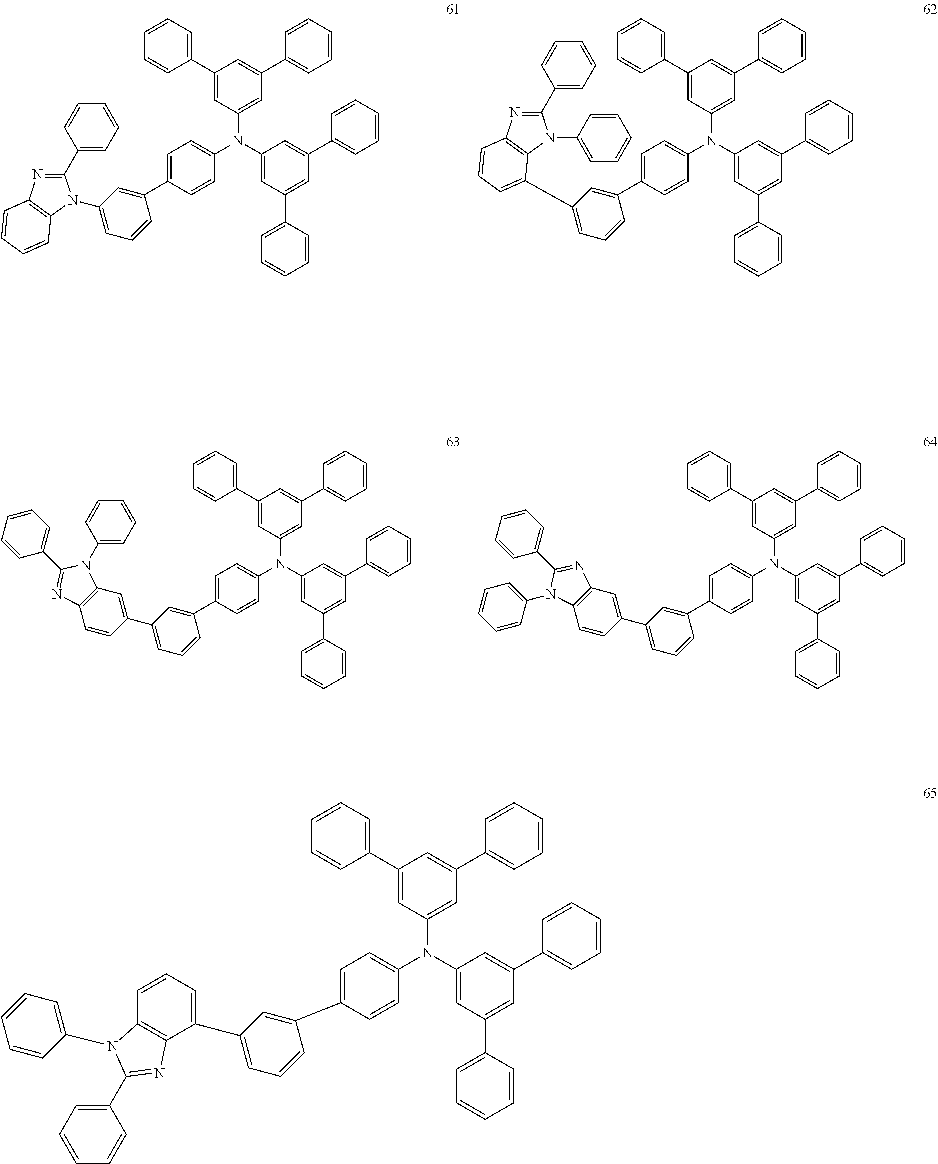 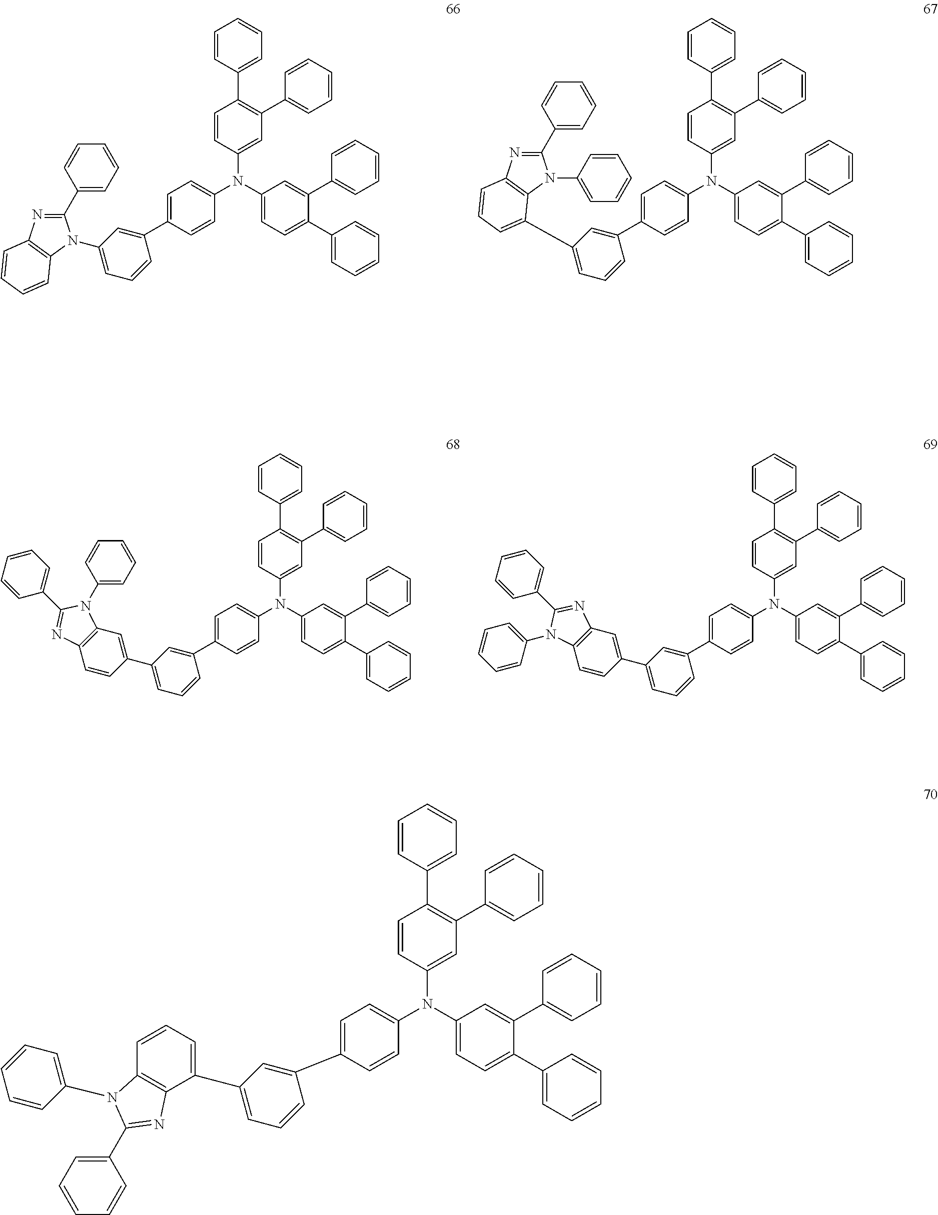 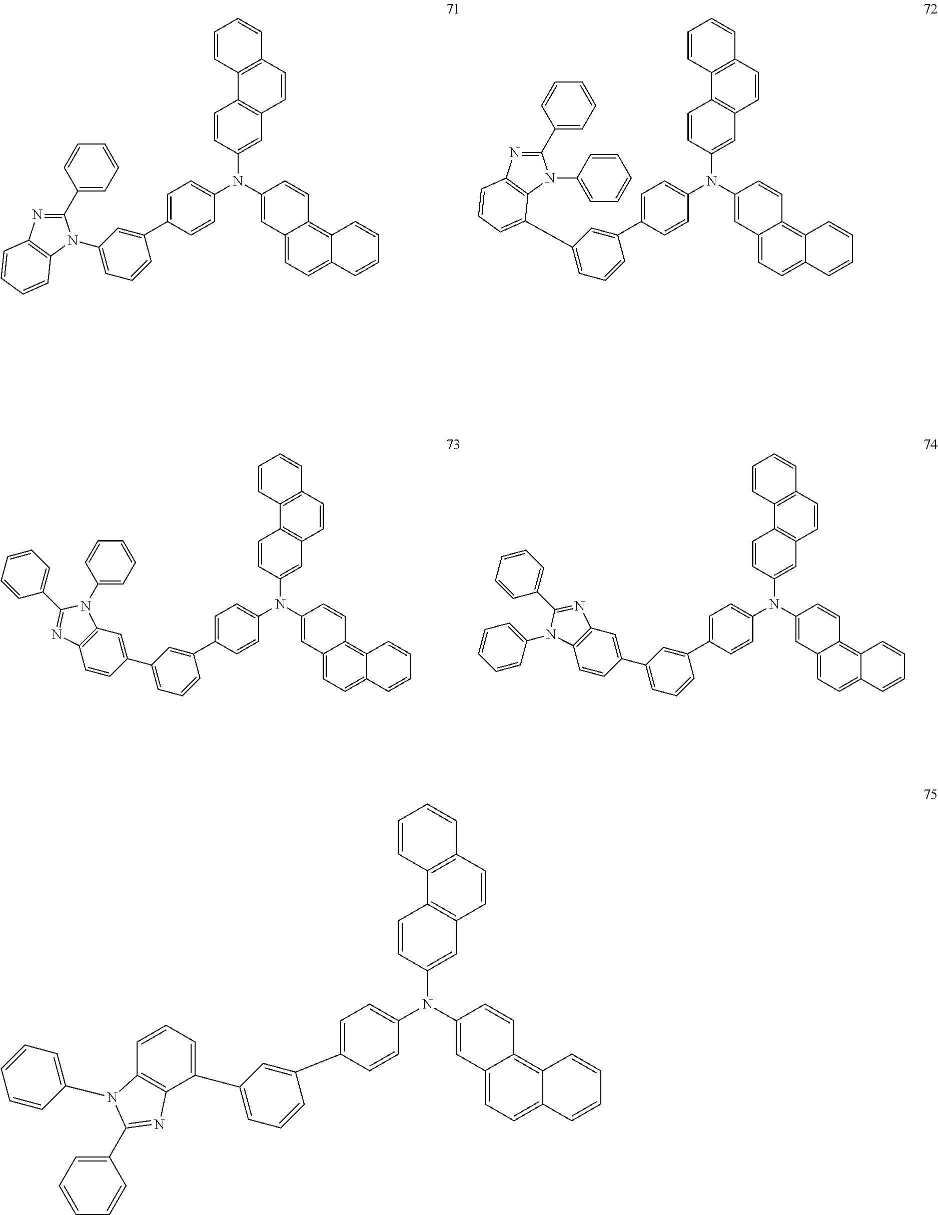 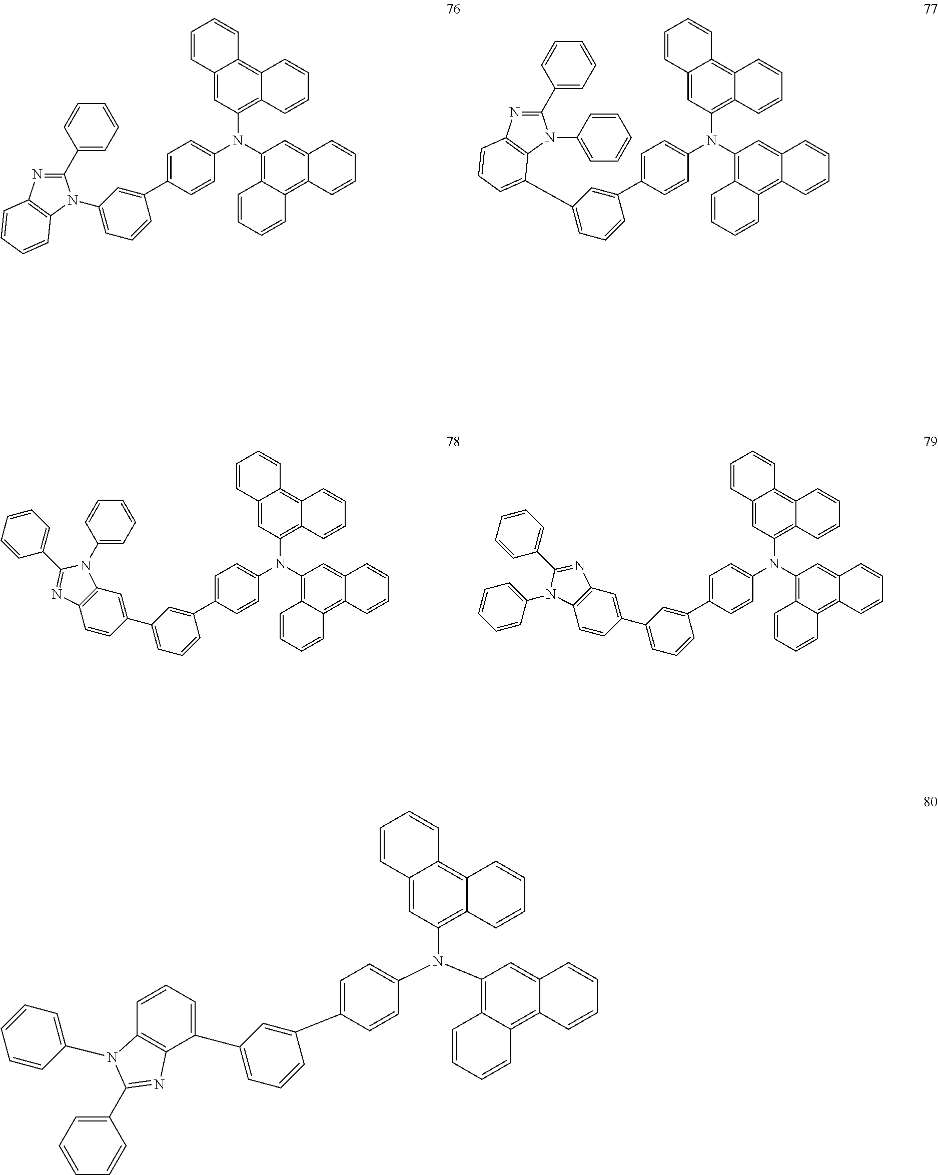 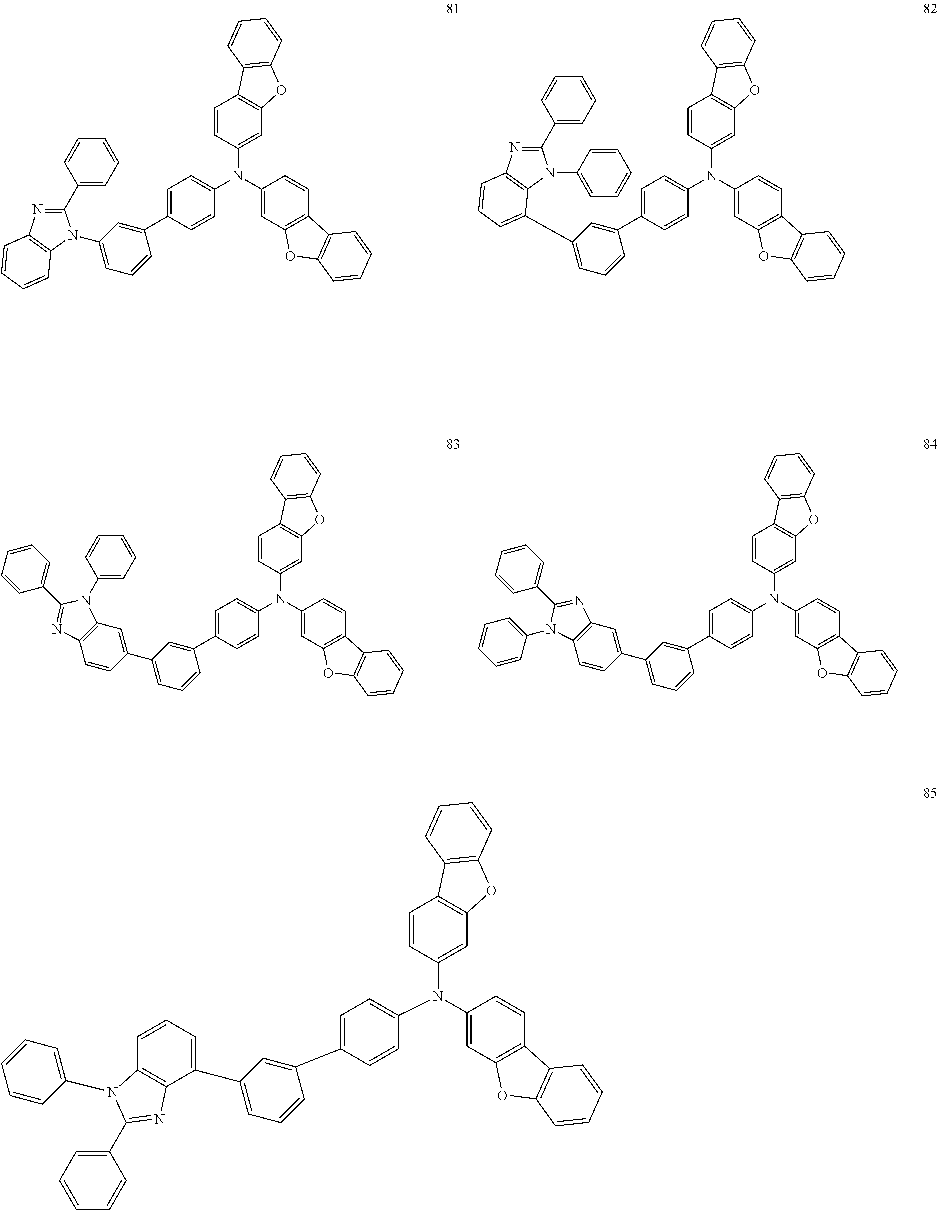 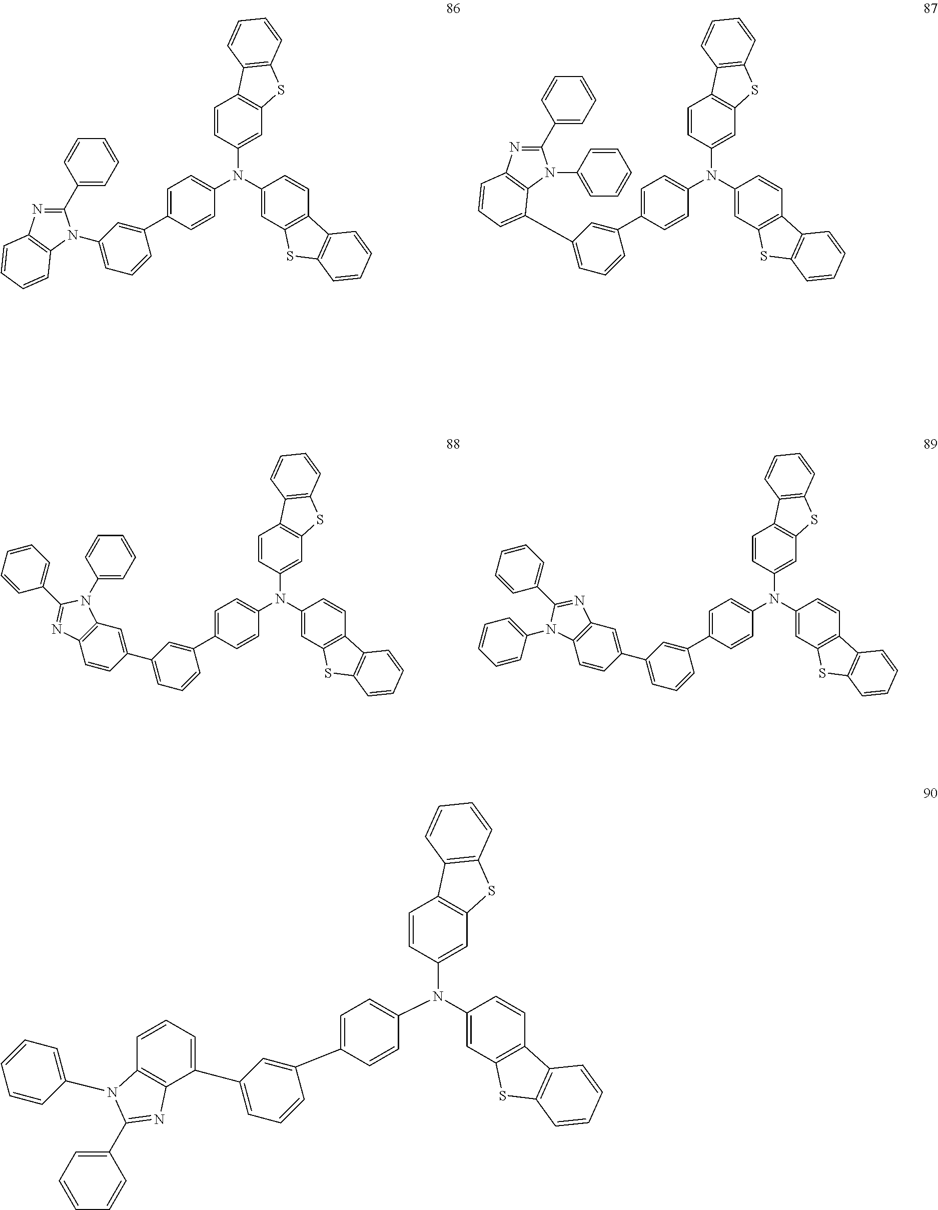 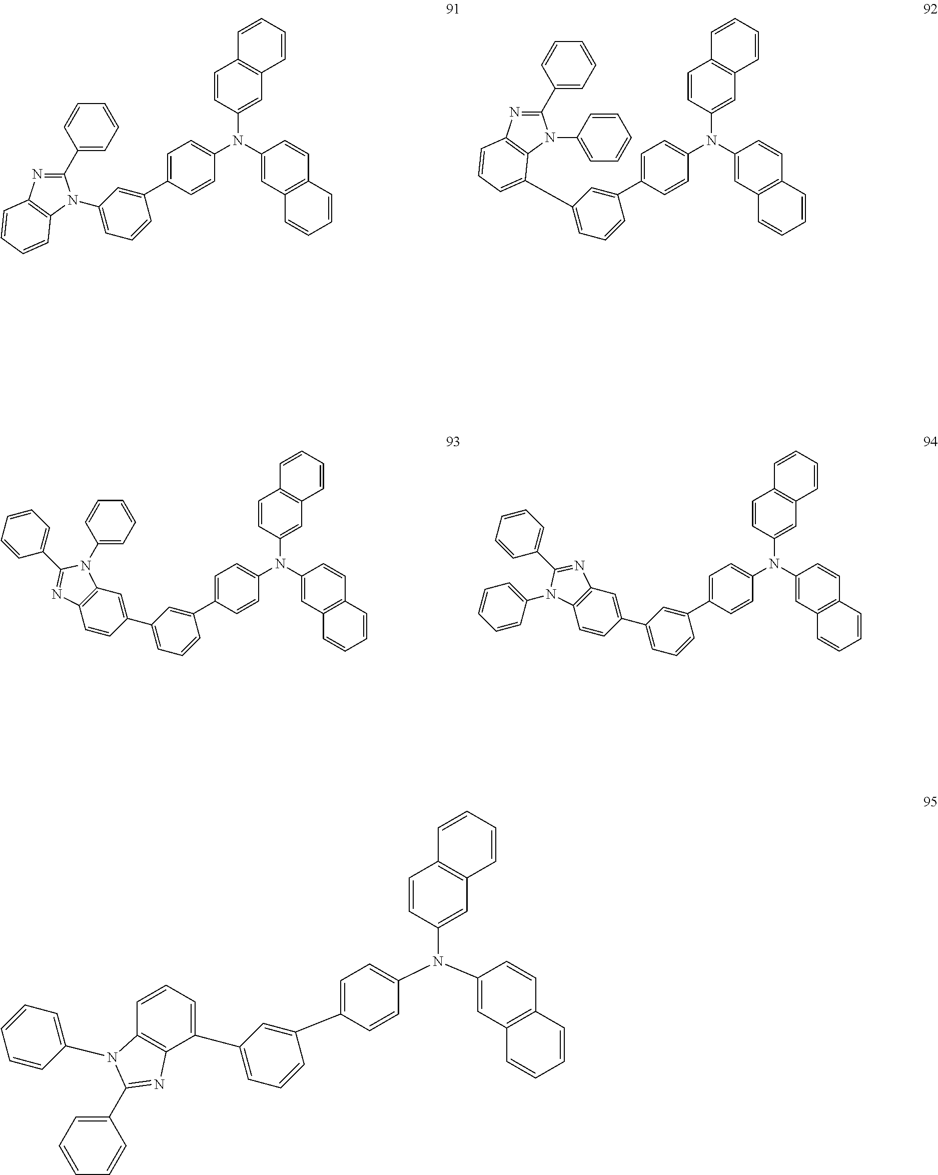 [SMILES: c1ccc(-c2nc3ccccc3n2-c2cccc(-c3ccc(N(c4ccc5ccccc5c4)c4ccc5ccccc5c4)cc3)c2)cc1 ; c1ccc(-c2nc3cccc(-c4cccc(-c5ccc(N(c6ccc7ccccc7c6)c6ccc7ccccc7c6)cc5)c4)c3n2-c2ccccc2)cc1 ; c1ccc(-c2nc3ccc(-c4cccc(-c5ccc(N(c6ccc7ccccc7c6)c6ccc7ccccc7c6)cc5)c4)cc3n2-c2ccccc2)cc1 ; c1ccc(-c2nc3cc(-c4cccc(-c5ccc(N(c6ccc7ccccc7c6)c6ccc7ccccc7c6)cc5)c4)ccc3n2-c2ccccc2)cc1 ; c1ccc(-c2nc3c(-c4cccc(-c5ccc(N(c6ccc7ccccc7c6)c6ccc7ccccc7c6)cc5)c4)cccc3n2-c2ccccc2)cc1] 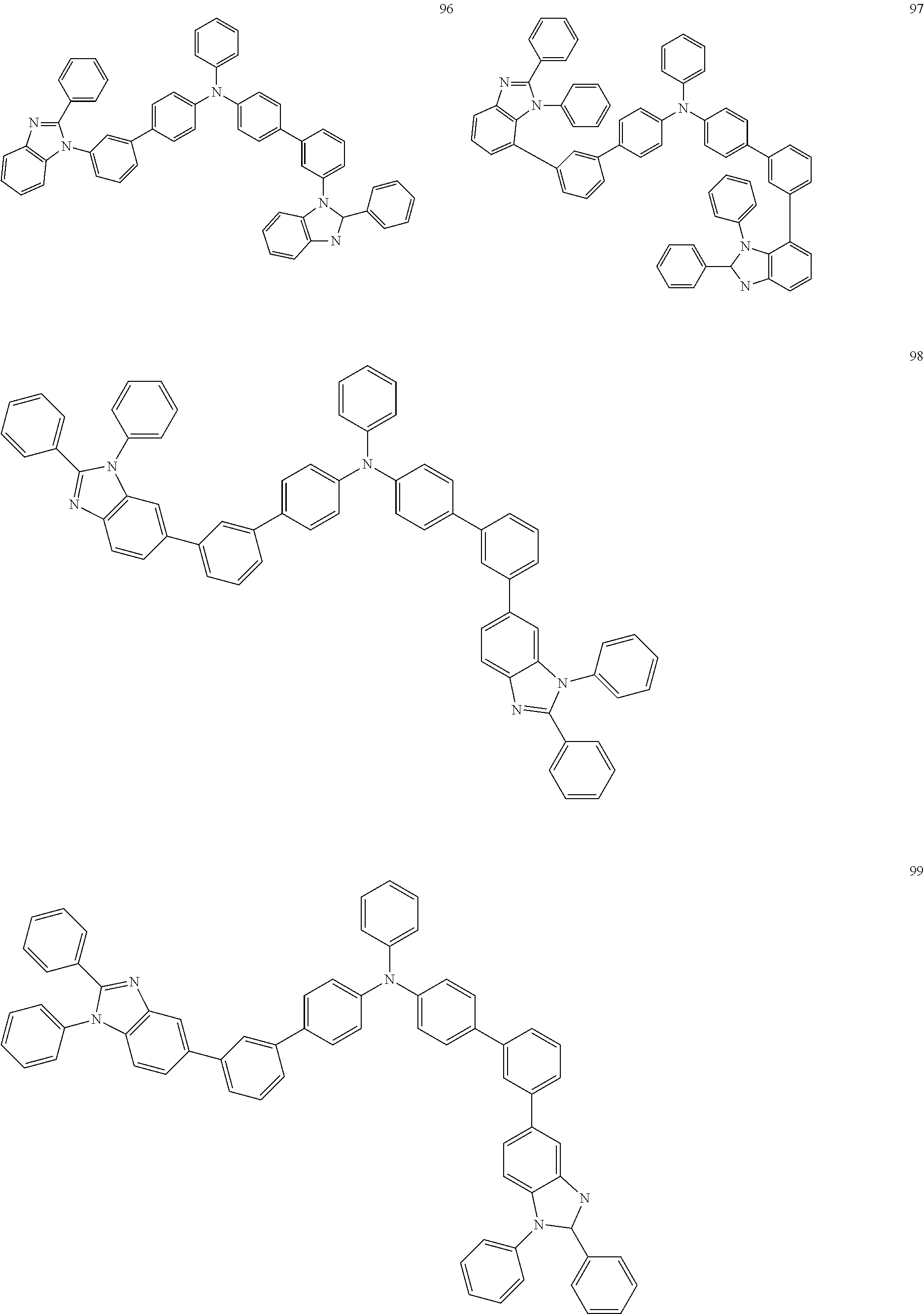 [SMILES: c1ccc(-c2nc3ccccc3n2-c2cccc(-c3ccc(N(c4ccccc4)c4ccc(-c5cccc(N6c7ccccc7NC6c6ccccc6)c5)cc4)cc3)c2)cc1 ; c1ccc(-c2nc3cccc(-c4cccc(-c5ccc(N(c6ccccc6)c6ccc(-c7cccc(-c8cccc9c8N(c8ccccc8)C(c8ccccc8)N9)c7)cc6)cc5)c4)c3n2-c2ccccc2)cc1 ; c1ccc(-c2nc3ccc(-c4cccc(-c5ccc(N(c6ccccc6)c6ccc(-c7cccc(-c8ccc9nc(-c%10ccccc%10)n(-c%10ccccc%10)c9c8)c7)cc6)cc5)c4)cc3n2-c2ccccc2)cc1 ; c1ccc(-c2nc3cc(-c4cccc(-c5ccc(N(c6ccccc6)c6ccc(-c7cccc(-c8ccc9c(c8)NC(c8ccccc8)N9c8ccccc8)c7)cc6)cc5)c4)ccc3n2-c2ccccc2)cc1] 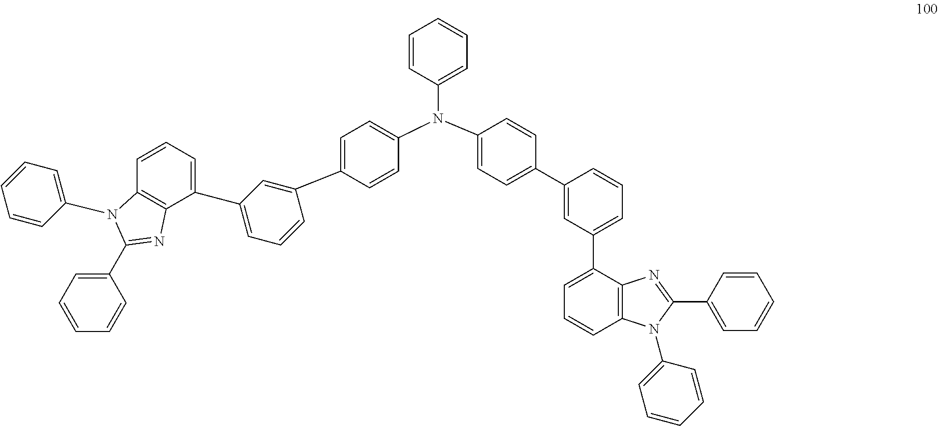 [SMILES: c1ccc(-c2nc3c(-c4cccc(-c5ccc(N(c6ccccc6)c6ccc(-c7cccc(-c8cccc9c8nc(-c8ccccc8)n9-c8ccccc8)c7)cc6)cc5)c4)cccc3n2-c2ccccc2)cc1] 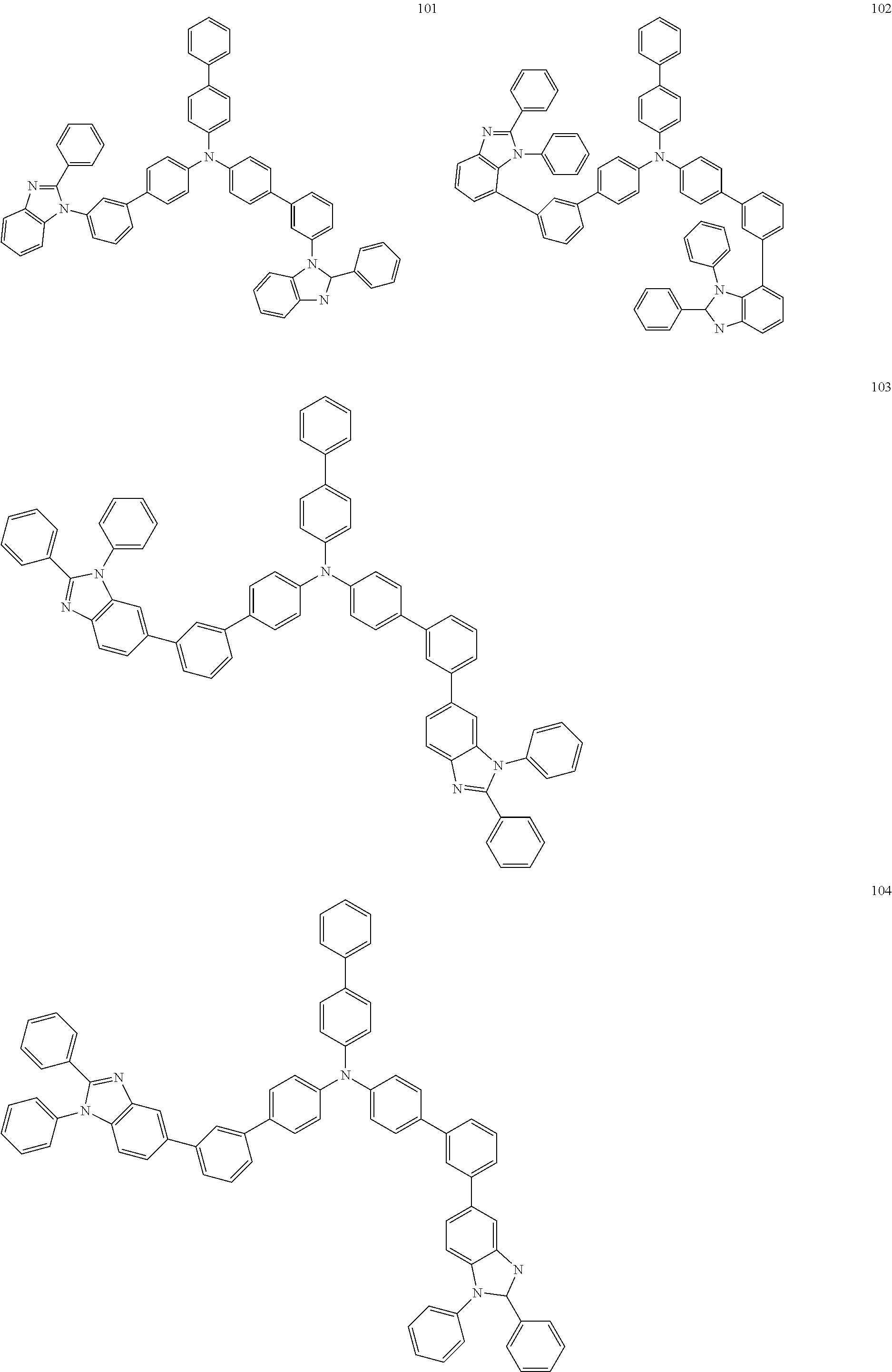 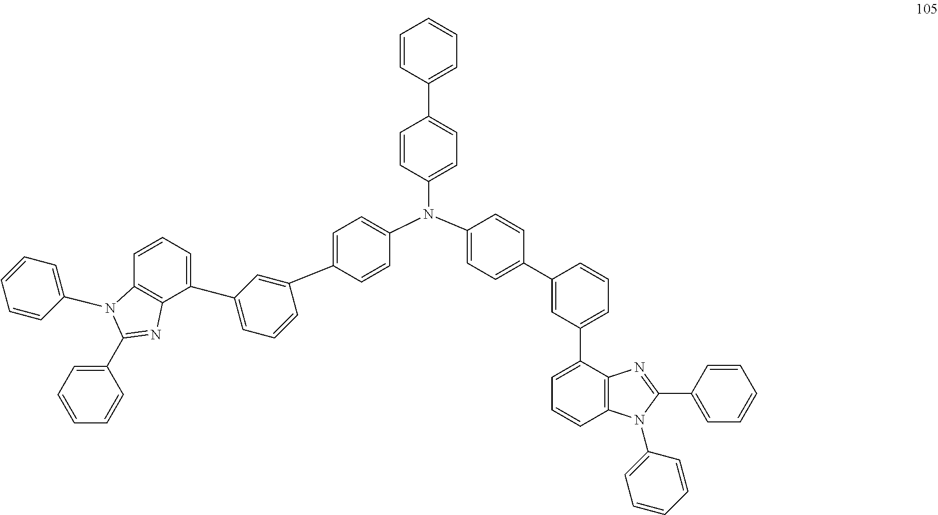 [SMILES: c1ccc(-c2ccc(N(c3ccc(-c4cccc(-c5cccc6c5nc(-c5ccccc5)n6-c5ccccc5)c4)cc3)c3ccc(-c4cccc(-c5cccc6c5nc(-c5ccccc5)n6-c5ccccc5)c4)cc3)cc2)cc1] 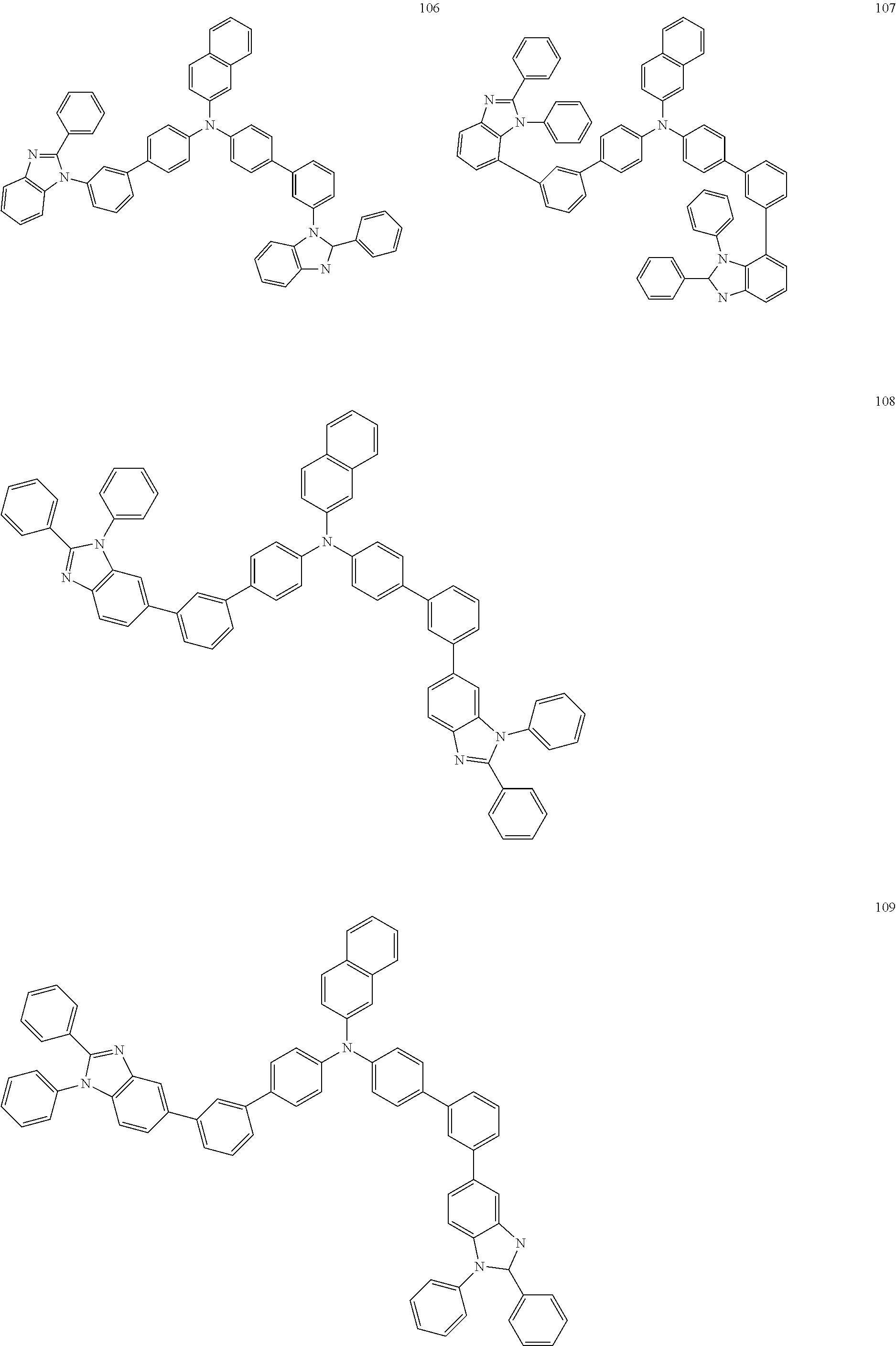 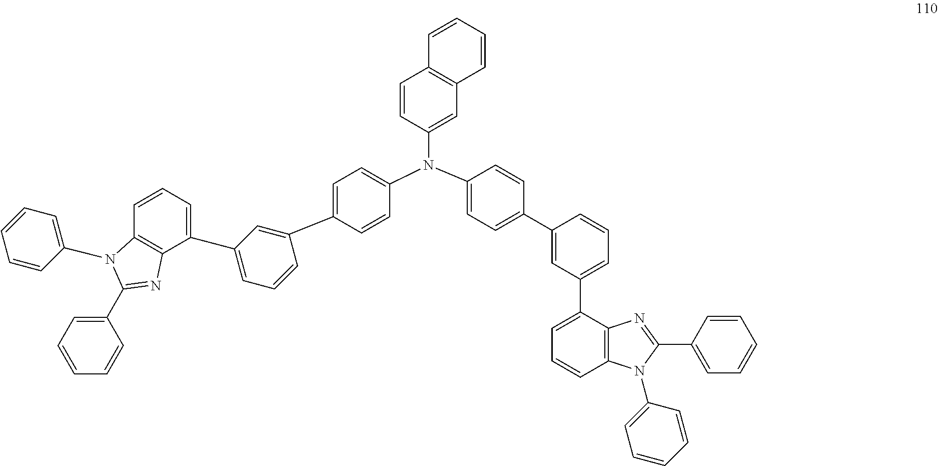 [SMILES: c1ccc(-c2nc3c(-c4cccc(-c5ccc(N(c6ccc(-c7cccc(-c8cccc9c8nc(-c8ccccc8)n9-c8ccccc8)c7)cc6)c6ccc7ccccc7c6)cc5)c4)cccc3n2-c2ccccc2)cc1] 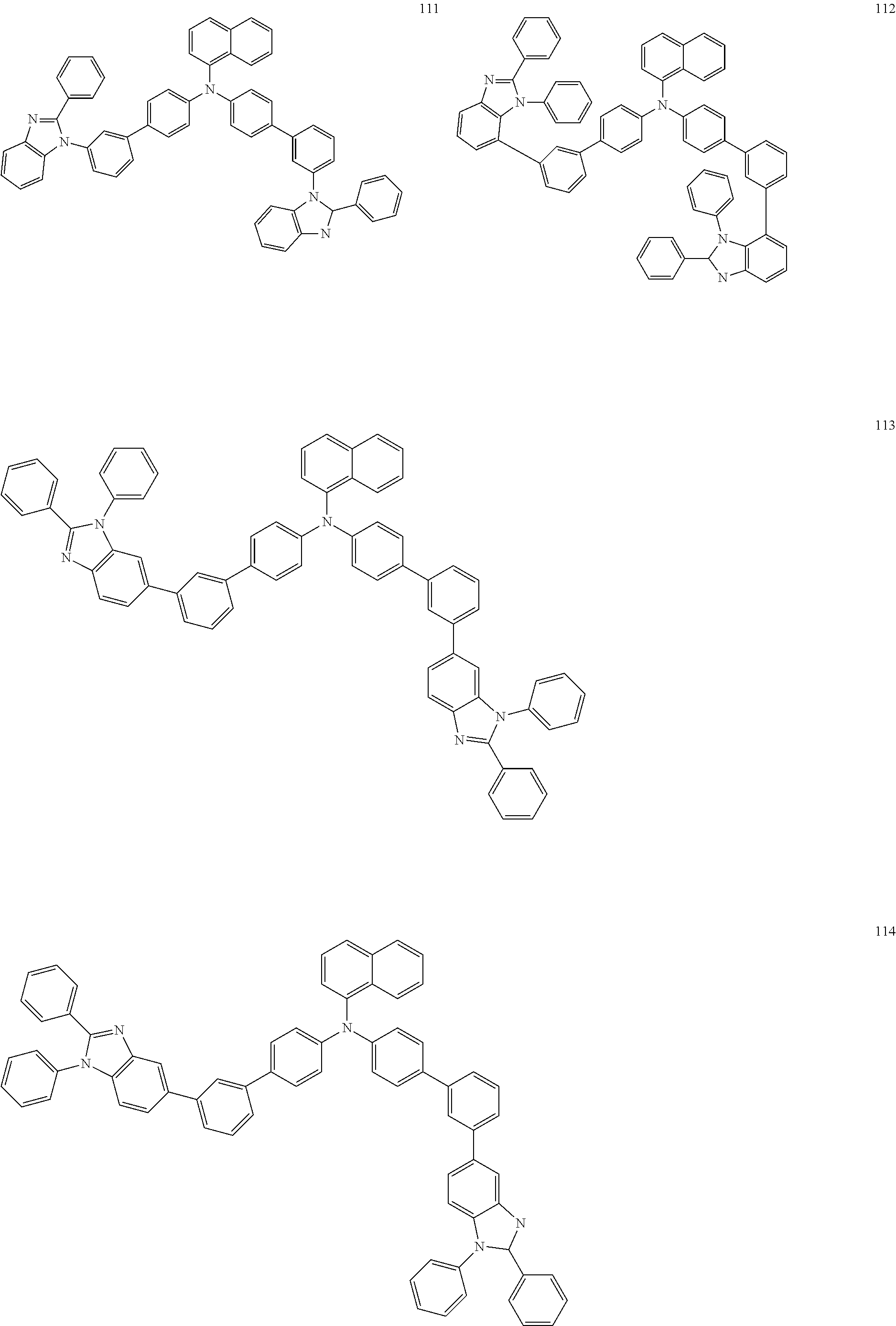 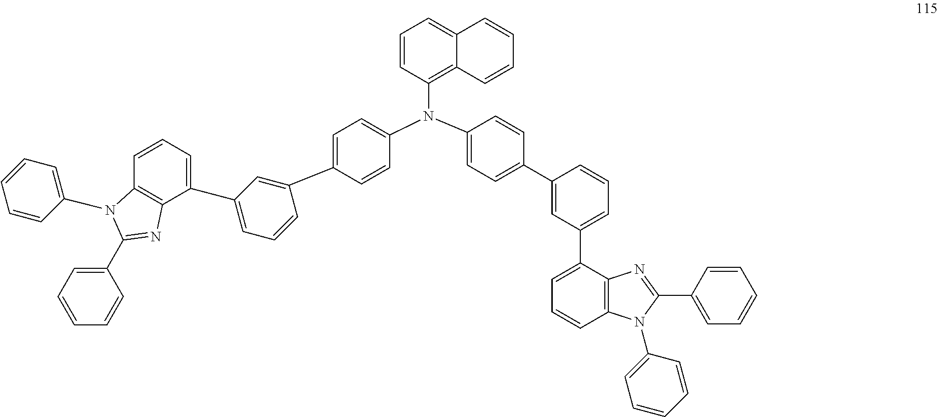 [SMILES: c1ccc(-c2nc3c(-c4cccc(-c5ccc(N(c6ccc(-c7cccc(-c8cccc9c8nc(-c8ccccc8)n9-c8ccccc8)c7)cc6)c6cccc7ccccc67)cc5)c4)cccc3n2-c2ccccc2)cc1] 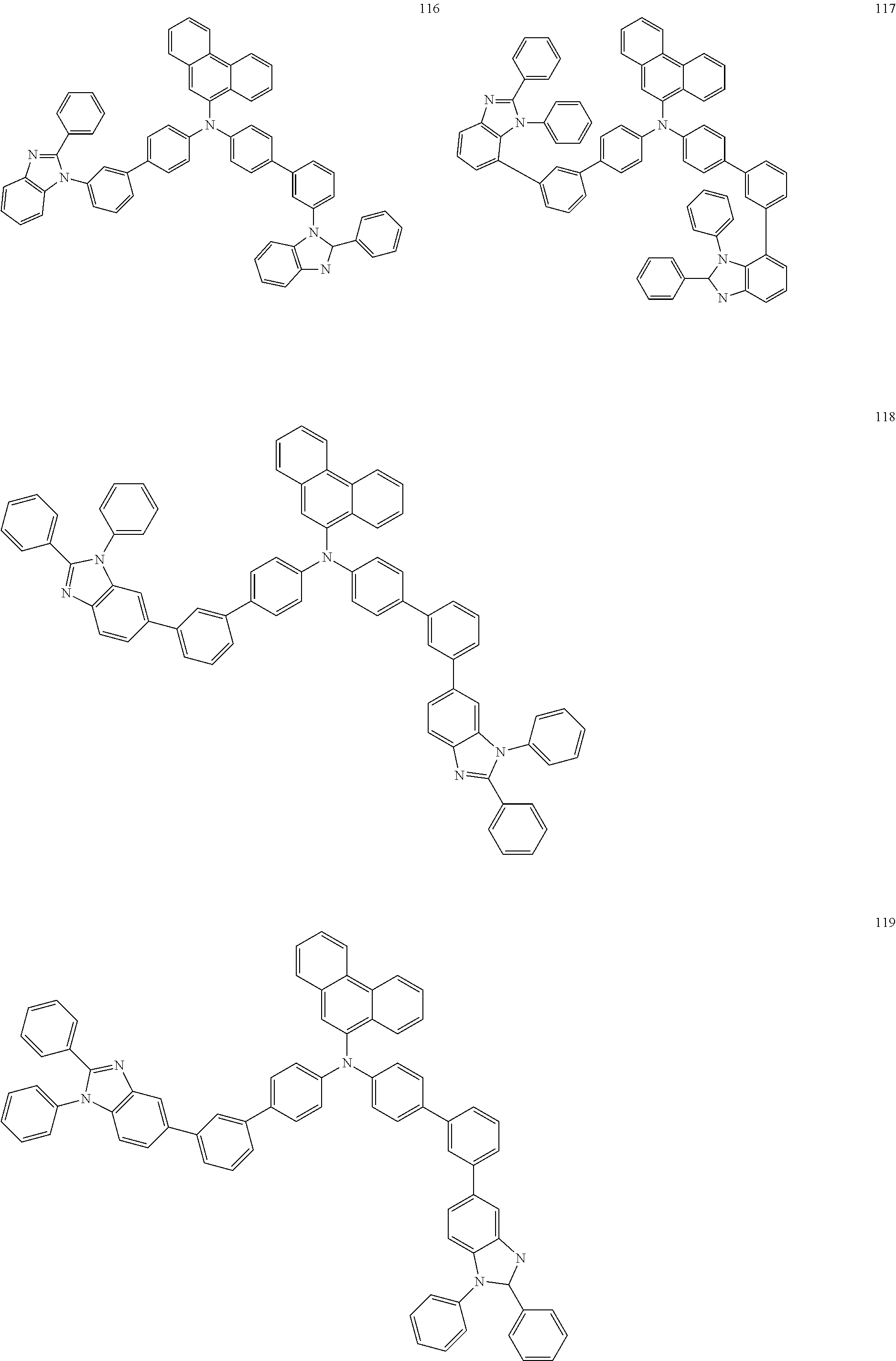 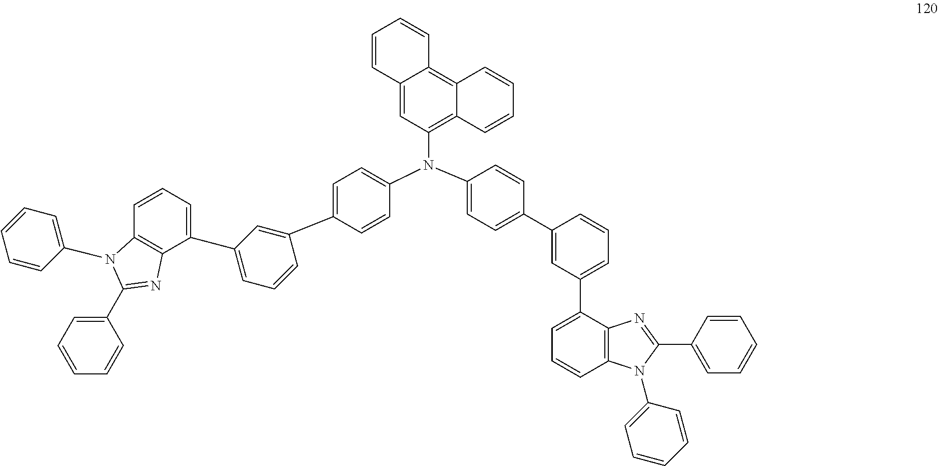 [SMILES: c1ccc(-c2nc3c(-c4cccc(-c5ccc(N(c6ccc(-c7cccc(-c8cccc9c8nc(-c8ccccc8)n9-c8ccccc8)c7)cc6)c6cc7ccccc7c7ccccc67)cc5)c4)cccc3n2-c2ccccc2)cc1] 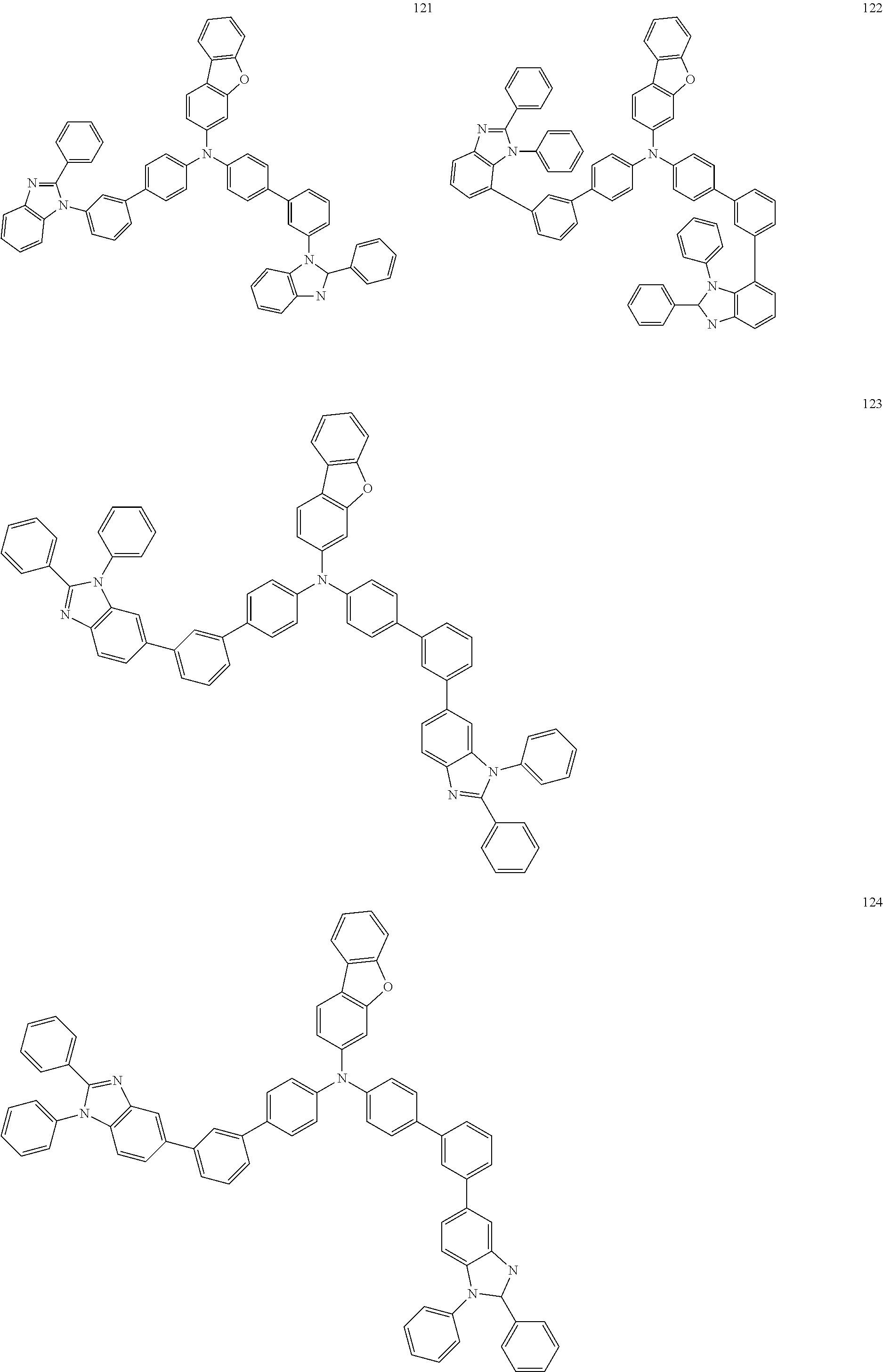 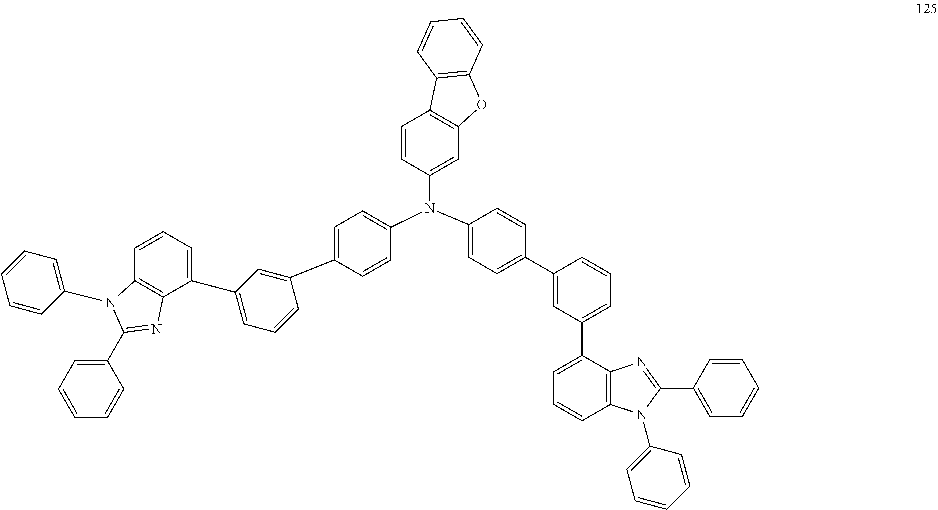 [SMILES: c1ccc(-c2nc3c(-c4cccc(-c5ccc(N(c6ccc(-c7cccc(-c8cccc9c8nc(-c8ccccc8)n9-c8ccccc8)c7)cc6)c6ccc7c(c6)oc6ccccc67)cc5)c4)cccc3n2-c2ccccc2)cc1] 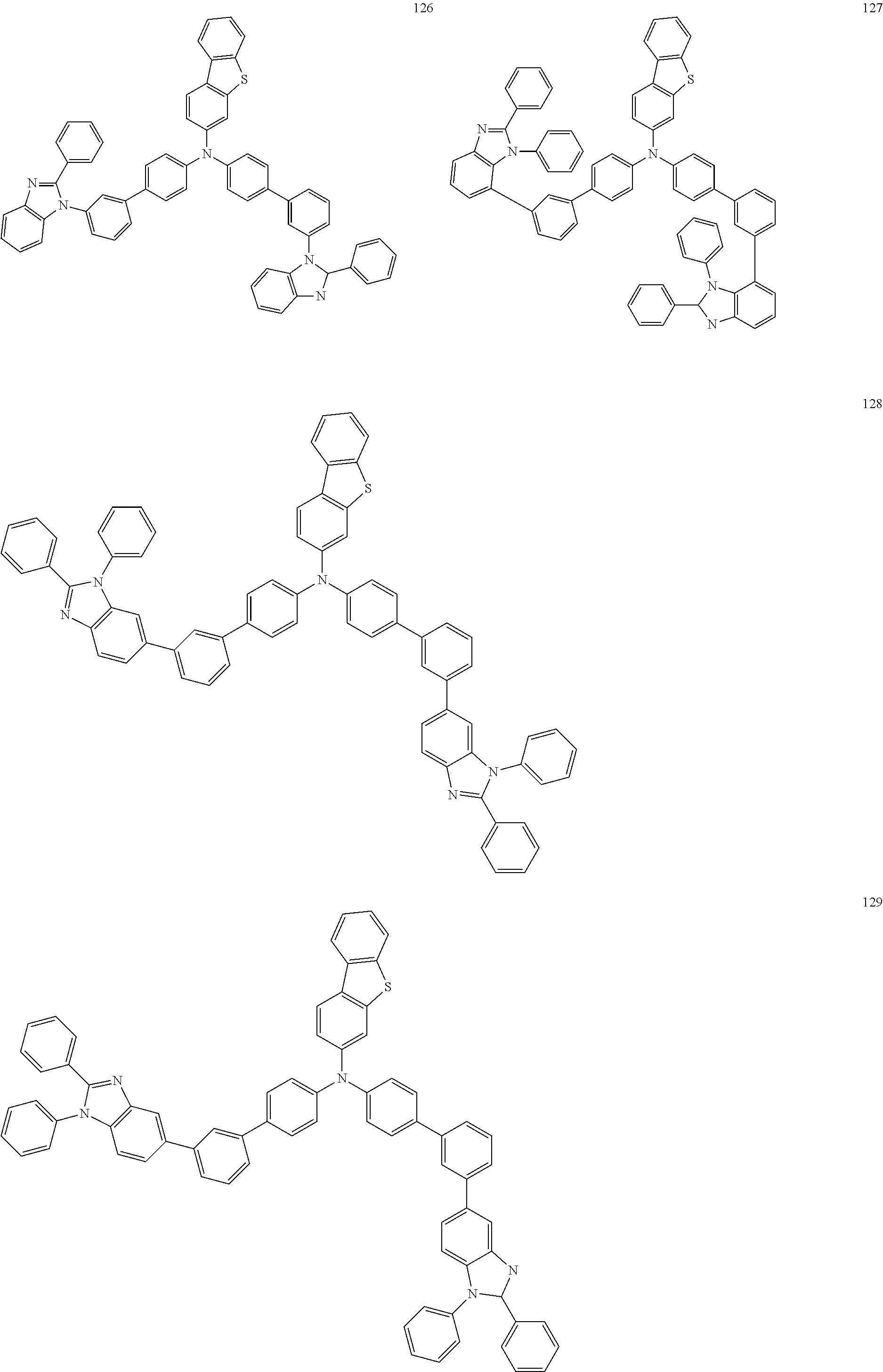 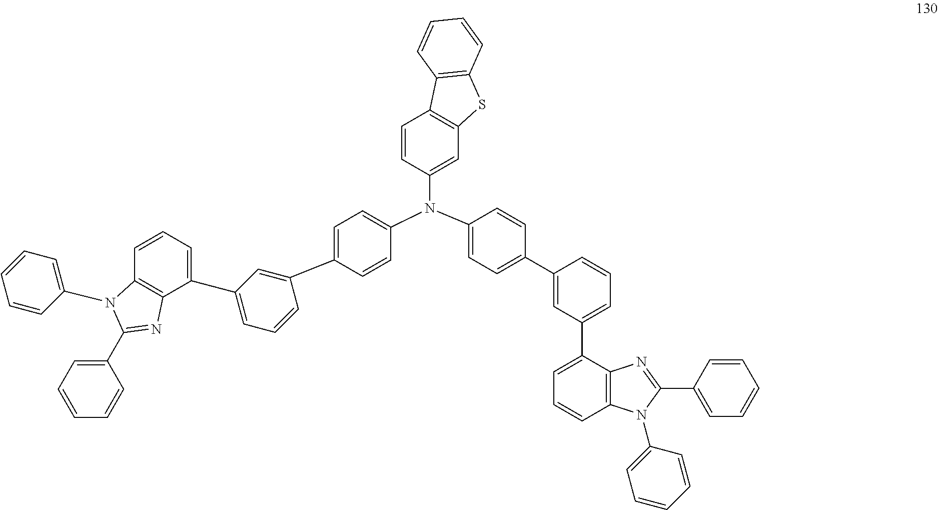 [SMILES: c1ccc(-c2nc3c(-c4cccc(-c5ccc(N(c6ccc(-c7cccc(-c8cccc9c8nc(-c8ccccc8)n9-c8ccccc8)c7)cc6)c6ccc7c(c6)sc6ccccc67)cc5)c4)cccc3n2-c2ccccc2)cc1]
